(19) United States
(12) Patent Application Publication
(10) Pub. No.: US 2023/0098332 A1
(43) Pub. Date: Mar. 30, 2023

(54) HETEROCYCLIC DERIVATIVES AS TRMP8 ANTAGONISTS

(71) Applicant: Givaudan SA, VERNIER (CH)

(72) Inventors: Nicolas COCITO ARMANINO, Baden (CH); Julie CHARPENTIER, Basel (CH); Chun CHEN, Shanghai (CN); Changming DING, Shanghai (CN); Roger EMTER, Zurich (CH); Andreas NATSCH, Uetikon (CH); Chao WANG, Shanghai (CN); Lijun ZHOU, Shanghai (CN)

(21) Appl. No.: 17/800,049

(22) PCT Filed: Mar. 4, 2021
(86) PCT No.: PCT/EP2021/055420

§ 371 Date: Aug. 16, 2022

(30) Foreign Application Priority Data
Mar. 5, 2020 (CN) PCT/CN2020/077896

(51) Int. Cl. C07D 295/096; C07D 309/04; C07D 335/02; C07D 279/12; C07D 241/04

(52) U.S. Cl. CPC C07D 295/096; C07D 309/04; C07D 335/02; C07D 279/12; C07D 241/04

(57) ABSTRACT

Disclosed are TRPM8 modulators as defined by formula (I) for achieving a cooling effect on skin and mucosa.

### TECHNICAL FIELD

The present invention relates to a particular class of compounds capable to activate TRPM8 ion channels. It further relates to the use of said compounds for inducing a sensation of coldness, and to consumer products comprising these compounds.

### BACKGROUND

TRPM8 (transient receptor potential melastatin member 8, also known as Trp-p8 or MCR1) is activated by innocuous cool and thus plays an important role as sensor for temperature. The channels are widely distributed in different tissues (such as human skin and mucosa (such as oral mucosa, throat mucosa, and nasal mucosa), male urogenital tract, lung epithelium cells and artery myoctes). They are Ca²⁺-permeable, nonselective cation channels that exhibit polymodal gating mechanisms, being activated by innocuous cool to cold temperature, membrane depolarization, and molecules which are known as cooling agents including natural and synthetic compounds. The receptor was described for the first time in 2002 as cold receptor in a number of publications.

The present invention is based on the finding that a particular class of compounds can be used to drive a cooling response when brought into contact with TRPM8 receptor in-vitro and in-vivo.

Compounds providing a cooling sensation have for a long time played an important role in the flavor and fragrance industry in order to produce an association with freshness and cleanliness. Cooling compounds are widely used in a variety of products such as foodstuffs, tobacco products, beverages, dentifrices, mouthwashes, toothpastes, and toiletries. The cooling sensation provided contributed to the appeal and acceptability of consumer products. In particular, oral care products, such as dentifrices and mouthwashes are formulated with coolants because they provide breath freshening effects and a clean, cool, fresh feeling in the mouth.

A large number of compounds providing cooling sensations have been described. The most well-known natural occurring compound is menthol, in particular L-menthol. Among the synthetic compounds providing cooling sensations, many are derivatives of or are structurally related to menthol and derivatized with functional groups including carboxamide, ketal, ester, ether and alcohols.

Applicant surprisingly found a new class of chemical compounds which differ significantly in structural terms from the TRPM8 modulators known hitherto. It was surprisingly found that this class of chemical compounds as herein further described can provide long lasting cooling on the human skin and/or mucosa at very low concentrations.

### SUMMARY

There is provided in a first aspect a method of modulating (in-vitro and in-vivo modulation) of transient receptor potential melastatin member 8 (TRPM8) comprising bringing the receptor into contact with a compound of formula (I), or a salt or solvate thereof

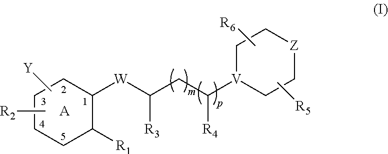

wherein

ring A represents a phenyl ring, cyclohexyl or cyclohexenyl ring;

R₁ is selected from hydrogen, C₁-C₆ alkyl (e.g. methyl, ethyl, isopropyl, isobutyl) and C₁-C₆ alkyloxy (e.g. methoxy, ethoxy, propoxy),

R₂ is connected to position 3, 4 or 5 of ring A and is selected from hydrogen, halogen (e.g. F, Cl, Br, I), C₁-C₆ alkyl (e.g. methyl, ethyl, isopropyl, isobutyl, isopropyl), C₁-C₆ alkyloxy (e.g. methoxy, ethoxy), and C(O)O—C₁-C₃-alkyl (e.g. C(O)OCH₃),

R₃ is selected from hydrogen, C₁-C₆ alkyl (e.g. methyl, ethyl), C₂-C₆ alkenyl (e.g. vinyl), OH, and ═O, and R₄ is selected from hydrogen, C₁-C₆ alkyl (e.g. methyl, ethyl), C₂-C₆ alkenyl (e.g. vinyl), and OH, with the proviso that at least one of R₃ and R₄ is hydrogen, or

R₃ and R₁ form together with the carbon atoms to which they are attached a sixth membered ring system (i.e. R₃ and R₁ is a bivalent residue —CH₂—CH₂—), and R₄ is hydrogen,

R₅ is selected from hydrogen, F and C₁-C₆ alkyl (e.g. methyl, ethyl, isobutyl),

R₆ is selected from hydrogen, F and C₁-C₆ alkyl (e.g. methyl, ethyl, isobutyl),

m is 0 or 1,

p is 0 or 1,

W is selected from —CH₂—, —O—, >C═O, —CH═, and >NR₁₃ wherein R₁₃ is selected from hydrogen and C₁-C₃ alkyl (e.g. methyl, or isopropyl);

V is selected from >CH—, and >N—,

Z is selected from —O—, >S═O, >CHR₁₂, >NR₁₂ wherein R₁₂ is hydrogen, C₁-C₃ alkyl, OH, or CH₂OH,

Y is connected to position 2 or 3 of ring A and is selected from C₄-C₆ alkyl (e.g. isobutyl),

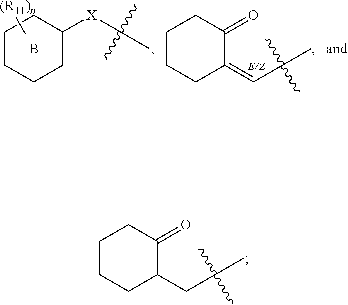

wherein

ring B represents a phenyl, thiophen, furan, or pyridine ring,

R₁₁ is selected from CN, halogen (e.g. F, CL, Br, I), CH₂CN, NO₂, NH₂, CF₃, C(O)OC₂H₅, C₁-C₃ alkyl (e.g. methyl, or ethyl), C₁-C₃ alkyloxy (e.g. methoxy or ethoxy), C₂-C₃ alkenyl (e.g. vinyl, or allyl), and OH, and n is an integer from 0-5 (including 1, 2, and 3) with the proviso that if n>1, R₁₁ can be the same or different, and

X is selected from —CH₂—, —O—, >CH—CH₃, >CH═CH₂, >C═O, and >CHOH.

In accordance with a second aspect there is provided a method of inducing a cooling sensation in a human or animal comprising contacting the human or animal with a compound of formula (I), or a salt or solvate thereof.

There is provided in a third aspect consumer products, in particular consumer products which get into contact with the human skin and/or mucosa comprising a compound as defined by formula (I), or a salt or solvate thereof.

There is provided in a fourth aspect a composition comprising a cool sensation wherein the composition comprises at least one compound of formula (I), a salt or solvate thereof, and a further cooling compound.

There is provided in a fifth aspect pharmaceutical composition comprising one or more compounds as defined by formula (I), or a salt or solvate thereof.

There is provided in a sixth aspect a compound of formula (I), or a salt or solvate thereof.

The details, examples and preferences provided in relation to any particular one or more of the stated aspects of the present invention will be further described herein and apply equally to all aspects of the present invention. Any combination of the embodiments, examples and preferences described herein in all possible variations thereof is encompassed by the present invention unless otherwise indicated herein, or otherwise clearly contradicted by context.

### DETAILED DESCRIPTION

The present invention is based, at least in part, on the surprising finding of a new class of chemical compounds which differ significantly in structural terms from the TRPM8 modulators known hitherto, that are capable to activate the TRPM8 ion channel, which brings about a Ca²⁺ influx into the cold-sensitive neurons. The electrical signal produced as a result is ultimately perceived as sensation of coldness. Applicant surprising fount that this class of chemical compounds as herein further described can provide long lasting cooling on the human skin and/or mucosa at very low concentrations.

The compounds as defined hereinbelow by formula (I) (which encompass the compounds of formula (Ia), and (Ib)) are “TRPM8 agonist”, which means that they have an agonistic effect on the cellular Ca²⁺ ion permeability of the TRPM8 channels. Accordingly, by “TRPM8 agonist” is meant any compound, which when brought into contact with the TRPM8 receptor, produces an increase in fluorescence over background, using the FLIPR method as described, e.g., by Klein et al., (Chem. Senses 36: 649-658, 2011), which is also described in more details in the experimental part.

Thus, there is provided in a first aspect a method of modulating (in-vitro and in-vivo modulation) of transient receptor potential melastatin member 8 (TRPM8) comprising bringing the receptor into contact with a compound of formula (I), a salt or solvate thereof

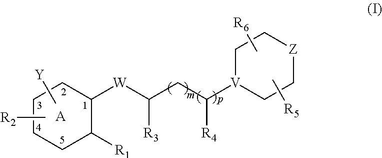

wherein

ring A represents a phenyl ring, cyclohexyl or cyclohexenyl ring;

R₁ is selected from hydrogen, C₁-C₆ alkyl (e.g. methyl, ethyl, isopropyl, isobutyl) and Cr C₆ alkyloxy (e.g. methoxy, ethoxy, propoxy),

R₂ is connected to position 3, 4 or 5 of ring A and is selected from hydrogen, halogen (e.g. F, Cl, Br, I), C₁-C₆ alkyl (e.g. methyl, ethyl, isopropyl, isobutyl, isopropyl), C₁-C₆ alkyloxy (e.g. methoxy, ethoxy), and C(O)O—C₁-C₃-alkyl (e.g. C(O)OCH₃), R₃ is selected from hydrogen, C₁-C₆ alkyl (e.g. methyl, ethyl), C₂-C₆ alkenyl (e.g. vinyl), OH, and ═O, and R₄ is selected from hydrogen, C₁-C₆ alkyl (e.g. methyl, ethyl), C₂-C₆ alkenyl (e.g. vinyl), and OH, with the proviso that at least one of R₃ and R₄ is hydrogen, or

R₃ and R₁ form together with the carbon atoms to which they are attached a sixth membered ring system (i.e. R₃ and R₁ is a bivalent residue —CH₂—CH₂—), and R₄ is hydrogen,

R₅ is selected from hydrogen, F and C₁-C₆ alkyl (e.g. methyl, ethyl, isobutyl),

R₆ is selected from hydrogen, F and C₁-C₆ alkyl (e.g. methyl, ethyl, isobutyl),

m is 0 or 1,

p is 0 or 1,

W is selected from —CH₂—, —O—, >C═O, —CH═, and >NR₁₃ wherein R₁₃ is selected from hydrogen and C₁-C₃ alkyl (e.g. methyl, or isopropyl);

V is selected from >CH—, and >N—,

Z is selected from —O—, >S═O, >CHR₁₂, >NR₁₂ wherein R₁₂ is hydrogen, C₁-C₃ alkyl, OH, or CH₂OH,

Y is connected to position 2 or 3 of ring A and is selected from C₄-C₆ alkyl (e.g. isobutyl),

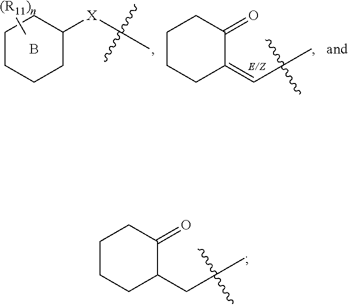

wherein

ring B represents a phenyl, thiophen, furan, or pyridine ring,

R₁₁ is selected from CN, halogen (e.g. F, CL, Br, I), CH₂CN, NO₂, NH₂, CF₃, C(O)OC₂H₅, C₁-C₅ alkyl (e.g. methyl, or ethyl), C₁-C₃ alkyloxy (e.g. methoxy or ethoxy), C₂-C₃ alkenyl (e.g. vinyl, or allyl), and OH, and n is an integer from 0-5 (including 1, 2, and 3) with the proviso that if n>1, R₁₁ can be the same or different, and

X is selected from —OH₂—, —O—, >CH—CH₃, >CH═CH₂, >C═O, and >CHOH.

Further, non-limiting examples are compounds of formula (I), a salt or solvate thereof wherein R₁₁ is selected from CN, halogen (e.g. F, C, Br, I), CH₂CN, NO₂, NH₂, CF₃, and methyl.

Further, non-limiting examples are compounds of formula (I), a salt or solvate thereof wherein R₁ is selected from H, and C₁-C₃ alkyl (including ethyl), and R₂ selected from methyl, ethyl and halogen (e.g. Cl).

Further, non-limiting examples are compounds of formula (I), a salt or solvate thereof wherein R₃ is selected from H, methyl, —OH and ═O, and R₄ is selected from hydrogen and methyl, with the proviso that at least one of R₃ and R₄ is hydrogen.

Further, non-limiting examples are compounds of formula (I), a salt or solvate thereof wherein R₁ is selected from H, and C₁-C₃ alkyl (including ethyl), R₂ selected from methyl, ethyl and Cl, R₃ is selected from H, methyl, —OH and ═O, and R₄ is selected from hydrogen and methyl, with the proviso that at least one of R₃ and R₄ is hydrogen.

Further, non-limiting examples are compounds of formula (I), a salt or solvate thereof wherein R₅ and R₆ are selected from hydrogen and methyl, e.g. at least one of R₅ and R₆ are hydrogen, or R₅═R₆ are hydrogen.

Further, non-limiting examples are compounds of formula (I), a salt or solvate thereof wherein R₁ is selected from H, and C₁-C₃ alkyl (including ethyl), R₂ selected from methyl, ethyl and Cl, R₃ is selected from H, methyl, —OH and ═O, and R₄ is selected from hydrogen and methyl, with the proviso that at least one of R₃ and R₄ is hydrogen, and R₅ and R₆ are selected from hydrogen and methyl, e.g. at least one of R₅ and R₆ are hydrogen, or R₅═R₆ are hydrogen.

Further, non-limiting examples are compounds of formula (I), a salt or solvate thereof wherein X is selected from —CH₂—, and —O—.

Further, non-limiting examples are compounds of formula (I), a salt or solvate thereof R₁ is selected from H, and C₁-C₃ alkyl (including ethyl), R₂ selected from methyl, ethyl and Cl, R₃ is selected from H, methyl, —OH and ═O, and R₄ is selected from hydrogen and methyl, with the proviso that at least one of R₃ and R₄ is hydrogen, R₅ and R₆ are selected from hydrogen and methyl, e.g. at least one of R₅ and R₆ are hydrogen, or R₅ ═R₆ are hydrogen, and X is selected from —CH₂—, and —O—.

Non-limiting examples are compounds of formula (Ia), a salt or solvate thereof,

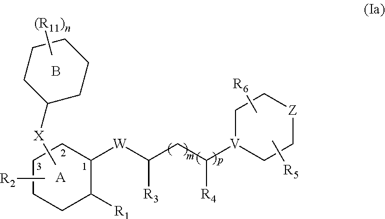

wherein

ring A represents a phenyl ring, cyclohexyl or cyclohexenyl ring;

R₁ is selected from hydrogen, C₁-C₆ alkyl (e.g. methyl, ethyl, isopropyl, isobutyl) and C₁-C₆ alkyloxy (e.g. methoxy, ethoxy, propoxy),

R₂ is selected from hydrogen, halogen (e.g. F, Cl, Br, I), C₁-C₆ alkyl (e.g. methyl, ethyl, isopropyl, isobutyl, isopropyl), C₁-C₆ alkyloxy (e.g. methoxy, ethoxy), and C(O)O—C₁-C₃-alkyl (e.g. C(O)OCH₃),

R₃ is selected from hydrogen, C₁-C₆ alkyl (e.g. methyl, ethyl), C₂-C₆ alkenyl (e.g. vinyl), OH, and ═O, and R₄ is selected from hydrogen, C₁-C₆ alkyl (e.g. methyl, ethyl), C₂-C₆ alkenyl (e.g. vinyl), and OH, with the proviso that at least one of R₃ and R₄ is hydrogen, or

R₃ and R₁ form together with the carbon atoms to which they are attached a sixth membered ring system (i.e. R₃ and R₁ is a bivalent residue —CH₂—CH₂—), and R₄ is hydrogen,

R₅ is selected from hydrogen, F and C₁-C₆ alkyl (e.g. methyl, ethyl, isobutyl), R_{b} is selected from hydrogen, F and C₁-C₆ alkyl (e.g. methyl, ethyl, isobutyl),

m is 0 or 1,

p is 0 or 1,

W is selected from —CH₂—, —O—, >C═O, —CH═, and >NR₁₃ wherein R₁₃ is selected from hydrogen and C₁-C₃ alkyl (e.g. methyl, or isopropyl);

V is selected from >CH—, and >N—,

Z is selected from —O—, >S═O, >CHR₁₂, >NR₁₂ wherein R₁₂ is hydrogen, C₁-C₃ alkyl, OH,

ring B represents a phenyl, thiophen, furan, or pyridine ring,

R₁₁ is selected from CN, halogen (e.g. F, CL, Br, I), CH₂CN, NO₂, NH₂, CF₃, C(O)OC₂H₅, C₁-C₃ alkyl (e.g. methyl, or ethyl), C₁-C₃ alkyloxy (e.g. methoxy or ethoxy), C₂-C₃ alkenyl (e.g. vinyl, or allyl), and OH, and n is an integer from 0-5 (including 1, 2, and 3) with the proviso that if n>1, R₁₁ can be the same or different, and

X is connected to position 2 or 3 of ring A and is selected from —CH₂—, —O—, >CH—CH₃, >CH═CH₂, >C═O, and >CHOH.

Further, non-limiting examples are compounds of formula (Ia), a salt or solvate thereof wherein R₁₁ is selected from CN, halogen (e.g. F, Cl, Br, I), CH₂CN, NO₂, NH₂, CF₃, and methyl.

Further, non-limiting examples are compounds of formula (Ia), a salt or solvate thereof wherein R₁ is selected from H, and C₁-C₃ alkyl (including ethyl), and R₂ selected from methyl, ethyl and halogen (e.g. Cl).

Further, non-limiting examples are compounds of formula (Ia), a salt or solvate thereof wherein R₃ is selected from H, methyl, —OH and ═O, and R₄ is selected from hydrogen and methyl, with the proviso that at least one of R₃ and R₄ is hydrogen.

Further, non-limiting examples are compounds of formula (Ia), a salt or solvate thereof wherein R₁ is selected from H, and C₁-C₃ alkyl (including ethyl), R₂ selected from methyl, ethyl and Cl, R₃ is selected from H, methyl, —OH and ═O, and R₄ is selected from hydrogen and methyl, with the proviso that at least one of R₃ and R₄ is hydrogen.

Further, non-limiting examples are compounds of formula (Ia), a salt or solvate thereof wherein R₅ and R₆ are selected from hydrogen and methyl, e.g. at least one of R₅ and R₆ are hydrogen, or R₅═R₆ are hydrogen.

Further, non-limiting examples are compounds of formula (Ia), a salt or solvate thereof wherein R₁ is selected from H, and C₁-C₃ alkyl (including ethyl), R₂ selected from methyl, ethyl and Cl, R₃ is selected from H, methyl, —OH and ═O, and R₄ is selected from hydrogen and methyl, with the proviso that at least one of R₃ and R₄ is hydrogen, and R₅ and R₆ are selected from hydrogen and methyl, e.g. at least one of R₅ and R₆ are hydrogen, or R₅═R₆ are hydrogen.

Further, non-limiting examples are compounds of formula (Ia), a salt or solvate thereof wherein X is selected from —CH₂—, and —O—.

Further, non-limiting examples are compounds of formula (Ia), a salt or solvate thereof R₁ is selected from H, and C₁-C₃ alkyl (including ethyl), R₂ selected from methyl, ethyl and Cl, R₃ is selected from H, methyl, —OH and ═O, and R₄ is selected from hydrogen and methyl, with the proviso that at least one of R₃ and R₄ is hydrogen, R₅ and R₆ are selected from hydrogen and methyl, e.g. at least one of R₅ and R₆ are hydrogen, or R₅ ═R₆ are hydrogen, and X is selected from —CH₂—, and —O—.

Further, non-limiting examples are compounds of formula (Ib), a salt or solvate thereof.

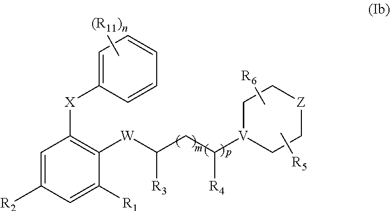

wherein

R₁ is selected from hydrogen, C₁-C₆ alkyl (e.g. methyl, ethyl, isopropyl, isobutyl) and C₁-C₆ alkyloxy (e.g. methoxy),

R₂ is selected from hydrogen, halogen (e.g. F, Cl, Br, I), C₁-C₆ alkyl (e.g. methyl, ethyl, isopropyl, isobutyl), C₁-C₆ alkyloxy (e.g. methoxy, ethoxy), and C(O)O—C₁-C₃-alkyl (e.g. C(O)OCH₃),

R₃ is selected from hydrogen, C₁-C₆ alkyl (e.g. methyl, ethyl, isopropyl), C₂-C₆ alkenyl (e.g. vinyl), OH, and ═O, and R₄ is selected from hydrogen, C₁-C₆ alkyl (e.g. methyl, ethyl, isopropyl), C₂-C₆ alkenyl (e.g. vinyl), and OH, with the proviso that at least one of R₃ and R₄ is hydrogen, or

R₃ and R₁ form together with the carbon atoms to which they are attached a sixth membered ring system (i.e. R₃ and R₁ is a bivalent residue —CH₂—CH₂—), and R₄ is hydrogen,

R₅ is selected from hydrogen, F and C₁-C₆ alkyl (e.g. methyl, ethyl),

R₆ is selected from hydrogen, F and C₁-C₆ alkyl (e.g. methyl, ethyl),

m is 0 or 1,

p is 0 or 1,

W is selected from —CH₂—, —O—, >C═O, —CH═, and >NR₁₃ wherein R₁₃ is selected from hydrogen and C₁-C₃ alkyl (e.g. methyl, or isopropyl);

V is selected from >CH—, and >N—,

Z is selected from —O—, >S═O, >CHR₁₂, >NR₁₂ wherein R₁₂ is hydrogen, C₁-C₃ alkyl, OH, or CH₂OH,

R₁₁ is selected from CN, halogen (e.g. F, CL, Br, I), CH₂CN, NO₂, NH₂, CF₃, C(O)OC₂H₅, C₁-C₃ alkyl (e.g. methyl, or ethyl), C₁-C₃ alkyloxy (e.g. methoxy or ethoxy), C₂-C₃ alkenyl (e.g. vinyl, or allyl), and OH, and n is an integer from 0-5 (including 1, 2, and 3) with the proviso that if n>1, R₁₁ can be the same or different, and

X is selected from —CH₂₋, —O—, >CH—CH₃, >CH═CH₂, >C═O, and >CHOH.

Further, non-limiting examples are compounds of formula (Ib), a salt or solvate thereof wherein R₁₁ is selected from CN, halogen (e.g. F, Cl, Br, I), CH₂CN, NO₂, NH₂, CF₃, and methyl.

Further, non-limiting examples are compounds of formula (Ib), a salt or solvate thereof wherein R₁ is selected from H, and C₁-C₃ alkyl (including ethyl), and R₂ selected from methyl, ethyl and halogen (e.g. Cl).

Further, non-limiting examples are compounds of formula (Ib), a salt or solvate thereof wherein R₃ is selected from H, methyl, —OH and ═O, and R₄ is selected from hydrogen and methyl, with the proviso that at least one of R₃ and R₄ is hydrogen.

Further, non-limiting examples are compounds of formula (Ib), a salt or solvate thereof wherein R₁ is selected from H, and C₁-C₃ alkyl (including ethyl), R₂ selected from methyl, ethyl and Cl, R₃ is selected from H, methyl, —OH and ═O, and R₄ is selected from hydrogen and methyl, with the proviso that at least one of R₃ and R₄ is hydrogen.

Further, non-limiting examples are compounds of formula (Ib), a salt or solvate thereof wherein R₅ and R₆ are selected from hydrogen and methyl, e.g. at least one of R₅ and R₆ are hydrogen, or R₅═R₆ are hydrogen.

Further, non-limiting examples are compounds of formula (Ib), a salt or solvate thereof wherein R₁ is selected from H, and C₁-C₃ alkyl (including ethyl), R₂ selected from methyl, ethyl and Cl, R₃ is selected from H, methyl, —OH and ═O, and R₄ is selected from hydrogen and methyl, with the proviso that at least one of R₃ and R₄ is hydrogen, and R₅ and R₆ are selected from hydrogen and methyl, e.g. at least one of R₅ and R₆ are hydrogen, or R₅═R₆ are hydrogen.

Further, non-limiting examples are compounds of formula (Ib), a salt or solvate thereof wherein X is selected from —CH₂—, and —O—.

Further, non-limiting examples are compounds of formula (Ib), a salt or solvate thereof R₁ is selected from H, and C₁-C₃ alkyl (including ethyl), R₂ selected from methyl, ethyl and Cl, R₃ is selected from H, methyl, —OH and ═O, and R₄ is selected from hydrogen and methyl, with the proviso that at least one of R₃ and R₄ is hydrogen, R₅ and R₆ are selected from hydrogen and methyl, e.g. at least one of R₅ and R₆ are hydrogen, or R₅ ═R₆ are hydrogen, and X is selected from —CH₂—, and —O—.

Further non-limiting examples are compounds of formula (I), (Ia) or (Ib) selected from the group consisting of (1-(2-(2-benzyl-4-methylphenoxy)ethyl)piperidin-4-yl)methanol,
(5-methyl-2-(2-(4-methylpiperazin-1-yl)ethoxy)phenyl)(phenyl)methanol,
(5-methyl-2-(2-(4-methylpiperazin-1-yl)ethoxy)phenyl)(phenyl)methanone,
(E)-1-(2-((6-benzylidene-2,4-dimethylcyclohex-1-en-1-yl)oxy)ethyl)-4-methylpiperazine,
(E)-1-(3-(2-benzyl-4-methylphenyl)allyl)-4-methylpiperazine,
1-(3-(2-benzyl-4-methylphenyl)propyl)-4-methylpiperazine,
(E)-2-(5-methyl-2-(2-(4-methylpiperazin-1-yl)ethoxy)benzylidene)cyclohexan-1-one,
(E)-2-(5-methyl-2-(2-morpholinoethoxy)benzylidene)cyclohexan-1-one,
2-(3,5-dimethyl-2-(2-morpholinopropoxy)benzyl)benzonitrile,
2-(3,5-dimethyl-2-((1-morpholinopropan-2-yl)oxy)benzyl)benzonitrile,
1-((8-benzylchroman-2-yl)methyl)-4-methylpiperazine,
1-(2-((2-benzyl-4-methylcyclohexyl)oxy)ethyl)-4-methylpiperazine,
1-(2-((2-benzyl-4-methylcyclohexyl)oxy)ethyl)-4-methylpiperazine dihydrochloride,
1-(2-(2-((5-chlorothiophen-2-yl)methyl)-4-methylphenoxy)ethyl)-4-methylpiperazine,
1-(2-(2-(2-bromobenzyl)-4,6-dimethylphenoxy)ethyl)-4-methylpiperazine,
1-(2-(2-(2-chlorobenzyl)-4-methylphenoxy)ethyl)-4-methylpiperazine,
1-(2-(2-(2-fluorobenzyl)-4,6-dimethylphenoxy)ethyl)-4-methylpiperazine,
1-(2-(2-(2-iodobenzyl)-4,6-dimethylphenoxy)ethyl)-4-methylpiperazine,
1-(2-(2-(3-bromobenzyl)-4,6-dimethylphenoxy)ethyl)-4-methylpiperazine,
1-(2-(2-(3-chlorobenzyl)-4-methylphenoxy)ethyl)-4-methylpiperazine,
1-(2-(2-(4-chlorobenzyl)-4-methylphenoxy)ethyl)-4-methylpiperazine hydrochloride,
1-(2-(2-(furan-2-ylmethyl)-4-methylphenoxy)ethyl)-4-methylpiperazine,
1-(2-(2,4-dimethyl-6-(2-(trifluoromethyl)benzyl)phenoxy)ethyl)-4-methylpiperazine,
1-(2-(2,4-dimethyl-6-(pyridin-2-ylmethyl)phenoxy)ethyl)-4-methylpiperazine,
1-(2-(2,4-dimethyl-6-(pyridin-3-ylmethyl)phenoxy)ethyl)-4-methylpiperazine,
1-(2-(2,4-dimethyl-6-(pyridin-4-ylmethyl)phenoxy)ethyl)-4-methylpiperazine,
1-(2-(2-benzyl-3,6-dimethylphenoxy)ethyl)-4-methylpiperazine,
1-(2-(2-benzyl-3-methoxyphenoxy)ethyl)-4-methylpiperazine,
1-(2-(2-benzyl-3-methylphenoxy)ethyl)-4-methylpiperazine,
1-(2-(2-benzyl-4-(tert-butyl)-6-methoxyphenoxy)ethyl)-4-methylpiperazine,
1-(2-(2-benzyl-4,6-dimethylphenoxy)ethyl)-4-methylpiperazine,
1-(2-(2-benzyl-4,6-dimethylphenoxy)ethyl)-4-methylpiperazine dihydrochloride,
1-(2-(2-benzyl-4-chlorophenoxy)ethyl)-4-ethylpiperazine hydrochloride,
1-(2-(2-benzyl-4-chlorophenoxy)ethyl)-4-methylpiperazine hydrochloride,
1-(2-(2-benzyl-4-ethylphenoxy)ethyl)-4-methylpiperazine hydrochloride,
1-(2-(2-benzyl-4-isopropylphenoxy)ethyl)-4-methylpiperazine,
1-(2-(2-benzyl-4-methylphenoxy)ethyl)-4-ethylpiperazine hydrochloride,
1-(2-(2-benzyl-4-methylphenoxy)ethyl)-4-methylpiperazine,
1-(2-(2-benzyl-4-methylphenoxy)ethyl)-4-methylpiperazine 2,3-dihydroxysuccinate,
1-(2-(2-benzyl-4-methylphenoxy)ethyl)-4-methylpiperazine 2-hydroxypropane-1,2,3-tricarboxylate,
1-(2-(2-benzyl-4-methylphenoxy)ethyl)-4-methylpiperazine bis(2,2,2-trifluoroacetate),
1-(2-(2-benzyl-4-methylphenoxy)ethyl)-4-methylpiperazine bis(2-hydroxysuccinate),
1-(2-(2-benzyl-4-methylphenoxy)ethyl)-4-methylpiperazine bis(phosphate),
1-(2-(2-benzyl-4-methylphenoxy)ethyl)-4-methylpiperazine dimaleate,
1-(2-(2-benzyl-4-methylphenoxy)ethyl)-4-methylpiperazine dimethanesulfonate,
1-(2-(2-benzyl-4-methylphenoxy)ethyl)-4-methylpiperazine hydrochloride,
1-(2-(2-benzyl-4-methylphenoxy)ethyl)-4-methylpiperazine oxalate,
1-(2-(2-benzyl-4-methylphenoxy)ethyl)-4-methylpiperazine sulfate,
1-(2-(2-benzyl-4-methylphenoxy)ethyl)piperazine,
1-(2-(2-benzyl-4-methylphenoxy)ethyl)piperidin-4-ol,
1-(2-(2-benzyl-4-methylphenoxy)propyl)-4-methylpiperazine,
1-(2-(2-benzyl-4-methylphenoxy)propyl)-4-methylpiperazine hydrochloride,
1-(2-(2-benzyl-6-methylphenoxy)ethyl)-4-methylpiperazine,
1-(2-(2-benzylphenoxy)ethyl)-4-methylpiperazine hydrochloride,
1-(2-(2-isobutyl-4-methylphenoxy)ethyl)-4-methylpiperazine,
1-(2-(2-isobutyl-4-methylphenoxy)ethyl)-4-methylpiperazine dihydrochloride,
1-(2-benzyl-4-methylphenethyl)-4-methylpiperazine,
1-(2-benzyl-4-methylphenyl)-3-(4-methylpiperazin-1-yl)propan-2-ol,
1-(2-benzyl-4-methylphenyl)-3-(4-methylpiperazin-1-yl)propan-2-one,
1-(3-(2-benzyl-4-methylphenoxy)propyl)-4-methylpiperazine hydrochloride,
1-(3-(2-benzyl-4-methylphenyl)propyl)-4-methylpiperazine,
1-methyl-4-(2-(4-methyl-2-(1-phenylethyl)phenoxy)ethyl)piperazine,
1-methyl-4-(2-(4-methyl-2-(1-phenylvinyl)phenoxy)ethyl)piperazine,
1-methyl-4-(2-(4-methyl-2-(2-methylbenzyl)phenoxy)ethyl)piperazine,
1-methyl-4-(2-(4-methyl-2-(3-methylbenzyl)phenoxy)ethyl)piperazine,
1-methyl-4-(2-(4-methyl-2-(thiophen-2-ylmethyl)phenoxy)ethyl)piperazine hydrochloride,
2-(2-((1-hydroxypropan-2-yl)oxy)-3,5-dimethylbenzyl)benzonitrile,
2-(2-(2-hydroxy-3-morpholinopropyl)-5-methylphenoxy)benzonitrile,
2-(2-(3,5-dimethyl-2-(2-morpholinoethoxy)benzyl)phenyl)acetonitrile,
2-(2,4-dimethyl-5-(2-(4-methylpiperazin-1-yl)ethoxy)benzyl)benzonitrile,
2-(2,6-difluorobenzyl)-4-methyl-N-(2-morpholinoethyl)aniline,
2-(2,6-difluorobenzyl)-N,4,6-trimethyl-N-(2-morpholinoethyl)aniline,
2-(2,6-difluorobenzyl)-N,4-dimethyl-N-(2-morpholinoethyl)aniline,
2-(2-fluorobenzyl)-N,4,6-trimethyl-N-(2-morpholinoethyl)aniline,
2-(2-fluorobenzyl)-N,4-dimethyl-N-(2-morpholinoethyl)aniline,
2-(3,5-dimethyl-2-((1-(4-methylpiperazin-1-yl)propan-2-yl)oxy)benzyl)benzonitrile,
2-(3,5-dimethyl-2-((1-(4-methylpiperazin-1-yl)propan-2-yl)oxy)benzyl)benzonitrile hydrochloride,
2-(3,5-dimethyl-2-(2-(4-methylpiperazin-1-yl)ethoxy)benzyl)benzonitrile,
2-(3,5-dimethyl-2-(2-(4-methylpiperazin-1-yl)ethoxy)benzyl)benzonitrile hydrochloride,
2-(3,5-dimethyl-2-(2-(tetrahydro-2H-pyran-4-yl)ethoxy)benzyl)benzonitrile,
2-(3,5-dimethyl-2-(2-morpholinoethoxy)benzyl)-3-fluorobenzonitrile,
2-(3,5-dimethyl-2-(2-morpholinoethoxy)benzyl)aniline,
2-(3,5-dimethyl-2-(2-morpholinoethoxy)benzyl)benzonitrile,
2-(3,5-dimethyl-2-(2-morpholinoethoxy)benzyl)benzonitrile hydrochloride,
2-(3,5-dimethyl-2-(3-morpholinopropoxy)benzyl)benzonitrile,
2-(3,5-dimethyl-2-(3-morpholinopropyl)phenoxy)benzonitrile,
2-(3,5-dimethyl-2-(methyl(2-morpholinoethyl)amino)benzyl)benzonitrile,
2-(4-methyl-2-(3-(4-methylpiperazin-1-yl)propyl)phenoxy)benzonitrile,
2-(5-methyl-2-(2-(1-methylpiperidin-4-yl)ethoxy)benzyl)benzonitrile,
2-(5-methyl-2-(2-(4-methylpiperazin-1-yl)ethoxy)benzyl)benzonitrile,
2-(5-methyl-2-(2-(4-methylpiperazin-1-yl)ethoxy)benzyl)benzonitrile dihydrochloride,
2-(5-methyl-2-(2-morpholinoethoxy)benzyl)cyclohexan-1-one,
2-(5-methyl-2-(2-morpholinopropoxy)benzyl)benzonitrile,
2-(5-methyl-2-(3-(4-methylpiperazin-1-yl)propyl)phenoxy)benzonitrile,
2-(5-methyl-2-(3-(4-methylpiperazin-1-yl)propyl)phenoxy)benzonitrile hydrochloride,
2-benzyl-4-methyl-N-(2-(4-methylpiperazin-1-yl)ethyl)aniline,
2-benzyl-4-methylphenyl 2-(4-methylpiperazin-1-yl)acetate,
2-benzyl-N,4-dimethyl-N-(2-(4-methylpiperazin-1-yl)ethyl)aniline,
2-benzyl-N,4-dimethyl-N-(2-morpholinoethyl)aniline,
3-(3,5-dimethyl-2-(2-(4-methylpiperazin-1-yl)ethoxy)benzyl)benzonitrile dihydrochloride,
3-(3,5-dimethyl-2-(2-morpholinoethoxy)benzyl)aniline,
3-bromo-2-(3,5-dimethyl-2-(2-morpholinoethoxy)benzyl)benzonitrile,
3-chloro-2-(3,5-dimethyl-2-(2-morpholinoethoxy)benzyl)benzonitrile,
4-(1-(2-(2,6-difluorobenzyl)-4,6-dimethylphenoxy)propan-2-yl)morpholine,
4-(2-(2-(1-(2-fluorophenyl)ethyl)-4,6-dimethylphenoxy)ethyl)morpholine,
4-(2-(2-(1-(2-fluorophenyl)vinyl)-4,6-dimethylphenoxy)ethyl)morpholine,
4-(2-(2-(2,4-difluorobenzyl)-4,6-dimethylphenoxy)ethyl)morpholine,
4-(2-(2-(2,5-difluorobenzyl)-4,6-dimethylphenoxy)ethyl)morpholine,
4-(2-(2-(2,6-dichlorobenzyl)-4,6-dimethylphenoxy)ethyl)morpholine,
4-(2-(2-(2,6-difluorobenzyl)-4,6-dimethylphenoxy)ethyl)morpholine,
4-(2-(2-(2,6-difluorobenzyl)-4,6-dimethylphenoxy)propyl)morpholine,
4-(2-(2-(2,6-difluorobenzyl)-4-methylphenoxy)ethyl)morpholine,
4-(2-(2-(2-chlorobenzyl)-4,6-dimethylphenoxy)ethyl)morpholine,
4-(2-(2-(2-fluorobenzyl)-4,6-dimethylphenoxy)ethyl)morpholine,
4-(2-(2-(3,5-difluorobenzyl)-4,6-dimethylphenoxy)ethyl)morpholine,
4-(2-(2,4-dimethyl-5-((perfluorophenyl)methyl)phenoxy)ethyl)morpholine,
4-(2-(2,4-dimethyl-6-(2,4,5-trifluorobenzyl)phenoxy)ethyl)morpholine,
4-(2-(2,4-dimethyl-6-(2-nitrobenzyl)phenoxy)ethyl)morpholine,
4-(2-(2-benzyl-4-methylphenoxy)ethyl)-1,2,6-trimethylpiperazine,
4-(2-(2-benzyl-4-methylphenoxy)ethyl)morpholine hydrochloride,
4-(2-(2-benzyl-4-methylphenoxy)ethyl)thiomorpholine 1-oxide,
4-(2-(2-ethyl-6-(2-fluorobenzyl)-4-methylphenoxy)ethyl)morpholine,
4-(3-(2-(2-fluorophenoxy)-4,6-dimethylphenyl)propyl)morpholine,
4-methyl-2-(5-methyl-2-(2-(4-methylpiperazin-1-yl)ethoxy)benzyl)phenol,
ethyl 4-(3,5-dimethyl-2-(2-(4-methylpiperazin-1-yl)ethoxy)benzyl)benzoate dihydrochloride, and
methyl 3-benzyl-4-(2-(4-methylpiperazin-1-yl)ethoxy)benzoate.

The compounds as defined by formula (I) (which encompass the compounds of formula (Ia), and (Ib)) comprise several chiral centers and as such may exist as a mixture of stereoisomers, or they may be resolved as isomerically pure forms. Resolving stereoisomers adds to the complexity of manufacture and purification of these compounds and so it is preferred to use the compounds as mixtures of their stereoisomers simply for economic reasons. However, if it is desired to prepare individual stereoisomers, this may be achieved according to methods known in the art, e.g. preparative HPLC and GC, crystallization or stereoselective synthesis. Accordingly, the chemical structures depicted herein encompass all possible stereoisomers forms of the illustrated compounds.

It is noted that some of the compounds as defined by formula (I) (which encompass the compounds of formula (Ia) and (IB)) are liquids and thus can be used, alone or in combination with other, well known solvents, to solubilize other compounds, e.g. cooling compounds (some of them are described herein below, such as those cooling compounds described in the international patent applications PCT/EP2020/079009 and PCT/EP2020/083453 of the applicant, N-(4-(cyanomethyl)phenyl)-2-isopropyl-5-methylcyclohexane-1-carboxamide, 2-isopropyl-5-methyl-N-(2-(pyridin-2-yl)ethyl)cyclohexane-1-carboxamide, 3-(benzo[d][1,3]dioxol-5-yl)-N,N-diphenylacrylamide, and N-(pyrazol-3-yl)-N-(thiophen-2-ylmethyl)-2-(p-tolyloxy)acetamide. This is particularly preferred because the handling of liquids, including solutions, facilitates the dosing and/or mixing of said compounds.

It is also noted that the compounds as defined by formula (I) (which encompass the compounds of formula (Ia), and (Ib)) may exist in unsolvated forms as well as solvated forms, including hydrated forms and as N-oxides. In general, compounds may be hydrated, solvated or N-oxides. Certain compounds may exist in multiple crystalline or amorphous forms. In general, all physical forms are equivalent for the uses contemplated herein and are intended to be within the scope of the present invention.

“Solvate” means a compound formed by solvation (the combination of solvent molecules with molecules or ions of the solute), or an aggregate that consists of a solute ion or molecule, i.e., a compound as defined by formula (I) (which encompass the compounds of formula (Ia), and (Ib)), with one or more solvent molecules. When water is the solvent, the corresponding solvate is “hydrate”. Further suitable solvents can be but are not limited to: acetone, acetonitrile, benzene, cyclohexane, dihydrolevoglucosenone, methyl-tetrahydrofuran, pentylene glycol, ethylene glycol, petroleum ether, ethyl lactate, methyl lactate, butyl lactate, propyl lactate, diethylether, tert-butyl methyl ether, dimethylsulfoxide, N,N-dimethylformamide, N,N-dimethylacetamide, dioxane, ethanol, ethyl acetate, ethylene glycol, diethylene glycol, para menthane (1-isopropyl-4-methylcyclohexane), propylene glycol, heptane, hexane, methanol, toluene and xylene.

“Salt” refers to a salt of a compound as defined by formula (I) (which encompass the compounds of formula (Ia), and (Ib)), which possesses the desired pharmacological activity of the parent compound. Such salts include: (1) acid addition salts, formed with inorganic acids such as hydrochloric acid, hydrobromic acid, sulfuric acid, nitric acid, phosphoric acid, and the like; or formed with organic acids such as amino acids, acetic acid, trifluoroacetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound is replaced by a metal ion, e.g., an alkali metal ion, an alkaline earth ion, or an aluminum ion; or coordinates with an organic base such as ethanolamine, diethanolamine, triethanolamine, N-methylglucamine and the like.

There is provided in a second aspect a non-medical method of inducing a cooling sensation in a human or animal comprising contacting the human or animal with a compound of formula (I) (which encompass the compounds of formula (Ia), and (Ib)), or a salt or solvate thereof.

In certain embodiments, the method is a method of achieving a cooling effect on the skin or mucosa comprising contacting the skin or mucosa with a product comprising one or more compounds of formula (I) (which encompass the compounds of formula (Ia) and (Ib)), or a salt or solvate thereof.

The compounds of formula (I) (which encompass the compounds of formula (Ia), and (Ib)), may be applied directly or as a solution or suspension, comprising an effective amount of a compound of formula (I). An amount to be effective depends, inter alia, upon the target TRPM8 area of the body but also on the cooling potency of compound or mixture of compounds.

There is provided in a third aspect consumer products, in particular consumer products which get into contact with the human skin and/or mucosa comprising a compound as defined by formula (I), which encompass the compounds of formula (Ia), and (Ib).

Consumer products which get in contact with the mucosa include, but are not limited to food products, beverages, chewing gum, tobacco and tobacco replacement products, dental care products, personal care products, including lip care products, sexual health and intimate care products.

In certain embodiments dental care products are oral care products, tooth care products, cleaners for dental prostheses, adhesives for dental prostheses, and the like.

In certain embodiments food products are iced consumable products such as ice cream, sorbet; confectioneries such as candies and chocolates; food products containing mint or mint flavour, sauces, dairy products such as milk-based drinks and yoghurts; and snacks.

In certain embodiments tobacco replacement products are liquids or solids which are suitable to be consumed by electrical means, e.g., liquids to vape.

In certain embodiments personal care products getting in contact with the mucosa are lip balms, nose sprays and eye drops.

Consumer products which get in contact with the human skin include, but are not limited to cosmetic products. In certain embodiments cosmetic products are skincare products, especially bath products, skin washing and cleansing products, skincare products, eye makeup, nail care products, foot care products, and the like. In certain embodiments cosmetic products are products with specific effects, especially sunscreens, insect repellent products, tanning products, de-pigmenting products, deodorants, antiperspirants, hair removers, and shaving products. In a certain embodiments cosmetic products are hair care products, especially hair shampoos, hair care products, hair setting products, hair-shaping products, and hair coloring products as well as scalp-care products such as scalp-cooling shampoos and creams.

The consumer products can be in any physical form, such as a solid, semi-solid, plaster, solution, suspension, lotion, cream, foam, gel, paste, or a combination thereof. The physical form of the consumer product suitable manly depends on the specific actions, such as cleaning, softening, caring, cooling, and the like, such a consumer product should fulfill.

In a certain embodiment consumer products getting in contact with the human skin are fabric care products (such as fabric detergents, fabric conditioner (including tumble dryer sheets), and scent boosters (liquid or solid)) which in a first step are applied to a fabric, e.g., when washing the fabric, said treaded fabrics then getting in contact with the human skin.

The level of use for compounds of the present invention (compounds as defined by formula (I), which encompass compounds of formula (Ia), and (Ib)) depend, inter alia, upon the target TRPM8 area of the body but also on the cooling potency of compound or mixture of compounds. For examples in an oral application of a compound of the present invention, such as dentifrice, floss, chewing gum, or white strip, the levels of use may be from about 0.00001% (0.01 ppm) to about 0.1% (1000 ppm); from about 0.00005% (0.5 ppm) to about 0.1% (1000 ppm); from about 0.0001% (1 ppm) to about 0.05% (500 ppm); or from about 0.001% (10 ppm) to about 0.01% (100 ppm) by weight of the composition. When a compound of the present invention is used in a mouthwash, the level of use may be from about 0.000001% (10 ppb) to about 0.01% (100 ppm) or from about 0.0001% (1 ppm) to about 0.001% (10 ppm) by weight of the composition. When a compound of the present invention is delivered topically, for example in shampoos and lotions the levels may be from about 0.001% (10 ppm) to about 0.5% (5000 ppm) by weight of the composition or from about 0.01% (100 ppm) to about 0.4% (4000 ppm) by weight of the composition.

The cooling potency (strength) of a compound is defined by its EC₅₀ value. EC₅₀ (half maximal effective concentration) refers to the concentration of a compound which induces a response halfway between the baseline and maximum after a specified exposure time. It is commonly used as a measure of potency. EC₅₀ is a measure of concentration, expressed in μM (μmolar) unites, where 1 μM is equivalent to 1 μmol/L.

Compounds with an EC₅₀ of 10 μM or less are perceived by the human as cooling. The lower the EC₅₀ value the higher the cooling potency. For example, compounds having an EC₅₀ value of about 0.1 μM are perceived as strong cooling compounds.

Cooling properties of a compound however are not only defined by its strength (potency; EC50) but also its longevity, which refers to the period of time (in minutes) over which a cooling effect is perceived. The longevity can range from a few seconds after rinsing to several hours or even days. In the context of oral care products, a preferred “long-lasting” effect ranges typically between 20 minutes after rinsing to 3 hours.

The compounds of formula (I) (which encompass the compounds of formula (Ia), and (Ib)) are very potent at relative low concentrations. Thus it is preferred to prepare a stock solution which is further diluted, before admixing it to a consumer product. Beside water, particular suitable solvents are triacetin and propylene glycol. But other solvent systems comprising surfactants may also be used.

To modify the cooling effect of a compound as defined herein by formula (I) (which encompass the compounds of formula (Ia), and (Ib)), the compound, a salt or solvate thereof may be combined with a compound selected from calcium ions and salts, magnesium ions and salts, arginine, or any chelating agent which is able to bind calcium or magnesium.

These compounds are known to be able to modulate the concentration of such ions in the extracellular space and therefore influence the response of the TRPM8 ion-channel, leading to a change in the perceived cooling effect.

According to Kizilbash et al. (WO2019/121193 A1) both, the cooling intensity and the flavour intensity may be enhanced when combined with agents which possess the property to potentiating said effects. Thus the compounds as defined herein by formula (I) may be combined in one particular embodiment with potentiating agents disclosed in WO2019/121193 which is incorporated by reference, in particular with regard to the potentiating agents.

The compounds of formula (I) (which encompass the compounds of formula (Ia), and (Ib)), might be used in combination with other cooling compounds known in the art.

Thus there is provided in a fourth aspect a composition comprising a cool sensation wherein the composition comprises at least one compound of formula (I), a salt or solvate thereof, and a further cooling compound.

In one particular embodiment the compounds of formula (I) (which encompass the compounds of formula (Ia), and (Ib)) may be combined with menthol (e.g., in form of peppermint oil), menthone, p-menthanecarboxamides, N-2,3-trimethyl-2-isopropyl-butanamide (WS-23), menthyl lactate (Frescolat® ML), menthone glycerol acetal (Frescolat® MGA), 3-(1-menthoxy)-propane-1,2-diol (TK-10), p-menthane-3,8-diol (known as Coolact 38D), isopulegol (known as Coolact P), monomenthyl succinate (Physcool®), monomenthyl glutarate, o-menthylglycerol, menthyl N,N-dimethylsuccinamate, 2-(sec-butyl)cyclohexan-1-one (Freskomenthe), N-(pyrazol-3-yl)-N-(thiophen-2-ylmethyl)-2-(p-tolyloxy)acetamide, 2-(4-ethylphenoxy)-N-(pyrazol-3-yl)-N-(thiophen-2-ylmethyl)acetamide, 3-(benzo[d][1,3]dioxol-5-yl)-N,N-diphenylacrylamide, 4-(2-(4-allyl-2,6-dimethoxyphenoxy)-1-ethoxypropyl)-2-methoxyphenol, 4-(2-(4-allyl-2,6-dimethoxyphenoxy)-1-((2-isopropyl-5-methylcyclohexyl)oxy)propyl)-2-methoxyphenol (including 4-(2-(4-allyl-2,6-dimethoxyphenoxy)-1-(((1S,2R,5S)-2-isopropyl-5-methylcyclohexyl)oxy)propyl)-2-methoxyphenol) and 4-(2-(4-allyl-2,6-dimethoxyphenoxy)-1-(((1R,2S,5R)-2-isopropyl-5-methylcyclohexyl)oxy)propyl)-2-methoxyphenol), N-(2-Hydroxy-2-phenylethyl)-2-isopropyl-5,5-dimethylcyclohexane-1-carboxamide, N-(4-(Cyanomethyl)phenyl)-2-isopropyl-5,5-dimethylcyclohexanecarboxamide and N-(3-Hydroxy-4-methoxyphenyl)-2-isopropyl-5,5-dimethylcyclohexanecarboxamide. Further cooling compounds with which the compounds of formula (I) may be combined are those described in the international patent applications PCT/EP2020/079009 and PCT/EP2020/083453 of the applicant.

Examples of p-methanecarboxamides include compounds such as N-ethyl-p-menthan-3-carboxamide (known commercially as WS-3), N-ethoxycarbonylmethyl-p-menthan-3-carboxamide (WS-5), N-(4-methoxyphenyl)-p-menthan-3-carboxamide (WS-12) and N-tert-butyl-p-menthan-3-carboxamide (WS-14), N-(4-(cyanomethyl)phenyl)-2-isopropyl-5-methylcyclohexane-1-carboxamide (known commercially as Evercool 180), 2-isopropyl-5-methyl-N-(2-(pyridin-2-yl)ethyl)cyclohexane-1-carboxamide (known commercially as Evercool 190), and (1R,2S,5R)—N—((S)-2-((R)-2-aminopropanamido)-2-phenylethyl)-2-isopropyl-5-methylcyclohexane-1-carboxamide.

In order to achieve more than just a cooling effect, the compounds of formula (I) (which encompass the compounds of formula (Ia), and (Ib)), a salt or solvate thereof, may be combined with other actives, such as, flavours, fragrances, and sweetening agents.

Examples of flavour ingredients include natural flavors, artificial flavors, spices, seasonings, and the like. Exemplary flavor ingredients include synthetic flavor oils and flavoring aromatics and/or oils, oleoresins, essences, and distillates, and a combination comprising at least one of the foregoing.

Flavor oils include spearmint oil, cinnamon oil, oil of wintergreen (methyl salicylate), peppermint oil, Japanese mint oil, clove oil, bay oil, anise oil, eucalyptus oil, thyme oil, cedar leaf oil, oil of nutmeg, allspice, oil of sage, mace, oil of bitter almonds, and cassia oil; useful flavoring agents include artificial, natural and synthetic fruit flavors such as vanilla, and citrus oils including lemon, orange, lime, grapefruit, yuzu, sudachi, and fruit essences including apple, pear, peach, grape, raspberry, blackberry, gooseberry, blueberry, strawberry, cherry, plum, prune, raisin, cola, guarana, neroli, pineapple, apricot, banana, melon, apricot, cherry, tropical fruit, mango, mangosteen, pomegranate, papaya, and so forth.

Additional exemplary flavors imparted by a flavoring composition include a milk flavor, a butter flavor, a cheese flavor, a cream flavor, and a yogurt flavor; a vanilla flavor; tea or coffee flavors, such as a green tea flavor, an oolong tea flavor, a tea flavor, a cocoa flavor, a chocolate flavor, and a coffee flavor; mint flavors, such as a peppermint flavor, a spearmint flavor, and a Japanese mint flavor; spicy flavors, such as an asafetida flavor, an ajowan flavor, an anise flavor, an angelica flavor, a fennel flavor, an allspice flavor, a cinnamon flavor, a chamomile flavor, a mustard flavor, a cardamom flavor, a caraway flavor, a cumin flavor, a clove flavor, a pepper flavor, a coriander flavor, a sassafras flavor, a savory flavor, a Zanthoxyli Fructus flavor, a perilla flavor, a juniper berry flavor, a ginger flavor, a star anise flavor, a horseradish flavor, a thyme flavor, a tarragon flavor, a dill flavor, a capsicum flavor, a nutmeg flavor, a basil flavor, a marjoram flavor, a rosemary flavor, a bayleaf flavor, and a wasabi (Japanese horseradish) flavor; a nut flavor such as an almond flavor, a hazelnut flavor, a macadamia nut flavor, a peanut flavor, a pecan flavor, a pistachio flavor, and a walnut flavor; alcoholic flavors, such as a wine flavor, a whisky flavor, a brandy flavor, a rum flavor, a gin flavor, and a liqueur flavor; floral flavors; and vegetable flavors, such as an onion flavor, a garlic flavor, a cabbage flavor, a carrot flavor, a celery flavor, mushroom flavor, and a tomato flavor.

Generally any flavoring or food additive (including food colors) such as those described in “Essential guide to food additives”, Third edition 2008, page 101-321 (ISBN: 978-1-905224-50-0) by Leatherhead Food International Ltd., can be used. The publication is incorporated herein by reference.

In one particular embodiment the compounds of formula (I) (which encompass the compounds of formula (Ia), and (Ib)) may be combined with anethole, menthol laevo, carvone laevo, ethyl maltol, vanillin, eucalyptol, eugenol, menthol racemic, cis-3-hexenol, linalol, mint oil (e.g. peppermint arvensis oil, peppermint piperita oil, spearmint native oil, spearmint scotch oil), corylone, ethyl butyrate, cis-3-hexenyl acetate, citral, eucalyptus oil, ethyl-vanillin, methyl salicylate, 2′-hydroxypropiophenone, ethyl acetate, methyl dihydro jasmonate, geraniol, lemon oil, iso amyl acetate, thymol, ionone beta, linalyl acetate, decanal, cis jasmone, ethyl hexanoate, melonal (2,6-dimethylhept-5-enal), citronellol, ethyl aceto acetate, nutmeg oil and clove oil, or mixtures thereof.

Examples of sweetening agents include, but are not limited to, sucrose, fructose, glucose, high fructose corn syrup, corn syrup, xylose, arabinose, rhamnose, erythritol, xylitol, mannitol, sorbitol, inositol, acesulfame potassium, aspartame, neotame, sucralose, and saccharine, and mixtures thereof; trilobatin, hesperetin dihydrochalcone glucoside, naringin dihydrochalcone, mogroside V, Luo Han Guo extract, rubusoside, rubus extract, glycyphyllin, isomogroside V, mogroside IV, siamenoside I, neomogroside, mukurozioside IIb, (+)-hernandulcin, 4 β-hydroxyhernandulcin, baiyunoside, phlomisoside I, bryodulcoside, bryoside bryonoside, abrusosides A-E, cyclocarioside A, cyclocaryoside I, albiziasaponins A-E, glycyrrhizin, araboglycyrrhizin, periandrins I-V, pterocaryosides A and B, osladin, polypodosides A and B, telosmoside A8-18, phyllodulcin, huangqioside E neoastilbin, monatin, 3-acetoxy-5,7-dihydroxy-4′-methoxyflavanone, 2R,3R-(+)-3-Acetoxy-5,7,4′-trihydroxyflavanone, (2R,3R)-dihydroquercetin 3-O-acetate, dihydroquercetin 3-O-acetate 4′-methyl ether, brazzein, curculin, mabinlin, monellin, neoculin, pentadin, thaumatin, and combinations thereof. Some of the compounds listed above are known sweetness enhancers as well as sweeteners. When used as sweetness enhancers they are normally used below their sweetness detection thresholds.

In certain embodiments, the compounds of formula (I) may be combined with additional ingredients collectively refereed to orally acceptable carrier materials.

In some aspects, the orally acceptable carrier may comprise one or more compatible solid or liquid excipients or diluents which are suitable for topical oral administration. By “compatible,” as used herein, is meant that the components of the composition are capable of being commingled without interaction in a manner which would substantially reduce stability and/or efficacy. The carriers can include the usual and conventional components of dentifrices, non-abrasive gels, subgingival gels, mouthwashes or rinses, mouth sprays, chewing gums, lozenges and breath mints. The choice of a carrier to be used is basically determined by the way the composition is to be introduced into the oral cavity. Carrier materials for toothpaste, tooth gel or the like include abrasive materials, sudsing agents, binders, humectants, flavoring and sweetening agents, etc. as disclosed in e.g., U.S. Pat. No. 3,988,433, to Benedict. Carrier materials for biphasic dentifrice formulations are disclosed in U.S. Pat. Nos. 5,213,790; 5,145,666 and 5,281,410 all to Lukacovic et al., and in U.S. Pat. Nos. 4,849,213 and 4,528,180 to Schaeffer. Mouthwash, rinse or mouth spray carrier materials typically include water, flavoring and sweetening agents, etc., as disclosed in, e.g., U.S. Pat. No. 3,988,433 to Benedict. Lozenge carrier materials typically include a candy base; chewing gum carrier materials include a gum base, flavoring and sweetening agents, as in, e.g., U.S. Pat. No. 4,083,955, to Grabenstetter et al. Sachet carrier materials typically include a sachet bag, flavoring and sweetening agents. For subgingival gels used for delivery of actives into the periodontal pockets or around the periodontal pockets, a “subgingival gel carrier” is chosen as disclosed in, e.g. U.S. Pat. Nos. 5,198,220 and 5,242,910 both to Damani. Carriers suitable for the preparation of compositions of the present disclosure are well known in the art. Their selection will depend on secondary considerations like taste, cost, and shelf stability, and the like.

Further suitable types of orally acceptable carrier materials or excipients are listed in WO2010/059289, in particular on page 17-31, which is incorporated by reference.

Scientific literature points out that the activation of TRPM8 channels may be useful for the treatment of most TRPM8-mediated pathologies (J. Med. Chem. 2016, 59 (22), 10006-10029). Thus one may assume that the compounds of formula (I) might also be suitable for treating prostate carcinomas, bladder weakness, inflammation, or pain comprising contacting a patient with one or more compounds of formula (I) as defined herein. One may also assume that the compounds of formula (I) as defined herein are suitable for alleviating the symptoms of coughs and colds, irritations, sore throat or hoarseness, as well as the treatment of laryngopharyngeal dysphagia (Int. J. Mol. Sci. 2018, 19, 4113).

Thus there is provided in a fifth aspect pharmaceutical composition comprising one or more compounds as defined by formula (I) (which encompass compounds of formula (Ia), and (Ib)), or a salt or solvate thereof.

Depending upon the particular treatment regimen contemplated, pharmaceutical compositions comprising one or more compounds of formula (I) may be administered parenterally, topically, orally, or locally. The pharmaceutical compositions may be a liquid, suspensions or a solid formulation.

In certain embodiments, the pharmaceutical composition is nasal spray, topical cream, skin sprays, throat spray, or eye drops.

While some of the compounds falling within the definition of the formula (I) above are known as such, others are novel.

Thus there is provided in a sixth aspect of the invention a compound of formula (I), a salt or solvate thereof

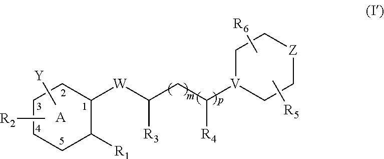

wherein

ring A represents a phenyl ring, cyclohexyl or cyclohexenyl ring;

R₁ is selected from hydrogen, C₁-C₆ alkyl (e.g. methyl, ethyl, isopropyl, isobutyl) and C₁-C₆ alkyloxy (e.g. methoxy, ethoxy, propoxy),

R₂ is connected to position 3, 4 or 5 of ring A and is selected from C₁-C₆ alkyl (e.g. methyl, ethyl, isopropyl, isobutyl, isopropyl), C₁-C₆ alkyloxy (e.g. methoxy, ethoxy), and C(O)O—C₁-C₃-alkyl (e.g. C(O)OCH₃),

R₃ is selected from hydrogen, C₁-C₆ alkyl (e.g. methyl, ethyl), C₂—C alkenyl (e.g. vinyl), OH, and ═O, and R₄ is selected from hydrogen, C₁-C₆ alkyl (e.g. methyl, ethyl), C₂-C₆ alkenyl (e.g. vinyl), and OH, with the proviso that at least one of R₃ and R₄ is hydrogen, or

R₃ and R₁ form together with the carbon atoms to which they are attached a sixth membered ring system (i.e. R₃ and R₁ is a bivalent residue —CH₂—CH₂—), and R₄ is hydrogen,

R₅ is selected from hydrogen, F and C₁-C₆ alkyl (e.g. methyl, ethyl, isobutyl),

R₆ is selected from hydrogen, F and C₁-C₆ alkyl (e.g. methyl, ethyl, isobutyl),

m is 0,

p is 1,

W is selected from —CH₂—, —O—, >C═O, —CH═, and >NR₁₃ wherein R₁₃ is selected from hydrogen and C₁-C₃ alkyl (e.g. methyl, or isopropyl);

V is selected from >CH—, and >N—,

Z is selected from —O—, >S═O, >CHR₁₂, >NR₁₂ wherein R₁₂ is hydrogen, C₁-C₃ alkyl, OH, or CH₂OH,

Y is connected to position 2 or 3 of ring A and is selected from C₄-C₆ alkyl (e.g. isobutyl),

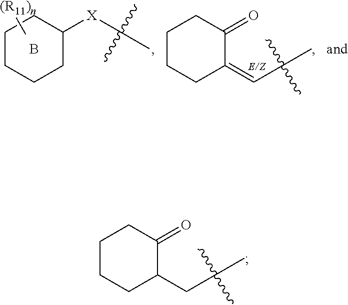

wherein

ring B represents a phenyl, thiophen, furan, or pyridine ring,

R₁₁ is selected from CN, halogen, CH₂CN, NO₂, NH₂, CF₃, C(O)OC₂HS, C₁-C₃ alkyl, C₁-C₃ alkyloxy, C₂-C₃ alkenyl, and OH, and n is an integer from 0-5, with the proviso that if n>1, R₁₁ can be the same or different,

X is selected from —CH₂—, —O—, >CH—CH₃, >CH═CH₂, >C═O, and >CHOH, with the proviso that compound of formula (I) is not 1-(3-(2-benzyl-4-methylphenyl)propyl)-4-methylpiperazine.

Further, non-limiting examples are compounds of formula (I) wherein Y is connected to position 2 and R₂ is connected to position 4.

Further, non-limiting examples are compounds of formula (Ia), a salt or solvate thereof

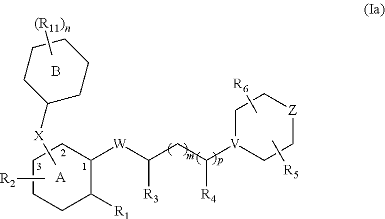

wherein

ring A represents a phenyl ring, cyclohexyl or cyclohexenyl ring;

R₁ is selected from hydrogen, C₁-C₆ alkyl (e.g. methyl, ethyl, isopropyl, isobutyl) and C₁-C₆ alkyloxy (e.g. methoxy, ethoxy, propoxy),

R₂ is selected from C₁-C₆ alkyl (e.g. methyl, ethyl, isopropyl, isobutyl, isopropyl), C₁-C₆ alkyloxy (e.g. methoxy, ethoxy), and C(O)O—C₁-C₃-alkyl (e.g. C(O)OCH₃),

R₃ is selected from hydrogen, C₁-C₆ alkyl (e.g. methyl, ethyl), C₂-C₆ alkenyl (e.g. vinyl), OH, and ═O, and R₄ is selected from hydrogen, C₁-C₆ alkyl (e.g. methyl, ethyl), C₂-C₆ alkenyl (e.g. vinyl), and OH, with the proviso that at least one of R₃ and R₄ is hydrogen, or

R₃ and R₁ form together with the carbon atoms to which they are attached a sixth membered ring system (i.e. R₃ and R₁ is a bivalent residue —CH₂—CH₂—), and R₄ is hydrogen,

R₅ is selected from hydrogen, F and C₁-C₆ alkyl (e.g. methyl, ethyl, isobutyl),

R₆ is selected from hydrogen, F and C₁-C₆ alkyl (e.g. methyl, ethyl, isobutyl),

m is 0,

p is 1,

W is selected from —CH₂—, —O—, >C═O, —CH═, and >NR₁₃ wherein R₁₃ is selected from hydrogen and C₁-C₃ alkyl (e.g. methyl, or isopropyl);

V is selected from >CH—, and >N—,

Z is selected from —O—, >S═O, >CHR₁₂, >NR₁₂ wherein R₁₂ is hydrogen, C₁-C₃ alkyl, OH,

ring B represents a phenyl, thiophen, furan, or pyridine ring,

R₁₁ is selected from CN, halogen (e.g. F, CL, Br, I), CH₂CN, NO₂, NH₂, CF₃, C(O)OC₂H₅, C₁-C₃ alkyl (e.g. methyl, or ethyl), C₁-C₃ alkyloxy (e.g. methoxy or ethoxy), C₂-C₃ alkenyl (e.g. vinyl, or allyl), and OH, and n is an integer from 0-5 (including 1, 2, and 3) with the proviso that if n>1, R₁₁ can be the same or different, and

X is connected to position 2 or 3 of ring A and is selected from —CH₂—, —O—, >CH—CH₃, >CH═CH₂, >C═O, and >CHOH,

with the proviso that compound of formula (I) is not 1-(3-(2-benzyl-4-methylphenyl)propyl)-4-methylpiperazine.

Further, non-limiting examples are compounds of formula (Ia) wherein X is connected to position 2 of ring A.

Further, non-limiting examples are compounds of formula (Ib), a salt or solvate thereof

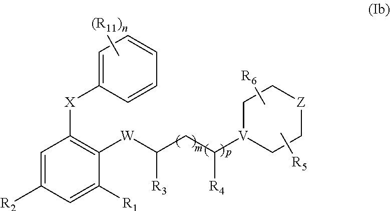

wherein

R₁ is selected from hydrogen, C₁-C₆ alkyl (e.g. methyl, ethyl, isopropyl, isobutyl) and C₁-C₆ alkyloxy (e.g. methoxy),

R₂ is selected from C₁-C₆ alkyl (e.g. methyl, ethyl, isopropyl, isobutyl), C₁-C₆ alkyloxy (e.g. methoxy, ethoxy), and C(O)O—C₁-C₃-alkyl (e.g. C(O)OCH₃),

R₃ is selected from hydrogen, C₁-C₆ alkyl (e.g. methyl, ethyl, isopropyl), C₂-C₆ alkenyl (e.g. vinyl), OH, and ═O, and R₄ is selected from hydrogen, C₁-C₆ alkyl (e.g. methyl, ethyl, isopropyl), C₂—C alkenyl (e.g. vinyl), and OH, with the proviso that at least one of R₃ and R₄ is hydrogen, or

R₃ and R₁ form together with the carbon atoms to which they are attached a sixth membered ring system (i.e. R₃ and R₁ is a bivalent residue —CH₂—CH₂—), and R₄ is hydrogen,

R₅ is selected from hydrogen, F and C₁-C₆ alkyl (e.g. methyl, ethyl),

R₆ is selected from hydrogen, F and C₁-C₆ alkyl (e.g. methyl, ethyl),

m is 0,

p is 1,

W is selected from —CH₂—, —O—, >C═O, —CH═, and >NR₁₃ wherein R₁₃ is selected from hydrogen and C₁-C₃ alkyl (e.g. methyl, or isopropyl);

V is selected from >CH—, and >N—,

Z is selected from —O—, >S═O, >CHR₁₂, >NR₁₂ wherein R₁₂ is hydrogen, C₁-C₃ alkyl, OH, or CH₂OH,

R₁₁ is selected from CN, halogen (e.g. F, CL, Br, I), CH₂CN, NO₂, NH₂, CF₃, C(O)OC₂H₅, C₁-C₃ alkyl (e.g. methyl, or ethyl), C₁-C₃ alkyloxy (e.g. methoxy or ethoxy), C₂-C₃ alkenyl (e.g. vinyl, or allyl), and OH, and n is an integer from 0-5 (including 1, 2, and 3) with the proviso that if n>1, R₁₁ can be the same or different, and

X is selected from —CH₂—, —O—, >CH—CH₃, >CH═CH₂, >C═O, and >CHOH,

with the proviso that compound of formula (Ib) is not 1-(3-(2-benzyl-4-methylphenyl)propyl)-4-methylpiperazine.

The compounds of formula (I) are either compounds known per se or may be prepared by a person skilled in the art using known synthesis methods.

Compounds of formula (I) wherein W is NR₁₃ (wherein R₁₃ is C₁-C₃ alkyl) may be prepared from a compound of formula (I) wherein W is NH by reacting it with a suitable carbonyl compound such as formaldehyde (e.g. paraformaldehyde or formalin), acetone, acetaldehyde or propanal under action of a reducing agent (e.g. sodium borohydride, sodium triacetoxy borohydride, sodium cyanoborohydride) in an appropriate solvent (e.g. acetic acid, THF, ethanol) at an appropriate temperature (25° C., 40° C.).

Compounds of formula (I) may be generally prepared by reaction of suitable carbonyl compound 2 (e.g. aldehyde, ketone or carboxylic acid) with an amine 3 under action of a reducing agent (e.g. sodium borohydride, sodium triacetoxy borohydride, sodium cyanoborohydride, lithium aluminum hydride, formic acid, or hydrogen in the presence of a homogenous or heterogenous metal catalyst such as palladium catalyst or ruthenium catalyst). The reaction may be carried out in a single step or in two operations where the amine 3 is first reacted with the carbonyl 2 and then subjected to the reducing agent. The reaction is carried out in an appropriate solvent such as but not limited to tetrahydrofuran (THF), dichloromethane (DCM), acetic acid, toluene, water, ethanol or no solvent, and may benefit from the presence of a base such as an amine base (e.g. triethylamine, pyridine or diisopropylethylamine (DIPEA)) or inorganic base (e.g. sodium hydroxide or potassium carbonate). The reaction is carried out at an appropriate temperature (e.g. 0° C., 25° C., 80° C. or 120° C.) which may vary depending on the protocol used, i.e. if the reaction is carried out in a single operation or involves isolation of the intermediate formed by the reaction between 2 and 3. In some cases, for instance when R₄ is OH, activation of 2 may be required by reacting it with an activating agent such as thionyl chloride or oxallyl chloride (optionally in the presence of catalytic N,N-Dimethylformamide), in an appropriate solvent such as dichloromethane or no solvent, at appropriate temperature (e.g. 0° C., 24° C. or 70° C.). The obtained intermediate can then be reacted with amine 3 as described above.

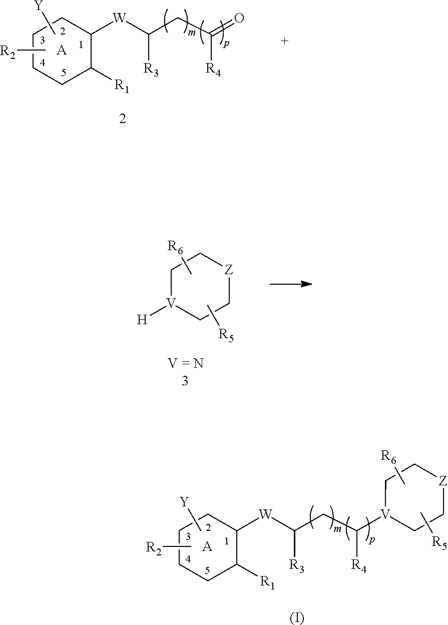

Compounds of formula (I) may also be prepared by reacting amines 3 with halides 4, optionally in the presence of a base such as an amine base (e.g. triethylamine, pyridine or diisopropylethylamine (DIPEA)) or an inorganic base (e.g. sodium hydride, sodium hydroxide, potassium carbonate or potassium phosphate). The reaction is carried out in a suitable solvent such as N,N-Dimethylformamide (DMF), tetrahydrofuran (THF) or toluene, at an appropriate temperature (e.g. 0° C., 25° C. or 100° C.). In some cases, it might be advantageous to use a catalytic amount of an additive such as potassium iodide, sodium iodide, or tetrabutylammonium iodide.

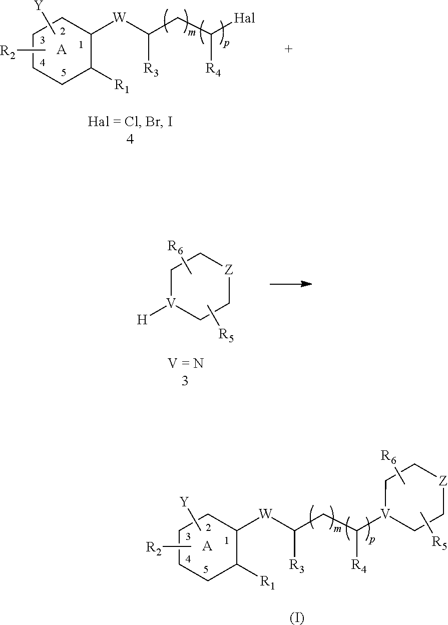

The halide 4 can in turn be prepared from carbonyl compound 3 by reduction of 3 with a suitable reducing agent (e.g. sodium borohydride, sodium triacetoxy borohydride, lithium aluminum hydride, or hydrogen in the presence of a homogenous or heterogenous metal catalyst such as palladium catalyst or ruthenium catalyst) in an appropriate solvent such as tetrahydrofuran (THF), dichloromethane (DCM), toluene, water, ethanol or methanol, at an appropriate temperature (e.g. 0° C. or 25° C.). The resulting alcohol from this reduction can then be replaced by the halogen by reacting it with an appropriate reagent such as but not limited to bromine, carbon tetrabromide, carbon tetrachloride, N-Bromosuccinimide or N-Chlorosuccinimide, and in the presence of a phosphine such as triphenylphosphine or tributylphosphine. This is done in an appropriate solvent such as dichloromethane, toluene, tetrahydrofuran or acetonitrile, at an appropriate temperature (e.g. 25° C., 40° C., 100° C.).

Alternatively, compounds of formula (I) may be prepared by the reaction of an intermediate 5 and halide 6 optionally in the presence of a base such as an amine base (e.g. triethylamine, pyridine or diisopropylethylamine (DIPEA)) or inorganic base (e.g. sodium hydroxide, potassium hydroxide or potassium carbonate) in an appropriate solvent (e.g. DMF, dimethylsulfoxide (DMSO), Acetonitrile, THF, Toluene, isopropanol or ethanol) at an appropriate temperature (e.g. 25° C., 80° C., 100° C., 150° C.). In some cases, it might be advantageous to use a catalytic amount of an additive such as potassium iodide, sodium iodide, or tetrabutylammonium iodide.

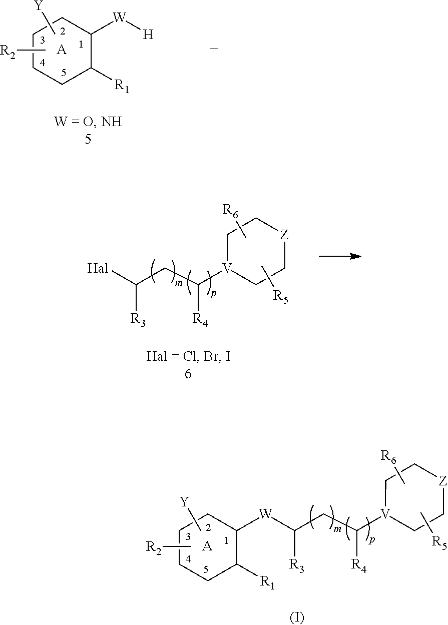

Compound 2 wherein W is O or NH, may be prepared in a similar way by reacting 5 with halide 7. As described above, this is optionally done in the presence of a base such as an amine base (e.g. triethylamine, pyridine or diisopropylethylamine (DIPEA)) or inorganic base (e.g. sodium hydroxide, potassium hydroxide, sodium hydride or potassium carbonate) in an appropriate solvent (e.g. DMF, dimethylsulfoxide (DMSO), Acetonitrile, THF, Toluene, isopropanol or ethanol) at an appropriate temperature (e.g. 0° C., 25° C., 80° C., 100° C., 150° C.). In some cases, it might be advantageous to use a catalytic amount of an additive such as potassium iodide, sodium iodide, or tetrabutylammonium iodide.

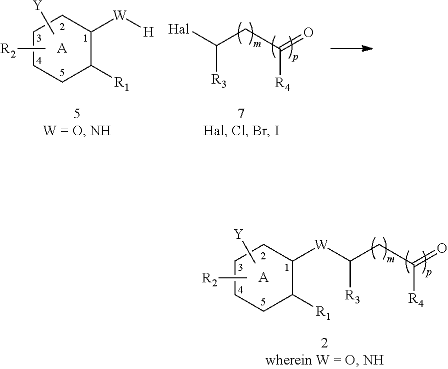

Compounds 2 wherein W is CH₂ may be synthesized reacting olefin 8 with hydrogen gas at an appropriate pressure (e.g. 1 bar or 10 bar) in the presence of a metal catalyst such as a palladium catalyst (e.g. palladium on charcoal or palladium acetate) or rhodium catalyst (e.g. rhodium on aluminum oxide) in an appropriate solvent (e.g. THF, ethyl acetate, ethanol or methanol) at an appropriate temperature (e.g. 25° C.).

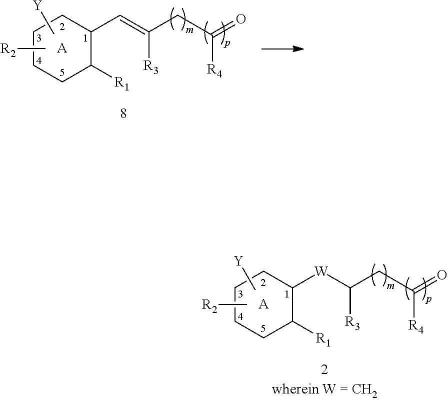

Compound 8 can be prepared by reaction of aldehyde 9 and compound 10 in the presence of an amine such as piperidine or pyrrolidine and a base such as pyridine or triethylamine, at an appropriate temperature (e.g. 100° C. or 140° C.).

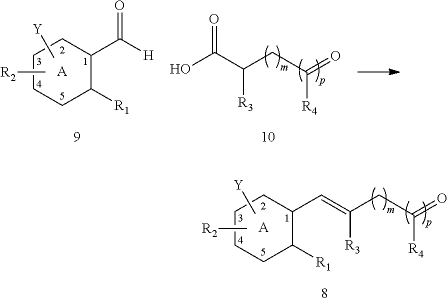

Compound 2 wherein W is CH₂ can also be prepared from triflate 11 by reacting it with an allylation reagent such as allyltributylstannane or allyl acetate, in the presence of a metal catalyst such as a palladium catalyst (e.g. Pd(Ph₃P)₄, palladium acetate or Tris(dibenzylideneacetone)dipalladium(0)), optionally in the presence of a salt such as lithium chloride. The reaction is performed in an appropriate solvent such as DMF or acetonitrile, at an appropriate temperature (e.g. 70° C. or 90° C.). The resulting intermediate can be functionalized, for instance by epoxidation by exposing it to an epoxidation reagent (e.g. 3-chloroperbenzoic acid (mCPBA), peracetic acid, dimethyldioxirane (DMDO) or methyl(trifluoromethyl)dioxirane (TFDO)) in an appropriate solvent (e.g. dichloromethane or diethyl ether) at an appropriate temperature (0° C. or 25° C.). The resulting epoxide can then be functionalized by reacting it with amine 3 in an appropriate solvent (e.g. EtOH, THF, DMF or MeOH) at a suitable temperature (e.g. 50° C. or 70° C.).

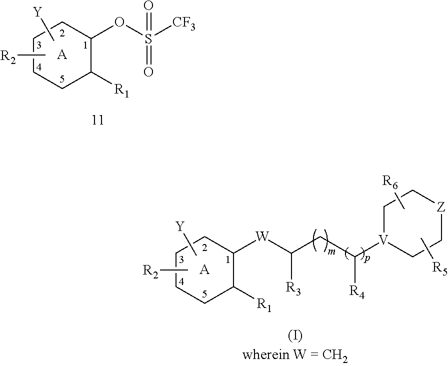

Triflate 11 can be prepared by reaction of 5 wherein W is O with a sulfonating agent such as trifluoromethylsulfonic anhydride, optionally in the presence of a base such as an amine base (e.g. triethylamine, pyridine or diisopropylethylamine (DIPEA)) or inorganic base (e.g. potassium carbonate), in an appropriate solvent such as dichloromethane, and at an appropriate temperature (e.g. 0° C., 25° C.).

The triflate 11 can also be used to prepare 5 wherein W is NH, by reacting 11 with an amine source such as benzylamine, optionally in the presence of a base such as an amine base (e.g. triethylamine, diisopropylethylamine (DIPEA)) or inorganic base (e.g. potassium carbonate, cesium carbonate), in the presence of a metal catalyst such as a palladium catalyst (e.g. Pd(Ph₃P)₄, palladium acetate or Tris(dibenzylideneacetone)dipalladium(0)), and optionally in the presence of a ligand such as triphenylphosphine or 2,2′-bis(diphenylphosphino)-1,1′-binaphthyl (which can be enantiomerically pure as R- or S-enantiomer or racemic). The reaction is carried out in an appropriate solvent such as Toluene, DMF or without solvent, and at the appropriate temperature (e.g. 70° C., 100° C. or 120° C.). The resulting benzyl-protected aniline can then be deprotected by reacting it with hydrogen gas at an appropriate pressure (e.g. 1 bar or 10 bar) in the presence of a metal catalyst such as a palladium catalyst (e.g. palladium on charcoal or palladium acetate) in an appropriate solvent (e.g. THF, ethyl acetate, ethanol or methanol or no solvent) at an appropriate temperature (e.g. 25° C.) to give 5 wherein W is NH.

Compound 12 wherein X is >C═O can be prepared by reaction of phenol 13 and acid halide 14, optionally in the presence of a base such as an amine base (e.g. triethylamine, diisopropylethylamine (DIPEA)) or inorganic base (e.g. potassium carbonate or sodium hydroxide), in an appropriate solvent (e.g. dichloromethane, tetrahydrofuran, water, dioxane, ethanol, xylenes, chlorobenzene or no solvent), at an appropriate temperature (e.g. 0° C., 25° C.). The resulting intermediate can be rearranged by reaction with a lewis acid such as aluminium trichloride, in an appropriate solvent (e.g. xylenes, chlorobenzene or no solvent), at an appropriate temperature (e.g. 120° C. or 150° C.). In some cases, both operations described above can be combined into a single step, in which case the base can be suppressed.

The resulting compound 12 wherein X is >C═O can be further transformed into 12 wherein X is >CHOH by reaction with a reducing agent (e.g. sodium borohydride, sodium triacetoxy borohydride, sodium cyanoborohydride, lithium aluminum hydride, or hydrogen in the presence of a homogenous or heterogenous metal catalyst such as palladium catalyst (e.g. palladium on charcoal) or ruthenium catalyst), in an appropriate solvent such as tetrahydrofuran (THF), dichloromethane (DCM), acetic acid, toluene, water or ethanol, at an appropriate temperature (e.g. 0° C., 25° C., 40° C.). The resulting compound 12 wherein X is >CHOH can be further converted to compound 12 wherein X is CH₂ by reacting the former with a hydride source such as triethylsilane, phenylsilane or Polymethylhydrosiloxane (PMHS), in the presence of an acid such as trifluoroacetic acid, in an appropriate solvent such as DCM at a suitable temperature (e.g. 0° C. or 25° C.).

Compound 12 wherein X is >C═CH₂ can be obtained directly from compound 12 wherein X is >C═O by reacting it with a grignard reagent such as methylmagnesium chloride, methylmagnesium iodide or methylmagnesium bromide in an appropriate solvent (e.g. diethyl ether, THF or Hexanes) at a suitable temperature (e.g. 0° C., 25° C., 40° C.). The resulting carbinol is then eliminated by treatment with an acid (e.g. acetic acid, trifluoroacetic acid or hydrochloric acid) in an appropriate solvent (e.g. DCM) at a suitable temperature (e.g. 0° C., 25° C., 40° C.). Compound 12 wherein X is >C═CH₂ can then be converted to compound 12 wherein X is >CH—CH₃ by reacting the former with hydrogen gas at an appropriate pressure (e.g. 1 bar or 10 bar), in the presence of a metal catalyst such as a palladium catalyst (e.g. palladium on charcoal or palladium acetate) in an appropriate solvent (e.g. THF, ethyl acetate, ethanol or methanol or no solvent) at an appropriate temperature (e.g. 25° C.).

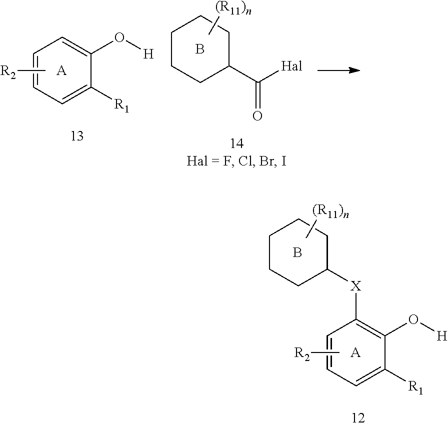

Compound 15 may be prepared by reaction of phenol 13 with benzyl halide 16 in the presence of zinc(II) chloride in an appropriate solvent (e.g. chlorobenzene, toluene or no solvent) at a suitable temperature (e.g. 120° C. or 140° C.). Alternatively, phenol 13 may be reacted with benzyl halide 16 in the presence of a base such as an amine base (e.g. triethylamine, pyridine or diisopropylethylamine (DIPEA)) or inorganic base (e.g. sodium hydroxide, potassium hydroxide or potassium carbonate) in an appropriate solvent (e.g. acetonitrile, acetone, THE or DMF) at an appropriate temperature (e.g. 25° C., 40° C. or 70° C.). The thus obtained intermediate can then be rearranged to 15 by reacting it with titanium(IV) chloride in an appropriate solvent (e.g. DCM) at a suitable temperature (e.g. −10° C., 0° C. or 25° C.).

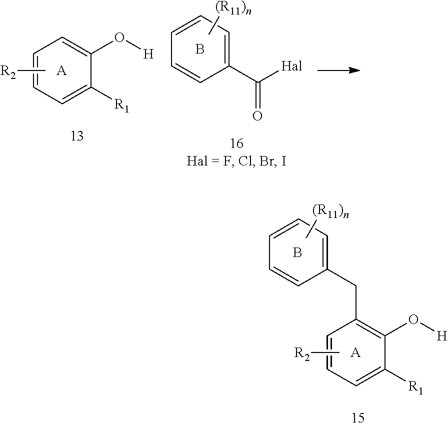

It must be mentioned that certain sensitive functional groups may be carried through the preparation sequence in protected or masked form and revealed at a later point of the synthetic sequence by standard methods known to those skilled in the art. For example, aryl halides 17 may be reacted with a cyanide source such as copper(I) cyanide in an appropriate solvent (e.g. N-methyl-2-pyrrolidone (NMP) or DMF) at a suitable temperature (e.g. 140° C. or 200° C.) to give cyanide 18.

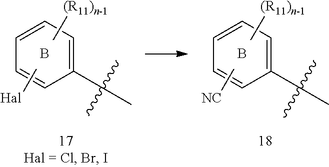

Bromide 19 may be reacted with an iodide source such as copper(I) iodide in the presence of sodium trifluoroacetate in an appropriate solvent (e.g. DMF and toluene) at a suitable temperature (120° C. or 150° C.) to give iodide 20.

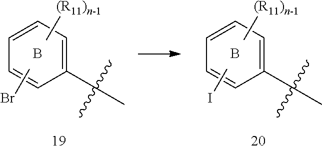

Nitro compound 21 may be reacted with hydrogen gas at an appropriate pressure (e.g. 1 bar or 10 bar), in the presence of a metal catalyst such as a palladium catalyst (e.g. palladium on charcoal or palladium acetate) in an appropriate solvent (e.g. THF, ethyl acetate, ethanol or methanol or no solvent) at an appropriate temperature (e.g. 25° C.) to give aniline 22.

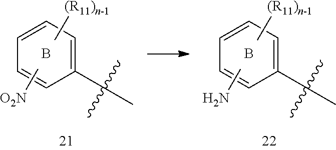

The abovementioned examples are in no way limiting or exhaustive and someone skilled in the art will recognize the general applicability of functional group manipulations in the preparation of compounds of formula (I).

Compound 23 may be prepared by the oxidation of compound 24 by reacting the latter with an oxidizing agent (e.g. pyridinium chloro chromate, dyridinium dichromate, Dess-martin periodinane (3-oxo-1,3-dihydro-1λ⁵,2-benziodoxole-1,1,1-triyl triacetate) or DMSO in the presence of an activator such as oxallyl chloride), optionally in the presence of a base (e.g. triethylamine, pyridine or diisopropylethylamine (DIPEA)) in an appropriate solvent (e.g. DCM, THF) at an appropriate temperature which may vary from −78° C. to 25° C. and further up to 40° C. depending on the selected oxidizing agent. Compound 24 may be prepared by the reaction of compound 25 with a borane source (e.g. borane dimethylsulfide or 9-Borabicyclo[3.3.1]nonane (9-BBN)) in an appropriate solvent (e.g. THF) at a suitable temperature (e.g. 0° C., 25° C.), followed by treatment with aqueous base (e.g. NaOH) and an oxidizing agent such as hydrogen peroxide at a suitable temperature (e.g. 0° C., 25° C.).

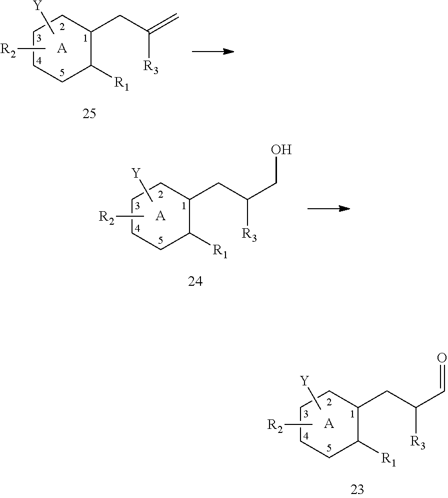

Compound 25 may be converted directly into certain compounds of formula (I) by epoxidation by exposing it to an epoxidation reagent (e.g. 3-chloroperbenzoic acid (mCPBA), peracetic acid, dimethyldioxirane (DMDO) or methyl(trifluoromethyl)dioxirane (TFDO)) in an appropriate solvent (e.g. dichloromethane or diethyl ether) at an appropriate temperature (0° C. or 25° C.), and then subjecting the formed epoxide to reaction with amine 3 in an appropriate solvent (e.g. EtOH, THF, DMF or MeOH) at a suitable temperature (e.g. 50° C. or 70° C.).

Compound 26 may be prepared by the reaction of alcohol 27 and halide 28 in the presence of a base such as an inorganic base (e.g. potassium phosphate), a copper catalyst (e.g. copper(I) iodide), and picolinic acid, in a suitable solvent (e.g. DMSO, DMF or N-methyl-2-pyrrolidone (NMP)) at an appropriate temperature (e.g. 90° C. or 120° C.).

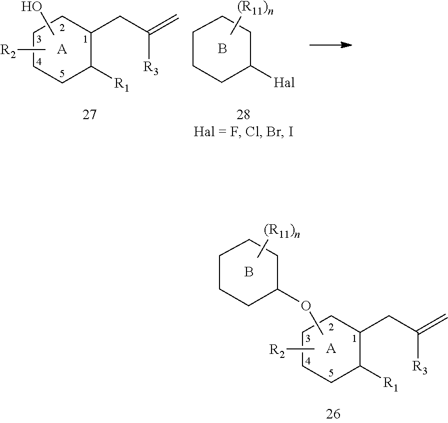

Analogous reaction conditions and reagents can be used for the preparation of 29 from 30 and 28 by reacting them in the presence of a base such as an inorganic base (e.g. potassium phosphate), a copper catalyst (e.g. copper(I) iodide), and picolinic acid, in a suitable solvent (e.g. DMSO, DMF or N-methyl-2-pyrrolidone (NMP)) at an appropriate temperature (e.g. 90° C. or 120° C.).

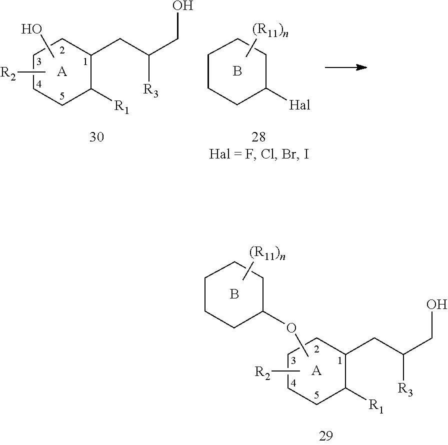

Compound 31 can be prepared by reduction of 32 by treating it with a reducing agent (e.g. lithium aluminum hydride, diisobutylaluminum hydride or hydrogen in the presence of a homogenous or heterogenous metal catalyst such as palladium catalyst (e.g. palladium on charcoal) or ruthenium catalyst), in an appropriate solvent such as tetrahydrofuran (THF), dichloromethane (DCM), toluene, water or ethanol, at an appropriate temperature (e.g. 0° C., 25° C., 40° C.).

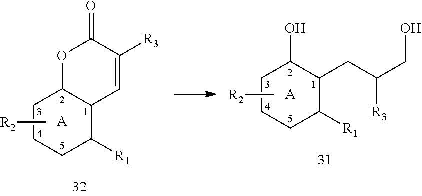

Compound 33 can be prepared by reacting alcohol 34 with allyl halide 35, optionally in the presence of a base such as an amine base (e.g. triethylamine, pyridine or diisopropylethylamine (DIPEA)) or inorganic base (e.g. sodium hydroxide, sodium hydride, potassium hydroxide or potassium carbonate) in an appropriate solvent (e.g. Acetone, DMF, DMSO, Acetonitrile, THF, Toluene, isopropanol or ethanol, water or no solvent) at an appropriate temperature (e.g. 0° C., 25° C. or 60° C.). The resulting ether intermediate can then be converted further to 33 by heating to an appropriate temperature (e.g. 150° C. or 200° C.) in a suitable solvent (e.g. DMF, DMSO or no solvent).

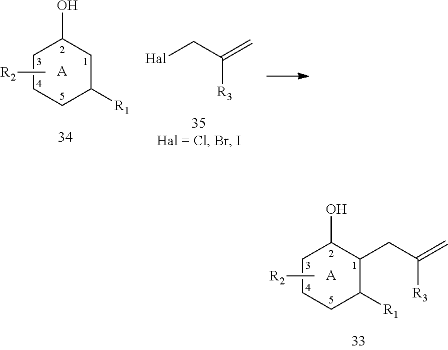

Compound 36 can be prepared by reduction of compound 37 by reacting it with hydrogen gas at an appropriate pressure (e.g. 1 bar, 5 bar or 10 bar), in the presence of a metal catalyst such as a palladium catalyst (e.g. palladium on charcoal or palladium acetate) or rhodium catalyst (e.g. rhodium on aluminum oxide), in an appropriate solvent (e.g. THF, ethyl acetate, ethanol or methanol or no solvent) at a suitable temperature (e.g. 25° C. or 50° C.). Compound 37 can be prepared by the reaction of aldehyde 38 with cyclohexanone in the presence of a base, such as an inorganic base (e.g. sodium hydroxide or potassium hydroxide) in an appropriate solvent (e.g. water alone or in mixture with the solvents such as THF or dioxane), at a suitable temperature (e.g. 0° C., 25° C. or 40° C.).

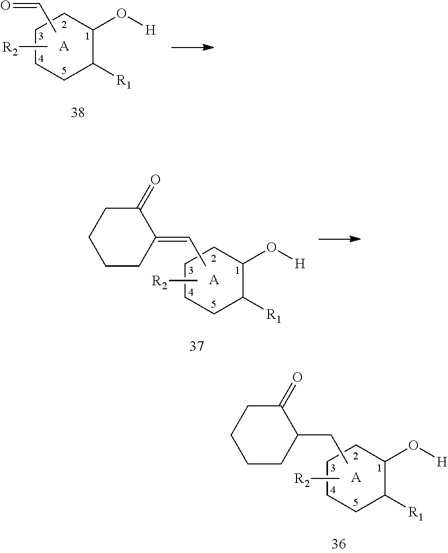

It must be noted that many of the general methods described above involving amines as substrates, may be conducted with the corresponding salts of the amines, such as hydrochlorides, hydrobromides, oxalates, phosphates, etc. In those cases, the use of additional base such as an amine base (e.g. triethylamine, pyridine or diisopropylethylamine (DIPEA)) or an inorganic base (e.g. sodium hydride, sodium hydroxide, potassium carbonate or potassium phosphate) may be required to allow the reaction to take place, a fact that will be recognized by someone skilled in the art.

It must also be noted that a different order of steps than the one described herein for the preparation of compounds of formula (I) may also be possible and in some cases advantageous. As someone skilled in the art will appreciate, this may obviate the need for protecting groups in carrying sensitive functional groups throughout the synthesis.

The invention is now further described with reference to the following non-limiting examples. These examples are for the purpose of illustration only and it is understood that variations and modifications can be made by one skilled in the art.

### EXAMPLES

### Example 1: 1-(2-(2-benzyl-4-methylphenoxy)ethyl)-4-methylpiperazine hydrochloride

### Example 1a: ethyl 2-(2-benzyl-4-methylphenoxy)acetate

To a solution of 2-benzyl-4-methylphenol (0.70 g, 3.53 mmol) in DMF (N,N-Dimethylformamide) (5 mL) was treated with potassium carbonate (1.46 g, 10.6 mmol, 3.0 equiv.), followed by ethyl 2-chloroacetate (0.45 mL, 4.24 mmol, 1.2 eq.). The resulting mixture was stirred at r.t. (room temperature, i.e. approximately 25° C.) overnight then diluted with MTBE (methyl tert-butyl ether) (50 mL) and filtered, washing with additional MTBE (50 mL). The filtrates were washed with water (2×50 mL), brine (50 mL), dried over MgSO₄ and concentrated under reduced pressure to give ethyl 2-(2-benzyl-4-methylphenoxy)acetate (1.0 g, 3.51 mmol, quant. yield) as a colorless liquid, which was used without purification in the next step. GC/MS (EI, 70 eV) m/z (%): 284 (70, [M⁺]₊), 197 (100), 196 (22), 195 (99), 181 (31), 166 (22), 165 (71), 152 (30), 91 (96), 29 (35).

### Example 1b: 2-(2-benzyl-4-methylphenoxy)acetic acid

To a solution of ethyl 2-(2-benzyl-4-methylphenoxy)acetate (1.00 g, 3.53 mmol) in THF (tetrahydrofuran) (15 mL) was added 2M aqueous NaOH solution (10 mL) and the mixture heated to reflux for 2 h. The mixture was cooled, poured into ice-cold 1M aqueous HCl solution (30 mL), extracted with MTBE (2×50 mL), washed with brine (50 mL), dried over MgSO4 and concentrated under reduced pressure to give 2-(2-benzyl-4-methylphenoxy)acetic acid (0.78 g, 3.04 mmol, 86% yield) as a white solid which was used in the next step without further purification. GC/MS (EI, 70 eV) m/z (%): 256 (82, [M⁺]₊), 197 (44), 196 (26), 195 (100), 181 (41), 166 (23), 165 (43), 91 (68), 77 (19), 31 (30). 1H NMR (400 MHz, CDCl3): δ=7.19-7.34 (m, 5H), 7.01 (s, 2H), 6.71 (d, J=8.1 Hz, 1H), 4.61 (s, 2H), 4.04 (s, 2H), 2.29 ppm (s, 3H). 13C NMR (101 MHz, CDCl3): δ=173.0, 153.1, 140.8, 131.8, 131.6, 129.9, 128.7, 128.4, 127.9, 126.0, 111.8, 65.4, 36.3, 20.6 ppm.

### Example 1c: 2-(2-benzyl-4-methylphenoxy)acetyl chloride

2-(2-benzyl-4-methylphenoxy)acetic acid (0.68 g, 2.65 mmol) was treated with thionyl chloride (1.36 mL, 18.6 mmol, 7 equiv.) and the mixture heated to 70° C. for 1 h. the mixture was evaporated under reduced pressure to give 2-(2-benzyl-4-methylphenoxy)acetyl chloride (0.73 g, 2.65 mmol, quant. yield) as a white solid which was used in the next step without further purification. 1H NMR (400 MHz, CDCl3): δ=7.10-7.33 (m, 5H), 6.96-7.03 (m, 2H), 6.66 (d, J=8.1 Hz, 1H), 4.89 (s, 2H), 4.02 (s, 2H), 2.28 ppm (s, 3H). 13C NMR (101 MHz, CDCl3): δ=170.3, 152.7, 140.7, 132.1, 131.9, 130.2, 128.9, 128.3, 127.8, 125.9, 111.9, 73.1, 35.9, 20.6 ppm.

### Example 1d: 2-(2-benzyl-4-methylphenoxy)-1-(4-methylpiperazin-1-yl)ethanone hydrochloride

A solution of 2-(2-benzyl-4-methylphenoxy)acetyl chloride (0.36 g, 1.30 mmol) in DCM (Dichloromethane) (7 mL) was cooled to 0° C. then treated with 1-methylpiperazine (0.143 g, 1.43 mmol, 1.1 equiv.) and allowed to stir for 40 h at room temperature. The resulting white solid was filtered and dried under vacuum to give 2-(2-benzyl-4-methylphenoxy)-1-(4-methylpiperazin-1-yl)ethanone hydrochloride (0.43 g, 1.15 mmol, 88% yield) as a white solid which was used in the following step without further purification. LC-MS (ESI+): 339 ([M+H]⁺). 13C NMR (101 MHz, CDCl3): δ=166.9, 153.5, 141.1, 132.5, 131.6, 128.6, 128.5, 128.4, 128.1, 126.2, 111.5, 68.6, 53.1, 52.8, 43.4, 42.1, 36.3, 20.5 ppm.

### Example 1e: 1-(2-(2-benzyl-4-methylphenoxy)ethyl)-4-methylpiperazine hydrochloride

A suspension of LAH (LiAlH₄) (26 mg, 0.680 mmol, 1.7 equiv.) in THF (tetrahydrofuran) (8 mL) was treated in portions at r.t. (room temperature) with solid 2-(2-benzyl-4-methylphenoxy)-1-(4-methylpiperazin-1-yl)ethanone hydrochloride (0.150 g, 0.400 mmol). After initial gas evolution subsided, the mixture was heated to 50° C. for 1 h. After cooling to r.t., the mixture was quenched by addition of EtOAc (Ethyl Acetate) (1 mL) followed by saturated aqueous sodium-potassium-tartrate solution (Rochelle's salt solution) (10 mL) and the slurry stirred overnight. The resulting mixture was diluted with water (20 mL) and saturated aqueous sodium bicarbonate solution (20 mL), extracted with EtOAc (2×50 mL), dried over MgSO₄ and concentrated under reduced pressure. The resulting pale yellow liquid was taken up in diethyl ether (10 mL) and treated with 2M HCl in diethyl ether (0.4 mL) resulting in formation of a white precipitate. The precipitate was filtered and dried under reduced pressure to give 1-(2-(2-benzyl-4-methylphenoxy)ethyl)-4-methylpiperazine hydrochloride (0.129 g, 0.357 mmol, 89% yield) as a white solid. LC-MS (ESI+): 325 ([M+H]⁺). 13C NMR (101 MHz, METHANOL-d4): δ=153.5, 141.1, 131.7, 131.0, 128.5, 128.1, 128.1, 127.8, 125.6, 111.7, 63.0, 56.0, 49.7, 49.1, 41.9, 357, 19.2 ppm.

### Example 2: 4-(2-(2-benzyl-4-methylphenoxy)ethyl)morpholine hydrochloride

### Example 2a: 2-(2-benzyl-4-methylphenoxy)-1-morpholinoethanone

A solution of 2-(2-benzyl-4-methylphenoxy)acetyl chloride (0.36 g, 1.3 mmol) in DCM (10 mL) was treated at 0° C. with pyridine (0.12 mL, 1.4 mmol, 1.1 equiv.) followed by morpholine (0.125 g, 1.4 mmol, 1.1 equiv.) and the resulting mixture stirred for 40 h at r.t. The resulting mixture was diluted with EtOAc (50 mL), washed with water (2×20 mL), brine (20 mL), dried over MgSO₄ and concentrated under reduced pressure. The material was then purified by silica gel flash column chromatography eluting with a gradient of EtOAc in hexanes to give 2-(2-benzyl-4-methylphenoxy)-1-morpholinoethanone (0.245 g, 0.75 mmol, 58% yield) as a colorless liquid. GC/MS (EI, 70 eV) m/z (%): 325 (9, [M]⁺), 198 (17), 197 (100), 195 (14), 193 (11), 165 (23), 152 (11), 100 (14), 91 (35), 70 (10), 56 (14). 1H NMR (400 MHz, CDCl3): δ=7.25-7.31 (m, 2H), 7.16-7.23 (m, 3H), 6.97-7.05 (m, 2H), 6.83 (d, J=8.3 Hz, 1H), 4.65 (s, 2H), 3.98 (s, 2H), 3.54-3.68 (m, 4H), 3.27-3.41 (m, 4H), 2.29 ppm (s, 3H). 13C NMR (101 MHz, CDCl3): δ=166.9, 153.6, 140.9, 131.9, 130.9, 129.0, 128.7, 128.3, 128.0, 125.9, 111.4, 68.4, 66.7, 66.6, 45.7, 42.4, 36.2, 20.5 ppm.

### Example 2b: 4-(2-(2-benzyl-4-methylphenoxy)ethyl)morpholine hydrochloride

A suspension of LAH (13 mg, 0.34 mmol, 1.1 equiv.) in THF (3 mL) was treated dropwise at r.t. with a solution of 2-(2-benzyl-4-methylphenoxy)-1-morpholinoethanone (0.100 g, 0.31 mmol) in THF (3 mL). After initial gas evolution subsided, the mixture was heated to 50° C. for 1 h. After cooling to r.t., the mixture was quenched by addition of EtOAc (Ethyl Acetate) (1 mL) followed by aqueous sodium-potassium-tartrate solution (Rochelle's salt solution) (10 mL) and the slurry stirred overnight. The resulting mixture was diluted with water (20 mL) and saturated aqueous sodium bicarbonate solution (20 mL), extracted with EtOAc (2×50 mL), dried over MgSO₄ and concentrated under reduced pressure. The resulting pale yellow liquid was taken up in diethyl ether (10 mL) and treated with 2M HCl in diethyl ether (0.4 mL) resulting in formation of a white precipitate. The precipitate was filtered and dried under reduced pressure to give 4-(2-(2-benzyl-4-methylphenoxy)ethyl)morpholine hydrochloride (29 mg, 0.083 mmol, 27% yield) as a white solid. LC-MS (ESI+): 312 ([M+H]⁺). 1H NMR (400 MHz, METHANOL-d4): δ=7.24-7.31 (m, 2H), 7.14-7.21 (m, 1H), 7.07-7.14 (m, 3H), 7.03 (d, J=2.0 Hz, 1H), 6.93 (d, J=8.3 Hz, 1H), 4.29-4.38 (m, 2H), 4.02 (s, 2H), 3.79-3.95 (m, 2H), 3.58-3.74 (m, 2H), 3.53 (dd, J=5.3, 4.0 Hz, 2H), 3.21-3.31 (m, 2H), 3.08 (brs, 2H), 2.29 ppm (s, 3H). 13C NMR (101 MHz, METHANOL-d4): δ=153.6, 141.2, 131.9, 131.0, 128.4, 128.0, 128.0, 127.9, 125.6, 111.5, 63.5, 62.7, 56.4, 52.5, 35.7, 19.2 ppm.

### Example 3: 1-(2-(2-benzyl-4-methylphenoxy)ethyl)-4-methylpiperazine

1-(2-(2-benzyl-4-methylphenoxy)ethyl)-4-methylpiperazine hydrochloride (0.10 g, 0.28 mmol) was dissolved in 2M aqueous NaOH solution (10 mL) and extracted with MTBE (3×20 mL). The combined extracts were washed with brine (20 mL), dried over MgSO₄ and concentrated under reduced pressure. The resulting material was purified by silica gel flash chromatography eluting with a gradient of a mixture of triethylamine, ethanol and EtOAc (ratio 1:9:90) in hexanes to give 1-(2-(2-benzyl-4-methylphenoxy)ethyl)-4-methylpiperazine (70 mg, 0.22 mmol, 77% yield) as a pale yellow liquid. GC/MS (EI, 70 eV) m/z (%): 324 (1, [M⁺]⁺), 127 (43), 126 (7), 114 (7), 113 (100), 91 (6), 70 (31), 58 (5), 56 (11), 43 (9), 42 (11).

### Example 4: 1-(2-(2-benzyl-4-methylphenoxy)ethyl)-4-methylpiperazine bis(phosphate)

A solution of phosphoric acid (85 wt %, 0.35 g, 3.1 mmol) in Ethanol (15 mL) was treated at r.t. with a solution of 1-(2-(2-benzyl-4-methylphenoxy)ethyl)-4-methylpiperazine (1.00 g, 3.08 mmol) in Ethanol (15 mL) and the resulting suspension stirred for 15 min. The formed solid was filtered and dried in a vacuum oven to give 1-(2-(2-benzyl-4-methylphenoxy)ethyl)-4-methylpiperazine bis(phosphate) (0.80 g, 1.5 mmol, 50% yield) as a white solid. Anal. Calcd. (%) for C₂₁H₃₄N₂O₉P₂ (520.46 g/mol): C 48.46, H 6.58, N 5.38, O 27.67, P 11.90 Found: C 48.21, H 6.50, N 5.41.

### Example 5: 1-(2-(2-benzyl-4-methylphenoxy)ethyl)-4-methylpiperazine bis(2,2,2-trifluoroacetate)

A solution of TFA (2,2,2-trifluoroacetic acid) (0.35 g, 3.1 mmol, 2 equiv.) in Ethanol (10 mL), was treated at r.t. with a solution of 1-(2-(2-benzyl-4-methylphenoxy)ethyl)-4-methylpiperazine (0.50 g, 1.54 mmol) in Ethanol (10 mL). The resulting mixture was evaporated to dryness and the resulting solid suspended in diethyl ether and filtered, washing with cold diethyl ether, followed by hexanes. The solid was dried in a vacuum oven to give 1-(2-(2-benzyl-4-methylphenoxy)ethyl)-4-methylpiperazine bis(2,2,2-trifluoroacetate) (0.715 g, 1.29 mmol, 84% yield) as a white solid. Anal. Calcd. (%) for C₂₅H₃₀N₂O₅F₆ (552.51 g/mol): C 54.35, H 5.47, N 5.07, O 14.48, F 20.63 Found: C 54.42, H 5.44, N 5.07.

### Example 6: 1-(2-(2-benzyl-4-methylphenoxy)ethyl)-4-methylpiperazine bis(2-hydroxysuccinate)

A solution of 2-hydroxysuccinic acid (0.33 g, 2.47 mmol) in Ethanol (10 mL) was treated at r.t. with a solution of 1-(2-(2-benzyl-4-methylphenoxy)ethyl)-4-methylpiperazine (0.80 g, 2.47 mmol) in Ethanol (10 mL). The resulting solid was filtered and washed with diethyl ether and hexanes then dried in a vacuum oven to give 1-(2-(2-benzyl-4-methylphenoxy)ethyl)-4-methylpiperazine bis(2-hydroxysuccinate) (0.715 g, 1.20 mmol, 48% yield) as a white solid. Anal. Calcd. (%) for C₂₉H₄₀N₂O₁₁ (592.64 g/mol): C 58.77, H 6.80, N 4.73, O 29.70 Found: C 57.87, H 6.64, N 4.71.

### Example 7: 1-(2-(2-benzyl-4-methylphenoxy)ethyl)-4-methylpiperazine 2,3-dihydroxysuccinate

A solution of 2,3-dihydroxysuccinic acid (0.46 g, 3.1 mmol) in Ethanol (10 mL) was treated at r.t. with a solution of 1-(2-(2-benzyl-4-methylphenoxy)ethyl)-4-methylpiperazine (1.0 g, 3.1 mmol) in Ethanol (10 mL). The resulting mixture was stirred for 30 min then evaporated to dryness and the resulting solid suspended in hexanes and filtered, washing with cold hexanes. The solid was dried in a vacuum oven to give 1-(2-(2-benzyl-4-methylphenoxy)ethyl)-4-methylpiperazine 2,3-dihydroxysuccinate (1.3 g, 2.74 mmol, 89% yield) as a white solid. 1H NMR (400 MHz, DMSO-d6): δ=7.37-8.54 (m, 4H), 7.19-7.29 (m, 4H), 7.12-7.18 (m, 1H), 6.93-7.01 (m, 2H), 6.85 (d, J=9.0 Hz, 1H), 4.13 (s, 2H), 4.02 (t, J=5.5 Hz, 2H), 3.86 (s, 2H), 2.53-2.92 (m, 10H), 2.45 (s, 3H), 2.19 ppm (s, 3H). 13C NMR (101 MHz, DMSO-d6): δ=174.5, 154.4, 141.6, 131.4, 129.4, 128.8, 126.1, 112.2, 72.4, 66.4, 56.6, 53.8, 51.6, 44.2, 35.9, 20.6 ppm.

### Example 8: 1-(2-(2-benzyl-4-methylphenoxy)ethyl)-4-methylpiperazine dimaleate

A solution of maleic acid (0.29 g, 2.47 mmol) in Ethanol (10 mL) was treated at r.t. with a solution of 1-(2-(2-benzyl-4-methylphenoxy)ethyl)-4-methylpiperazine (0.80 g, 2.47 mmol) in Ethanol (10 mL). The resulting solid was filtered and washed with diethyl ether and hexanes then dried in a vacuum oven to give 1-(2-(2-benzyl-4-methylphenoxy)ethyl)-4-methylpiperazine dimaleate (0.65 g, 1.17 mmol, 47% yield) as a white solid. Anal. Calcd. (%) for C₂₉H₃N₂O₉ (556.61 g/mol): C 62.58, H 6.52, N 5.03, O 25.87 Found: C 62.34, H 6.50, N 5.05.

### Example 9: 1-(2-(2-benzyl-4-methylphenoxy)ethyl)-4-methylpiperazine dimethanesulfonate

A solution of methanesulfonic acid (0.35 mL, 5.42 mmol) in Ethanol (10 mL) was treated at r.t. with a solution of 1-(2-(2-benzyl-4-methylphenoxy)ethyl)-4-methylpiperazine (0.88 g, 2.71 mmol) in Ethanol (10 mL). The resulting mixture was stirred for 30 min then evaporated to dryness and the resulting solid suspended in diethyl ether and filtered, washing with cold diethyl ether and then hexanes. The solid was dried in a vacuum oven to give 1-(2-(2-benzyl-4-methylphenoxy)ethyl)-4-methylpiperazine dimethanesulfonate (1.22 g, 2.36 mmol, 87% yield) as a white solid. Anal. Calcd. (%) for C₂₃H₃₆N₂O₇S₂ (516.68 g/mol): C 53.47, H 7.02, N 5.42, O 21.68, S 12.41 Found: C 52.72, H 6.95, N 5.40.

### Example 10: 1-(2-(2-benzyl-4-methylphenoxy)ethyl)-4-methylpiperazine sulfate

A solution of sulfuric acid (0.16 mL, 3.1 mmol) in Ethanol (10 mL) was treated at r.t. with a solution of 1-(2-(2-benzyl-4-methylphenoxy)ethyl)-4-methylpiperazine (1.0 g, 3.1 mmol) in Ethanol (10 mL). The resulting solid was filtered and washed with diethyl ether and hexanes then dried in a vacuum oven to give 1-(2-(2-benzyl-4-methylphenoxy)ethyl)-4-methylpiperazine sulfate (1.21 g, 2.86 mmol, 93% yield) as a white solid. LC-MS (ESI−): 97 ([HSO₄]⁻). LC-MS (ESI+): 324 ([M+H]⁺).

### Example 11: 1-(2-(2-benzyl-4-methylphenoxy)ethyl)-4-methylpiperazine citrate

A solution of citric acid (0.59 g, 3.1 mmol) in Ethanol (10 mL) was treated at r.t. with a solution of 1-(2-(2-benzyl-4-methylphenoxy)ethyl)-4-methylpiperazine (1.0 g, 3.1 mmol) in Ethanol (10 mL). The resulting mixture was stirred for 30 min then evaporated to dryness and the resulting solid suspended in diethyl ether and filtered, washing with cold diethyl ether and then hexanes. The solid was dried in a vacuum oven to give 1-(2-(2-benzyl-4-methylphenoxy)ethyl)-4-methylpiperazine citrate (1.22 g, 2.36 mmol, 77% yield) as a white solid. LC-MS (ESI+): 324 ([M+H]⁺). 1H NMR (400 MHz, DMSO-d6): δ=8.02-13.16 (m, 4H), 7.23-7.29 (m, 2H), 7.18-7.22 (m, 2H), 7.13-7.18 (m, 1H), 6.94-7.00 (m, 2H), 6.86 (d, J=8.1 Hz, 1H), 4.03 (t, J=5.5 Hz, 2H), 3.86 (s, 2H), 2.81-3.02 (range, 4H), 2.76 (t, J=5.4 Hz, 2H), 2.54-2.72 (m, 10H), 2.20 ppm (s, 3H). 13C NMR (101 MHz, DMSO-d6): δ=177.0, 171.8, 154.4, 141.5, 131.4, 129.5, 129.4, 129.0, 128.6, 128.2, 126.2, 112.2, 72.0, 66.4, 56.4, 53.6, 51.2, 44.5, 437, 35.9, 20.6 ppm.

### Example 12: 1-(2-(2-benzyl-4-methylphenoxy)propyl)-4-methylpiperazine

### Example 12a: ethyl 2-(2-benzyl-4-methylphenoxy)propanoate

Prepared analogously to Example 1a, from 2-benzyl-4-methylphenol (5.0 g, 25.2 mmol) and ethyl 2-bromopropanoate (5.48 g, 30.3 mmol, 1.2 equiv) to give, after silica gel flash column chromatography eluting with EtOAc in Hexanes, ethyl 2-(2-benzyl-4-methylphenoxy)propanoate (5.55 g, 18.6 mmol, 74% yield) as a colorless liquid. GC/MS (EI, 70 eV) m/z (%): 298 (39, [M⁺]⁺), 198 (20), 197 (100), 195 (18), 166 (17), 165 (43), 121 (46), 120 (22), 105 (40), 91 (25). 1H NMR (400 MHz, CDCl3): δ=7.30-7.36 (m, 4H), 7.20-7.28 (m, 1H), 6.96-7.02 (m, 2H), 6.67-6.72 (m, 1H), 4.76 (q, J=6.8 Hz, 1H), 4.26 (q, J=7.1 Hz, 2H), 4.10-4.16 (m, 1H), 4.07 (s, 1H), 2.30 (s, 3H), 1.62 (d, J=6.8 Hz, 3H), 1.30 ppm (t, J=7.1 Hz, 3H). 13C NMR (101 MHz, CDCl3): δ=172.5, 153.4, 141.3, 131.5, 130.6, 130.2, 129.1, 128.2, 127.6, 125.7, 112.0, 73.0, 61.1, 36.1, 20.6, 18.6, 14.2 ppm.

### Example 12b: 1-(2-(2-benzyl-4-methylphenoxy)propyl)-4-methylpiperazine

A solution of ethyl 2-(2-benzyl-4-methylphenoxy)propanoate (5.50 g, 18.4 mmol) in DCM (40 mL) was cooled to −78° C. and treated dropwise with a solution of DIBAL-H (Diisobutyl aluminum hydride) (1M in Toluene, 38.7 mL, 38.7 mmol, 2.1 equiv.). The resulting mixture was allowed to warm to r.t. over 1 h then cooled to 5° C. and quenched with EtOAc (3 mL), treated with Rochelle's salt solution (50 mL) and stirred for 3 h at r.t. The mixture was extracted with DCM (3×100 mL), washed with water (100 mL), dried over MgSO₄ and concentrated under reduced pressure to give the crude intermediate alcohol 2-(2-benzyl-4-methylphenoxy)propan-1-ol (4.4 g, 15 mmol), which was taken up in DCM (30 mL) and added slowly at r.t. to a suspension of Celite (3.9 g) and PCC (Pyridinium chlorochromate) (3.88 g, 18.0 mmol) in DCM (20 mL). The resulting mixture was stirred for 5 h at r.t. then filtered through a plug of silica gel and concentrated to give the crude intermediate aldehyde 2-(2-benzyl-4-methylphenoxy)propanal (3.85 g, 10.2 mmol). This was dissolved in DCM (40 mL) and treated with 1-methylpiperazine (1.12 g, 11.2 mmol) followed by portionwise addition of sodium triacetoxyborohydride (2.38 g, 11.2 mmol). The resulting mixture was stirred for 3 h at r.t. and then poured into iced saturated aqueous NaHCO₃ solution (100 mL), extracted with DCM (3×50 mL), washed with water (100 mL), dried over MgSO₄ and concentrated under reduced pressure. The material was then purified by silica gel flash column chromatography eluting with a gradient of a mixture of triethylamine, ethanol and EtOAc (ratio 1:9:90) in hexanes to give 1-(2-(2-benzyl-4-methylphenoxy)propyl)-4-methylpiperazine (1.55 g, 4.58 mmol, 25% yield) as a colorless liquid. GC/MS (EI, 70 eV) m/z (%): 338 (1, [M⁺]⁺), 141 (13), 140 (9), 114 (7), 113 (100), 100 (6), 98 (6), 70 (29), 56 (6), 43 (6), 42 (10). 1H NMR (400 MHz, CDCl3): δ=7.21-7.31 (m, 4H), 7.16-7.21 (m, 1H), 6.99 (dd, J=8.3, 2.0 Hz, 1H), 6.94 (d, J=2.0 Hz, 1H), 6.83 (d, J=8.3 Hz, 1H), 4.50-4.59 (m, 1H), 3.96 (s, 2H), 2.67 (dd, J=13.1, 6.2 Hz, 1H), 2.49 (dd, J=13.1, 4.8 Hz, 1H), 2.33-2.71 (m, 8H), 2.29 (s, 3H), 2.27 (s, 3H), 1.23 ppm (d, J=6.4 Hz, 3H). 13C NMR (101 MHz, CDCl3): δ=153.3, 141.5, 131.5, 130.4, 129.5, 129.0, 128.1, 127.6, 125.6, 112.9, 72.4, 63.7, 55.3, 53.8, 46.1, 36.2, 20.5, 18.6 ppm.

### Example 13: 1-(2-(2-benzyl-4-methylphenoxy)propyl)-4-methylpiperazine hydrochloride

A solution of 1-(2-(2-benzyl-4-methylphenoxy)propyl)-4-methylpiperazine (0.63 g, 1.86 mmol) in diethyl ether was treated dropwise at r.t. with 2M HCl in diethyl ether (2.5 mL, 5 mmol) the resulting solid was filtered and washed with diethyl ether, followed by hexanes and then dried in a vacuum oven to give 1-(2-(2-benzyl-4-methylphenoxy)propyl)-4-methylpiperazine hydrochloride (0.52 g, 1.39 mmol, 75% yield) as a white solid. LC-MS (ESI+): 339 ([M+H]⁺). 13C NMR (101 MHz, DMSO-d6): δ=151.8, 141.6, 131.7, 131.1, 130.4, 129.3, 128.6, 128.2, 126.2, 114.5, 69.3, 49.4-50.1 (br), 36.1, 20.7, 17.3 ppm.

### Example 14: 1-(2-(2-benzyl-4-ethylphenoxy)ethyl)-4-methylpiperazine hydrochloride

Was prepared in an analogous sequence as Example 12, from 2-benzyl-4-ethylphenol, ethyl 2-chloroacetate and 1-methylpiperazine to give 1-(2-(2-benzyl-4-ethylphenoxy)ethyl)-4-methylpiperazine, which was precipitated as the hydrochloride according to the procedure described in Example 13, to give 1-(2-(2-benzyl-4-ethylphenoxy)ethyl)-4-methylpiperazine hydrochloride as a white solid. LC-MS (ESI+): 339 ([M+H]⁺). 1H NMR (400 MHz, DMSO-d6): δ=11.48-13.47 (m, 1H), 7.26-7.34 (m, 2H), 7.15-7.25 (m, 3H), 7.06 (dd, J=8.3, 2.2 Hz, 1H), 7.00 (s, 1H), 6.93 (d, J=8.3 Hz, 1H), 4.40 (br s, 2H), 3.95 (s, 2H), 3.39-3.78 (m, 10H), 2.82 (br s, 3H), 2.46-2.57 (m, 3H), 1.13 ppm (t, J=7.6 Hz, 3H). 13C NMR (101 MHz, DMSO-d6): δ=153.9, 141.3, 136.8, 130.4, 129.4, 129.1, 128.8, 127.0, 126.3, 112.3, 63.3, 49.7-50.6 (br), 48.9-49.6 (br), 35.9, 27.8, 16.4 ppm.

### Example 15: 1-methyl-4-(2-(4-methyl-2-(thiophen-2-ylmethyl)phenoxy)ethyl)piperazine hydrochloride

### Example 15a: 4-methyl-2-(thiophen-2-ylmethyl)phenol

A mixture of p-cresol (20.8 g, 192 mmol, 5 equiv.) and trifluoromethanesulfonic acid (0.404 g, 2.69 mmol, 0.07 equiv.) was treated dropwise at r.t. with thiophen-2-ylmethyl acetate (6 g, 38.4 mmol) over 5 minutes and the resulting mixture heated to 100° C. for 1 h. The resulting mixture was cooled to r.t. and poured onto water (200 mL), extracted with EtOAc (3×100 mL), washed with water (100 mL), brine (100 mL), dried over MgSO₄ and concentrated under reduced pressure. Purification of the crude by silica gel flash column chromatography, eluting with a gradient of EtOAc in Hexanes gave 4-methyl-2-(thiophen-2-ylmethyl)phenol (1.3 g, 5.98 mmol, 16% yield) as a colorless liquid. LC-MS (ESI+): 331 ([M+H]⁺).

### Example 15b: 1-methyl-4-(2-(4-methyl-2-(thiophen-2-ylmethyl)phenoxy)ethyl)piperazine hydrochloride

Was prepared in an analogous sequence as Example 12, from 4-methyl-2-(thiophen-2-ylmethyl)phenol, ethyl 2-chloroacetate and 1-methylpiperazine to give 1-methyl-4-(2-(4-methyl-2-(thiophen-2-ylmethyl)phenoxy)ethyl)piperazine, which was precipitated as the hydrochloride according to the procedure described in Example 13, to give 1-methyl-4-(2-(4-methyl-2-(thiophen-2-ylmethyl)phenoxy)ethyl)piperazine hydrochloride as a white solid. LC-MS (ESI+): 331 ([M+H]⁺). 13C NMR (101 MHz, DMSO-d6): δ=153.5, 144.1, 131.1, 130.3, 129.0, 128.6, 127.3, 125.6, 124.5, 112.3, 63.3, 48.98-50.57 (br), 30.2, 20.6 ppm.

### Example 16: 4-(2-(2-benzyl-4-methylphenoxy)ethyl)thiomorpholine 1-oxide

### Example 16a: 4-(2-(2-benzyl-4-methylphenoxy)ethyl)thiomorpholine

Was prepared in an analogous sequence as Example 12, from 2-benzyl-4-methylphenol, ethyl 2-chloroacetate and thiomorpholine to give 4-(2-(2-benzyl-4-methylphenoxy)ethyl)thiomorpholine as a colorless liquid. GC/MS (EI, 70 eV) m/z (%): 327 (3, [M⁺]⁺), 165 (4), 130 (6), 118 (5), 117 (7), 116 (100), 91 (7), 88 (8), 56 (7), 55 (3), 42 (4). 1H NMR (400 MHz, CDCl3): δ=7.12-7.27 (m, 5H), 6.97 (dd, J=8.3, 1.8 Hz, 1H), 6.91 (d, J=2.1 Hz, 1H), 6.73 (d, J=8.2 Hz, 1H), 4.01 (t, J=5.6 Hz, 2H), 3.93 (s, 2H), 2.73-2.80 (m, 6H), 2.59-2.64 (m, 4H), 2.24 ppm (s, 3H). 13C NMR (101 MHz, CDCl3): δ=154.5, 141.3, 131.4, 129.9, 129.4, 128.8, 128.2, 127.7, 125.8, 111.4, 66.4, 58.2, 55.3, 36.2, 28.0, 20.5 ppm.

### Example 16b: 4-(2-(2-benzyl-4-methylphenoxy)ethyl)thiomorpholine 1-oxide

A solution of 4-(2-(2-benzyl-4-methylphenoxy)ethyl)thiomorpholine (0.66 g, 2.02 mmol) in Ethanol (10 mL) was treated at 5° C. with NaIO₄ (0.45 g, 2.1 mmol, 1.04 equiv.) and allowed to warm to r.t. After 15 h, the mixture was filtered, diluted with EtOAc (50 mL), washed with water (50 mL), brine (50 mL), dried over MgSO₄ and concentrated under reduced pressure. The crude was purified by silica gel flash column chromatography eluting with a gradient of EtOAc in Hexanes to give 4-(2-(2-benzyl-4-methylphenoxy)ethyl)thiomorpholine 1-oxide (0.36 g, 1.05 mmol, 52% yield) as a yellow liquid. GC/MS (EI, 70 eV) m/z (%): 343 (9, [M⁺]⁺), 326 (16), 165 (12), 132 (16), 116 (100), 91 (18), 88 (26), 77 (11), 56 (32), 55 (10), 42 (11). 1H NMR (400 MHz, CDCl3): δ=7.12-7.28 (m, 5H), 6.99 (dd, J=8.2, 1.7 Hz, 1H), 6.93 (d, J=2.0 Hz, 1H), 6.74 (d, J=8.2 Hz, 1H), 4.03 (t, J=5.3 Hz, 2H), 3.94 (s, 2H), 3.07-3.16 (m, 2H), 2.81 (t, J=5.3 Hz, 2H), 2.66-2.78 (m, 6H), 2.25 ppm (s, 3H). 13C NMR (101 MHz, CDCl3): δ=154.4, 141.2, 131.6, 130.1, 129.1, 128.6, 128.2, 127.8, 125.8, 111.4, 66.7, 57.2, 46.8, 44.9, 36.2, 20.5 ppm.

### Example 17: 1-(2-(2-(4-chlorobenzyl)-4-methylphenoxy)ethyl)-4-methylpiperazine hydrochloride

Was prepared in an analogous sequence as Example 12, from 2-(4-chlorobenzyl)-4-methylphenol, ethyl 2-chloroacetate and 1-methylpiperazine to give 1-(2-(2-(4-chlorobenzyl)-4-methylphenoxy)ethyl)-4-methylpiperazine, which was precipitated as the hydrochloride according to the procedure described in Example 13, to give 1-(2-(2-(4-chlorobenzyl)-4-methylphenoxy)ethyl)-4-methylpiperazine hydrochloride as a white solid. LC-MS (ESI+): 359 ([M+H]⁺). 13C NMR (101 MHz, CDCl3): δ=152.9, 139.8, 132.6, 131.6, 131.3, 129.8, 128.8, 128.7, 127.3, 111.4, 62.8, 56.4, 49.7, 49.0, 42.9, 36.0, 20.5 ppm.

### Example 18: 1-(2-(2-benzyl-4-methylphenoxy)ethyl)-4-ethylpiperazine hydrochloride

Was prepared in an analogous sequence as Example 12, from 2-benzyl-4-methylphenol, ethyl 2-chloroacetate and 1-ethylpiperazine to give 1-(2-(2-benzyl-4-methylphenoxy)ethyl)-4-ethylpiperazine, which was precipitated as the hydrochloride according to the procedure described in Example 13, to give 1-(2-(2-benzyl-4-methylphenoxy)ethyl)-4-ethylpiperazine hydrochloride as a white solid. LC-MS (ESI+): 339 ([M+H]⁺). 1H NMR (400 MHz, DMSO-d6): δ=11.82-12.38 (m, 1H), 7.27-7.36 (m, 2H), 7.15-7.26 (m, 3H), 7.03 (dd, J=8.2, 1.8 Hz, 1H), 6.96 (d, J=1.7 Hz, 1H), 6.91 (d, J=8.3 Hz, 1H), 4.39 (br s, 2H), 3.95 (s, 2H), 3.34-3.80 (m, 10H), 3.17 (br s, 2H), 2.21 (s, 3H), 1.28 ppm (t, J=7.2 Hz, 3H).

### Example 19: 4-(2-(2-ethyl-6-(2-fluorobenzyl)-4-methylphenoxy)ethyl)morpholine

### Example 19a: 2-ethyl-4-methylphenyl 2-fluorobenzoate

A solution of 2-ethyl-4-methylphenol (4.16 g, 30.5 mmol) in DCM (30 ml) was treated at 0° C. with 2-fluorobenzoyl chloride (4.01 ml, 33.6 mmol) followed dropwise by triethylamine (4.26 ml, 30.5 mmol). The resulting mixture was stirred at r.t. for 2 h. The mixture was then poured into iced 1M aqueous HCl solution (100 mL), extracted with MTBE (2×100 mL), washed with brine (50 mL), dried over MgSO₄ and concentrated under reduced pressure to give 2-ethyl-4-methylphenyl 2-fluorobenzoate (7.9 g, 30.5 mmol, quant.) as a pale yellow liquid which was used in the next step without further purification. GC/MS (EI, 70 eV) m/z (%): 258 (6, [M⁺]⁺), 135 (2), 124 (8), 123 (100), 115 (2), 95 (17), 91 (6), 77 (3), 75 (6), 65 (2). 1H NMR (400 MHz, CDCl3): δ=8.14 (td, J=7.5, 1.8 Hz, 1H), 7.59-7.66 (m, 1H), 7.31 (td, J=7.6, 1.2 Hz, 1H), 7.24 (ddd, J=10.8, 8.3, 1.0 Hz, 1H), 7.14 (d, J=1.2 Hz, 1H), 7.07-7.09 (m, 2H), 2.62 (q, J=7.6 Hz, 2H), 2.39 (s, 3H), 1.24 ppm (t, J=7.6 Hz, 3H).

### Example 19b: (3-ethyl-2-hydroxy-5-methylphenyl)(2-fluorophenyl)methanone

A solution of 2-ethyl-4-methylphenyl 2-fluorobenzoate (1.00 g, 3.87 mmol) in Chlorobenzene (1 ml) was treated in one portion with aluminum trichloride (1.03 g, 7.7 mmol) and the mixture heated to reflux (oil bath at 150° C.) for 1 h. The resulting mixture was cooled to r.t. and poured into iced 1M aqueous HCl solution (20 mL), extracted with MTBE (3×20 mL), washed with brine (30 mL), dried over MgSO₄ and concentrated under reduced pressure to give a material that was purified by silica gel flash column chromatography eluting with a gradient of EtOAc in Hexanes to give (3-ethyl-2-hydroxy-5-methylphenyl)(2-fluorophenyl)methanone (1.0 g, 3.87 mmol, quant.) as a pale yellow liquid which was used in the next step without further purification. GC/MS (EI, 70 eV) m/z (%): 258 (82, [M⁺]₊), 257 (36), 163 (37), 147 (67), 135 (29), 134 (83), 123 (100), 95 (64), 91 (45), 75 (24).

### Example 19c: 2-ethyl-6-(2-fluorobenzyl)-4-methylphenol

A Solution of (3-ethyl-2-hydroxy-5-methylphenyl)(2-fluorophenyl)methanone (1.00 g, 3.87 mmol) in Ethanol (12 ml) was treated at 0° C. with sodium borohydride (0.146 g, 3.87 mmol) and allowed to reach r.t. After 30 minutes the mixture was poured into saturated aqueous NH₄Cl solution (50 mL), extracted with MTBE (2×100 mL), washed with brine (50 mL), dried over MgSO₄ and concentrated under reduced pressure. The resulting material was dissolved in DCM (10 ml) and treated at 0° C. with triethylsilane (1.841 ml, 11.52 mmol) followed by TFA (0.888 ml, 11.52 mmol). The resulting yellow mixture was stirred at r.t. for 15 h then poured into iced saturated aqueous NaHCO₃ solution (50 mL), extracted with MTBE (2×100 mL), washed with brine (50 mL), dried over MgSO₄ and concentrated under reduced pressure. The resulting crude was purified by silica gel flash column chromatography eluting with a gradient of EtOAc in Hexanes to give 2-ethyl-6-(2-fluorobenzyl)-4-methylphenol (0.94 g, 3.84 mmol, quant.) as a yellow liquid. GC/MS (EI, 70 eV) m/z (%): 244 (100, [M⁺]₊), 215 (25), 148 (63), 135 (41), 133 (98), 109 (61), 105 (52), 96 (48), 91 (33), 77 (24). 1H NMR (400 MHz, CDCl3): δ=7.03-7.28 (m, 4H), 6.88 (d, J=2.0 Hz, 1H), 6.83 (s, 1H), 4.63 (d, J=2.2 Hz, 1H), 3.99 (s, 2H), 2.61 (q, J=7.6 Hz, 2H), 2.26 (s, 3H), 1.25 ppm (t, J=7.6 Hz, 3H).

### Example 19d: 4-(2-(2-ethyl-6-(2-fluorobenzyl)-4-methylphenoxy)ethyl)morpholine

A solution of 2-ethyl-6-(2-fluorobenzyl)-4-methylphenol (0.939 g, 3.84 mmol) in Isopropanol (7 ml) was treated with 4-(2-chloroethyl)morpholine hydrochloride (1.073 g, 5.77 mmol) followed by KOH (0.647 g, 11.53 mmol) at r.t. The resulting pale yellow mixture was heated to reflux for 15 h then cooled to r.t., poured into dilute aqueous NH₄Cl solution (100 mL), extracted with MTBE (2×100 mL), washed with water (50 mL), brine (50 mL), dried over MgSO₄ and concentrated under reduced pressure. The resulting material was subjected to silica gel flash column chromatography eluting with a gradient of EtOAc in Hexanes to give 4-(2-(2-ethyl-6-(2-fluorobenzyl)-4-methylphenoxy)ethyl)morpholine (0.83 g, 2.33 mmol, 61% yield) as a colorless liquid. GC/MS (EI, 70 eV) m/z (%): 357 (1, [M⁺]₊), 270 (5), 114 (21), 109 (10), 101 (6), 100 (100), 70 (3), 56 (7), 55 (4), 42 (5), 28 (4). 1H NMR (400 MHz, CDCl3): δ=7.16-7.24 (m, 1H), 7.02-7.15 (m, 3H), 6.93 (d, J=1.7 Hz, 1H), 6.73 (d, J=2.0 Hz, 1H), 4.06 (s, 2H), 3.88 (t, J=5.7 Hz, 2H), 3.71-3.78 (m, 4H), 2.77 (t, J=5.7 Hz, 2H), 2.69 (q, J=7.6 Hz, 2H), 2.50-2.60 (m, 4H), 2.26 (s, 3H), 1.26 ppm (t, J=7.6 Hz, 3H).

### Example 20: 1-(2-(2-benzyl-4-chlorophenoxy)ethyl)-4-methylpiperazine hydrochloride

Was prepared in an analogous sequence as Example 12, from 2-benzyl-4-chlorophenol, ethyl 2-chloroacetate and 1-methylpiperazine to give 1-(2-(2-benzyl-4-chlorophenoxy)ethyl)-4-methylpiperazine, which was precipitated as the hydrochloride according to the procedure described in Example 13, to give 1-(2-(2-benzyl-4-chlorophenoxy)ethyl)-4-methylpiperazine hydrochloride as a white solid.

### Example 21: 1-(2-(2-benzyl-4-chlorophenoxy)ethyl)-4-ethylpiperazine hydrochloride

Was prepared in an analogous sequence as Example 12, from 2-benzyl-4-chlorophenol, ethyl 2-chloroacetate and 1-ethylpiperazine to give 1-(2-(2-benzyl-4-chlorophenoxy)ethyl)-4-ethylpiperazine, which was precipitated as the hydrochloride according to the procedure described in Example 13, to give 1-(2-(2-benzyl-4-chlorophenoxy)ethyl)-4-ethylpiperazine hydrochloride as a white solid.

### Example 22: 1-(2-(2-benzylphenoxy)ethyl)-4-methylpiperazine hydrochloride

Was prepared in an analogous sequence as Example 12, from 2-benzylphenol, ethyl 2-chloroacetate and 1-methylpiperazine to give 1-(2-(2-benzylphenoxy)ethyl)-4-methylpiperazine, which was precipitated as the hydrochloride according to the procedure described in Example 13, to give 1-(2-(2-benzylphenoxy)ethyl)-4-methylpiperazine hydrochloride as a white solid.

### Example 23: 4-(2-(2-benzyl-4-methylphenoxy)ethyl)-1,2,6-trimethylpiperazine

### Example 23a: Ethyl 2-(2-benzyl-4-methylphenoxy)acetate

A solution of 2-benzyl-4-methylphenol (3.00 g, 15.0 mmol, 1.0 equiv.) in 35 ml DMF (dimethylformamide) was treated with potassium carbonate (6.22 g, 45.0 mmol, 3.0 equiv.), followed by the addition of 2.0 ml ethyl 2-chloroacetate (2.206 g, 18.0 mmol, 1.2 equiv.). The yellow suspension was heated to 60° C. for 3 h. The reaction mixture was then poured into iced water and extracted twice with MTBE. The organic layers were washed twice with water, dried over MgSO4 and the volatiles removed under vacuum. The crude product was purified by flash column chromatography using a gradient of 0-40% of a EtOAc in hexane to afford ethyl 2-(2-benzyl-4-methylphenoxy)acetate (3.93 g, 88% yield). GC/MS (EI): m/z (%): (EI, 70 eV): 284 (62, [M⁺]+.), 197 (100), 196 (23), 195 (91), 182 (16), 181 (27), 166 (18), 165 (56), 152 (24), 91 (79).

### Example 23b: 2-(2-benzyl-4-methylphenoxy)ethan-1-ol

A 250 ml 2-neck round bottom flask was charged with ethyl 2-(2-benzyl-4-methylphenoxy)acetate (3.9 g, 13.72 mmol, 1.0 equiv) and 32 ml methanol. The mixture was cooled to 5° C. and NaBH₄ (0.315 gm 0.5 equiv, 8.32 mmol) was added and stirred for 1 h at ambient temperature. The reaction was poured onto 50 ml sat. NaHCO₃. The aqueous layer was diluted with H₂O and it was extracted with MTBE twice. The combined organic layers were dried over MgSO₄, filtered, the volatiles evaporated and the product dried under high vacuum to yield 3.59 g of 2-(2-benzyl-4-methylphenoxy)ethanol (89% yield). GC/MS (EI): m/z (%): (EI, 70 eV): 242 (24, [M⁺]+.), 198 (24), 165 (31), 152 (18), 120 (74), 92 (46), 91 (100), 77 (26), 65 (24), 45 (41).

### Example 23c: 2-benzyl-1-(2-bromoethoxy)-4-methylbenzene

In a 250 mL 2-neck round bottom flask was added CBr₄ (5.75 g, 17.33 mmol, 1.2 equiv 0-5° C. to 2-(2-benzyl-4-methylphenoxy)ethanol (3.5 g, 14.44 mmol, 1.0 equiv.) and PPh₃ (4.55 g, 17.33 mmol, 1.2 equiv) in 45 ml CH₃CN. The reaction was stirred at ambient temperature for 3 h. The volatiles were then removed and the residue was dissolved in heptane and a 4:1 MeOH/H₂O mixture. The organic layer was washed three times with the MeOH/H₂O 4:1 mixture and the aqueous layers were reextracted with heptane. The combined organic layers were dried over MgSO₄, filtered and the volatiles removed under reduced pressure. The product was then dried under high vacuum to afford 4.88 g of 2-benzyl-1-(2-bromoethoxy)-4-methylbenzene (90% purity, 100% yield) which was used without further purification. GC/MS (EI): m/z (%): (EI, 70 eV): 306 (50), 304 (51, [M⁺]+.), 197 (100), 165 (48), 154 (36), 153 (34), 152 (41), 120 (88), 117 (46), 91 (65).

### Example 23d: 4-(2-(2-benzyl-4-methylphenoxy)ethyl)-1,2,6-trimethylpiperazine

To a suspension of NaH (0.15 g, 3.28 mmol, 1.2 equiv.) in 3 ml DMF was added 1,2,6-trimethylpiperazine (0.35 g, 2.72 mmol, 1.0 equiv.) in 1 ml DMF at 3° C., the solution was stirred at ambient temperature for 20 min. 2-benzyl-1-(2-bromoethoxy)-4-methylbenzene (1.0 g, 3.28 mmol, 1.2 equiv.) in 2 ml DMF was added at 3° C. and the reaction mixture further stirred at ambient temperature for 5 h. The mixture was then cooled to 3° C., diluted with sat. NaHCO₃, and poured onto 25 ml H₂O. The product was extracted twice with EtOAc and washed with H₂O. The organic layers were dried over MgSO₄, and the volatiles evaporated. Purification by flash column chromatography using a gradient of 10-100% of a mixture of EtOAc/EtOH/NH₄OH (90:9:1) in hexane afforded 4-(2-(2-benzyl-4-methylphenoxy)ethyl)-1,2,6-trimethylpiperazine (0.37 g, 32% yield) as a pale yellow liquid. ¹H NMR (500 MHz, CDCl₃) δ (ppm)=7.32-7.22 (m, 4H), 7.21-7.15 (m, 1H), 7.02-6.96 (m, 1H), 6.93 (d, J=2.1 Hz, 1H), 6.77 (d, J=8.2 Hz, 1H), 4.07 (t, J=5.8 Hz, 2H), 3.96 (s, 2H), 2.88-2.81 (m, 2H), 2.72 (t, J=5.8 Hz, 2H), 2.31-2.24 (m, 2H), 2.29 (s, 3H), 2.26 (s, 3H), 2.05 (t, J=11.0 Hz, 2H), 1.08 (d, J=6.4 Hz, 6H). ¹³C NMR (101 MHz, CDCl₃) δ (ppm)=154.5, 141.3, 131.2, 129.8, 129.6, 128.9, 128.2, 127.6, 125.7, 111.6, 66.5, 61.7, 57.9, 57.1, 37.9, 36.0, 20.5, 18.3. GC/MS (EI): m/z (%): (EI, 70 eV): 352 (1, [M⁺]+.), 155 (66, [M⁺]+.), 142 (10), 141 (100), 126 (18), 98 (9), 91 (10), 84 (27), 57 (16), 56 (23), 42 (18).

### Example 24: 2-benzyl-4-methylphenyl trifluoromethanesulfonate

In a 250 ml 3-neck round bottom flask, a solution of 2-benzyl-4-methylphenol (3.20 g, 16.14 mmol, 1.0 equiv) and a catalytic amount of DMAP (N,N-dimethyl-4-aminopyridine, 0.3 g, 2.421 mmol, 0.15 equiv.) in 54 ml CH₂Cl₂ was cooled to 0° C. 3.4 ml (24.21 mmol, 1.5 equiv.) of Et₃N were added, followed by dropqise addition of 4.1 ml triflic anhydride (6.83 g, 24.21 mmol, 1.5 equiv) via a syringe, resulting in a dark-red solution. The ice-bath was then removed, and the reaction was stirred at ambient temperature for 2 h. Then, the reaction mixture was concentrated, and rediluted in CH₂Cl₂ and the volatiles re-evaporated (in total three times). The crude material was then purified by column chromatography using a gradient of 0-15% of EtOAc in cyclohexane to afford 2-benzyl-4-methylphenyl trifluoromethanesulfonate (4.83, 95% purity, 86% yield). GC/MS (EI): m/z (%): (EI, 70 eV): 330 (53, [M⁺]+.), 197 (100), 182 (24), 181 (29), 169 (56), 165 (31), 154 (47), 153 (32), 152 (33), 91 (38).

### Example 25: N,2-dibenzyl-4-methylaniline

Cs₂CO₃ (1.97 g, 6.05 mmol, 2.0 equiv.) 0.29 g (S)-BINAP (0.29 g, 0.45 mmol, 0.15 equiv) and Pd(OAc)₂ (70 mg, 0.303 mmol, 0.1 equiv.) were added to a solution of 2-benzyl-4-methylphenyl trifluoromethanesulfonate (1.00 g, 3.03 mmol, 1.0 equiv.) benzylamine (0.97 g, 9.08 mmol, 3.0 equiv.) in 15 ml toluene. The resulting red suspension was then purged with nitrogen at ambient and then heated to 100° C. under vigorous stirring. After 3 hours the reaction mixture was cooled to ambient temperature. The mixture was then diluted with EtOAc and poured onto H₂O, washed with 12 ml 1 M HCl, H₂O and brine. The aqueous layer was reextracted with EtOAc and the organic layers were dried over MgSO₄, filtered and the volatiles evaporated. The crude material was then purified by column chromatography using a gradient of 0-100% of EtOAc in heptane to afford 0.67 g (76% yield) of N,2-dibenzyl-4-methylaniline. GC/MS (EI): m/z (%): (EI, 70 eV): 288 (10), 287 (41, [M⁺]+.), 197 (16), 196 (100), 194 (21), 181 (33), 180 (37), 165 (11), 91 (66), 65 (16).

### Example 26: 2-benzyl-4-methylaniline

In a 250 ml 3-neck round bottom flask, 150 mg Pd/C was added to N,2-dibenzyl-4-methylaniline in 25 mL THF (tetrahydrofurane). The resulting solution was purged three times with argon, then another three times using a balloon filled with H₂. The reaction was then left to stir at ambient temperature, under a hydrogen atmosphere from a freshly filled balloon. The mixture was then filtered over Celite and washed with hexane. The volatiles of the mother liquor were evaporated and the crude product dried under high vacuum. Purification by column chromatography using a gradient of 0-20% of EtOAc in heptane afforded 2-benzyl-4-methylaniline (0.8 g, 78% yield). GC/MS (EI): m/z (%): (EI, 70 eV): 198 (15), 197 (100, [M⁺]+.), 196 (25), 182 (46), 181 (17), 180 (22), 165 (26), 120 (34), 106 (19), 91 (19).

### Example 27: N-(2-benzyl-4-methylphenyl)-2-bromoacetamide

2-bromoacetyl bromide (0.20 g, 1.0 mmol, 1.0 equiv) in 3 mL Et₂O was added at 5° C. to 2-benzyl-4-methylaniline (0.20 g, 1.0 mmol, 1.0 equiv.) in 3 mL Et₂O, whereupon a white solid precipitated. The reaction was stirred at ambient temperature for 2 h. Then, the suspension was dissolved in CH₂Cl₂ and washed twice with HCl (2M), and reextracted with CH₂Cl₂. The organic layers were dried over MgSO₄, filtered and the volatiles removed under vacuum. The crude was suspended in hexane, the solvent filtered off and the solid further washed with hexane. Drying of the off-white solid material yielded 0.25 g of N-(2-benzyl-4-methylphenyl)-2-bromoacetamide (77% yield). GC/MS (EI): m/z (%): (EI, 70 eV): 317 (38, [M⁺]+.), 224 (55), 196 (100), 194 (63), 181 (51), 180 (81), 160 (83), 146 (57), 142 (38), 91 (77).

### Example 28: N-(2-benzyl-4-methylphenyl)-2-morpholinoacetamide

In a 100 mL 3-neck round-bottom flask, a mixture of N-(2-benzyl-4-methylphenyl)-2-bromoacetamide (0.35 g, 1.163 mmol, 1.0 equiv), morpholine (1.0 mL, 1.1013 g, 11.63 mmol, 10 equiv.) and K₂CO₃ (0.32 g, 2.325 mmol, 2.0 equiv.) in 20 mL of toluene was refluxed overnight. The reaction mixture was then cooled to ambient temperature, filtered, and the volatiles removed under reduced pressure. The crude product was then purified by flash column chromatography using a gradient of 10-100% of EtOAc in heptane to afford N-(2-benzyl-4-methylphenyl)-2-morpholinoacetamide (0.35 g, 93% yield) as an off-white solid. GC/MS (EI): m/z (%): (EI, 70 eV): 324 (2, [M⁺]+.), 194 (3), 180 (3), 101 (7), 100 (100), 91 (3), 86 (3), 70 (3), 56 (10), 42 (5).

### Example 29: 2-benzyl-4-methyl-N-(2-morpholinoethyl)aniline

A 250 mL 3-neck round-bottom flask was charged with LiAlH₄ (0.16 g, 4.32 mmol, 4.0 equiv.) and 4 mL THF and cooled to 3-8° C. N-(2-benzyl-4-methylphenyl)-2-morpholinoacetamide (0.35 g, 1.1 mmol, 1.0 equiv) in 6 ml THF was added dropwise before heating to 60° C. for 3 hours. As the reaction did not go to completion another 3 equivalents (120 mg) of LiAlH₄ were added at ambient temperature before heating the reaction again overnight to 60° C. The reaction was cooled to 3° C., and 0.3 mL H₂O were added slowly, followed by 0.3 mL of NaOH (NaOH) and another 0.9 mL of H₂O. The ice-bath was then removed and the mixture stirred for 10 min at ambient temperature, before adding sufficient MgSO₄ and stirring another 5 min. The mixture was the filtered and the volatiles evaporated. Purification by flash column chromatography using a gradient of 40-100% of EtOAc in heptane afforded 2-benzyl-4-methyl-N-(2-morpholinoethyl)aniline (0.28 g, 84% yield) as an off-white solid. ¹H NMR (400 MHz, CDCl₃) δ (ppm)=7.35-7.28 (m, 2H), 7.27-7.19 (m, 3H), 7.07-7.00 (m, 1H), 6.94 (d, J=2.0 Hz, 1H), 6.59 (d, J=8.1 Hz, 1H), 4.17 (br s, 1H), 3.92 (s, 2H), 3.55 (t, J=4.4 Hz, 4H), 3.11 (t, J=5.6 Hz, 2H), 2.57 (dd, J=5.3, 6.5 Hz, 2H), 2.35 (t, J=4.4 Hz, 4H), 2.29 (s, 3H). ¹³C NMR (101 MHz, CDCl₃) δ (ppm)=144.2, 139.8, 131.6, 128.6, 128.4, 128.1, 126.3, 126.2, 124.8, 110.9, 66.9, 57.0, 53.2, 40.2, 38.0, 20.4. GC/MS (EI): m/z (%): (EI, 70 eV): 310 (6, [M⁺]+.), 211 (6), 210 (33), 132 (9), 117 (7), 105 (4), 101 (18), 100 (100), 91 (8), 56 (10).

### Example 30: 2-benzyl-N,4-dimethyl-N-(2-morpholinoethyl)aniline

Paraformaldehyde (0.275 g, 8.7 mmol, 10 equiv.) was added at ambient temperature to 2-benzyl-4-methyl-N-(2-morpholinoethyl)aniline (0.27 g, 0.87 mmol, 1.0 equiv) in 6 ml AcOH (acetic acid) and the mixture was stirred at ambient temperature for 1 h. Sodium cyanoborohydride (0.26 g, 4.17 mmol, 4.8 equiv.) was added portionwise and the reaction was stirred over night. The reaction was poured onto 40 ml icecold NaOH (2 M) and extracted twice with DCM, washed with 40 ml pH 7 phosphate buffer, dried over MgSO₄, and the volatiles evaporated. Purification by flash column chromatography using a gradient of 10-100% of a mixture of EtOAc/EtOH/NH₄OH (90:9:1) in hexane afforded 2-benzyl-N,4-dimethyl-N-(2-morpholinoethyl)aniline (0.22 g, 78% yield) as a pale yellow liquid. ¹H NMR (400 MHz, CDCl₃) δ (ppm)=7.22-7.29 (m, 2H), 7.13-7.20 (m, 3H), 7.08 (d, J=8.1 Hz, 1H), 7.00 (dd, J=8.2, 1.8 Hz, 1H), 6.91 (d, J=2.0 Hz, 1H), 4.05 (s, 2H), 3.64 (m, 4H), 2.93-3.04 (m, 2H), 2.61 (s, 3H), 2.37-2.41 (m, 2H), 2.33-2.36 (m, 4H), 2.24 (s, 3H). ¹³C NMR (101 MHz, CDCl₃) δ (ppm)=149.6, 141.9, 136.8, 133.5, 131.6, 129.0, 128.2, 127.7, 125.6, 121.3, 66.9, 56.9, 54.4, 54.0, 43.8, 36.6, 20.9. GC/MS (EI): m/z (%): (EI, 70 eV): 324 (2, [M⁺]+.), 225 (18), 224 (100), 208 (6), 165 (8), 132 (12), 131 (4), 117 (4), 100 (24), 91 (29), 56 (7).

### Example 31: 2-benzyl-4-methyl-N-(2-(4-methylpiperazin-1-yl)ethyl)aniline

Prepared according to Example 29 replacing morpholine with N-Methylpiperazine. N-(2-benzyl-4-methylphenyl)-2-(4-methylpiperazin-1-yl)acetamide (0.88 g, 2.61 mmol, 1.0 equiv.) in 20 ml THF was added at 3-8° C. to LiAlH₄ (0.44 g, 11.47 mmol, 4.4 equiv.) in 25 ml THF. The reaction mixture was stirred at ambient temperature for 2 h, and further stirred at 60° C. for 7 h. The reaction mixture was then cooled with an ice bath and 0.5 ml H₂O were added slowly at 5° C., followed by 0.5 ml 3M NaOH, then again 1.5 ml H₂O was added to the mixture. The mixture was stirred for 10 minutes at ambient temperature, followed by the addition of MgSO₄ and further stirring for 5 min. After filtration, the volatiles were removed under reduced pressure. Purification by flash column chromatography using a gradient of 10-100% of a mixture of EtOAc/EtOH/NH₄OH (90:9:1) in hexane afforded 2-benzyl-4-methyl-N-(2-(4-methylpiperazin-1-yl)ethyl)aniline (0.7 g, 87% purity, 72% yield). ¹H NMR (400 MHz, CDCl₃) δ (ppm)=7.32-7.27 (m, 2H), 7.24-7.18 (m, 3H), 7.01 (dd, J=1.7, 8.1 Hz, 1H), 6.90 (d, J=2.0 Hz, 1H), 6.61-6.54 (m, 1H), 4.20 (br s, 1H), 3.92-3.85 (m, 2H), 3.09 (t, J=5.9 Hz, 2H), 2.56 (t, J=5.9 Hz, 2H), 2.52-2.30 (m, 8H), 2.27 (s, 3H), 2.27 (s, 3H). ¹³C NMR (101 MHz, CDCl₃) δ (ppm)=144.3, 139.8, 131.4, 128.6, 128.5, 128.1, 126.2, 126.0, 124.9, 110.9, 56.5, 55.0, 52.7, 46.0, 40.6, 38.0, 20.4. GC/MS (EI): m/z (%): (EI, 70 eV): 323 (3, [M⁺]+.), 223 (14), 210 (8), 132 (6), 117 (5), 114 (16), 113 (100), 91 (8), 70 (37), 43 (7), 42 (10).

### Example 32: 2-benzyl-N,4-dimethyl-N-(2-(4-methylpiperazin-1-yl)ethyl)aniline

Prepared according to Example 30 replacing morpholine with N-Methylpiperazine. To a solution of 2-benzyl-4-methyl-N-(2-(4-methylpiperazin-1-yl)ethyl)aniline (0.17 g, 0.53 mmol, 1.0 equiv.) in 4 mL AcOH was added paraformaldehyde (0.17 g, 5.26 mmol, 10.0 equiv.) and the mixture was stirred at ambient temperature for 1 h. Then NaBH₃CN (0.16 g, 2.52 mmol, 4.8 equiv.) was added portionwise and the reaction was stirred at ambient temperature overnight. The reaction was then poured onto 40 mL ice-cold 2M NaOH (pH 14) and extracted twice with CH₂Cl₂, the organic layers were then washed with 30 mL of pH 7 phosphate buffer (K2HPO4 buffer/KH2PO4 buffer 2:1), dried over MgSO₄, filtered and the volatiles removed under reduced pressure. Purification by flash column chromatography using a gradient of 10-100% of a mixture of EtOAc/EtOH/NH₄OH (90:9:1) in heptane afforded 2-benzyl-N,4-dimethyl-N-(2-(4-methylpiperazin-1-yl)ethyl)aniline (0.14 g, 79% yield) as a yellow liquid. ¹H NMR (400 MHz, CDCl₃) δ (ppm)=7.24-7.31 (m, 2H), 7.16-7.23 (m, 3H), 7.10 (d, J=8.1 Hz, 1H), 7.02 (dd, J=8.1, 2.0 Hz, 1H), 6.93 (d, J=2.0 Hz, 1H), 4.08 (s, 2H), 2.97-3.04 (m, 2H), 2.63 (s, 3H), 2.40-2.45 (m, 2H), 2.33-2.51 (br m, 8H), 2.27-2.29 (m, 3H), 2.26-2.27 (m, 3H). ¹³C NMR (101 MHz, CDCl₃) δ (ppm)=149.7, 141.9, 136.8, 133.4, 131.5, 129.0, 128.2, 127.6, 125.6, 121.3, 56.5, 55.1, 54.7, 53.4, 46.0, 43.8, 36.6, 20.9. GC/MS (EI): m/z (%): (EI, 70 eV): 337 (1, [M⁺]+.), 237 (23), 225 (17), 224 (92), 165 (10), 132 (14), 113 (100), 91 (30), 70 (45), 43 (11), 42 (15).

### Example 33: N-(2,6-difluorobenzyl)-4-methylaniline

In a 100 mL 3-neck round-bottom flask, p-toluidine (8.6 g, 80 mmol, 4.0 equiv.), NaHCO₃ (2.11 g, 25 mmol, 1.25 equiv.) and 2 mL H₂O were heated to 95° C. 2,6-difluoro benzylchloride (3.26 g, 20 mmol, 1.0 equiv.) was added portionwise over 45 min at 95° C. The brown mixture was then allowed to stir at 95° C. for 2 h and subsequently cooled to 50° C. The hot mixture was filtered and washed with EtOAc and brine. The organic layer was dried over MgSO₄, filtered and the volatiles evaporated, and further dried under high vacuum. Purification by flash column chromatography using a gradient of 0-10% of EtOAc in heptane afforded N-(2,6-difluorobenzyl)-4-methylaniline (4.32 g, 92% yield) as a yellow liquid. GC/MS (EI): m/z (%): (EI, 70 eV): 233 (48, [M⁺]+.), 232 (21), 127 (100), 120 (17), 107 (9), 106 (31), 101 (10), 91 (10), 79 (13), 77 (24).

### Example 34: 2-(2,6-difluorobenzyl)-4-methylaniline

In a 15 mL Ace pressure tube was placed N-(2,6-difluorobenzyl)-4-methylaniline (1.0 g, 4.29 mmol, 1.0 equiv.) and 1.2 mL p-toluidine. The mixture was heated to 190° C., then AlCl₃ (0.17 g, 1.29 mmol, 0.3 equiv.) was added at 190° C., whereupon the solution turned to green-black. The reaction was heated to 200° C. overnight after which the resulting dark blue mixture was cooled to ambient temperature. The mixture was poured into an excess NaOH (2M) and was extracted with EtOAc twice, then washed with brine. The combined organic layers were dried over MgSO₄, filtered and the volatiles evaporated. Purification by flash column chromatography using a gradient of 0-10% of EtOAc in heptane afforded 2-(2,6-difluorobenzyl)-4-methylaniline (0.31 g, 20% yield) as a red solid. GC/MS (EI): m/z (%): (EI, 70 eV): 234 (15), 233 (100, [M⁺]+.), 232 (15), 218 (12), 212 (39), 198 (31), 127 (27), 120 (25), 106 (47), 77 (18).

### Example 35: 2-bromo-N-(2-(2,6-difluorobenzyl)-4-methylphenyl)acetamide

Prepared according to Example 27 replacing 2-benzyl-4-methylaniline with 2-(2,6-difluorobenzyl)-4-methylaniline. GC/MS (EI): m/z (%): (EI, 70 eV): 355 (33), 353 (30, [M⁺]+.), 274 (24), 260 (50), 212 (100), 211 (25), 146 (46), 127 (55), 93 (21), 42 (30).

### Example 36: N-(2-(2,6-difluorobenzyl)-4-methylphenyl)-2-morpholinoacetamide

Prepared according to Example 28 replacing N-(2-benzyl-4-methylphenyl)-2-bromoacetamide with 2-bromo-N-(2-(2,6-difluorobenzyl)-4-methylphenyl)acetamide. GC/MS (EI): m/z (%): (EI, 70 eV): 360 (2, [M⁺]+.), 212 (4), 127 (4), 101 (6), 100 (100), 86 (3), 70 (3), 56 (6), 43 (2), 42 (6), 28 (3).

### Example 37: 2-(2,6-difluorobenzyl)-4-methyl-N-(2-morpholinoethyl)aniline

Prepared according to Example 29 replacing N-(2-benzyl-4-methylphenyl)-2-morpholinoacetamide with N-(2-(2,6-difluorobenzyl)-4-methylphenyl)-2-morpholinoacetamide (0.72 g, 2.0 mmol, 1.0 equiv.) affording 0.22 g (32% yield) of 2-(2,6-difluorobenzyl)-4-methyl-N-(2-morpholinoethyl)aniline. 1H NMR (400 MHz, CDCl₃) δ (ppm)=7.21 (tt, J=6.4, 8.4 Hz, 1H), 6.96 (dd, J=1.5, 8.5 Hz, 1H), 6.90 (t, J=7.9 Hz, 2H), 6.80 (s, 1H), 6.57 (d, J=8.3 Hz, 1H), 4.53 (br s, 1H), 3.85 (s, 2H), 3.77-3.71 (m, 4H), 3.19 (t, J=5.9 Hz, 2H), 2.74-2.66 (m, 2H), 2.56-2.46 (m, 4H), 2.21 (s, 3H). 13C NMR (101 MHz, CDCl₃) δ (ppm)=161.7, 143.8, 141.1, 130.0, 128.0, 126.2, 122.9, 115.4, 111.3, 110.9, 67.1, 57.1, 53.4, 40.6, 23.8, 20.5. GC/MS (EI): m/z (%): (EI, 70 eV): 346 (5, [M⁺]+.), 247 (5), 246 (30), 245 (4), 132 (6), 127 (9), 117 (6), 101 (19), 100 (100), 56 (5).

### Example 38: 2-(2,6-difluorobenzyl)-N,4-dimethyl-N-(2-morpholinoethyl)aniline

Prepared according to Example 30 replacing benzyl-4-methyl-N-(2-morpholinoethyl)aniline with 2-(2,6-difluorobenzyl)-4-methyl-N-(2-morpholinoethyl)aniline (0.21 g, 0.61 mmol, 1.0 equiv.) affording 0.184 g (84% yield) of 2-(2,6-difluorobenzyl)-N,4-dimethyl-N-(2-morpholinoethyl)aniline. 1H NMR (400 MHz, CDCl₃) δ (ppm)=7.24-7.15 (m, 1H), 7.09 (d, J=8.1 Hz, 1H), 7.00 (dd, J=1.7, 8.1 Hz, 1H), 6.94-6.84 (m, 2H), 6.71 (s, 1H), 4.12 (s, 2H), 3.76-3.66 (m, 4H), 3.14-3.04 (m, 2H), 2.67 (s, 3H), 2.44 (s, 6H), 2.21 (s, 3H). 13C NMR (101 MHz, CDCl₃) δ (ppm)=161.9, 149.1, 135.2, 133.7, 129.5, 127.7, 127.6, 121.3, 117.0, 111.1, 66.9, 56.9, 54.0, 44.2, 23.2, 21.0. GC/MS (EI): m/z (%): (EI, 70 eV): 360 (1, [M⁺]+.), 261 (18), 260 (100), 244 (4), 201 (4), 133 (4), 132 (18), 128 (3), 127 (43), 117 (4), 100 (19).

### Example 39: N-(2,6-difluorobenzyl)-2,4-dimethylaniline

Prepared according to Example 33 replacing p-toluidine with 2,4-dimethylaniline. GC/MS (EI): m/z (%): (EI, 70 eV): MS (EI, 70 eV): 248 (6), 247 (35, [M⁺]+.), 246 (7), 134 (7), 127 (35), 121 (9), 120 (100), 118 (5), 91 (12), 77 (14).

### Example 40: 2-(2,6-difluorobenzyl)-4,6-dimethylaniline

Prepared according to Example 34 replacing N-(2,6-difluorobenzyl)-4-methylaniline with N-(2,6-difluorobenzyl)-2,4-dimethylaniline. GC/MS (EI): m/z (%): (EI, 70 eV): 248 (16), 247 (100, [M⁺]+.), 246 (13), 232 (20), 226 (29), 212 (33), 134 (20), 127 (15), 120 (63), 91 (14).

### Example 41: 2-bromo-N-(2-(2,6-difluorobenzyl)-4,6-dimethylphenyl)acetamide

Prepared according to Example 27 replacing 2-benzyl-4-methylaniline with 2-(2,6-difluorobenzyl)-4,6-dimethylaniline. ¹H NMR (400 MHz, CDCl₃) δ (ppm)=7.88-7.76 (m, 1H), 7.19 (tt, J=6.6, 8.3 Hz, 1H), 0.00 (d, J=12.7 Hz, 2H), 0.00 (t, J=7.8 Hz, 2H), 4.11 (s, 2H), 3.93 (s, 2H), 3.87 (s, 1H), 2.26 (s, 3H), 2.21 (s, 3H).

### Example 42: N-(2-(2,6-difluorobenzyl)-4,6-dimethylphenyl)-2-morpholinoacetamide

Prepared according to Example 28 replacing N-(2-benzyl-4-methylphenyl)-2-bromoacetamide with 2-bromo-N-(2-(2,6-difluorobenzyl)-4,6-dimethylphenyl)acetamide. ¹H NMR (400 MHz, CDCl₃) δ (ppm)=8.70 (s, 1H), 7.18 (tt, J=8.3, 6.6 Hz, 1H), 6.93 (s, 1H), 6.84-6.91 (m, 3H), 3.91 (s, 2H), 3.74-3.79 (m, 4H), 3.20-3.27 (m, 2H), 2.68-2.74 (m, 4H), 2.23 (s, 3H), 2.18 (s, 3H)¹³C NMR (101 MHz, CDCl₃) δ (ppm)=168.9, 161.4 (2), 137.3, 135.7, 135.3, 130.4, 130.0, 128.3, 128.0 (2), 111.3 (2), 67.1 (2), 62.1, 54.2 (2), 24.5, 21.0, 18.8.

### Example 43: 2-(2,6-difluorobenzyl)-4,6-dimethyl-N-(2-morpholinoethyl)aniline

Prepared according to Example 29 replacing benzyl-4-methyl-N-(2-morpholinoethyl)aniline with N-(2-(2,6-difluorobenzyl)-4,6-dimethylphenyl)-2-morpholinoacetamide (0.34 g, 0.91 mmol, 1.0 equiv.) affording 0.143 g (43% yield) of 2-(2,6-difluorobenzyl)-4,6-dimethyl-N-(2-morpholinoethyl)aniline. GC/MS (EI): m/z (%): (EI, 70 eV): 360 (6, [M⁺]+.), 261 (9), 260 (44), 259 (16), 131 (6), 127 (11), 101 (14), 100 (100), 56 (8), 42 (7).

### Example 44: 2-(2,6-difluorobenzyl)-N,4,6-trimethyl-N-(2-morpholinoethyl)aniline

Prepared according to Example 30 replacing 2-benzyl-4-methylaniline with 2-(2,6-difluorobenzyl)-4,6-dimethylaniline (0.14 g, 0.39 mmol, 1.0 equiv.) afford 0.104 g (70% yield) of 2-(2,6-difluorobenzyl)-N,4,6-trimethyl-N-(2-morpholinoethyl)aniline. ¹H NMR (400 MHz, CDCl₃) δ (ppm)=7.13-7.26 (m, 1H), 6.91 (t, J=7.8 Hz, 2H), 6.83 (d, J=1.0 Hz, 1H), 6.53 (s, 1H), 4.04-4.18 (m, 2H), 3.73 (t, J=4.7 Hz, 4H), 2.81 (s, 3H), 2.37-2.61 (m, 8H), 2.30 (s, 3H), 2.18 (s, 3H). ¹³C NMR (101 MHz, CDCl₃) δ (ppm)=161.7 (2), 146.1, 137.1, 134.9, 134.8, 130.5, 127.6, 127.0, 117.3, 111.1 (2), 66.8 (2), 58.0, 53.9 (2), 53.0, 40.8, 24.3, 20.9, 19.2. GC/MS (EI): m/z (%): (EI, 70 eV): 374 (1, [M⁺]+.), 275 (20), 274 (100), 201 (4), 146 (14), 131 (5), 127 (14), 100 (35), 56 (7), 42 (7), 28 (5).

### Example 45: N-(2-fluorobenzyl)-4-methylaniline

Prepared according to Example 33 replacing 2,6-difluoro benzylchloride with 2-fluoro benzylchloride. GC/MS (EI): m/z (%): (EI, 70 eV): 215 (44, [M⁺]+.), 214 (19), 120 (19), 110 (8), 109 (100), 106 (16), 91 (11), 83 (14), 79 (8), 77 (16).

### Example 46: 2-(2-fluorobenzyl)-4-methylaniline

Prepared according to Example 34 replacing N-(2,6-difluorobenzyl)-4-methylaniline with N-(2-fluorobenzyl)-4-methylaniline. GC/MS (EI): m/z (%): (EI, 70 eV): 216 (15), 215 (100, [M⁺]+.), 200 (17), 194 (52), 183 (16), 180 (29), 120 (30), 109 (18), 106 (30), 77 (16).

### Example 47: 2-bromo-N-(2-(2-fluorobenzyl)-4-methylphenyl)acetamide

Prepared according to Example 27 replacing 2-benzyl-4-methylaniline with 2-(2-fluorobenzyl)-4-methylaniline. GC/MS (EI): m/z (%): (EI, 70 eV): 337 (28), 335 (28, [M⁺]+.), 242 (50), 198 (29), 194 (100), 146 (43), 123 (29), 121 (32), 109 (52), 42 (27).

### Example 48: N-(2-(2-fluorobenzyl)-4-methylphenyl)-2-morpholinoacetamide

Prepared according to Example 28 replacing N-(2-benzyl-4-methylphenyl)-2-bromoacetamide with 2-bromo-N-(2-(2-fluorobenzyl)-4-methylphenyl)acetamide. GC/MS (EI): m/z (%): (EI, 70 eV): 342 (3, [M⁺]+.), 194 (4), 146 (3), 109 (3), 101 (6), 100 (100), 70 (2), 56 (6), 42 (5), 28 (3).

### Example 49: 2-(2-fluorobenzyl)-4-methyl-N-(2-morpholinoethyl)aniline

Prepared according to Example 29 replacing benzyl-4-methyl-N-(2-morpholinoethyl)aniline with N-(2-(2-fluorobenzyl)-4-methylphenyl)-2-morpholinoacetamide (0.38 g, 1.11 mmol, 0.1 equiv.) affording 0.092 g (25% yield) of 2-(2-fluorobenzyl)-4-methyl-N-(2-morpholinoethyl)aniline. GC/MS (EI): m/z (%): (EI, 70 eV): 328 (5, [M⁺]+.), 229 (5), 228 (28), 132 (6), 117 (6), 109 (8), 105 (4), 101 (18), 100 (100), 56 (7).

### Example 50: 2-(2-fluorobenzyl)-N,4-dimethyl-N-(2-morpholinoethyl)aniline

Prepared according to Example 30 replacing benzyl-4-methyl-N-(2-morpholinoethyl)aniline with 2-(2-fluorobenzyl)-4-methyl-N-(2-morpholinoethyl)aniline (0.37 g, 0.013 mmol, 1.0 equiv.) affording 0.38 g (98% yield) of 2-(2-fluorobenzyl)-N,4-dimethyl-N-(2-morpholinoethyl)aniline. ¹H NMR (400 MHz, CDCl₃) δ (ppm)=7.22-7.14 (m, 1H), 7.12-7.09 (m, 1H), 7.08-7.00 (m, 4H), 6.92-6.87 (m, 1H), 4.09 (s, 2H), 3.70-3.64 (m, 4H), 3.04-2.98 (m, 2H), 2.63 (s, 3H), 2.44-2.35 (m, 6H), 2.26 (s, 3H)¹³C NMR (101 MHz, CDCl₃) δ (ppm)=161.2, 149.6, 135.7, 133.6, 131.2, 131.1, 128.7, 127.9, 127.4, 123.8, 121.4, 115.1, 66.9, 56.9, 54.4, 54.0, 43.8, 29.5, 20.9 GC/MS (EI): m/z (%): (EI, 70 eV): 342 (2, [M⁺]+.), 243 (19), 242 (100), 226 (5), 183 (8), 132 (16), 131 (4), 117 (4), 109 (34), 100 (25), 56 (4).

### Example 51: N-(2-fluorobenzyl)-2,4-dimethylaniline

Prepared according to Example 33 replacing 2,6-difluoro benzylchloride with 2-fluoro benzylchloride and p-toluidine with 2,4-dimethylaniline. GC/MS (EI): m/z (%): (EI, 70 eV): 229 (54, [M⁺]+.), 228 (12), 134 (14), 121 (10), 120 (100), 118 (10), 109 (96), 91 (19), 83 (17), 77 (20).

### Example 52: 2-(2-fluorobenzyl)-4,6-dimethylaniline

Prepared according to Example 34 replacing N-(2,6-difluorobenzyl)-4-methylaniline with N-(2-fluorobenzyl)-2,4-dimethylaniline and p-toluidine with 2,4-dimethylaniline. GC/MS (EI): m/z (%): (EI, 70 eV): 230 (16), 229 (100, [M⁺]+.), 214 (18), 208 (32), 194 (32), 134 (24), 120 (34), 109 (24), 91 (17), 77 (11).

### Example 53: 2-bromo-N-(2-(2-fluorobenzyl)-4,6-dimethylphenyl)acetamide

Prepared according to Example 27 replacing 2-benzyl-4-methylaniline with 2-(2-fluorobenzyl)-4,6-dimethylaniline. GC/MS (EI): m/z (%): (EI, 70 eV): 351 (38), 349 (36, [M⁺]+.), 256 (86), 212 (31), 208 (100), 160 (77), 123 (38), 121 (39), 109 (63), 42 (32).

### Example 54: N-(2-(2-fluorobenzyl)-4,6-dimethylphenyl)-2-morpholinoacetamide

Prepared according to Example 28 replacing N-(2-benzyl-4-methylphenyl)-2-bromoacetamide with 2-bromo-N-(2-(2-fluorobenzyl)-4,6-dimethylphenyl)acetamide. GC/MS (EI): m/z (%): (EI, 70 eV): 356 (3, [M⁺]+.), 208 (3), 109 (3), 101 (6), 100 (100), 86 (2), 70 (3), 56 (6), 42 (4), 28 (2).

### Example 55: 2-(2-fluorobenzyl)-4,6-dimethyl-N-(2-morpholinoethyl)aniline

Prepared according to Example 29 replacing benzyl-4-methyl-N-(2-morpholinoethyl)aniline with N-(2-(2-fluorobenzyl)-4,6-dimethylphenyl)-2-morpholinoacetamide (1.0 g, 2.8 mmol, 1.0 equiv.) affording 0.47 g (47% yield) of 2-(2-fluorobenzyl)-4,6-dimethyl-N-(2-morpholinoethyl)aniline. GC/MS (EI): m/z (%): (EI, 70 eV): 342 (6, [M⁺]+.), 243 (10), 242 (58), 241 (21), 183 (6), 146 (7), 131 (11), 109 (16), 101 (14), 100 (100), 56 (8).

### Example 56: 2-(2-fluorobenzyl)-N,4,6-trimethyl-N-(2-morpholinoethyl)aniline

Prepared according to Example 30 replacing benzyl-4-methyl-N-(2-morpholinoethyl)aniline with 2-(2-fluorobenzyl)-4,6-dimethyl-N-(2-morpholinoethyl)aniline (0.46 g, 1.34 mmol, 1.0 equiv.) affording 0.38 g (79% yield) of 2-(2-fluorobenzyl)-N,4,6-trimethyl-N-(2-morpholinoethyl)aniline. ¹H NMR (400 MHz, CDCl₃) δ (ppm)=7.21-7.14 (m, 1H), 7.04 (d, J=9.0 Hz, 1H), 7.03-6.99 (m, 2H), 6.87 (d, J=2.0 Hz, 1H), 6.74 (d, J=1.5 Hz, 1H), 4.07 (s, 2H), 3.68 (t, J=4.6 Hz, 4H), 3.22-3.02 (m, 2H), 2.69 (s, 3H), 2.50-2.40 (m, 2H), 2.41-2.34 (m, 4H), 2.29 (s, 4H), 2.23 (s, 3H). ¹³C NMR (101 MHz, CDCl₃) δ (ppm)=161.1, 146.9, 138.9, 137.4, 134.9, 130.9, 130.7, 129.1, 128.9, 127.3, 123.8, 115.0, 67.0, 58.0, 54.0, 53.3, 40.9, 30.7, 20.8, 19.2. GC/MS (EI): m/z (%): (EI, 70 eV): 356 (1, [M⁺]+.), 257 (19), 256 (100), 240 (4), 183 (8), 146 (14), 131 (5), 109 (15), 100 (32), 56 (6), 42 (4).

### Example 57: N-(2-bromobenzyl)-2,4-dimethylaniline

Prepared according to Example 33 replacing 2,6-difluoro benzylchloride with 2-bromo benzylchloride and p-toluidine with 2,4-dimethylaniline. GC/MS (EI): m/z (%): (EI, 70 eV): 291 (28), 289 (28, [M⁺]+.), 171 (36), 169 (38), 134 (26), 120 (100), 91 (27), 90 (22), 89 (19), 77 (23).

### Example 58: 2-(2-bromobenzyl)-4,6-dimethylaniline

Prepared according to Example 34 replacing N-(2,6-difluorobenzyl)-4-methylaniline N-(2-bromobenzyl)-2,4-dimethylaniline and p-toluidine with 2,4-dimethylaniline. GC/MS (EI): m/z (%): (EI, 70 eV): 291 (39), 289 (38, [M⁺]+.), 210 (91), 208 (75), 195 (47), 194 (100), 180 (32), 97 (21), 96 (34), 91 (21).

### Example 59: 2-(2-bromobenzyl)-4,6-dimethyl-N-(2-morpholinoethyl)aniline

2-(2-bromobenzyl)-4,6-dimethylaniline (2.2 g, 7.58 mmol, 1.0 equiv.), 4-(2-bromoethyl)morpholine (1.471, 7.58 mmol, 1.0 equiv.) and tetrabutylammoniumiodide (0.56 g, 1.516 mmol, 0.2 equiv.) were combined in 40 mL DMF (dimethylformamide). 3.2 mL of N-ethyldiisopropylamine (2.45 g, 18.95 mmol, 2.5 equiv.) and the reaction was heated overnight. The reaction mixture was then cooled to ambient temperature, poured onto an aqueous saturated NaHCO₃ solution and the product was extracted twice with EtOAc. The combined organic layers were washed three times with H₂O, dried over MgSO₄, filtered and the volatiles evaporated. Purification by flash column chromatography using a gradient 0-10% of EtOAc in afforded 0.55 g (17% yield) of 2-(2-bromobenzyl)-4,6-dimethyl-N-(2-morpholinoethyl)aniline. GC/MS (EI): m/z (%): (EI, 70 eV): 402 (4, [M⁺]+.), 302 (15), 222 (17), 100 (100).

### Example 60: 2-(2-bromobenzyl)-N,4,6-trimethyl-N-(2-morpholinoethyl)aniline

Prepared according to Example 30 replacing benzyl-4-methyl-N-(2-morpholinoethyl)aniline with 2-(2-bromobenzyl)-4,6-dimethyl-N-(2-morpholinoethyl)aniline (0.54 g, 1.34 mmol, 1.0 equiv.) to afford 0.42 g (69% yield) of 2-(2-bromobenzyl)-N,4,6-trimethyl-N-(2-morpholinoethyl)aniline. GC/MS (EI): m/z (%): (EI, 70 eV): 416 (1, [M⁺]+.), 319 (17), 318 (94), 317 (19), 316 (100), 207 (14), 179 (18), 178 (17), 146 (20), 100 (93), 56 (15).

### Example 61: 2-(3,5-dimethyl-2-(methyl(2-morpholinoethyl)amino)benzyl)benzonitrile

In a 25 mL 3-neck round-bottom flask, 2-(2-bromobenzyl)-N,4,6-trimethyl-N-(2-morpholinoethyl)aniline (50 mg, 0.12 mmol, 1.0 equiv) and copper(I) cyanide (13 mg, 0.14 mmol, 1.2 equiv.) were suspended in 4 mL DMF (dimethylformamide) and the suspension was heated overnight to reflux. After cooling to room temperature, the mixture was quenched with aqueous saturated NH₄Cl and extracted twice with EtOAc. The organic layers were dried over MgSO₄, filtered and the volatiles removed under reduced pressure. Purification by flash column chromatography using a gradient of 0-10% of a mixture of EtOAc/EtOH/NH₄OH (90:9:1) in heptane afforded 16 mg (37% yield) of 2-(3,5-dimethyl-2-(methyl(2-morpholinoethyl)amino)benzyl)benzonitrile as an orange viscous liquid. ¹H NMR (400 MHz, CDCl₃) δ (ppm)=7.65 (dd, J=1.1, 7.7 Hz, 1H), 7.43 (dt, J=1.5, 7.7 Hz, 1H), 7.27 (dt, J=1.0, 7.6 Hz, 1H), 7.08 (d, J=7.8 Hz, 1H), 6.89 (d, J=1.7 Hz, 1H), 6.74 (d, J=1.5 Hz, 1H), 4.26 (d, J=7.6 Hz, 2H), 3.73-3.58 (m, 4H), 3.21-2.92 (m, 2H), 2.63 (s, 3H), 2.33 (br s, 4H), 2.28 (s, 3H), 2.25 (s, 3H), 2.19-2.03 (m, 2H). ¹³C NMR (101 MHz, CDCl₃) δ (ppm)=146.8, 146.5, 138.3, 137.6, 135.1, 132.6, 132.5, 131.4, 129.7, 129.2, 126.2, 118.4, 112.7, 66.9, 57.9, 54.0, 53.1, 41.0, 36.7, 20.8, 19.3. GC/MS (EI): m/z (%): (EI, 70 eV): 363 (0, [M⁺]+.), 264 (20), 263 (100), 247 (4), 190 (5), 146 (12), 131 (5), 116 (4), 100 (38), 56 (6), 42 (6).

### Example 62: 1-allyl-2-benzyl-4-methylbenzene

Pd(PPh₃)₄ (0.5 g, 0.30 mmol, 0.1 equiv.), LiCl (0.22 g, 1.50 mmol, 1.5 equiv.) and 1.2 mL allyltributylstannane (12 g, 3.60 mmol, 1.2 equiv.) were added to 2-benzyl-4-methylphenyl trifluoromethanesulfonate in 30 mL DMF, and the resulting yellow suspension was purged with nitrogen at ambient temperature. The reaction mixture was then heated to 90° C. overnight. The mixture was diluted with H₂O and EtOAc and filtered over Celite, and further washed with heptane. The organic layer was separated and washed with H₂O, and the aqueous layers were reextracted with heptane. The organic layers were dried over MgSO₄, filtered and the volatiles removed under reduced pressure. Purification by flash column chromatography using a gradient of 0-10% of EtOAc in heptane afforded 1-allyl-2-benzyl-4-methylbenzene in quantitative yield as a light yellow liquid. GC/MS (EI): m/z (%): (EI, 70 eV): 222 (57, [M⁺]+.), 193 (72), 179 (44), 178 (77), 165 (47), 131 (55), 129 (100), 128 (47), 115 (47), 91 (59).

### Example 63: 2-(2-benzyl-4-methylbenzyl)oxirane

3-chloroperbenzoic acid (1.21 g, 5.4 mmol, 1.2 equiv.) was added at 3° C. to 1-allyl-2-benzyl-4-methylbenzene in 22 mL CH₂Cl₂, and the reaction was stirred at ambient temperature for 2.5 hours upon which the mixture was diluted with H₂O and EtOAc and was filtered over Celite, then washed with heptane. The organic layer was separated and the aqueous layer was reextracted with heptane. The organic layers were dried over MgSO4, filtered and the volatiles removed under reduced pressure. Purification by flash column chromatography using a gradient of 0-10% of EtOAc in heptane afforded 2-(2-benzyl-4-methylbenzyl)oxirane in quantitative yield as a light yellow liquid. GC/MS (EI): m/z (%): (EI, 70 eV): 238 (1, [M⁺]+.), 220 (76, [M⁺]+.), 205 (75), 193 (55), 192 (38), 179 (91), 178 (100), 165 (77), 129 (54), 115 (70), 91 (71).

### Example 64: 1-(2-benzyl-4-methylphenyl)-3-(4-methylpiperazin-1-yl)propan-2-ol

2-(2-benzyl-4-methylbenzyl)oxirane (0.46 g, 1.93 mmol, 1.0 equiv.) was dissolved in 10 mL EtOH and 1-methylpiperazine (0.21 g, 2.085 mmol, 1.1 equiv.) was added. The solution was stirred at 70° C. (oil bath) for 20 h. The reaction was then cooled to ambient temperature and the volatiles removed. Purification by flash column chromatography using a gradient 10-100% of a mixture of EtOAc/EtOH/NH₄OH (90:9:1) in heptane afforded 1-(2-benzyl-4-methylphenyl)-3-(4-methylpiperazin-1-yl)propan-2-ol (0.45 g, 69% yield) as a yellow liquid. ¹H NMR (400 MHz, CDCl₃) δ (ppm)=7.12-7.18 (m, 2H), 7.03-7.09 (m, 2H), 6.98-7.03 (m, 2H), 6.91 (dd, J=7.7, 1.3 Hz, 1H), 6.85-6.88 (m, 1H), 3.95 (d, J=26.7 Hz, 1H), 3.88 (d, J=26.4 Hz, 1H), 3.54-3.65 (m, 1H), 2.66 (dd, J=14.2, 7.3 Hz, 1H), 2.51 (dd, J=14.2, 5.4 Hz, 1H), 2.39-2.47 (m, 2H), 2.21-2.38 (m, 5H), 2.20 (s, 3H), 2.15 (s, 3H), 2.12 (d, J=6.1 Hz, 1H), 2.10 (d, J=1.2 Hz, 1H). ¹³C NMR (100 MHz, CDCl₃) δ (ppm)=141.1, 138.6, 136.0, 133.8, 131.6, 130.6, 128.7, 128.4, 127.3, 125.9, 66.8, 63.5, 55.2, 46.0, 39.1, 37.7, 21.0. GC/MS (EI): m/z (%): (EI, 70 eV): 338 (0, [M⁺]+.), 178 (5), 165 (6), 115 (5), 114 (48), 113 (100), 91 (8), 70 (30), 56 (5), 43 (7), 42 (8).

### Example 65: 1-(2-benzyl-4-methylphenyl)-3-(4-methylpiperazin-1-yl)propan-2-one

A 25 mL 3-neck round-bottom flask was charged with 1-(2-benzyl-4-methylphenyl)-3-(4-methylpiperazin-1-yl)propan-2-ol (0.15 g, 044 mmol, 1.0 equiv.), 4-methylmorpholine N-oxide (80 mg, 0.665 mmol, 1.5 equiv.) and 0.28 g of 4 Å molecular sieves in 3 mL CH₂Cl₂. To this solution, tetrapropylammonium perruthenate (8 mg, 0.022 mmol, 0.05 equiv.) was added. The resulting brown solution was allowed to stir at ambient temperature for 40 minutes. The reaction mixture was then filtered using a short silicagel pad and washed with a mixture of EtOAc/EtOH/NH4OH 90:9:1. The filtrate was concentrated under reduced pressure and dried under high vacuum. Purification by flash column chromatography using a gradient 10-100% of a mixture of EtOAc/EtOH/NH₄OH (90:9:1) in heptane afforded 61 mg of a mixture containing a 1:1 mixture of 1-(2-benzyl-4-methylphenyl)-3-(4-methylpiperazin-1-yl)propan-2-ol along with the starting material as a yellow liquid. 3 mg of the title compound were isolated using preparative RP-HPLC (reverse phase high pressure liquid chromatography) on a Kinetex C18 column (5 μm particle size, 4.6×150 mm, equipped with a pre-column), and an eluent system consisting of A (CH₃CN:H₂O, 5:95+0.1% trifluoroacetic acid) and B (ACN:H₂O, 95:5+0.085% trifluoroacetic acid) using a gradient from 10% to 100% B with 10% B/min. ¹H NMR (500 MHz, DMSO-d6) δ (ppm)=7.27 (t, J=7.2 Hz, 2H), 7.19 (t, J=7.6 Hz, 1H), 7.10 (d, J=7.3 Hz, 2H), 7.04 (d, J=7.9 Hz, 1H), 7.01 (d, J=7.6 Hz, 1H), 6.96 (s, 1H), 3.86 (s, 2H), 3.73 (s, 2H), 3.30-3.41 (m, 4H), 2.96-3.07 (m, 2H), 2.82-2.91 (m, 2H), 2.77 (s, 3H), 2.39-2.48 (m, 2H), 2.25 (s, 3H). ¹³C NMR (151 MHz, DMSO-d6) δ ppm=205.8, 140.8, 139.6, 136.3, 131.6, 131.3, 130.5, 129.1 (2), 128.9 (2), 127.5, 126.4, 65.1, 53.0 (2), 49.6 (2), 44.3 42.6, 38.7, 21.1. GC/MS (EI): m/z (%): MS (EI, 70 eV): 336 (2, [M⁺]+.), 179 (3), 178 (4), 165 (4), 114 (7), 113 (100), 98 (4), 70 (27), 56 (3), 43 (2), 42 (5).

### Example 66: (E)-3-(2-benzyl-4-methylphenyl)acrylic acid

2-benzyl-4-methylbenzaldehyde (1.0 g, 3.8 mmol, 1.0 equiv.), malonic acid (1.0 g, 9.5 mmol, 2.5 equiv.) and 80 mL piperidine in 4 mL pyridine were heated to 105° C. until no more CO₂ formation was observed (4 hours). The reaction was then poured into 40 ml ice-cold aqueous 2M HCl under continuous stirring. The resulting suspension (pH 1) was filtered and the solid (E)-3-(2-benzyl-4-methylphenyl)acrylic acid was washed with 25 ml HCl (2M). The solid was then transferred to a round-bottom flask and dried at ambient temperature and 0.10 mbar overnight. The product (1.2 g, 62% purity, 78% yield) was used without further purification in the next step. GC/MS (EI): m/z (%): (EI, 70 eV): 252 (9, [M⁺]+.), 206 (29), 193 (24), 192 (100), 191 (37), 178 (32), 161 (54), 129 (22), 128 (21), 115 (34), 91 (22).

### Example 67: (E)-3-(2-benzyl-4-methylphenyl)-1-(piperidin-1-yl)prop-2-en-1-one

(E)-3-(2-benzyl-4-methylphenyl)acrylic acid (0.38 g, 1.5 mmol, 1.2 equiv) was dissolved in 0.35 mL SOCl₂ (0.59 g, 5.0 mmol, 4.0 equiv.) and stirred for 1 hour at room temperature under an inert atmosphere. The reaction mixture was concentrated in vacuum and the residue was diluted with 5 mL CH₂Cl₂, and addet at 5° C. to 1-methylpiperazine (0.13 g, 1.25 mmol, 1.0 equiv.) and 0.4 mL Et₃N (2.5 mmol, 2.0 equiv.) in 10 mL CH₂Cl₂. The reaction was stirred at ambient temperature for 5 min and next, the brown solution was heated to reflux for 2 h. After cooling to ambient temperature, the reaction was poured onto an aqueous saturated NaHCO₃ solution and extracted twice with CH₂Cl₂ The combined organic layers were dried over MgSO₄, filtered and the volatiles removed under reduced pressure. Purification by flash column chromatography using a gradient 10-100% of a mixture of EtOAc/EtOH/NH₄OH (90:9:1) in heptane afforded (E)-3-(2-benzyl-4-methylphenyl)-1-(4-methylpiperazin-1-yl)prop-2-en-1-one (0.32 g, 38% yield) as a viscous yellow liquid.

LC/MS (ESI): 335 (M+H)+, 357 (M+Na)+, 669 (2M+H)+.

### Example 68: (E)-1-(3-(2-benzyl-4-methylphenyl)allyl)-4-methylpiperazine and 1-(3-(2-benzyl-4-methylphenyl)propyl)-4-methylpiperazine

(E)-3-(2-benzyl-4-methylphenyl)-1-(4-methylpiperazin-1-yl)prop-2-en-1-one (0.3 g, 0.9 mmol, 1.0 equiv.) in 3 mL THF was added slowly to a suspension of LiAlH4 (37 mg, 1.0 mmol, 1.1 equiv.) in 5 mL THF. The reaction was stirred at ambient temperature for 45 min. After this, the reaction mixture was cooled to 3° C., and 0.1 mL H₂O were added slowly followed by 0.1 mL NaOH (3M) and another 0.3 mL H₂O. The white suspension was stirred 20 minutes at ambient temperature, then MgSO4 was and stirring was continued for another 5 minutes. The mixture was then filtered and the volatiles evaporated. Purification by flash column chromatography using a gradient 20-100% of a mixture of EtOAc/EtOH/NH₄OH (90:9:1) in heptane afforded 96 mg of a 4:1 mixture of (E)-1-(3-(2-benzyl-4-methylphenyl)allyl)-4-methylpiperazine and 1-(3-(2-benzyl-4-methylphenyl)propyl)-4-methylpiperazine. Major: GC/MS (EI): m/z (%): (EI, 70 eV): 320 (64, [M⁺]+.), 179 (25), 158 (29), 128 (28), 99 (46), 91 (100), 70 (27), 56 (34), 43 (23), 42 (25) Minor: GC/MS (EI): m/z (%): (EI, 70 eV): 322 (17, [M⁺]+.), 178 (10), 165 (10), 127 (10), 113 (100), 100 (13), 91 (12), 70 (25), 43 (19), 42 (13)

### Example 69: 1-(3-(2-benzyl-4-methylphenyl)propyl)-4-methylpiperazine

A sample of pure 1-(3-(2-benzyl-4-methylphenyl)propyl)-4-methylpiperazine was obtained by separation of the above mentioned mixture by preparative HPLC. ¹H NMR (400 MHz, CHLOROFORM-d) δ ppm 7.38 (d, J=7.8 Hz, 1H), 7.24 (t, J=7.6 Hz, 2H), 7.06-7.19 (m, 3H), 6.99-7.05 (m, 1H), 6.95 (s, 1H), 6.67 (d, J=15.6 Hz, 1H), 6.04 (dt, J=15.6, 6.8 Hz, 1H), 4.02 (s, 2H), 3.06 (dd, J=6.8, 1.2 Hz, 2H), 2.52-2.57 (m, 2H), 2.30 (s, 3H), 2.28 (s, 3H). ¹³C NMR (101 MHz, CHLOROFORM-d) δ ppm 140.8, 137.4, 137.3, 133.5, 131.3, 130.8, 128.6 (2), 128.37 (2), 127.4, 125.8, 61.3, 55.2 (2), 53.1 (2), 46.1, 39.2, 21.2.

### Example 70: 1-(3-(2-benzyl-4-methylphenyl)propyl)-4-methylpiperazine

76 mg of a 4:1 mixture of (E)-1-(3-(2-benzyl-4-methylphenyl)allyl)-4-methylpiperazine and 1-(3-(2-benzyl-4-methylphenyl)propyl)-4-methylpiperazine in 10 ml EtOH was hydrogenated with 40 mg Pd/C at ambient temperature for 6 h. The mixture was then filtered through celite and the celite pad was washed thoroughly with ethanol. The filtrate was evaporated at 50° C./20 mbar. Purification by flash column chromatography using a gradient 10-100% of a mixture of EtOAc/EtOH/NH₄OH (90:9:1) in heptane afforded 1-(3-(2-benzyl-4-methylphenyl)propyl)-4-methylpiperazine (36 mg, 43% yield) as a colorless liquid. ¹H NMR (400 MHz, CDCl₃) δ (ppm)=7.25 (t, J=7.3 Hz, 2H), 7.16 (t, J=7.3 Hz, 1H), 7.11 (d, J=7.8 Hz, 2H), 7.06 (d, J=7.8 Hz, 1H), 6.99 (d, J=7.9 Hz, 1H), 6.92 (s, 1H), 3.99 (s, 2H), 2.54 (t, J=8.0 Hz, 2H), 2.18-2.52 (m, 10H), 2.28 (s, 3H), 2.27 (s, 3H), 1.65 (br quin, J=7.6 Hz, 2H). ¹³C NMR (101 MHz, CDCl₃) δ (ppm)=141.12, 138.09, 137.54, 135.42, 131.28, 129.31, 128.67 (2), 128.37 (2), 127.30, 125.88, 58.23, 55.21 (2), 53.20 (2), 46.11, 38.87, 30.22, 28.27, 21.01. GC/MS (EI): m/z (%): MS (EI, 70 eV): 322 (18, [M⁺]+.), 178 (10), 127 (11), 113 (100), 100 (13), 91 (12), 70 (28), 58 (10), 43 (20), 42 (14).

### Example 71: (E)-2-benzyl-1-(2-methoxyvinyl)-4-methylbenzene

5.7 mL (5.7 mmol, 1.2 equiv.) of potassium tert-butoxide (1M in THF) was added at 5-15° C. to methoxymethyl triphenylphosphoniumchloride (1.96 g, 5.71 mmol, 1.2 equiv) in 15 mL THF. The resulting bright red mixture was stirred for 15 min at ambient temperature. Then 2-benzyl-4-methylbenzaldehyde (1.0 g, 4.76 mmol, 1.0 equiv.) 10 mL THF was added reaction was stirred at ambient temperature for 45 min. The reaction mixture was then diluted with heptane and washed three times with a 4:1 MeOH/H₂O mixture. The aqueous layers were reextracted with heptane. The combined organic layers were dried over MgSO₄, filtered and the volatiles evaporated to afford 1.0 g of (E)-2-benzyl-1-(2-methoxyvinyl)-4-methylbenzene (71% yield, 80% purity) as a red liquid, which was used without further purification in the next step. GC/MS (EI): m/z (%): MS (EI, 70 eV): 238 (79, [M⁺]+.), 206 (100), 205 (38), 193 (44), 191 (63), 179 (35), 178 (93), 165 (50), 115 (39), 91 (44).

### Example 72: 2-(2-benzyl-4-methylphenyl)acetaldehyde

Perchloric acid (0.3 mL, 3.3 mmol, 1.0 equiv.) was added at 5° C. (E)-2-benzyl-1-(2-methoxyvinyl)-4-methylbenzene (0.79 g, 3.3 mmol, 1.0 equiv.) in 15 mL Et₂O. The reaction was stirred at 5° C. for 5 minutes. After this, the mixture was diluted with MTBE (methyl tert-butyl ether), poured onto 2M NaOH and washed with brine. The aqueous layers were reextracted with MTBE. The combined organic layers were dried over MgSO₄, filtered and the volatiles removed under reduced pressure. Purification by flash column chromatography using a gradient 0-10% of EtOAc in heptane afforded 2-(2-benzyl-4-methylphenyl)acetaldehyde (0.57 g, 67% purity, 51% yield) as a yellow liquid.

GC/MS (EI): m/z (%): MS (EI, 70 eV): 224 (43, [M⁺]+.), 206 (38), 195 (73), 181 (40), 180 (46), 179 (41), 178 (60), 166 (35), 165 (100), 91 (34).

### Example 73: 1-(2-benzyl-4-methylphenethyl)-4-methylpiperazine

A 100 mL 3-neck round-bottom flask was charged with 2-(2-benzyl-4-methylphenyl)acetaldehyde (0.57 g, 2.54 mmol, 1.0 equiv.) 1-methylpiperazine (0.28 g, 2.80 mmol, 1.1 equiv.) in 20 mL CH₂Cl₂. Sodium triacetoxyborohydride (0.60 g, 2.8 mmol, 1.1 equiv.) was added in portions in order to keep the inner temperature below 30° C. The light yellow mixture was stirred overnight at ambient temperature. The reaction mixture was then cooled to 5° C. and 20 mLaqueous saturated NaHCO₃ was added dropwise. The mixture was extracted with DCM and the organic layers were washed with water, dried over MgSO₄ and concentrated. Purification by flash column chromatography using a gradient 10-100% of mixture of EtOAc/EtOH/NH4OH (90:9:1) in heptane afforded 1-(2-benzyl-4-methylphenethyl)-4-methylpiperazine (0.29 mg, 29% yield) as a colorless liquid. ¹H NMR (400 MHz, CDCl₃) δ (ppm)=7.25 (br t, J=7.8 Hz, 2H), 7.17 (br t, J=7.3 Hz, 1H), 7.08-7.12 (m, 3H), 7.00 (dd, J=7.8, 1.3 Hz, 1H), 6.94 (s, 1H), 3.99 (s, 2H), 2.71-2.76 (m, 2H), 2.30-2.54 (m, 10H), 2.28 (s, 6H). ¹³C NMR (101 MHz, CDCl₃) δ (ppm)=140.98, 138.35, 135.80, 129.83, 128.67 (2) 128.40 (2), 127.41, 128.68, 59.85, 55.12 (2), 53.06 (2), 46.07, 38.95, 29.87, 21.00. GC/MS (EI): m/z (%): MS (EI, 70 eV): 308 (1, [M⁺]+.), 179 (4), 178 (4), 165 (5), 114 (8), 113 (100), 91 (5), 70 (29), 56 (4), 43 (4), 42 (8).

### Example 74: 2-benzyl-4-methylphenyl 2-chloroacetate

A solution of 2-benzyl-4-methylphenol (2.0 g, 10.1 mmol, 1.0 equiv) and a catalytic amount of DMAP (0.06 g, 0.5 mmol, 0.05 equiv.) in 7.0 mL Et₃N (50.4 mmol, 5 equiv.) was cooled to 0° C. 2-chloroacetyl chloride (1.0 mL, 1.37 g, 21.1 mmol, 1.2 equiv.) was added dropwise upon which the color turned to dark-brown. The ice bath was removed and the reaction was allowed to stirred at ambient temperature over night. The reaction mixture was then diluted with MTBE, poured onto H₂O and extracted twice with MTBE. The combined organic layers were washed with brine, dried over MgSO₄, filtered and the volatiles evaporated. Purification by flash column chromatography using a gradient 0-20% of EtOAc in heptane afforded 2-benzyl-4-methylphenyl 2-chloroacetate (0.75 g, 88% purity, 34% yield) as a yellow liquid. GC/MS (EI): m/z (%): MS (EI, 70 eV): 274 (22, [M⁺]+.), 198 (93), 197 (95), 183 (28), 181 (23), 165 (41), 153 (24), 152 (29), 120 (100), 91 (43), 77 (33).

### Example 75: 2-benzyl-4-methylphenyl 2-(4-methylpiperazin-1-yl)acetate

2-benzyl-4-methylphenyl 2-chloroacetate (0.75 g, 2.73 mmol, 1.0 equiv.) in 5 mL CH₃CN was added dropwise to a mixture of 1-methylpiperazine (0.55 g, 5.46 mmol, 2.0 equiv.), KI (0.91 g, 5.46 mmol, 2.0 equiv.), potassium carbonate (0.76 g, 5.46 mmol, 2.0 equiv.), and sodium hydrogencarbonate (0.46 g, 5.46 mmol, 2.0 equiv.) in 10 mL CH₃CN. The light yellow suspension was stirred at ambient temperature overnight. The reaction was then diluted with EtOAc, filtered and the volatiles evaporated. Purification by flash column chromatography using a gradient 10-100% of mixture of EtOAc/EtOH/NH₄OH (90:9:1) in heptane afforded 2-benzyl-4-methylphenyl 2-(4-methylpiperazin-1-yl)acetate (0.55 g, 60% yield) as a viscous yellow liquid. ¹H NMR (400 MHz, CDCl₃) δ (ppm)=7.29-7.25 (m, 2H), 7.20 (s, 1H), 7.18-7.12 (m, 2H), 7.10-7.04 (m, 1H), 7.00 (d, J=1.7 Hz, 1H), 6.99-6.95 (m, 1H), 3.88 (s, 2H), 3.34 (s, 2H), 2.72-2.56 (m, 4H), 2.56-2.44 (m, 4H), 2.31 (s, 6H). ¹³C NMR (101 MHz, CDCl₃) δ (ppm)=168.7, 146.5, 139.9, 135.8, 132.2, 131.7, 128.7, 128.5, 128.1, 126.2, 122.2, 59.0, 54.8, 52.9, 46.0, 36.4, 20.9. GC/MS (EI): m/z (%): MS (EI, 70 eV): 338 (4, [M⁺]+.), 165 (4), 114 (9), 113 (100), 98 (5), 91 (4), 71 (4), 70 (31), 56 (5), 43 (5), 42 (12).

### Example 76: (8-benzylchroman-2-yl)methyl 4-methylbenzenesulfonate

In a 250 ml 3-neck round-bottom flask, a solution of 2.20 g (6.58 mmol, 1.0 equiv.) of (8-hydroxychroman-2-yl)methyl 4-methylbenzenesulfonate and 1.6 mL (19.74 mmol, 3.0 equiv.) of pyridine in 45 mL CH₂Cl₂ was treated with triflic anhydride (2.2 mL, 3.71 g, 13.16 mmol, 2.0 equiv.) at 0° C. The ice bath was removed and the reaction was stirred at ambient temperature for 2 h. The reaction was then poured onto 50 mL 0.5 M HCl and was extracted twice with CH₂Cl₂. The combined organic layers were washed with brine, dried over MgSO₄, filtered and the volatiles evaporated. Drying of the crude under high vacuum afforded crude (8-(((trifluoromethyl)sulfonyl)oxy)chroman-2-yl)methyl 4-methylbenzenesulfonate (2.55 g, 83% yield) which was used in the next step without further purification. GC/MS (EI): m/z (%): MS (EI, 70 eV): 466 (0, [M⁺]+.), 161 (17), 155 (18), 133 (12), 105 (13), 92 (15), 91 (100), 77 (13), 69 (20), 65 (41), 39 (13).

### Example 77: (8-(((trifluoromethyl)sulfonyl)oxy)chroman-2-yl)methyl 4-methylbenzenesulfonate

A mixture of benzylboronic acid pinacol ester (0.45 g, 2.1 mmol, 3.0 equiv.), (8-(((trifluoromethyl)sulfonyl)oxy)chroman-2-yl)methyl 4-methylbenzenesulfonate (0.32 g, 0.67 mmol, 1.0 equiv.), K₃PO₄ (0.29 g, 1.37 mmol, 2.0 equiv.), SPhos (2-dicyclohexylphosphino-2′,6′-dimethoxybiphenyl, 62 mg, 0.15 mmol, 0.22 equiv.), and Pd(OAc)₂ (23 mg, 0.1 mmol, 0.15 equiv.) in 5.5 mL toluene/H₂O 10:1 was purged with nitrogen for 10 min. The reaction was then heated to 110° C. for 2 hours. After cooling to room temperature, the mixture was partitioned between EtOAc (20 mL) and brine. The aqueous layer was reextracted with EtOAc and the organic layers were dried over MgSO₄, filtered and the volatiles evaporated. Purification by flash column chromatography using a gradient 10-100% of EtOAc in heptane afforded (8-benzylchroman-2-yl)methyl 4-methylbenzenesulfonate acetate (0.25 g, 86% yield) as a viscous, yellow liquid. GC/MS (EI): m/z (%): MS (EI, 70 eV): 408 (5, [M⁺]+.), 236 (20), 235 (13), 223 (13), 195 (19), 194 (12), 178 (24), 165 (19), 145 (55), 91 (100), 65 (17).

### Example 78: 1-((8-benzylchroman-2-yl)methyl)-4-methylpiperazine

1-Methylpiperazine (0.18 g, 1.76 mmol, 3.0 equiv.) in 1.5 mL THF was added at ambient temperature to (8-benzylchroman-2-yl)methyl 4-methylbenzenesulfonate (0.24 g, 0.59 mmol, 1.0 equiv.) in 3.5 mL DMSO (dimethylsulfoxide). The resulting mixture was stirred at 65° C. overnight Then, the reaction mixture was cooled to ambient temperature, partitioned between EtOAc (20 mL) and water (3×20 mL). The aqueous layers were reextracted with EtOAc and the organic layers were dried over MgSO₄, filtered and the volatiles evaporated. Purification by flash column chromatography using a gradient 10-100% of mixture of EtOAc/EtOH/NH₄OH (90:9:1) in heptane afforded (8-benzylchroman-2-yl)methyl 4-methylbenzenesulfonate (125 mg, 95% purity, 60% yield) as a viscous, yellow liquid. ¹H NMR (400 MHz, CDCl₃) δ (ppm)=7.19-7.27 (m, 4H), 7.15 (br t, J=6.8 Hz, 1H), 6.86-6.93 (m, 2H), 6.74 (t, J=7.5 Hz, 1H), 4.12-4.22 (m, 1H), 3.92 (d, J=15.6 Hz, 1H), 3.91 (d, J=15.6 Hz, 1H), 2.81-2.92 (m, 1H), 2.33-2.78 (m, 12H), 2.27 (s, 3H), 1.96-2.05 (m, 1H), 1.70-1.82 (m, 1H). ¹³C NMR (101 MHz, CDCl₃) δ (ppm)=152.5, 141.2, 128.9 (3), 128.8, 128.2 (2), 127.7, 125.7, 121.7, 119.7, 74.7, 62.9, 55.2, 53.9, 46.1, 35.9, 26.3, 24.8. GC/MS (EI): m/z (%): MS (EI, 70 eV): 336 (2, [M⁺]+.), 165 (4, [M⁺]+.), 139 (20), 114 (8), 113 (100), 98 (4), 91 (11), 70 (28), 56 (5), 43 (7), 42 (9).

### Example 79: 2-(2,6-difluorobenzyl)-4,6-dimethylphenol

To the mixture of 2-(chloromethyl)-1,3-difluorobenzene 1 (3.0 g, 18.5 mmol) and 2,4-dimethylphenol (6.8 g, 55.4 mmol) under Ar was added zinc(II) chloride (0.5 g, 3.7 mmol) and the resulting mixture was heated to 140° C. for 5 hours. After the reaction mixture was cooled to rt., water (100 mL) was added to quench the solution and MTBE (100 mL×3) was used to extract the solution. The combined organic phase was washed with water (100 mL), dried over MgSO₄ and concentrated by rotary evaporation. The obtained liquid was further concentrated by kugelrohr distillation (0.2 mbar, 120° C.) to remove unconsumed starting material 2,4-dimethylphenol and the residue was purified by column chromatography on silica gel (PE:MTBE=92:8) to get the title product (1.6 g, yield 32%) as a viscous liquid. GC/MS (EI): m/z (%): 248 (11) [M⁺], 233 (25), 215 (10), 201 (4), 183 (11), 134 (50), 121 (20), 106 (18), 91 (20), 77 (10).

### Example 80: 4-(2-(2-(2,6-difluorobenzyl)-4,6-dimethylphenoxy)ethyl)morpholine

The mixture of 2-(2,6-difluorobenzyl)-4,6-dimethylphenol (0.37 g, 1.5 mmol), 4-(2-chloroethyl)morpholine hydrochloride (0.56 g, 3.0 mmol), potassium iodide (0.025 g, 0.15 mmol) and potassium carbonate (2.10 g, 14.9 mmol) in DMF (20 mL) was heated to reflux for 3 hours. After the mixture was cooled to rt., water (80 mL) was added, dichloromethane (50 mL×3) was used to extract the solution, the combined organic phase was washed with water (50 mL) and dried over MgSO₄. After the solvent was removed, the residue was purified by column chromatography on silica gel (PE:MTBE=3:2) to get the title product (0.40 g, yield 73%) as a viscous liquid. GC/MS (EI): m/z (%): 361 (11) [M⁺], 274 (20), 227 (7), 201 (4), 183 (4), 127 (7), 114 (28), 100 (100), 85 (1), 70 (4). ¹H NMR (300 MHz, CDCl₃) δ 7.26-7.14 (m, 1H), 6.95-6.82 (m, 3H), 6.54 (s, 1H), 4.07 (s, 2H), 3.97 (t, J=5.8 Hz, 2H), 3.83-3.69 (m, 4H), 2.84 (t, J=5.8 Hz, 2H), 2.69-2.53 (m, 4H), 2.29 (s, 3H), 2.17 (s, 3H). ¹³C NMR (75 MHz, CDCl₃) δ 163.6 (q), 163.5 (q), 160.3 (q), 160.2 (q), 153.3 (q), 133.4 (q), 131.5 (q), 130.5 (q), 130.3 (t), 128.2 (t), 128.0 (t), 127.9 (t), 127.4 (t), 116.6 (q), 116.4 (q), 116.1 (q), 111.5 (t), 111.4 (t), 111.2 (t), 111.1 (t), 69.9 (d), 67.1 (d), 58.8 (d), 54.4 (d), 22.4 (d), 22.3 (d), 22.3 (d), 20.9 (s), 16.4 (s).

### Example 81: 4-(2-(2-(2,6-difluorobenzyl)-4-methylphenoxy)ethyl)morpholine

Following the general procedure from Example 80: The mixture of 2-(2,6-difluorobenzyl)-4-methylphenol (0.25 g, 1.1 mmol), 4-(2-chloroethyl)morpholine hydrochloride (0.40 g, 2.1 mmol), potassium carbonate (1.50 g, 10.7 mmol) and potassium iodide (0.035 g, 0.20 mmol) in DMF (13 mL) were reacted to give the title product (0.30 g, yield 65%) as a viscous liquid. GC/MS (EI): m/z (%): 347 (8) [M⁺], 260 (4), 213 (4), 201 (4), 165 (2), 127 (7), 114 (8), 100 (100), 85 (1), 70 (4). ¹H NMR (300 MHz, CDCl₃) δ 7.23-7.11 (m, 1H), 6.96-6.82 (m, 3H), 6.80-6.71 (m, 2H), 4.11 (t, J=5.8 Hz, 2H), 3.99 (s, 2H), 3.73 (t, J=5.8 Hz, 4H), 2.82 (t, J=5.8 Hz, 2H), 2.59 (t, J=5.8 Hz, 4H), 2.21 (s, 3H). ¹³C NMR (75 MHz, CDCl₃) δ 163.6 (q), 163.5 (q), 160.4 (q), 160.2 (q), 154.4 (q), 130.2 (t), 129.9 (q), 127.9 (t), 127.9 (t), 127.8 (t), 127.7 (t), 127.3 (q), 116.4 (q), 111.4 (t), 111.3 (t), 111.2 (t), 111.0 (t), 67.2 (d), 66.6 (d), 57.8 (d), 54.3 (d), 22.6 (d), 20.7 (s).

### Example 82: 4-(2-(2-(2-fluorobenzyl)-4,6-dimethyl phenoxy)ethyl)morpholine

Following the general procedure from Example 80: The mixture of 2-(2-fluorobenzyl)-4,6-dimethylphenol (0.40 g, 1.7 mmol) (prepared in an analogous procedure to Example 79), 4-(2-chloroethyl)morpholine hydrochloride (0.60 g, 3.5 mmol), potassium carbonate (2.40 g, 17.4 mmol) and potassium iodide (0.060 g, 0.30 mmol) in DMF (15 mL) were reacted to give the title product (0.40 g, 64% yield) as a viscous liquid. GC/MS (EI): m/z (%): 343 (10) [M⁺], 256 (26), 227 (7), 183 (5), 165 (3), 133 (1), 114 (22), 100 (100), 87 (1), 70 (3). ¹H NMR (300 MHz, CDCl₃) δ 7.21-7.10 (m, 1H), 7.10-6.97 (m, 3H), 6.86 (s, 1H), 6.71 (s, 1H), 4.02 (s, 2H), 3.85 (t, J=5.7 Hz, 2H), 3.76-3.67 (m, 4H), 2.73 (t, J=5.7 Hz, 2H), 2.57-2.46 (m, 4H), 2.27 (s, 3H), 2.20 (s, 3H). ¹³C NMR (75 MHz, CDCl₃) δ 162.7 (q), 159.4 (q), 153.6 (q), 133.4 (q), 132.1 (q), 131.1 (t), 131.0 (t), 130.7 (q), 130.4 (t), 129.0 (t), 128.2 (q), 128.0 (q), 127.7 (t), 127.7 (t), 124.1 (t), 124.1 (t), 115.3 (t), 115.0 (t), 70.0 (d), 67.0 (d), 58.8 (d), 54.3 (d), 28.6 (d), 28.6 (d), 20.9 (s), 16.5 (s).

### Example 83: 4-(1-(2-(2,6-difluorobenzyl)-4,6-dimethylphenoxy)propan-2-yl)morpholine and 4-(2-(2-(2,6-difluorobenzyl)-4,6-dimethylphenoxy)propyl)morpholine

Following the general procedure from Example 80: The mixture of 2-(2,6-difluorobenzyl)-4,6-dimethylphenol (0.4 g, 1.6 mmol), 4-(2-chloropropyl)morpholine hydrochloride (0.6 g, 3.2 mmol), potassium carbonate (2.3 g, 16.1 mmol) and potassium iodide (0.05 g, 0.3 mmol) in DMF (20 mL) were reacted to give 4-(1-(2-(2,6-difluorobenzyl)-4,6-dimethylphenoxy)propan-2-yl)morpholine (0.20 g, 0.50 mmol, yield 31%) as a viscous liquid and 4-(2-(2-(2,6-difluorobenzyl)-4,6-dimethylphenoxy)propyl)morpholine (0.12 g, 0.32 mmol, yield 19%) as a viscous liquid. 4-(1-(2-(2,6-difluorobenzyl)-4,6-dimethylphenoxy)propan-2-yl)morpholine: GC/MS (EI): m/z (%): 375 (1) [M⁺], 245 (1), 227 (2), 201 (2), 183 (2), 128 (7), 114 (100), 98(1), 84 (1), 70 (3). ¹H NMR (300 MHz, CDCl₃) δ 7.25-7.15 (m, 1H), 6.94-6.88 (m, 2H), 6.84 (s, 1H), 6.51 (s, 1H), 4.07 (s, 2H), 3.96-3.91 (m, 1H), 3.82-3.66 (m, 5H), 3.12-2.96 (m, 1H), 2.76-2.61 (m, 4H), 2.29 (s, 3H), 2.16 (s, 3H), 1.25 (d, J=6.7 Hz, 3H). ¹³C NMR (75 MHz, CDCl₃) δ 163.6 (q), 163.5 (q), 160.3 (q), 160.2 (q), 153.2 (q), 133.4 (q), 131.4 (q), 130.6 (q), 130.3 (t), 128.2 (t), 128.1 (t), 127.9 (t), 127.3 (t), 116.6 (q), 116.3 (q), 116.7 (q), 111.5 (t), 111.4 (t), 111.3 (t), 111.2 (t), 74.2 (d), 67.5 (d), 59.8 (t), 50.0 (d), 22.2 (d), 20.9 (s), 16.6 (s), 12.8 (s). 4-(2-(2-(2,6-difluorobenzyl)-4,6-dimethylphenoxy)propyl)morpholine: GC/MS (EI): m/z (%): 375 (1) [M⁺], 341 (1), 288 (15), 227 (5), 201 (5), 128 (30), 100 (100), 91(1), 77 (1), 56 (7). ¹H NMR (300 MHz, CDCl₃) δ 7.25-7.13 (m, 1H), 6.94-6.85 (m, 2H), 6.82 (s, 1H), 6.49 (s, 1H), 4.40-4.24 (m, 1H), 4.07 (s, 2H), 3.76-3.61 (m, 4H), 2.87-2.72 (m, 1H), 2.64-2.45 (m, 5H), 2.27 (s, 3H), 2.15 (s, 3H), 1.27 (d, J=6.2 Hz, 3H). ¹³C NMR (75 MHz, CDCl₃) δ 163.61 (q), 163.49 (q), 160.33 (q), 160.21 (q), 151.82 (q), 132.62 (q), 131.97 (q), 130.52 (q), 130.36 (t), 128.07 (t), 127.93 (t), 127.79 (t), 127.12 (t), 116.79 (q), 116.53 (q), 116.26 (q), 111.43 (t), 111.32 (t), 111.20 (t), 111.09 (t), 75.77 (t), 67.17 (d), 64.95 (d), 54.77 (d), 23.06 (d), 20.88 (s), 18.84 (s), 17.40 (s).

### Example 84: 4-(2-(2-(2-chlorobenzyl)-4,6-dimethylphenoxy)ethyl)morpholine

Following the general procedure from Example 80: The mixture of 2-(2-chlorobenzyl)-4,6-dimethylphenol (0.38 g, 1.5 mmol) (prepared in an analogous procedure to Example 79), 4-(2-chloroethyl)morpholine hydrochloride (0.57 g, 3.1 mmol), potassium carbonate (2.13 g, 15.4 mmol) and potassium iodide (0.050 g, 0.31 mmol) in DMF (15 mL) were reacted to give the title product (0.20 g, yield 35%) as a viscous liquid. GC/MS (EI): m/z (%): 359 (1) [M⁺], 272 (20), 209 (1), 141 (30), 100 (100), 87 (1), 56 (5). ¹H NMR (300 MHz, CDCl₃) δ 7.44-7.34 (m, 1H), 7.20-7.10 (m, 2H), 7.08-6.98 (m, 1H), 6.89 (s, 1H), 6.65 (s, 1H), 4.11 (s, 2H), 3.84 (t, J=5.7 Hz, 2H), 3.72-3.69 (m, 4H), 2.71 (t, J=5.7 Hz, 2H), 2.55-2.43 (m, 4H), 2.27 (s, 3H), 2.22 (s, 3H). ¹³C NMR (75 MHz, CDCl₃) δ 153.8 (q), 138.8 (q), 134.2 (q), 133.5 (q0, 131.9 (q), 130.8 (q), 130.8 (t), 130.5 (t), 129.4 (t), 129.1 (t), 127.5 (t), 126.9 (t), 69.9 (d), 67.0 (d), 58.7 (d), 54.2 (d), 33.4 (d), 20.9 (s), 16.5 (s).

### Example 85: 4-(2-(2-(2,5-difluorobenzyl)-4,6-dimethylphenoxy)ethyl)morpholine

Following the general procedure from Example 80: The mixture of 2-(2,5-difluorobenzyl)-4,6-dimethylphenol (0.30 g, 1.2 mmol) (prepared in an analogous procedure to Example 79), 4-(2-chloroethyl)morpholine hydrochloride (0.45 g, 2.4 mmol), potassium carbonate (1.67 g, 12.1 mmol) and potassium iodide (0.040 g, 0.24 mmol) in DMF (13 mL) were reacted to give the title product (0.36 g, yield 80%) as a viscous liquid. GC/MS (EI): m/z (%): 361 (1) [M⁺], 346 (1), 274 (20), 227 (5), 201 (5), 127 (7), 100 (100), 87(2), 70 (5), 56 (10). ¹H NMR (300 MHz, CDCl₃) δ 7.02-6.92 (m, 1H), 6.88 (s, 1H), 6.87-6.78 (m, 2H), 6.75 (s, 1H), 3.99 (s, 2H), 3.85 (t, J=5.7 Hz, 2H), 3.77-3.69 (m, 4H), 2.73 (t, J=5.7 Hz, 2H), 2.60-2.47 (m, 4H), 2.28 (s, 3H), 2.23 (s, 3H): ¹³C NMR (75 MHz, CDCl₃) δ 160.4 (q), 160.4 (q), 158.6 (q), 158.5 (q), 157.2 (q), 157.2 (q), 155.4 (q), 155.3 (q), 153.7 (q), 133.6 (q), 131.4 (q), 131.0 (q), 130.9 (t), 130.3 (q), 130.2 (q), 130.1 (q), 130.0 (q), 129.1 (t), 117.6 (t), 117.5 (t), 117.3 (t), 117.2 (t), 116.2 (t), 116.1 (s), 115.9 (t), 115.8 (t), 114.2 (t), 114.1 (t), 113.9 (t), 113.7 (t), 70.1 (d), 67.0 (d), 58.8 (d), 54.3 (d), 28.8 (d), 20.9 (s), 16.5 (s).

### Example 86: 1-(2-(2-(2-chlorobenzyl)-4-methylphenoxy)ethyl)-4-methylpiperazine

Following the general procedure from Example 80: The mixture of 2-(2-chlorobenzyl)-4-methylphenol (0.50 g, 2.2 mmol) (prepared in an analogous procedure to Example 79), 1-(2-chloroethyl)-4-methylpiperazine dihydrochloride (1.01 g, 4.3 mmol), potassium carbonate (2.97 g, 21.5 mmol) and potassium iodide (0.070 g, 0.43 mmol) in DMF (Volume: 15 mL) were reacted to give the title product (0.63 g, yield 78%) as a viscous liquid. GC/MS (EI): m/z (%): 358 (2) [M⁺], 281 (2), 253 (2), 207 (2), 183 (10), 195 (8), 165 (7), 127(40), 113 (100), 70 (40). ¹H NMR (300 MHz, CDCl₃) δ 7.41-7.31 (m, 1H), 7.16-7.07 (m, 2H), 7.07-6.96 (m, 2H), 6.86 (s, 1H), 6.76 (d, J=8.2 Hz, 1H), 4.12-4.00 (m, 4H), 2.73 (t, J=5.6 Hz, 2H), 2.53 (s, 4H), 2.40 (s, 4H), 2.27 (s, 3H), 2.23 (s, 3H). ¹³C NMR (75 MHz, CDCl₃) δ 154.7 (q), 138.8 (q), 134.3 (q), 131.4 (t), 130.6 (t), 129.9 (q), 129.3 (t), 128.0 (t), 127.8 (q), 127.2 (t), 126.6 (t), 111.4 (t), 66.8 (d), 57.3 (d), 55.2 (d), 53.6 (d), 46.1 (s), 33.5 (d), 20.6 (s).

### Example 87: 4-(2-(2-(2,4-difluorobenzyl)-4,6-dimethylphenoxy)ethyl)morpholine

Following the general procedure from Example 80: The mixture of 2-(2,4-difluorobenzyl)-4,6-dimethylphenol (0.28 g, 1.1 mmol) (prepared in an analogous procedure to Example 79), 4-(2-chloroethyl)morpholine hydrochloride (0.41 g, 2.2 mmol), potassium carbonate (1.53 g, 11.1 mmol) and potassium iodide (0.040 g, 0.22 mmol) in DMF (13 mL) were reacted to give the title product (0.30 g, yield 73%) as a viscous liquid. GC/MS (EI): m/z (%): 361 (1) [M⁺], 341 (1), 274 (10), 227 (5), 183 (2), 127 (7), 114 (20), 100 (100), 87 (1), 70 (3). ¹H NMR (300 MHz, CDCl₃) δ 7.11-7.04 (m, 1H), 6.88 (s, 1H), 6.84-6.72 (m, 2H), 6.70 (s, 1H), 3.97 (s, 2H), 3.86 (t, J=5.7 Hz, 2H), 3.74-3.71 (m, 4H), 2.75 (t, J=5.7 Hz, 2H), 2.60-2.48 (m, 4H), 2.28 (s, 3H), 2.22 (s, 3H). ¹³C NMR (75 MHz, CDCl₃) δ 163.2 (q), 163.1 (q), 162.5 (q), 162.4 (q), 160.0 (q), 159.8 (q), 159.2 (q), 159.1 (q), 153.6 (q), 133.6 (q), 131.9 (q), 131.7 (t), 131.7 (t), 131.6 (t), 131.5 (t), 130.9 (q), 130.6 (t), 128.9 (t), 124.1 (q), 124.1 (q), 123.9 (q), 123.9 (q), 111.3 (t), 111.2 (t), 111.0 (t), 110.9 (t), 103.9 (t), 103.6 (t), 103.2 (t), 70.0 (d), 67.0 (d), 58.8 (d), 54.3 (d), 28.1 (d), 28.1 (d), 20.9 (s), 16.5 (s).

### Example 88: 4-(2-(2-(2,6-dichlorobenzyl)-4,6-dimethylphenoxy)ethyl)morpholine

Following the general procedure from Example 80: The mixture of 2-(2,6-dichlorobenzyl)-4,6-dimethylphenol (0.50 g, 0.89 mmol) (prepared in an analogous procedure to Example 79), 4-(2-chloroethyl)morpholine hydrochloride (0.33 g, 1.9 mmol), potassium carbonate (1.23 g, 8.9 mmol) and potassium iodide (0.030 g, 0.18 mmol) in DMF (15 mL) were reacted to give the title product (0.21 g, yield 57%) as a viscous liquid. GC/MS (EI): m/z (%): 393 (1) [M⁺], 241 (1), 306 (6), 243 (5), 193 (5), 165 (5), 114 (400), 100 (100), 84 (1), 70 (3). ¹H NMR (300 MHz, CDCl₃) δ 7.36 (d, J=8.0 Hz, 2H), 7.23-7.12 (m, 1H), 6.85 (s, 1H), 6.17 (d, J=7.5 Hz, 1H), 4.35 (s, 2H), 4.02 (t, J=5.8 Hz, 2H), 3.83-3.71 (m, 4H), 2.86 (t, J=5.8 Hz, 2H), 2.72-2.55 (m, 4H), 2.30 (s, 3H), 2.12 (s, 3H). ¹³C NMR (75 MHz, CDCl₃) δ 153.4 (q), 136.6 (q), 136.4 (q), 133.3 (q), 130.5 (q), 130.4 (q), 130.2 (t), 128.4 (t), 128.3 (t), 125.9 (t), 69.7 (d), 67.1 (d), 58.9 (d), 54.4 (d), 31.3 (d), 21.0 (s), 16.4 (s).

### Example 89: 4-(2-(2-(3,5-difluorobenzyl)-4,6-dimethylphenoxy)ethyl)morpholine

Following the general procedure from Example 80: The mixture of 2-(3,5-difluorobenzyl)-4,6-dimethylphenol (0.27 g, 1.1 mmol) (prepared in an analogous procedure to Example 79), 4-(2-chloroethyl)morpholine hydrochloride (0.41 g, 2.2 mmol), potassium carbonate (1.50 g, 10.9 mmol) and potassium iodide (0.040 g, 0.22 mmol) in DMF (15 mL) were reacted to give the title product (0.33 g, yield 81%) as a viscous liquid. GC/MS (EI): m/z (%): 361 (1) [M⁺], 346 (1), 274 (10), 201 (5), 183 (2), 127 (7), 114 (15), 100 (100), 87 (1), 70 (3). ¹H NMR (300 MHz, CDCl₃) δ 6.90 (s, 1H), 6.79-6.69 (m, 3H), 6.67-6.55 (m, 1H), 3.96 (s, 2H), 3.80 (t, J=5.7 Hz, 2H), 3.76-3.69 (m, 4H), 2.72 (t, J=5.7 Hz, 2H), 2.58-2.47 (m, 4H), 2.26 (d, J=10.2 Hz, 6H). ¹³C NMR (75 MHz, CDCl₃) δ 164.8 (q), 164.6 (q), 161.5 (q), 161.4 (q), 153.6 (q), 145.7 (q), 145.6 (q), 145.5 (q), 133.7 (q), 132.0 (q), 131.1 (q), 130.9 (t), 129.2 (t), 111.8 (t), 111.7 (t), 111.6 (t), 111.5 (t), 101.8 (t), 101.5 (t), 101.2 (t), 70.2 (d), 67.0 (d), 58.8 (d), 54.3 (d), 35.9 (d), 20.8 (s), 16.5 (s).

### Example 90: 4-(2-(2,4-dimethyl-6-(2,4,5-trifluorobenzyl)phenoxy)ethyl)morpholine

Following the general procedure from Example 80: The mixture of 2,4-dimethyl-6-(2,4,5-trifluorobenzyl)phenol (0.30 g, 1.1 mmol) (prepared in an analogous procedure to Example 79), 4-(2-chloroethyl)morpholine hydrochloride (0.42 g, 2.2 mmol), potassium carbonate (1.56 g, 11.3 mmol) and potassium iodide (0.040 g, 0.23 mmol) in DMF (15 mL) were reacted to give the title product (0.36 g, yield 80%) as a viscous liquid. GC/MS (EI): m/z (%): 379 (20) [M⁺], 292 (17), 245 (8), 219 (6), 201 (5), 145 (7), 114 (14), 100 (100), 85 (1), 70 (3). ¹H NMR (300 MHz, CDCl₃) δ 7.04-6.83 (m, 3H), 6.73 (s, 1H), 3.94 (s, 2H), 3.85 (t, J=5.6 Hz, 2H), 3.78-3.67 (m, 4H), 2.74 (t, J=5.6 Hz, 2H), 2.60-2.49 (m, 4H), 2.26 (d, J=7.1 Hz, 3H), 2.23 (s, 3H). ¹³C NMR (75 MHz, CDCl₃) δ 153.6 (q), 133.7 (q), 131.3 (q), 131.1 (q), 131.0 (t), 129.0 (t), 124.9 (q), 124.9 (q), 124.8 (q), 124.7 (q), 124.6 (q), 124.6 (q), 118.8 (t), 118.7 (t), 118.6 (t), 118.5 (t), 105.4 (t), 105.2 (t), 105.1 (t), 104.8 (t), 70.1 (d), 67.0 (d), 58.79 (d), 54.3 (d), 28.3 (d), 20.8 (s), 16.5 (s).

### Example 91: 1-(2-(2-(2-fluorobenzyl)-4,6-dimethylphenoxy)ethyl)-4-methylpiperazine

Following the general procedure from Example 80: The mixture of 2-(2-fluorobenzyl)-4,6-dimethylphenol (0.25 g, 1.0 mmol) (prepared in an analogous procedure to Example 79), 1-(2-chloroethyl)-4-methylpiperazine hydrochloride (0.41 g, 2.1 mmol), potassium carbonate (1.43 g, 10.3 mmol) and potassium iodide (0.030 g, 0.21 mmol) in DMF (10 mL) were reacted to give the title product (0.10 g, yield 26%) as a viscous liquid. GC/MS (EI): m/z (%): 356 (2) [M⁺], 227 (2), 209 (2), 183 (5), 165 (3), 127 (57), 113 (100), 100 (17), 84 (7), 70 (38). ¹H NMR (300 MHz, CDCl₃) δ 7.21-6.98 (m, 4H), 6.86 (s, 1H), 6.71 (s, 1H), 4.00 (s, 2H), 3.83 (t, J=5.8 Hz, 2H), 2.76 (t, J=5.8 Hz, 2H), 2.54 (s, 8H), 2.36 (s, 3H), 2.26 (s, 3H), 2.20 (s, 3H). ¹³C NMR (75 MHz, CDCl₃) δ 162.7 (q), 159.4 (q), 153.6 (q), 133.5 (q), 132.1 (t), 131.1 (t), 131.1 (q), 130.8 (q), 130.5 (t), 129.1 (t), 128.2 (q), 128.0 (q), 127.8 (t), 127.7 (t), 124.1 (t), 124.1 (t), 115.3 (t), 115.0 (t), 70.2 (d), 58.1 (d), 54.9 (d), 53.2 (d), 45.7 (s), 28.6 (d), 28.5 (d), 20.9 (s), 16.5 (s).

### Example 92: 1-(2-(2-(2-bromobenzyl)-4,6-dimethylphenoxy)ethyl)-4-methylpiperazine

Following the general procedure from Example 80: The mixture of 2-(2-bromobenzyl)-4,6-dimethylphenol (0.15 g, 0.52 mmol) (prepared in an analogous procedure to Example 79), 1-(2-chloroethyl)-4-methylpiperazine dihydro-chloride (0.18 g, 0.77 mmol), potassium carbonate (0.57 g, 4.1 mmol) and potassium iodide (0.020 g, 0.10 mmol) in DMF (10 mL) were reacted to give the title product (0.050 g, yield 23%) as a viscous liquid. GC/MS (EI): m/z (%): 416 (2) [M⁺], 360 (1), 209 (10), 195 (8), 179 (9), 165 (7), 127 (75), 113 (100), 100 (20), 70 (40). CDCl₃) δ 7.56-7.51 (m, 1H), 7.23-6.82 (m, 4H), 6.63 (s, 1H), 4.09 (s, 2H), 3.82 (s, 2H), 3.73 (s, 2H), 2.92-2.46 (m, 8H), 2.27 (s, 3H), 2.22 (s, 3H), 2.20 (s, 3H). ¹³C NMR (75 MHz, CDCl₃) δ 153.8 (q), 140.6 (q), 133.5 (q), 132.7 (t), 131.9 (q), 130.8 (t), 130.6 (t), 129.1 (t), 127.8 (t), 127.5 (t), 124.9 (q), 70.2 (d), 58.1 (d), 54.9 (d), 53.1 (d), 45.7 (s), 36.2 (d), 20.9 (s), 16.5 (s).

### Example 93: 1-(2-(2,4-dimethyl-6-(2-(trifluoromethyl)benzyl)phenoxy)ethyl)-4-methylpiperazine

Following the general procedure from Example 80: The mixture of 2,4-dimethyl-6-(2-(trifluoromethyl)benzyl)phenol (85 mg, 0.24 mmol, 80% purity) (prepared in an analogous procedure to Example 79), 1-(2-chloroethyl)-4-methylpiperazine dihydrochloride (114 mg, 0.49 mmol), potassium carbonate (335 mg, 2.43 mmol) and potassium iodide (8 mg, 0.05 mmol) in DMF (10 mL) were reacted to give the title product (14 mg, 0.03 mmol, yield 14%) as a viscous liquid. GC/MS (EI): m/z (%): 406 (1) [M⁺], 343 (4), 256 (18), 227 (5), 183 (4), 114 (25), 100 (100), 91(1), 84 (1), 70 (3). ¹H NMR (300 MHz, CDCl₃) δ 7.65 (d, J=7.7 Hz, 1H), 7.38 (t, J=7.4 Hz, 1H), 7.32-7.24 (m, 1H), 7.06 (d, J=7.7 Hz, 1H), 6.89 (s, 1H), 6.60 (s, 1H), 4.19 (s, 2H), 3.81 (t, J=5.9 Hz, 2H), 2.69 (t, J=5.9 Hz, 2H), 2.75-2.37 (m, 8H), 2.31 (s, 3H), 2.28 (s, 3H), 2.19 (s, 3H). ¹³C NMR (75 MHz, CDCl₃) δ 153.8 (q), 140.0 (q), 133.6 (q), 132.2 (q), 131.9 (t), 131.2 (t), 130.9 (q), 130.7 (t), 129.5 (t), 126.1 (t), 125.9 (t), 125.8 (t), 70.3 (d), 58.0 (d), 54.9 (d), 53.3 (d), 45.8 (s), 31.9 (d), 20.9 (s), 16.6 (s).

### Example 94: 1-methyl-4-(2-(4-methyl-2-(2-methylbenzyl)phenoxy)ethyl)piperazine

To sodium hydride (1.27 g, 29.7 mmol) in DMF (20 mL) were added 4-methyl-2-(2-methylbenzyl)phenol (1.00 g, 4.2 mmol) (prepared in an analogous procedure to Example 79), 1-(2-chloroethyl)-4-methylpiperazine dihydrochloride (1.49 g, 6.4 mmol) and potassium iodide (0.14 g, 0.85 mmol). The resulting mixture was stirred at 60° C. overnight. Water was added to quench the reaction, EA (ethyl acetate) was used to extract the solution, the solvent was removed and the residue was purified very carefully by column chromatography on silica gel (PE:MTBE=1:1 to MTBE to DCM:Methanol=10:1) to give the title product (0.30 mg, yield 20%) as a viscous liquid. GC/MS (EI): m/z (%): 338 (5) [M⁺], 195 (4), 165 (5), 127 (55), 113 (100), 98 (7), 70 (55). ¹H NMR (300 MHz, CDCl₃) δ 7.20-7.05 (m, 3H), 6.97-6.94 (m, 2H), 6.80-6.69 (m, 2H), 4.08 (t, J=5.6 Hz, 2H), 3.90 (s, 2H), 2.77 (t, J=5.6 Hz, 2H), 2.59-2.47 (m, 8H), 2.32 (s, 3H), 2.28 (s, 3H), 2.21 (s, 3H). ¹³C NMR (75 MHz, CDCl₃) δ 154.5 (q), 139.1 (q), 136.6 (q), 130.9 (t), 130.0 (t), 129.9 (t), 129.4 (q), 128.7 (t), 127.6 (t), 126.0 (t), 125.9 (t), 111.3 (t), 66.7 (d), 57.2 (d), 54.9 (d), 53.2 (d), 45.8 (s), 33.3 (d), 20.6 (s), 19.7 (s).

### Example 95: 1-(2-(2-(furan-2-ylmethyl)-4-methylphenoxy)ethyl)-4-methylpiperazine

Following the general procedure from Example 80: The mixture of 2-(furan-2-ylmethyl)-4-methylphenol (0.83 g, 4.2 mmol) (prepared in an analogous procedure to Example 79), 1-(2-chloroethyl)-4-methylpiperazine dihydrochloride (1.18 g, 5.0 mmol), potassium carbonate (2.89 g, 20.9 mmol) and potassium iodide (0.14 g, 0.84 mmol) in DMF (20 mL) was heated to 100° C. for 5 hours. Water was added to quench the reaction, EA was used to extract the solution three times, the solvent was removed and the residue was purified by column chromatography on silica gel (MTBE to DCM:Methanol=10:1) to get the title product (0.47 g, yield 34%) as a viscous liquid. GC/MS (EI): m/z (%): 314 (30) [M⁺], 185 (2), 127 (35), 113 (100), 98 (7), 84 (5), 70 (50). ¹H NMR (300 MHz, CDCl₃) δ 7.30 (s, 1H), 6.98 (d, J=8.2 Hz, 1H), 6.93 (d, J=8.2 Hz, 1H), 6.74 (d, J=8.2 Hz, 1H), 6.26 (d, J=2.8 Hz, 1H), 5.95 (d, J=2.8 Hz, 1H), 4.07 (t, J=5.6 Hz, 2H), 3.92 (s, 2H), 2.80 (t, J=5.6 Hz, 2H), 2.68-2.60 (m, 4H), 2.56-2.48 (m, 4H), 2.33 (s, 3H), 2.24 (s, 3H). ¹³C NMR (75 MHz, CDCl₃) δ 154.7 (q), 154.4 (q), 141.1 (t), 131.1 (t), 130.1 (q), 128.2 (t), 126.7 (q), 111.7 (t), 110.3 (t), 106.0 (t), 66.9 (d), 57.3 (d), 55.1 (d), 53.3 (d), 45.9 (s), 28.7 (d), 20.6 (s).

### Example 96: 1-(2-(2-benzyl-4,6-dimethylphenoxy)ethyl)-4-methylpiperazine dihydrochloride

1-(2-(2-benzyl-4,6-dimethylphenoxy)ethyl)-4-methylpiperazine (160 mg, 0.47 mmol) was dissolved in 5 mL THF and 5 mL 3M HCl aqueous solution was added. The resulting mixture was stirred at rt (rt=room temperature). for half an hour and then the mixture was concentrated to get a the title product (170 mg, 21%) as a white solid. GC/MS (EI): m/z (%): 338 (5) [M⁺], 209 (3), 178 (2), 165 (5), 127 (50), 113 (100), 100 (20), 84(5), 70 (40).

### Example 97: 4-(2-(2,4-dimethyl-6-(2-nitrobenzyl)phenoxy)ethyl)morpholine

Following the general procedure from Example 80: 4-(2-chloroethyl)morpholine hydrochloride (0.21 g, 1.1 mmol), 2,4-dimethyl-6-(2-nitrobenzyl)phenol (0.15 g, 0.6 mmol) (prepared in an analogous procedure to Example 79), potassium carbonate (0.77 g, 5.5 mmol) and potassium iodide (0.018 g, 0.11 mmol) in DMF (10 mL) were reacted to give the title product (70 mg, yield 41%) as a viscous liquid. GC/MS (EI): m/z (%): 370 (1) [M⁺], 238 (2), 224 (5), 111 (15), 100 (100), 93 (2), 84 (1), 70 (3). ¹H NMR (300 MHz, CDCl₃) δ 7.80 (d, J=8.9 Hz, 1H), 7.58 (d, J=9.1 Hz, 1H), 7.39 (s, 1H), 7.35-7.27 (m, 1H), 7.16 (s, 1H), 7.03-6.94 (m, 1H), 3.72-3.64 (m, 3H), 3.63-3.55 (m, 4H), 2.48 (t, J=5.6 Hz, 3H), 2.36 (s, 3H), 2.33 (s, 3H), 2.34-2.28 (m, 4H). ¹³C NMR (75 MHz, CDCl₃) δ 164.1 (q), 157.7 (q), 153.1 (q), 134.5 (t), 134.1 (t), 132.3 (t), 130.9 (q), 128.7 (q), 123.6 (t), 122.5 (t), 121.7 (q), 115.8 (q), 115.1 (t), 70.8 (d), 66.9 (d), 58.3 (d), 53.9 (d), 20.8 (s), 16.5 (s).

### Example 98: 1-(2-(2-benzyl-3-methoxyphenoxy)ethyl)-4-methylpiperazine

Following the general procedure from Example 80: The mixture of 2-benzyl-3-methoxyphenol (0.15 g, 0.70 mmol) (prepared in an analogous procedure to Example 79), 1-(2-chloroethyl)-4-methylpiperazine dihydrochloride (0.33 g, 1.4 mmol), potassium carbonate (0.97 g, 7.00 mmol) and potassium iodide (0.020 g, 0.14 mmol) in DMF (10 mL) were reacted to give the title product (0.090 g, yield 37%) as a viscous liquid. GC/MS (EI): m/z (%): 340 (3) [M⁺], 249 (1), 197 (3), 165 (5), 152 (5), 127 (50), 113 (100), 100(10), 91 (15), 70 (45). ¹H NMR (300 MHz, CDCl₃) δ 7.25-7.05 (m, 6H), 6.58-6.51 (m, 2H), 4.07 (t, J=5.6 Hz, 2H), 4.01 (s, 2H), 3.80 (s, 3H), 2.78 (t, J=5.6 Hz, 2H), 2.70-2.35 (m, 8H), 2.32 (s, 3H). ¹³C NMR (75 MHz, CDCl₃) δ 158.4 (q), 157.4 (q), 141.8 (q), 128.6 (t), 127.9 (t), 127.4 (t), 125.3 (t), 117.7 (q), 104.6 (t), 103.9 (t), 66.7 (d), 57.2 (d), 55.8 (s), 54.9 (d), 53.2 (d), 45.8 (s), 28.8 (d).

### Example 99: 1-(2-(2-((5-chlorothiophen-2-yl)methyl)-4-methylphenoxy)ethyl)-4-methylpiperazine

Following the general procedure from Example 80: The mixture of 2-((5-chlorothiophen-2-yl)methyl)-4-methylphenol (1.00 g, 4.2 mmol) (prepared in an analogous procedure to Example 79), 1-(2-chloroethyl)-4-methylpiperazine dihydrochloride (1.28 g, 5.4 mmol), potassium carbonate (4.63 g, 33.5 mmol) and potassium iodide (0.14 g, 0.84 mmol) in DMF (20 mL) were reacted to give the title product (0.62 g, yield 40%) as a viscous liquid. GC/MS (EI): m/z (%): 364 (8) [M⁺], 331 (4), 281 (10), 253 (8), 207 (80), 127 (40), 113 (100), 96(5), 84 (3), 70 (40). ¹H NMR (300 MHz, CDCl₃) δ 7.06-6.93 (m, 2H), 6.75 (d, J=8.7 Hz, 1H), 6.66 (d, J=3.5 Hz, 1H), 6.54 (d, J=3.5 Hz, 1H), 4.07 (t, J=11.0, 5.4 Hz, 2H), 4.01 (s, 2H), 2.85-2.75 (m, 2H), 2.62-2.51 (m, 8H), 2.33 (s, 3H), 2.24 (s, 3H). ¹³C NMR (75 MHz, CDCl₃) δ 154.2 (q), 143.4 (q), 130.9 (t), 130.1 (q), 128.5 (t), 128.1 (q), 125.6 (t), 124.0 (t), 111.6 (t), 66.6 (d), 57.3 (d), 55.1 (d), 53.3 (d), 45.9 (s), 31.1 (d), 20.6 (s).

### Example 100: 1-(2-(2-benzyl-6-methylphenoxy)ethyl)-4-methylpiperazine

Following the general procedure from Example 80: The mixture of 2-benzyl-6-methylphenol (0.20 g, 1.0 mmol) (prepared in an analogous procedure to Example 79), 1-(2-chloroethyl)-4-methylpiperazine dihydrochloride (0.48 g, 2.0 mmol), potassium carbonate (1.39 g, 10.1 mmol) and potassium iodide (0.030 g, 0.20 mmol) in DMF (10 mL) were reacted to give the title product (0.040 g, yield 11%) as a viscous liquid. GC/MS (EI): m/z (%): 324 (5) [M⁺], 268 (1), 195 (5), 165 (15), 127 (2), 128 (55), 113 (100), 100(20), 84 (5), 70 (40). ¹H NMR (300 MHz, CDCl₃) δ 7.26-6.85 (m, 8H), 4.10-4.00 (m, 2H), 3.90-3.75 (m, 2H), 2.70-2.55 (m, 10H), 2.44-2.24 (m, 6H). ¹³C NMR (75 MHz, CDCl₃) δ 155.8 (q), 141.2 (q), 133.8 (q), 131.1 (q), 129.7 (t), 128.9 (t), 128.5 (t), 126.1 (t), 124.2 (t), 70.1 (d), 57.9 (d), 54.7 (d), 52.8 (d), 45.5 (s), 35.9 (d), 16.6 (s).

### Example 101: 1-(2-(2-benzyl-4-(tert-butyl)-6-methoxyphenoxy)ethyl)-4-methylpiperazine

A mixture of 2-benzyl-4-(tert-butyl)-6-methoxyphenol (0.25 g, 0.93 mmol) (prepared in an analogous procedure to Example 79), 1-(2-chloroethyl)-4-methylpiperazine dihydrochloride (0.44 g, 1.8 mmol), potassium carbonate (1.28 g, 9.2 mmol) and potassium iodide (0.030 g, 0.19 mmol) in DMF (10 mL) was heated to reflux for overnight. Water was added, DCM was used to extract the solution for three times, the solvent was removed and the residue was purified by column chromatography on silica gel (MTBE, and then DCM:Methanol=10:1) to get the title product (0.28 g, yield 74%) as a viscous liquid. GC/MS (EI): m/z (%): 396 (5) [M⁺], 281 (2), 255 (3), 181 (2), 165 (3), 127 (100), 113 (92), 91 (12), 84 (9), 70 (37). ¹H NMR (300 MHz, CDCl₃) δ 7.25-7.10 (m, 5H), 6.79 (d, J=2.1 Hz, 1H), 6.75 (d, J=2.0 Hz, 1H), 4.00 (s, 2H), 3.91 (t, J=5.9 Hz, 2H), 3.83 (s, 3H), 2.66 (t, J=5.9 Hz, 2H), 2.55-2.38 (m, 8H), 2.30 (s, 3H), 1.27 (s, 9H). ¹³C NMR (75 MHz, CDCl₃) δ 152.1 (q), 146.8 (q), 144.1 (q), 141.6 (q), 133.7 (q), 128.8 (t), 128.3 (t), 125.8 (t), 119.7 (t), 108.3 (t), 70.0 (d), 58.1 (d), 55.8 (s), 55.0 (d), 53.2 (d), 45.9 (s), 36.5 (d), 34.7 (q), 31.6 (s).

### Example 102: 1-(2-(2-(3-chlorobenzyl)-4-methylphenoxy)ethyl)-4-methylpiperazine

Following the general procedure from Example 80: The mixture of 2-(3-chlorobenzyl)-4-methylphenol (0.80 g, 3.4 mmol) (prepared in an analogous procedure to Example 79), 1-(2-chloroethyl)-4-methylpiperazine dihydrochloride (1.62 g, 6.9 mmol), potassium carbonate (4.75 g, 34.4 mmol) and potassium iodide (0.11 g, 0.69 mmol) in DMF (15 mL) were reacted to give the title product (0.67 g, 52% yield) as a viscous liquid. GC/MS (EI): m/z (%): 358 (3) [M⁺], 253 (1), 229 (1), 195 (4), 165 (7), 127 (52), 113 (100), 70 (33). ¹H NMR (300 MHz, CDCl₃) δ 7.23-7.05 (m, 4H), 6.98 (d, J=8.2 Hz, 1H), 6.92 (s, 1H), 6.73 (d, J=8.2 Hz, 1H), 4.04 (t, J=5.7 Hz, 2H), 3.89 (s, 2H), 2.75 (t, J=5.7 Hz, 2H), 2.6-2.54 (m, 4H), 2.52-2.40 (m, 4H), 2.30 (s, 3H), 2.25 (s, 3H). ¹³C NMR (75 MHz, CDCl₃) δ 154.5 (q), 143.5 (q), 133.9 (q), 131.4 (t), 129.9 (q), 129.5 (t), 128.9 (t), 128.6 (q), 128.1 (t), 127.1 (t), 125.9 (t), 111.6 (t), 66.5 (d), 57.3 (d), 55.1 (d), 53.5 (d), 45.9 (s), 36.1 (d), 20.6 (s).

### Example 103: Methyl 3-benzyl-4-(2-(4-methylpiperazin-1-yl)ethoxy)benzoate

A mixture of methyl 3-benzyl-4-hydroxybenzoate (0.15 g, 0.62 mmol) (prepared in an analogous procedure to Example 79), 1-(2-chloroethyl)-4-methylpiperazine dihydrochloride (0.29 g, 1.2 mmol), potassium carbonate (0.86 g, 6.2 mmol) and potassium iodide (0.021 g, 0.12 mmol) in DMF (10 mL) was heated to reflux for 4 hours. Water was added, DCM was used to extract the solution for three times, the solvent was removed and the residue was purified by column chromatography on silica gel (MTBE to DCM:Methanol=10:1) to get the title product (0.090 g, yield 40%) as a viscous liquid. GC/MS (EI): m/z (%): 368 (1) [M⁺], 239 (2), 207 (5), 181 (5), 165 (10), 127 (55), 113 (100), 91 (53), 70 (40). ¹H NMR (300 MHz, CDCl₃) δ 7.90 (d, J=8.6 Hz, 1H), 7.84 (s, 1H), 7.25-7.20 (m, 2H), 7.19-7.11 (m, 3H), 6.83 (d, J=8.6 Hz, 1H), 4.11 (t, J=5.6 Hz, 2H), 3.96 (s, 2H), 3.85 (s, 3H), 2.78 (t, J=5.6 Hz, 2H), 2.70-2.54 (m, 4H), 2.52-2.40 (m, 4H), 2.32 (s, 3H). ¹³C NMR (75 MHz, CDCl₃) δ 167.1 (q), 160.5 (q), 140.5 (q), 132.3 (t), 130.1 (t), 129.6 (q), 128.8 (t), 128.4 (t), 126.1 (t), 122.5 (q), 110.7 (t), 66.8 (d), 56.9 (d), 55.0 (d), 53.2 (d), 52.0 (s), 45.8 (s), 36.4 (d).

### Example 104: 1-(2-(2,4-dimethyl-6-(pyridin-2-ylmethyl)phenoxy)ethyl)-4-methylpiperazine

A mixture of 2,4-dimethyl-6-(pyridin-2-ylmethyl)phenol hydrochloride (0.35 g, 0.98 mmol) (prepared in an analogous procedure to Example 79, and isolated as the hydrochloride salt in an analogous procedure to Example 96), 1-(2-chloroethyl)-4-methylpiperazine dihydrochloride (0.46 g, 2.0 mmol), potassium carbonate (2.03 g, 14.7 mmol) and potassium iodide (16 mg, 0.098 mmol) in DMF (30 mL) was heated to reflux for 3 hours. Water was added, DCM was used to extract the solution for three times, the solvent was removed and the residue was purified by column chromatography on silica gel (DCM:Methanol=10:1) to get the title product (0.58 g, yield 66%) as a viscous liquid. GC/MS (EI): m/z (%): 339 (1) [M⁺], 268 (4), 221 (10), 181 (2), 165 (5), 127(60), 113 (100), 98 (10), 70 (85). ¹H NMR (300 MHz, CDCl₃) δ 8.50 (d, J=4.7 Hz, 1H), 7.55-7.50 (m, 1H), 7.15-7.00 (m, 2H), 6.85 (s, 1H), 6.80 (s, 1H), 4.16 (s, 2H), 3.83 (t, J=5.7 Hz, 2H), 2.72 (t, J=5.7 Hz, 2H), 2.67-2.38 (m, 8H), 2.32 (s, 3H), 2.25 (s, 3H), 2.19 (s, 3H). ¹³C NMR (75 MHz, CDCl₃) δ 161.4 (q), 153.6 (q), 149.2 (t), 136.5 (t), 133.5 (q), 131.9 (q), 130.8 (q), 130.6 (t), 129.4 (t), 123.1 (t), 121.1 (t), 70.3 (d), 58.1 (d), 54.9 (d), 53.2 (d), 45.8 (s), 38.8 (d), 20.8 (s), 16.5 (s).

### Example 105: 4-(2-(2,4-dimethyl-5-((perfluorophenyl)methyl)phenoxy)ethyl)morpholine

A mixture of 2,4-dimethyl-6-((perfluorophenyl)methyl)phenol (1.00 g, 3.3 mmol) (isolated as an isomeric by-product in the preparation of 2,4-dimethyl-6-((perfluorophenyl)methyl)phenol by an analogous procedure to Example 79), 4-(2-chloroethyl)morpholine hydrochloride (1.23 g, 6.6 mmol), potassium carbonate (4.57 g, 33.1 mmol) and potassium iodide (0.11 g, 0.66 mmol) in DMF (13 mL) was heated to reflux for 3 hours. The color of the reaction solution turned very deep color. Water was added, DCM was used to extract the solution for three times, the solvent was removed and the residue was purified by column chromatography on silica gel (PE:MTBE=1:1) to get the title product (0.15 g, yield 10%). GC/MS (EI): m/z (%): 415 (1) [M⁺], 237 (2), 219 (2), 181 (5), 114 (5), 100 (100), 91 (3), 70 (4). ¹H NMR (300 MHz, CDCl₃) δ 6.95 (s, 1H), 6.55-6.44 (m, 1H), 4.07-3.89 (m, 4H), 3.80-3.65 (m, 4H), 2.87-2.69 (m, 2H), 2.66-2.50 (m, 4H), 2.28 (s, 3H), 2.15 (s, 3H). ¹³C NMR (75 MHz, CDCl₃) δ 155.3 (q), 133.7 (q), 133.4 (q), 133.0 (q), 132.9 (t), 127.9 (q), 125.7 (q), 112.0 (t), 67.1 (d), 66.7 (d), 57.8 (d), 54.3 (d), 25.8 (d), 25.8 (d), 18.6 (s), 15.8 (s).

### Example 106: 1-(2-(2-(3-bromobenzyl)-4,6-dimethyl phenoxy)ethyl)-4-methylpiperazine

A mixture of 2-(3-bromobenzyl)-4,6-dimethylphenol (0.40 g, 1.4 mmol) (prepared in an analogous procedure to Example 79), 1-(2-chloroethyl)-4-methylpiperazine dihydrochloride (0.49 g, 2.1 mmol), potassium carbonate (1.52 g, 11.0 mmol) and potassium iodide (0.050 g, 0.28 mmol) in DMF (10 mL) was heated to 100° C. for overnight. Water was added, DCM was used to extract the solution for three times, the solvent was removed and the residue was purified by column chromatography on silica gel (MTBE to DCM:Methanol=10:1) to get the title product (0.25 g, yield 42%) as a viscous liquid. GC/MS (EI): m/z (%): 416 (2) [M⁺], 209 (5), 195 (4), 179 (4), 165 (4), 127(65), 113 (100), 100 (15), 84 (5), 70 (40). ¹H NMR (300 MHz, CDCl₃) δ 7.35 (s, 1H), 7.34-7.26 (m, 1H), 7.20-7.07 (m, 2H), 6.87 (s, 1H), 6.73 (s, 1H), 3.95 (s, 2H), 3.87-3.72 (m, 2H), 2.81-2.70 (m, 2H), 2.70-2.52 (m, 8H), 2.26 (s, 3H), 2.23 (s, 3H), 2.22 (s, 3H). ¹³C NMR (75 MHz, CDCl₃) δ 153.6 (q), 143.9 (q), 133.5 (q), 132.6 (q), 131.9 (t), 130.9 (q), 130.6 (t), 129.9 (t), 129.2 (t), 129.1 (t), 127.6 (t), 122.5 (q), 70.3 (d), 58.2 (d), 55.0 (d), 53.5 (d), 45.9 (s), 35.7 (d), 20.9 (s), 16.5 (s).

### Example 107: 1-(2-(2-benzyl-3,6-dimethylphenoxy)ethyl)-4-methylpiperazine

The whole mixture of 2-benzyl-3,6-dimethylphenol (0.22 g, 1.0 mmol) (prepared in an analogous procedure to Example 79), 1-(2-chloroethyl)-4-methylpiperazine dihydrochloride (0.49 g, 2.1 mmol), potassium carbonate (1.43 g, 10.4 mmol) and potassium iodide (0.030 g, 0.21 mmol) in DMF (10 mL) was heated to 100° C. for 3 hours. Water was added, DCM was used to extract the solution for three times, the solvent was removed and the residue was purified by column chromatography on silica gel (MTBE to DCM:Methanol=10:1) to get the title product (0.15 g, yield 42%) as a viscous liquid. GC/MS (EI): m/z (%): 338 (3) [M⁺], 209 (5), 195 (4), 178 (45), 165 (10), 127(45), 113 (100), 100 (35), 84 (10), 70 (40). ¹H NMR (300 MHz, CDCl₃) δ 7.25-7.18 (m, 2H), 7.16-7.04 (m, 3H), 7.00 (d, J=7.6 Hz, 1H), 6.87 (d, J=7.7 Hz, 1H), 4.10 (s, 2H), 3.75 (t, J=5.9 Hz, 2H), 2.67 (t, J=5.9 Hz, 2H), 2.62-2.40 (m, 8H), 2.31 (s, 3H), 2.29 (s, 3H), 2.14 (s, 3H). ¹³C NMR (75 MHz, CDCl₃) δ 156.3 (q), 140.9 (q), 136.6 (q), 131.7 (q), 129.3 (t), 128.4 (t), 128.4 (t), 128.2 (t), 126.0 (t), 125.7 (t), 70.4 (d), 58.1 (d), 55.0 (d), 53.4 (d), 45.9 (s), 32.5 (d), 19.8 (s), 16.6 (s).

### Example 108: 1-(2-(2-benzyl-4-isopropylphenoxy)ethyl)-4-methylpiperazine

The whole mixture of 2-benzyl-4-isopropylphenol (0.25 g, 1.1 mmol) (prepared in an analogous procedure to Example 79), 1-(2-chloroethyl)-4-methylpiperazine dihydrochloride (0.52 g, 2.2 mmol), potassium carbonate (1.53 g, 11.1 mmol) and potassium iodide (0.040 g, 0.22 mmol) in DMF (10 mL) was heated to reflux for 4 hours. Water was added, DCM was used to extract the solution for three times, the solvent was removed and the residue was purified by column chromatography on silica gel (MTBE to DCM:Methanol=10:1) to get the title product (0.16 g, yield 41%) as a viscous liquid. GC/MS (EI): m/z (%): 352 (2) [M⁺], 207 (4), 195 (1), 178 (2), 165 (4), 127(45), 113 (100), 100 (5), 91 (10), 70 (45). ¹H NMR (300 MHz, CDCl₃) δ 7.29-7.11 (m, 5H), 7.10-6.94 (m, 2H), 6.84-6.71 (m, 1H), 4.07 (s, 2H), 3.96 (s, 2H), 2.90-2.71 (m, 3H), 2.70-2.36 (m, 8H), 2.33 (s, 3H), 1.21 (d, J=3.7, 6H). ¹³C NMR (75 MHz, CDCl₃) δ 154.7 (q), 141.3 (q), 141.1 (q), 129.2 (q), 129.1 (t), 128.8 (t), 128.2 (t), 125.7 (t), 124.9 (t), 111.3 (t), 66.6 (d), 57.3 (d), 54.9 (d), 53.3 (d), 45.8 (s), 36.5 (d), 33.3 (t), 24.3 (s).

### Example 109: 1-(2-((6-benzylbenzo[d][1,3]dioxol-5-yl)oxy)ethyl)-4-methylpiperazine

A mixture of 6-benzylbenzo[d][1,3]dioxol-5-ol (0.10 g, 0.44 mmol) (prepared in an analogous procedure to Example 79), 1-(2-chloroethyl)-4-methylpiperazine dihydrochloride (0.21 g, 0.88 mmol), potassium carbonate (0.61 g, 4.4 mmol) and potassium iodide (0.020 g, 0.090 mmol) in DMF (10 mL) was heated to reflux for 4 hours. Water was added, DCM was used to extract the solution for three times, the solvent was removed and the residue was purified by column chromatography on silica gel (MTBE to DCM:Methanol=10:1) to get the title product (0.09 g, 0.24 mmol, yield 54.3%) as a viscous liquid. GC/MS (EI): m/z (%): 354 (15) [M⁺], 254 (5), 225 (5), 169 (4), 152 (5), 127(100), 113 (85), 98 (10), 84 (12), 70 (45). ¹H NMR (300 MHz, CDCl₃) δ 7.26-7.20 (m, 2H), 7.18-7.11 (m, 3H), 6.58 (s, 1H), 6.50 (s, 1H), 5.86 (s, 2H), 3.98 (t, J=5.6 Hz, 2H), 3.86 (s, 2H), 2.72 (t, J=5.6 Hz, 2H), 2.70-3.38 (m, 8H), 2.32 (s, 3H). ¹³C NMR (75 MHz, CDCl₃) δ 151.4 (q), 146.5 (q), 141.4 (q), 128.8 (t), 128.4 (t), 125.9 (t), 122.2 (q), 110.4 (t), 101.1 (d), 96.1 (t), 67.8 (d), 57.3 (d), 54.9 (d), 53.3 (d), 45.8 (s), 35.9 (d).

### Example 110: 1-(2-((6-benzyl-2,3-dihydro-1H-inden-5-yl)oxy)ethyl)-4-methylpiperazine

A mixture of 6-benzyl-2,3-dihydro-1H-inden-5-ol (0.22 g, 0.98 mmol) (prepared in an analogous procedure to Example 79), 1-(2-chloroethyl)-4-methylpiperazine dihydrochloride (0.46 g, 2.0 mmol), potassium carbonate (1.36 g, 9.8 mmol) and potassium iodide (0.030 g, 0.20 mmol) in DMF (10 mL) was heated to 100° C. (heat on position at 150° C.) for overnight. Water was added, DCM was used to extract the solution for three times, the solvent was removed and the residue was purified by column chromatography on silica gel (MTBE to DCM:Methanol=10:1) to get the title product (0.11 g, yield 30%) as a viscous liquid. GC/MS (EI): m/z (%): 350 (10) [M⁺], 221 (4), 207 (4), 178 (5), 165 (4), 127(55), 113 (100), 100 (5), 91 (10), 70 (435). ¹H NMR (300 MHz, CDCl₃) δ 7.35-7.14 (m, 5H), 6.95 (s, 1H), 6.75 (s, 1H), 4.18-3.87 (m, 4H), 2.92-2.75 (m, 6H), 2.66-2.42 (m, 8H), 2.39-2.28 (m, 3H), 2.05 (m, 2H). ¹³C NMR (75 MHz, CDCl₃) δ 155.4 (q), 143.4 (q), 141.7 (q), 136.0 (q), 128.9 (t), 128.3 (t), 127.5 (q), 126.3 (t), 125.7 (t), 108.0 (t), 66.7 (d), 57.3 (d), 54.9 (d), 53.3 (d), 45.8 (s), 36.3 (d), 33.3 (d), 32.2 (d), 25.8 (d).

### Example 111: 1-(2-(2,4-dimethyl-6-(pyridin-3-ylmethyl)phenoxy)ethyl)-4-methylpiperazine

A mixture of 2,4-dimethyl-6-(pyridin-3-ylmethyl)phenol hydrochloride (0.35 g, 1.1 mmol) (prepared in an analogous procedure to Example 79, and isolated as the hydrochloride salt in an analogous procedure to Example 96), 1-(2-chloroethyl)-4-methylpiperazine dihydrochloride (0.53 g, 2.2 mmol), potassium carbonate (2.32 g, 16.8 mmol) and potassium iodide (0.020 g, 0.11 mmol) in DMF (30 mL) was heated to reflux for 5 hours. Water was added, DCM was used to extract the solution for three times, the solvent was removed and the residue was purified by column chromatography on silica gel DCM:Methanol:TEA=10:1) to get the title product (0.22 g, yield 56%) as a viscous liquid. GC/MS (EI): m/z (%): 339 (30) [M⁺], 324 (5), 296 (15), 283 (20), 269 (85), 127(60), 113 (100), 98 (15), 84 (20), 70 (90). ¹H NMR (300 MHz, CDCl₃) δ 8.49 (s, 1H), 8.41 (d, J=4.7, 1H), 7.46 (d, J=7.8 Hz, 1H), 7.16 (dd, J=7.8, 4.7 Hz, 1H), 6.85 (s, 1H), 6.70 (s, 1H), 3.96 (s, 2H), 3.81 (t, J=5.7 Hz, 2H), 2.74 (t, J=5.7 Hz, 2H), 2.70-2.44 (m, 8H), 2.35 (s, 3H), 2.24 (s, 3H), 2.19 (s, 3H). ¹³C NMR (75 MHz, CDCl₃) δ 153.5 (q), 150.3 (t), 147.5 (t), 136.8 (q), 136.3 (t), 133.6 (q), 132.3 (q), 130.9 (q), 130.7 (t), 129.0 (t), 123.4 (t), 70.4 (d), 58.1 (d), 54.9 (d), 53.1 (d), 45.6 (s), 33.3 (d), 20.8 (s), 16.5 (s).

### Example 112: 1-(2-(2-benzyl-3-methylphenoxy)ethyl)-4-methylpiperazine

A mixture of 2-benzyl-3-methylphenol (0.15 g, 0.76 mmol) (prepared in an analogous procedure to Example 79 and carefully separated from other regioisomers by chromatography), 1-(2-chloroethyl)-4-methylpiperazine dihydrochloride (0.36 g, 1.5 mmol), potassium carbonate (1.05 g, 7.6 mmol) and potassium iodide (0.030 g, 0.15 mmol) in DMF (10 mL) was heated to reflux for 4 hours. Water was added, DCM was used to extract the solution for three times, the solvent was removed and the residue was purified by column chromatography on silica gel (MTBE to DCM:Methanol=10:1) to get the title product (0.10 g, yield 40%) as a viscous liquid. GC/MS (EI): m/z (%): 324 (5) [M⁺], 209 (4), 195 (4), 178 (4), 165 (10), 127(75), 113 (100), 100 (15), 91 (13), 70 (80). ¹H NMR (300 MHz, CDCl₃) δ 7.25-7.17 (m, 2H), 7.16-7.05 (m, 4H), 6.85-6.71 (m, 2H), 4.12-4.01 (m, 4H), 2.75 (t, J=5.5 Hz, 2H), 2.70-2.33 (m, 8H), 2.29 (s, 3H), 2.25 (s, 3H). ¹³C NMR (75 MHz, CDCl₃) δ 156.9 (q), 140.9 (q), 138.6 (q), 128.2 (t), 127.6 (q), 127.1 (t), 125.6 (t), 122.9 (t), 109.2 (t), 66.8 (d), 57.2 (d), 54.9 (d), 53.3 (d), 45.8 (s), 31.9 (d), 19.9 (s).

### Example 113: 4-methyl-2-(5-methyl-2-(2-(4-methylpiperazin-1-yl)ethoxy)benzyl)phenol

To a suspension of sodium hydride (56 wt %, 0.95 g, 22.2 mmol) in DMF (10 mL) was added 2,2′-methylenebis(4-methylphenol) (1.27 g, 5.6 mmol) and 1-(2-chloroethyl)-4-methylpiperazine dihydrochloride (1.7 g, 7.2 mmol) subsequently and the resulting mixture was stirred at rt for 2 days. Water was added, EA was used to extract the solution, the solvent was removed and the residue was purified by column chromatography (DCM:Methanol=10:1) to get 150 mg brown solid. The obtained material was mixed with diluted HCl and then the whole mixture was concentrated to remove water to get the title product (0.11 g, yield 6%) as a white solid. GC/MS (EI): m/z (%): 354 (26) [Mf], 339 (3), 209 (4), 165 (4), 127 (30), 113 (100), 98 (5), 84 (3), 70 (25).

### Example 114: 1-(2-(2,4-dimethyl-6-(pyridin-4-ylmethyl)phenoxy)ethyl)-4-methylpiperazine

A mixture of 2,4-dimethyl-6-(pyridin-4-ylmethyl)phenol hydrochloride (0.19 g, 0.46 mmol) (prepared in an analogous procedure to Example 79, and isolated as the hydrochloride salt in an analogous procedure to Example 96), 1-(2-chloroethyl)-4-methylpiperazine dihydrochloride (0.22 g, 0.91 mmol), potassium carbonate (0.95 g, 6.8 mmol) and potassium iodide (7.6 mg, 0.050 mmol) in DMF (Volume: 30 mL) was heated to reflux for overnight. Water was added, DCM was used to extract the solution for three times, the solvent was removed and the residue was purified by column chromatography on silica gel (DCM:Methanol:TEA=10:1) to get the title product (0.060 g, yield 38%) as a viscous liquid. GC/MS (EI): m/z (%): 339 (10) [M⁺], 324 (53), 283 (5), 260 (8), 240 (4), 213(75), 127 (40), 113 (100), 98 (8), 84 (8), 70 (50). ¹H NMR (300 MHz, CDCl₃) δ 8.43 (d, J=5.7 Hz, 2H), 7.08 (d, J=5.7 Hz, 2H), 6.82 (s, 1H), 6.69 (s, 1H), 3.93 (s, 2H), 3.76 (t, J=5.5 Hz, 2H), 3.07 (q, J=7.3 Hz, 1H), 2.86-2.68 (m, 9H), 2.52 (s, 3H), 2.22 (s, 3H), 2.18 (s, 3H). ¹³C NMR (75 MHz, CDCl₃) δ 153.4 (q), 150.5 (q), 149.7 (t), 133.8 (q), 131.2 (q), 130.9 (t), 130.9 (q), 129.3 (t), 124.1 (t), 70.2 (d), 57.6 (d), 54.3 (d), 51.8 (d), 44.7 (s), 35.5 (d), 20.7 (s), 16.5 (s), 8.7 (s).

### Example 115: 1-((2-bromobenzyl)oxy)-2,4-dimethylbenzene

A suspension of potassium carbonate (42.4 g, 307 mmol), 1-bromo-2-(bromomethyl)benzene (48.6 g, 194 mmol) and 2,4-dimethylphenol (25.0 g, 205 mmol) in acetone (300 mL) was heated at reflux for 14 hours. Then the solvent was removed by rotary evaporation, water (200 mL) was added, MTBE (100 mL×3) was used to extract the solution, the organic phase was dried over Na₂SO₄ and the solvent was removed to get the title product (56.0 g, yield 89%) as a white solid, which was used for next step directly. GC/MS (EI): m/z (%): 292 (98) [M⁺], 290 (100) [M⁺], 211 (5), 171 (24), 121 (5), 90 (45), 77 (25).

### Example 116: 2-(2-bromobenzyl)-4,6-dimethylphenol

To 1-((2-bromobenzyl)oxy)-2,4-dimethylbenzene in dichloromethane (200 mL) was added dropwise titanium (IV) chloride (39.1 g, 206 mmol) at 0° C. and the mixture was stirred at 0° C. for 10 minutes. Water (150 mL) was added to quench the reaction, dichloromethane (100 mL×3) was used to extract the solution, the organic phase was dried over MgSO₄ and the solvent was removed. The residue liquid was purified by column chromatography on silica gel (PE:MTBE=95:5) to get the title product (11.5 g, yield 17%) as a colorless liquid. GC/MS (EI): m/z (%): 292 (95) [M⁺], 290 (98) [M⁺], 211 (90), 196 (80), 181 (40), 165 (50), 134 (100), 91 (40).

### Example 117: 2-(2-hydroxy-3,5-dimethylbenzyl)benzonitrile

A mixture of 2-(2-bromobenzyl)-4,6-dimethylphenol (1.0 g, 3.4 mmol) and Copper(I) cyanide (0.33 g, 3.7 mmol) in N-methyl-2-pyrrolidone (10 mL) was heated to reflux for 1 hour. The cooled reaction mixture was diluted with water (20 mL), extracted with MTBE (50 mL×3), dried over Na₂SO₄, and concentrated under reduced pressure. The residue was purified by column chromatography on silica gel (PE:MTBE=85:15) to afford the title product (0.46 g, yield 56%) as a light red solid. GC/MS (EI): m/z (%): 237 (100) [M⁺], 222 (158), 208 (55), 194 (50), 182 (35), 165 (15), 121(40), 91 (20), 77 (15).

### Example 118: 2-(3,5-dimethyl-2-(2-morpholinoethoxy)benzyl)benzonitrile

Following the general procedure from Example 80: The mixture of 2-(2-hydroxy-3,5-dimethylbenzyl)benzonitrile (0.65 g, 2.74 mmol), 4-(2-chloroethyl)morpholine (0.82 g, 5.48 mmol), potassium carbonate (3.79 g, 27.4 mmol) and potassium iodide (0.09 g, 0.55 mmol) in DMF (15 mL) were reacted to give the title product (0.83 g, yield 84%) as a viscous liquid. GC/MS (EI): m/z (%): 350 (7) [M⁺], 263 (23), 234 (5), 208 (3), 190 (4), 165 (3), 140 (1), 100 (100), 56 (14). ¹H NMR (300 MHz, CDCl₃) δ 7.64 (d, J=7.7 Hz, 1H), 7.45 (t, J=7.3 Hz, 1H), 7.32-7.19 (m, 2H), 6.90 (s, 1H), 6.71 (s, 1H), 4.23 (s, 2H), 3.88 (t, J=5.7 Hz, 2H), 3.77-3.63 (m, 4H), 2.74 (t, J=5.7 Hz, 2H), 2.62-2.46 (m, 4H), 2.28 (s, 3H), 2.22 (s, 3H). ¹³C NMR (75 MHz, CDCl₃) δ 153.7 (q), 145.3 (q), 133.7 (q), 132.9 (t), 132.8 (t), 131.5 (q), 131.1 (t), 131.0 (q), 130.1 (t), 129.2 (t), 126.7 (t), 118.5 (q), 112.6 (q), 70.2 (d), 67.1 (d), 58.8 (d), 54.3 (d), 34.4 (d), 20.9 (s), 16.5 (s).

### Example 119: 2-(3,5-dimethyl-2-(2-morpholinoethoxy)benzyl)benzonitrile hydrochloride

To the solution of 2-(3,5-dimethyl-2-(2-morpholinoethoxy)benzyl)benzonitrile (40 mg, 0.11 mmol) in methanol (15 mL) was added hydrogen chloride (224 mg, 1.1 mmol) and the resulting mixture was stirred room temperature for 30 min. The solvent was removed and the residue was dried at 90° C. under reduced pressure to get the title product (40 mg, yield 89%) as a viscous liquid. GC/MS (EI): m/z (%): 350 (3) [M⁺], 263 (14), 234 (4), 190 (3), 165 (2), 140 (1), 114 (15), 100 (100), 87 (1), 70 (3).

### Example 120: 2-(3,5-dimethyl-2-(2-(4-methylpiperazin-1-yl)ethoxy)benzyl)benzonitrile

Following the general procedure from Example 80: The mixture of 2-(2-hydroxy-3,5-dimethylbenzyl)benzonitrile (0.2 g, 0.843 mmol), 1-(2-chloroethyl)-4-methylpiperazine dihydrochloride (0.298 g, 1.264 mmol), potassium carbonate (0.93 g, 6.7 mmol) and potassium iodide (0.028 g, 0.17 mmol) in DMF (10 mL) were reacted to give the title product (0.10 g, yield 31%) as a viscous liquid. GC/MS (EI): m/z (%): 363 (1) [M⁺], 234 (1), 208 (4), 190 (4), 165 (3), 127 (69), 113 (100), 100 (9), 84 (6), 70 (37). ¹H NMR (300 MHz, CDCl₃) δ 7.63 (d, J=7.7, 1H), 7.49-7.38 (m, 1H), 7.32-7.17 (m, 2H), 6.88 (s, 1H), 6.71 (s, 1H), 4.22 (s, 2H), 3.86 (t, J=5.8 Hz, 2H), 2.74 (t, J=5.8 Hz, 2H), 2.70-2.34 (m, 8H), 2.28 (s, 3H), 2.27 (s, 3H), 2.21 (s, 3H). ¹³C NMR (75 MHz, CDCl₃) δ 153.7 (q), 145.4 (q), 133.6 (q), 132.9 (t), 132.7 (t), 131.5 (q), 131.0 (t), 130.1 (t), 129.1 (t), 126.6 (t), 118.5 (q), 112.5 (q), 70.4 (d), 58.2 (d), 55.1 (d), 53.7 (d), 46.1 (s), 34.3 (d), 20.9 (s), 16.5 (s).

### Example 121: 2-(3,5-dimethyl-2-(2-(4-methylpiperazin-1-yl)ethoxy)benzyl)benzonitrile hydrochloride

To 2-(3,5-dimethyl-2-(2-(4-methylpiperazin-1-yl)ethoxy)benzyl)benzonitrile (0.60 g, 1.6 mmol) in Methanol (10 mL) was added hydrogen chloride (0.50 g, 4.8 mmol) and the resulting mixture was standing for half an hour. Then the solvent was removed by rotary evaporation and the residue was dried over Kugelrohr (90° C., 0.1 mbar) to get the title product (0.65 g, yield 96%) as a light brown solid. GC/MS (EI): m/z (%): 363 (1) [M⁺], 248 (1), 208 (4), 190 (4), 165 (3), 127 (69), 113 (100), 100 (9), 84 (6), 70 (37).

### Example 122: 2-(5-methyl-2-(2-(4-methylpiperazin-1-v)ethoxy)benzyl)benzonitrile

Following the general procedure from Example 80: The mixture of 2-(2-hydroxy-5-methylbenzyl)benzonitrile (0.50 g, 2.2 mmol) (prepared in an analogous sequence as described in

Example 115, Example 116 and Example 117), 1-(2-chloroethyl)-4-methylpiperazine dihydrochloride (1.06 g, 4.5 mmol), potassium carbonate (3.10 g, 22.4 mmol) and potassium iodide (0.070 g, 0.45 mmol) in DMF (15 mL) were reacted to give the title product (0.48 g, yield 58%) as a brown viscous liquid. GC/MS (EI): m/z (%): 349 (1) [M⁺], 248 (1), 220 (2), 190 (6), 165 (3), 127 (58), 113 (100), 98 (5), 84 (4), 70 (34). ¹H NMR (300 MHz, CDCl₃) δ 7.62 (d, J=7.6 Hz, 1H), 7.44 (t, J=7.6 Hz, 1H), 7.30-7.20 (m, 2H), 7.02 (d, J=8.2 Hz, 1H), 6.99 (s, 1H), 6.76 (d, J=8.2 Hz, 1H), 4.17 (s, 2H), 4.06 (t, J=5.7 Hz, 2H), 2.75 (t, J=5.7 Hz, 2H), 2.66-2.42 (m, 8H), 2.32 (s, 3H), 2.27 (s, 3H). ¹³C NMR (75 MHz, CDCl₃) δ 154.5 (q), 145.3 (q), 132.7 (t), 131.7 (t), 130.1 (q), 129.8 (t), 128.6 (t), 127.2 (q), 126.4 (t), 118.5 (q), 112.6 (q), 111.6 (t), 66.4 (d), 57.2 (d), 55.0 (d), 53.3 (d), 45.9 (s), 34.7 (d), 20.6 (s).

### Example 123: 2-(3,5-dimethyl-2-(2-morpholinoethoxy)benzyl)-3-fluorobenzonitrile

Following the general procedure from Example 80: The mixture of 3-fluoro-2-(2-hydroxy-3,5-dimethylbenzyl)benzonitrile (0.15 g, 0.59 mmol) (prepared in an analogous sequence as described in

Example 115, Example 116 and Example 117), 4-(2-chloroethyl)morpholine hydrochloride (0.22 g, 1.2 mmol), potassium carbonate (0.81 g, 5.9 mmol) and potassium iodide (0.020 g, 0.12 mmol) in DMF (15 mL) were reacted to give the title product (0.060 g, yield 26%) as a brown viscous liquid. GC/MS (EI): m/z (%): 368 (2) [M⁺], 281 (11), 208 (2), 183 (2), 158 (1), 134 (3), 114 (19), 100 (100), 85 (1), 70 (4). ¹H NMR (300 MHz, CDCl₃) δ 7.50 (d, J=7.2 Hz, 1H), 7.41-7.27 (m, 2H), 6.86 (s, 1H), 6.37 (s, 1H), 4.27 (s, 2H), 3.99 (t, J=5.8 Hz, 2H), 3.82-3.67 (m, 4H), 2.83 (t, J=5.8 Hz, 2H), 2.68-2.53 (m, 4H), 2.28 (s, 3H), 2.15 (s, 3H). ¹³C NMR (75 MHz, CDCl₃) δ 153.4 (q), 133.4 (q), 132.1 (q), 131.8 (q), 130.8 (t), 130.7 (q), 130.4 (q), 129.1 (t), 129.0 (t), 128.9 (t), 128.9 (t), 127.1 (t), 120.7 (t), 120.4 (t), 115.6 (q), 115.5 (q), 70.0 (d), 67.1 (d), 58.8 (d), 54.4 (d), 28.1 (d), 20.9 (s), 16.5 (s).

### Example 124: 2-(3,5-dimethyl-2-(2-morpholinopropoxy)benzyl)benzonitrile and 2-(3,5-dimethyl-2-((1-morpholinopropan-2-yl)oxy)benzyl)benzonitrile

Following the general procedure from Example 80: 4-(2-chloropropyl)morpholine hydrochloride (0.40 g, 2.0 mmol), 2-(2-hydroxy-3,5-dimethylbenzyl)benzonitrile (0.240 g, 0.99 mmol), potassium carbonate (1.37 g, 9.9 mmol) and potassium iodide (0.033 g, 0.20 mmol) in DMF (10 mL) were reacted to give 2-(3,5-dimethyl-2-(2-morpholinopropoxy)benzyl)benzonitrile (0.080 g, yield 22%) as a brown viscous liquid and 2-(3,5-dimethyl-2-((1-morpholinopropan-2-yl)oxy)benzyl)benzonitrile (0.040 g, yield 11%) as a brown viscous liquid.
2-(3,5-dimethyl-2-(2-morpholinopropoxy)benzyl)benzonitrile: GC/MS (EI): m/z (%): 364 (1) [M⁺], 277 (1), 234 (2), 190 (2), 165 (1), 128 (5), 114 (100), 98 (1), 84 (1), 70 (3). ¹H NMR (300 MHz, CDCl₃) δ 7.64 (d, J=7.7 Hz, 1H), 7.46 (t, J=7.2 Hz, 1H), 7.32-7.24 (m, 1H), 7.20 (d, J=7.9 Hz, 1H), 6.90 (s, 1H), 6.70 (s, 1H), 4.23 (s, 2H), 3.88-3.78 (m, 1H), 3.73-3.58 (m, 5H), 3.01-2.88 (m, 1H), 2.66-2.52 (m, 4H), 2.28 (s, 3H), 2.22 (s, 3H), 1.13 (d, J=6.7 Hz, 3H). ¹³C NMR (75 MHz, CDCl₃) δ 153.6 (q), 145.3 (q), 133.7 (q), 132.9 (t), 132.7 (t), 131.3 (q), 131.1 (t), 131.0 (q), 130.0 (t), 129.2 (t), 126.6 (t), 118.4 (q), 112.6 (q), 74.5 (d), 67.4 (d), 59.6 (t), 49.8 (d), 34.4 (d), 20.9 (s), 16.6 (s), 12.5 (s).
2-(3,5-dimethyl-2-((1-morpholinopropan-2-yl)oxy)benzyl)benzonitrile: GC/MS (EI): m/z (%): 364 (1) [M⁺], 349 (2), 277 (3), 234 (10), 190 (10), 165 (1), 128 (5), 114 (100), 98 (1), 84 (1), 70 (5).

### Example 125: 2-(3,5-dimethyl-2-(2-(tetrahydro-2H-pyran-4-yl)ethoxy)benzyl)benzonitrile

Following the general procedure from Example 80: The mixture of 2-(2-hydroxy-3,5-dimethylbenzyl)benzonitrile (0.15 g, 0.63 mmol), 4-(2-bromoethyl)tetrahydro-2H-pyran (0.24 g, 1.3 mmol), potassium carbonate (0.87 g, 6.3 mmol) and potassium iodide (0.020 g, 0.13 mmol) in DMF (15 mL) were reacted to give the title product (0.18 g, yield 79%) as a viscous liquid. GC/MS (EI): m/z (%): 349 (1) [M⁺], 236 (18), 222 (5), 208 (18), 182 (13), 165 (4), 130 (2), 118 (100), 83 (7), 69 (7). ¹H NMR (300 MHz, ¹H NMR (300 MHz, CDCl₃) δ 7.64 (d, J=7.7, 1H), 7.48-7.44 (m, 1H), 7.36-7.23 (m, 1H), 7.16 (d, J=7.8 Hz, 1H), 6.91 (s, 1H), 6.72 (s, 1H), 4.19 (s, 2H), 3.98-3.92 (m, 2H), 3.77 (t, J=6.4 Hz, 2H), 3.40-3.30 (m, 2H), 2.30 (s, 3H), 2.20 (s, 3H), 1.72 (t, J=5.4 Hz, 3H), 1.67-1.50 (m, 2H), 1.47-1.10 (m, 2H). ¹³C NMR (75 MHz, CDCl₃) δ 153.7 (s), 145.2 (s), 133.5 (s), 132.9 (d), 132.6 (d), 131.2 (s), 131.0 (d), 129.8 (d), 129.1 (d), 126.5 (d), 118.3 (s), 112.5 (s), 70.4 (t), 68.0 (t), 37.3 (t), 34.3 (t), 33.2 (t), 31.9 (d), 20.8 (q), 16.4 (q).

### Example 126: 2-(5-methyl-2-(2-(4-methylpiperazin-1-yl)ethoxy)benzyl)benzonitrile dihydrochloride

To a solution of 2-(5-methyl-2-(2-(4-methylpiperazin-1-yl)ethoxy)benzyl)benzonitrile (0.62 g, 1.7 mmol) in Methanol (10 mL) was added dropwise hydrogen chloride (0.52 g, 5.2 mmol) and the resulting mixture was allowed to stand room temperature for 1 hour. Then the solvent was removed under reduced pressure by rotary evaporation and the resulting solid was dried under vacuum at 80° C. by kugelrohr for 1 hour. The target product (0.67 g, yield 89%) was obtained as a light brown solid. GC/MS (EI): m/z (%): 349 (4) [M⁺], 279 (1), 220 (2), 190 (5), 165 (3), 127 (55), 113 (100), 98 (5), 84 (4), 70 (35).

### Example 127: 2-(2-(3,5-dimethyl-2-(2-morpholinoethoxy)benzyl)phenyl)acetonitrile

Following the general procedure from Example 80: The mixture of 2-(2-(2-hydroxy-3,5-dimethylbenzyl)phenyl)acetonitrile (0.18 g, 0.72 mmol) (prepared in an analogous procedure to Example 79), 4-(2-chloroethyl)morpholine hydrochloride (0.27 g, 1.4 mmol), potassium carbonate (0.99 g, 7.2 mmol) and potassium iodide (0.020 g, 0.14 mmol) in DMF (15 mL) were reacted to give the title product (0.050 g, yield 11%) as a viscous liquid. GC/MS (EI): m/z (%): 364 (2) [Ml], 277 (15), 234 (8), 209 (5), 191 (1), 165 (3), 130 (2), 100 (100), 77 (1), 56 (6).

### Example 128: 3-bromo-2-(3,5-dimethyl-2-(2-morpholinoethoxy)benzyl)benzonitrile

Following the general procedure from Example 80: The mixture of 3-bromo-2-(2-hydroxy-3,5-dimethylbenzyl)benzonitrile (0.25 g, 0.79 mmol) (prepared in an analogous sequence as described in

Example 115, Example 116 and Example 117, starting from 1,3-dibromo-2-(bromomethyl)benzene instead of 1-bromo-2-(bromomethyl)benzene), 4-(2-chloroethyl)morpholine hydrochloride (0.29 g, 1.58 mmol), potassium carbonate (1.09 g, 7.91 mmol) and potassium iodide (0.03 g, 0.16 mmol) in DMF (15 mL) was heated to reflux for 3 hours. Water was added, DCM was used to extract the solution for three times, the solvent was removed and the residue was purified by column chromatography on silica gel (PE (petroleum ether):MTBE=1:1) to get the title product (0.22 g, 0.48 mmol, yield 60%) as a viscous liquid. GC/MS (EI): m/z (%): 430 (2) [M⁺], 428 (2) [M⁺], 345 (10), 343 (10), 234 (8), 204 (8), 234 (8), 114 (25), 100 (100), 88 (3), 79 (2), 70 (10), 56 (15).

### Example 129: 2-(5-methyl-2-(2-morpholinopropoxy)benzyl)benzonitrile

Following the general procedure from Example 80: 2-(2-hydroxy-5-methylbenzyl)benzonitrile (0.30 g, 1.3 mmol), 4-(2-chloropropyl)morpholine hydrochloride (0.52 g, 2.6 mmol), potassium carbonate (1.8 g, 13 mmol) and potassium iodide (0.043 g, 0.26 mmol) in DMF (13 mL) were reacted to give the title product (0.010 g, yield 2%) as a viscous liquid. GC/MS (EI): m/z (%): 350 (2) [M⁺], 263 (52), 220 (4), 204 (2), 190 (10), 178 (5), 128 (10), 1146 (5), 100 (100), 91 (3), 77 (2), 70 (5), 56 (10). ¹H NMR (300 MHz, CDCl₃) δ 7.63 (d, J=7.6 Hz, 1H), 7.42 (t, J=7.2 Hz, 1H), 7.29-7.22 (m, 2H), 7.17 (d, J=7.8 Hz, 1H), 7.03 (d, J=8.2 Hz, 1H), 6.97 (s, 1H), 6.76 (d, J=8.3 Hz, 1H), 4.18 (s, 2H), 4.05-3.94 (m, 1H), 3.88-3.78 (m, 1H), 3.70-3.60 (m, 4H), 2.97-2.80 (m, 1H), 2.60-2.46 (m, 4H), 2.27 (s, 3H), 1.67 (s, 1H), 1.05 (d, J=6.7 Hz, 3H). ¹³C NMR (75 MHz, CDCl₃) δ 154.6 (q), 145.4 (q), 132.7 (t), 131.8 (t), 130.1 (q), 129.7 (t), 128.6 (t), 127.0 (q), 126.5 (t), 118.4 (q), 112.6 (q), 111.4 (t), 70.2 (d), 67.5 (d), 58.8 (t), 49.9 (d), 34.8 (d), 20.6 (s), 13.1 (s).

### Example 130: 3-chloro-2-(3,5-dimethyl-2-(2-morpholinoethoxy)benzyl)benzonitrile

Following the general procedure from Example 80: The mixture of 3-chloro-2-(2-hydroxy-3,5-dimethylbenzyl)benzonitrile (0.080 g, 0.28 mmol) (prepared in an analogous sequence as described in

Example 115, Example 116 and Example 117), 4-(2-chloroethyl)morpholine hydrochloride (0.10 g, 0.55 mmol), potassium carbonate (0.38 g, 2.8 mmol) and potassium iodide (9.2 mg, 0.06 mmol) in DMF (10 mL) were reacted to give the title product (0.070 g, yield 64%) as a viscous liquid. GC/MS (EI): m/z (%): 384 (1) [M⁺], 297 (10), 234 (5), 204(2), 190 (2), 128 (7), 114 (20), 100(100), 88 (1), 70 (3). ¹H NMR (300 MHz, CDCl₃) δ 7.64 (d, J=7.9 Hz, 2H), 7.35 (t, J=7.9 Hz, 1H), 6.85 (s, 1H), 6.10 (s, 1H), 4.41 (s, 2H), 4.01 (t, J=5.7 Hz, 2H), 3.82-3.69 (m, 4H), 2.85 (t, J=5.7 Hz, 2H), 2.69-2.55 (m, 4H), 2.30 (s, 3H), 2.12 (s, 3H). ¹³C NMR (75 MHz, CDCl₃) δ 153.4 (q), 142.1 (q), 136.6 (q), 134.4 (t), 133.4 (q), 131.7 (t), 130.6 (t), 129.9 (q), 128.4 (t), 126.2 (t), 117.5 (q), 116.0 (q), 70.0 (d), 67.1 (d), 58.9 (d), 54.4 (d), 32.5 (d), 21.0 (s), 16.4 (s).

### Example 131: 2-(3,5-dimethyl-2-(3-morpholinopropoxy)benzyl)benzonitrile

Following the general procedure from Example 80: The mixture of 2-(2-hydroxy-3,5-dimethylbenzyl)benzonitrile (0.13 g, 0.55 mmol), 4-(3-chloropropyl)morpholine (0.13 g, 0.82 mmol), potassium carbonate (0.76 g, 5.5 mmol) and potassium iodide (0.020 g, 0.11 mmol) in DMF (10 mL) was heated to reflux for 3 hours. Water (50 mL) was added, DCM (3×30 mL) was used to extract the solution for three times, the solvent was removed and the residue was puri-fied by column chromatography on silica gel (PE:MTBE=1:1) to get the title product (0.15 g, yield 71%) as a colorless liquid. GC/MS (EI): m/z (%): 364 (1) [M⁺], 349 (1), 234 (2), 208 (5), 128 (25), 116 (5), 100 (100), 86(3), 70 (10). ¹H NMR (300 MHz, CDCl₃) δ 7.63 (s, 1H), 7.43 (s, 1H), 7.26 (s, 1H), 7.18 (s, 1H), 6.89 (s, 1H), 6.70 (s, 1H), 4.19 (s, 2H), 3.78 (d, J=3.7 Hz, 2H), 3.68 (s, 4H), 2.59-2.35 (m, 6H), 2.26 (s, 2H), 2.21 (s, 2H), 1.95 (s, 2H). ¹³C NMR (75 MHz, CDCl₃) δ 153.6 (q), 145.4 (q), 133.6 (q), 132.9 (t), 132.7 (t), 131.5 (q), 131.2 (q), 131.0 (t), 130.0 (t), 129.1 (t), 126.6 (t), 118.4 (q), 112.5 (q), 70.9 (d), 67.1 (d), 55.4 (d), 53.8 (d), 34.4 (d), 27.4 (d), 20.9 (s), 16.4 (s).

### Example 132: ethyl 4-(3,5-dimethyl-2-(2-(4-methylpiperazin-1-yl)ethoxy)benzyl)benzoate dihydrochloride

A solution of 1-(2-(2-(4-bromobenzyl)-4,6-dimethylphenoxy)ethyl)-4-methylpiperazine (0.30 g, 0.72 mmol) (prepared according in analogous sequence of steps as described in Example 79 and Example 80) in anhydrous THF (20 mL) was cooled to −78° C., and n-Butyllithium (0.5 ml, 1.6 N in hexane, 0.79 mmol) was added dropwise under Argon and the solution was stirred at the same temperature for 1 h before ethyl carbonochloridate (0.31 g, 2.9 mmol) was added. Saturated aq. NH₄Cl was added after another 4 h and the mixture was warmed to room temperature. The THF was evaporated on a rotary evaporator. The residue was extracted with CH₂Cl₂ (20 mL×3), the combined organic layers were dried over Na₂SO₄, filtered, and concentrated in vacuo to afford crude liquid. The crude product was added to a Biotage to get the title product (0.15 g, yield 50%) as a white solid. GC/MS (EI): m/z (%): 410 (4) [M⁺], 365 (5), 209 (5), 195 (3), 127 (55), 113 (100), 100 (15), 84(5), 70 (25).

### Example 133: 3-(3,5-dimethyl-2-(2-(4-methylpiperazin-1-yl)ethoxy)benzyl)benzonitrile dihydrochloride

To 3-(3,5-dimethyl-2-(2-(4-methylpiperazin-1-yl)ethoxy)benzyl)benzonitrile (55 mg, 0.15 mmol) (prepared in an analogous sequence as described in

Example 115, Example 116 and Example 117, followed by Example 80) in Methanol (10 mL) was added hydrogen chloride (153 mg, 1.47 mmol) and the resulting mixture was standing for half an hour. Then the solvent was removed by rotary evaporation, the title product (55 mg, 0.14 mmol, yield 91%) a light brown solid was obtained. GC/MS (EI): m/z (%): 363 (1) [M⁺], 248 (1), 204 (3), 190 (5), 165 (2), 127 (65), 113 (100), 100 (7), 84 (6), 70 (34).

### Example 134: 2-(2,4-dimethyl-5-(2-(4-methylpiperazin-1-yl)ethoxy)benzyl)benzonitrile

Following the general procedure from Example 80: The mixture of 2-(5-hydroxy-2,4-dimethylbenzyl)benzonitrile (0.16 g, 0.66 mmol) (isolated as an isomeric side-product in the preparation of 2-(2-hydroxy-3,5-dimethylbenzyl)benzonitrile according to the sequence described in

Example 115, Example 116 and Example 117), 1-(2-chloroethyl)-4-methylpiperazine dihydrochloride (0.31, 1.3 mmol), potassium carbonate (0.91 g, 6.6 mmol) and potassium iodide (0.022 g, 0.13 mmol) in DMF (10 mL) were reacted to give the title product (0.13 g, yield 52%) as a viscous liquid. GC/MS (EI): m/z (%): 361 (1) [M⁺], 294 (1), 204 (2), 190 (2), 165 (2), 127 (40), 113 (100), 98 (5), 84 (3), 70 (32). ¹H NMR (300 MHz, CDCl₃) δ 7.65 (d, J=7.0 Hz, 1H), 7.44 (t, J=7.7 Hz, 1H), 7.29 (d, J=7.5 Hz, 1H), 7.01 (d, J=7.8 Hz, 1H), 6.95 (s, 1H), 6.55 (s, 1H), 4.14 (s, 2H), 4.02 (t, J=5.6 Hz, 2H), 2.82 (t, J=5.7 Hz, 2H), 2.78-2.64 (m, 4H), 2.63-2.46 (m, 4H), 2.35 (s, 3H), 2.17 (s, 3H), 2.13 (s, 3H). ¹³C NMR (75 MHz, CDCl₃) δ 155.3 (q), 144.7 (q), 134.7 (q), 133.1 (t), 133.0 (t), 132.9 (t), 129.4 (t), 128.5 (q), 126.7 (t), 125.5 (q), 118.2 (q), 113.5 (t), 112.7 (q), 66.8 (d), 57.3 (d), 55.1 (d), 53.3 (d), 45.8 (s), 37.8 (d), 18.7 (s), 15.9 (s).

### Example 135: 2-(5-methyl-2-(2-(1-methylpiperidin-4-yl)ethoxy)benzyl)benzonitrile

Thionyl chloride (2.99 g, 25 mmol) was added to 2-(1-methylpiperidin-4-yl)ethan-1-ol (0.20 g, 1.3 mmol) in CDCl₃ (10 mL) and the resulting mixture was heated to reflux for 5 hours. The solvent was removed by rotary evaporation to get crude chloride. This intermediate was mixed with 2-(2-hydroxy-5-methylbenzyl)benzonitrile (0.15 g, 0.63 mmol), potassium carbonate (1.74 g, 12 mmol) and potassium iodide (0.020 g, 0.13 mmol) in DMF (10 mL) and heated to reflux for 2 hours. After it was cooled to rt., water was added to dilute the solution, DCM was used to extract the solution, the solvent including DMF was removed and the residue was purified by column chromatography on silica gel (DCM:Methanol=10:1) to get the title product (0.050 g, yield 22%) as a dark liquid. GC/MS (EI): m/z (%): 348 (60) [M⁺], 333 (5), 222 (5), 190 (10), 142 (10), 126(100), 112 (20), 96 (20), 70 (15). ¹H NMR (300 MHz, CDCl₃) δ 7.57 (d, J=7.6 Hz, 1H), 7.40 (t, J=7.3 Hz, 1H), 7.25-7.17 (m, 1H), 7.10 (d, J=7.8 Hz, 1H), 6.98 (d, J=8.2 Hz, 1H), 6.91 (s, 1H), 6.68 (d, J=8.3 Hz, 1H), 4.07 (s, 2H), 3.86 (t, J=5.5 Hz, 2H), 3.26 (d, J=11.4 Hz, 2H), 2.60 (s, 3H), 2.55-2.49 (m, 1H), 2.22 (s, 3H), 1.88-1.45 (m, 8H). ¹³C NMR (75 MHz, CDCl₃) δ 154.4 (q), 145.3 (q), 132.9 (t), 132.6 (t), 131.7 (q), 129.9 (t), 129.6 (t), 128.7 (q), 126.8 (t), 126.6 (q), 118.6 (q), 111.2 (t), 64.5 (d), 54.8 (d), 44.3 (s), 34.9 (d), 30.2 (t), 29.4 (d), 20.6 (s).

### Example 136: 2-(3,5-dimethyl-2-(3-morpholinopropyl)phenoxy)benzonitrile

### Example 136a: 1-(allyloxy)-3,5-dimethylbenzene

The mixture of 3,5-dimethylphenol (25.0 g, 205 mmol), 3-bromoprop-1-ene (32.2 g, 266 mmol) and potassium carbonate (56.6 g, 409 mmol) in acetone (400 mL) was heated to reflux for overnight. The solvent was removed by rotary evaporation, water (200 mL) was added, MTBE (100 mL×3) was used to extract the mixture, the combined organic phase was dried over MgSO₄ and the solvent was removed by rotary evaporation to get the title product (33.0 g, yield 96%) as a colorless liquid. GC/MS (EI): m/z (%): 162 (100) [M⁺], 147 (98), 119 (60), 107 (30), 91 (75), 77 (50), 65 (15).

### Example 136b: 1-(allyloxy)-3,5-dimethylbenzene

The neat 1-(allyloxy)-3,5-dimethylbenzene (5.0 g, 29.3 mmol) was heated to 200° C. under Ar for half an hour. After the reaction was cooled to rt., it was purified by column chromatography on silica gel (PE:MTBE=92:8) to get the title product (3.6 g, yield 72%) as a colorless liquid. GC/MS (EI): m/z (%): 162 (85) [M⁺], 147 (100), 135 (25), 119 (50), 91 (55), 77 (35), 65 (20).

### Example 136c: 2-(2-allyl-3,5-dimethylphenoxy)benzonitrile

To the mixture of 2-allyl-3,5-dimethylphenol (2.0 g, 11.7 mmol), 2-iodobenzonitrile (4.0 g, 17.5 mmol), potassium phosphate (5.0 g, 23.4 mmol) and picolinic acid (0.4 g, 3.5 mmol) in DMSO (40 mL) under Ar was added copper(I) iodide (0.34 g, 1.8 mmol). The mixture was evacuated and flushed with Ar for three times and the resulting mixture was heated to 90° C. for overnight. After the reaction mixture was cooled to rt., diluted NaCl solution (100 mL) was added, MTBE (100 mL×3) was used to extract the solution, the combined organic phase was dried over MgSO₄, the solvent was removed and the residue was purified by column chromatography on silica gel (PE:MTBE=9:1) to get the title product (2.5 g, yield 77%) as a colorless liquid. GC/MS (EI): m/z (%): 263 (75) [M⁺], 248 (100), 231 (40), 220 (25), 128 (40), 115 (35), 91 (25).

### Example 136d: 2-(2-(3-hydroxypropyl)-3,5-dimethylphenoxy)benzonitrile

To the solution of 2-(2-allyl-3,5-dimethylphenoxy)benzonitrile (0.5 g, 1.9 mmol) in tetrahedrofuran (20 mL) at 0° C. under Ar was added dropwise BH₃.Me₂S (2 M solution in THF, 2.4 mL, 4.8 mmol). After the mixture was stirred at room temperature for 2 hours, it was cooled to 0° C. and treated carefully with NaOH solution (3 M, 3 mL, 6.6 mmol) followed by addition of H₂O₂ (30% solution, 2.6 g, 22.8 mmol). The solution was stirred at room temperature for 15 mins and then heated to reflux for 2 hours. After the solution was cooled to rt., water (30 mL) was added, ethyl acetate (40 mL×3) was used to extract the solution, the combined organic phase was dried over MgSO₄, the solvent was removed by rotary evaporation and the residue was purified by column chromatography on silica gel (PE:MTBE=3:2) to get the title product (0.19 g, yield 36%) as a white solid. GC/MS (EI): m/z (%): 281 (1) [M⁺], 250 (5), 236 (100), 220 (15), 208 (65), 193 (15), 165 (10), 115 (10), 103 (5), 91 (20).

### Example 136e: 2-(3,5-dimethyl-2-(3-oxopropyl)phenoxy)benzonitrile

To the suspension of 2-(2-(3-hydroxypropyl)-3,5-dimethylphenoxy)benzonitrile (0.18 g, 0.6 mmol), sodium acetate (0.10 g, 1.2 mmol) and silica gel (0.5 g) in dichloromethane (25 mL) at 0° C. was added portionwise Pyrindium Chlorochromate (0.26 g, 1.2 mmol). Then the resulting mixture was stirred at room temperature for 2 hours. MTBE (60 mL) was used to dilute the reaction solution, the mixture was filtered through a small pad of silica gel, the solvent was removed by rotary evaporation and the residue was purified by column chromatography on silica gel (PE:MTBE=4:1) to get the title product (0.12 g, yield 67%) as a colorless liquid. GC/MS (EI): m/z (%): 279 (10) [M⁺], 260 (25), 236 (30), 222 (100), 208 (45), 193 (15), 165 (10), 115 (25), 103 (10), 91 (35).

### Example 136f: 2-(3,5-dimethyl-2-(3-morpholinopropyl)phenoxy)benzonitrile

To the solution of 2-(3,5-dimethyl-2-(3-oxopropyl)phenoxy)benzonitrile (0.11 g, 0.38 mmol) and morpholine (0.040 g, 0.4 mmol) in dichloromethane (10 mL) was added sodium triacetoxyborohydride (0.11 g, 0.5 mmol) at 0° C. under Ar. Then the resulting mixture was stirred at room temperature for overnight. After the reaction mixture was cooled to 5° C., diluted NaHCO₃ solution (20 mL) was added dropwise, MTBE (30 mL×3) was used to extract the solution, the combined organic phase was dried over MgSO₄ and concentrated by rotary evaporation, and then the residue was purified by column chromatography on silica gel (PE:MTBE=7:3) to get the title product (0.06 g, yield 42%) as a liquid. GC/MS (EI): m/z (%): 350 (8) [M⁺], 207 (15), 193 (3), 127 (11), 115 (15), 100 (100), 91 (10), 56 (10). ¹H NMR (300 MHz, CDCl₃) δ 7.64 (d, J=7.6 Hz, 1H), 7.42 (t, J=7.5 Hz, 1H), 7.08 (t, J=7.5 Hz, 1H), 6.87 (s, 1H), 6.73 (d, J=8.5 Hz, 1H), 6.63 (s, 1H), 3.83-3.50 (m, 4H), 2.74-2.49 (m, 2H), 2.45-2.35 (m, 6H), 2.33 (s, 3H), 2.26 (s, 3H), 1.77-1.58 (m, 2H). ¹³C NMR (75 MHz, ¹³C NMR (75 MHz, CDCl₃) δ 160.4 (s), 152.5 (s), 138.5 (s), 136.8 (s), 134.2 (d), 133.8 (d), 129.8 (s), 128.3 (d), 122.1 (d), 118.7 (d), 116.1 (s), 115.7 (d), 102.7 (s), 67.0 (t), 58.8 (t), 53.6 (t), 29.8 (s), 26.3 (d), 24.4 (d), 20.9 (q), 19.4 (q).

### Example 137: 4-(3-(2-(2-fluorophenoxy)-4,6-dimethylphenyl)propyl)morpholine

Prepared in an analogous sequence as described in Example 136: 3-(2-(2-fluorophenoxy)-4,6-dimethylphenyl)propanal (0.17 g, 0.59 mmol), NaBH(AcO)₃ (0.19 g, 0.89 mmol) and morpholine (0.070 g, 0.77 mmol) in Dichloromethane (10 mL) were reacted to give the title product (0.17 g, yield 79%) as a brown viscous liquid. GC/MS (EI): m/z (%): 343 (20) [M⁺], 256 (5), 227 (5), 165 (4), 127 (8), 100 (100), 91 (5), 56 (6). ¹H NMR (300 MHz, CDCl₃) δ 7.21-7.10 (m, 1H), 7.08-7.00 (m, 2H), 6.96-6.88 (m, 1H), 6.76 (s, 1H), 6.45 (s, 1H), 3.85-3.50 (m, 4H), 2.87-2.57 (m, 2H), 2.40 (s, 6H), 2.32 (s, 3H), 2.20 (s, 3H), 1.88-1.60 (m, 2H). ¹³C NMR (75 MHz, CDCl₃) δ 155.3 (s), 154.6 (s), 152.1 (s), 145.1 (s), 144.8 (s), 137.9 (s), 136.3 (s), 128.3 (s), 126.5 (d), 124.5 (d), 124.0 (d), 123.7 (d), 123.6 (d), 120.3 (d), 117.0 (d), 116.8 (d), 115.8 (d), 67.0 (t), 58.9 (t), 53.7 (t), 26.3 (t), 24.2 (t), 21.0 (q), 19.4 (q).

### Example 138: 2-(5-methyl-2-(3-(4-methylpiperazin-1-yl)propyl)phenoxy)benzonitrile

### Example 138a: 2-(3-hydroxypropyl)-5-methylphenol

At 0° C., to the suspension of LiAlH₄ (3.8 g, 100 mmol in Tetrahydrofuran (150 mL) was added dropwise the solution of 7-methyl-2H-chromen-2-one (10.0 g, 62.4 mmol in Tetrahydrofuran (30 mL) carefully. After addition, the resulting mixture was allowed to stir at room temperature for 2 hours. Diluted HCl solution (100 mL) was added to quench the reaction carefully, ethyl acetate (100 mL×2) was used to extract the solution, the combined organic phase was dried over Na₂SO₄, the solvent was removed and the residue was purified by column chromatography on silica gel (PE:MTBE=1:1) to get the title product (2.8 g, yield 26%) as a white solid. GC/MS (EI): m/z (%): 166 (40) [M⁺], 148 (25), 133 (45), 121 (100), 105 (15), 91 (50), 77 (35), 65 (10).

### Example 138b: 2-(2-(3-hydroxypropyl)-5-methylphenoxy)benzonitrile

The whole mixture of 2-(3-hydroxypropyl)-5-methylphenol (0.8 g, 4.67 mmol), 2-iodobenzonitrile (1.6 g, 7.0 mmol), potassium phosphate (1.98 g, 9.3 mmol), copper(I) iodide (0.13 g, 0.7 mmol) and picolinic acid (0.17 g, 1.4 mmol) in DMSO (25 mL) was evacuated and flushed with Ar for three times. Then it was allowed to stir at 90° C. for overnight. Water (100 mL) was added, EA (100 mL×3) was used to extract the solution, the organic phase was dried over MgSO₄, the solvent was removed and the residue was purified by column chromatography on silica gel (PE:MTBE=2:3) to get the title product (0.70 g, yield 56%) as a white solid. GC/MS (EI): m/z (%): 267 (4) [M⁺], 249 (100), 234 (90), 222 (30), 206 (40), 194 (35), 115 (20), 91 (35), 77 (20), 65 (10).

### Example 138c: 2-(5-methyl-2-(3-oxopropyl)phenoxy)benzonitrile

At 0° C., to the suspension of pyridium chlorochromate (0.39 g, 1.8 mmol), sodium acetate (0.15 g, 1.8 mmol) and 1.0 g silica gel in dichloromethane (25 mL) was added portionwise 2-(2-(3-hydroxypropyl)-5-methylphenoxy)benzonitrile (0.25 g, 0.91 mmol) and the resulting mixture was stirred at room temperature for 3 hours. MTBE (60 mL) was added, the mixture was filtered through a small pad of silica gel, MTBE (50 mL) was used to flush the pad and the solvent was removed to get the title product (0.26 g, yield 65%) as a viscous liquid, which was used for next step directly. GC/MS (EI): m/z (%): 265 (45) [M⁺], 236 (100), 222 (75), 208 (100), 194 (40), 121 (50), 115 (20), 91 (35), 77 (20), 65 (10).

### Example 138d: 2-(5-methyl-2-(3-(4-methylpiperazin-1-yl)propyl)phenoxy)benzonitrile

A flask was charged with 2-(5-methyl-2-(3-oxopropyl)phenoxy)benzonitrile (0.22 g, 0.50 mmol) and 1-methylpiperazine (0.05 g, 0.5 mmol) in dichloromethane (15 mL). And sodium triacetoxyborohydride (0.13 g, 0.60 mmol) was added and the resulting mixture was stirred at ambient temperature for overnight. The reaction mixture was cooled to 5° C., a diluted NaHCO₃ solution (30 mL) was added dropwise, the mixture was extracted with dichloromethane (30 mL×3), the organic phase was concentrated and the residue was purified by column chromatography on silica gel (DCM:Methanol=10:1) to get the title product (0.14 g, yield: 78%) as a viscous liquid. GC/MS (EI): m/z (%): 349 (7) [M⁺], 293 (6), 248 (2), 194 (5), 160 (6), 127 (45), 113 (100), 98 (5), 85 (8), 70 (40). ¹H NMR (300 MHz, CDCl₃) δ 7.63 (dd, J=7.7, 1.5 Hz, 1H), 7.46-7.38 (m, 1H), 7.16 (d, J=7.7 Hz, 1H), 7.07 (t, J=7.5 Hz, 1H), 6.97 (d, J=7.8 Hz, 1H), 6.76 (s, 1H), 6.69 (d, J=8.5 Hz, 1H), 2.67-2.34 (m, 12H), 2.30 (d, J=5.9 Hz, 6H), 1.86-1.69 (m, 2H). ¹³C NMR (75 MHz, CDCl₃) δ 160.3 (q), 152.3 (q), 137.9 (q), 134.3 (t), 133.9 (t), 130.9 (q), 130.9 (t), 126.5 (t), 122.3 (t), 121.2 (t), 116.2 (q), 115.8 (t), 102.8 (q), 57.7 (d), 54.6 (d), 52.5 (d), 45.6 (s), 27.6 (d), 27.1 (d), 21.1 (s).

### Example 139: 2-(5-methyl-2-(3-(4-methylpiperazin-1-yl)propyl)Phenoxy)benzonitrile hydrochloride

To a solution of 2-(5-methyl-2-(3-(4-methylpiperazin-1-yl)propyl)phenoxy)benzonitrile (0.53 g, 1.44 mmol) in methanol (25 mL) was added hydrogen chloride (0.75 g, 7.20 mmol) and the resulting mixture was stirred at room temperature for half an hour. Then the solvent was removed and the mixture was dried at 90° C. under reduced pressure to get the title product (550 mg, 1.35 mmol, yield 94%) as a white solid. GC/MS (EI): m/z (%): 349 (10) [M⁺], 293 (8), 235 (5), 194 (6), 160 (7), 127 (42), 113 (100), 98 (5), 85 (8), 70 (39).

### Example 140: 2-(4-methyl-2-(3-(4-methylpiperazin-1-yl)propyl)phenoxy)benzonitrile

Prepared in an analogous sequence as described in Example 138 (starting from 6-methyl-2H-chromen-2-one instead of 7-methyl-2H-chromen-2-one): In the final step, a flask was charged with 2-(4-methyl-2-(3-oxopropyl)phenoxy)benzonitrile (0.18 g, 0.54 mmol) and 1-methylpiperazine (0.050 g, 0.54 mmol) in Dichloromethane (15 mL). Sodium triacetoxyborohydride (0.14 g, 0.65 mmol) was added and the resulting mixture was stirred at ambient temperature for overnight. The reaction mixture was cooled to 5° C., NaHCO₃ solution was added dropwise, the mixture was extracted with DCM, the organic phase was concentrated and the residue was purified by column chromatography on silica gel (DCM:Methanol=10:1) to get the title product (0.14 g, yield 72%) as a viscous liquid. GC/MS (EI): m/z (%): GC/MS (EI): m/z (%): 349 (25) [M⁺], 293 (11), 194 (5), 160 (15), 127 (60), 113 (100), 85 (10), 70 (40). ¹H NMR (300 MHz, CDCl₃) δ 7.63 (d, J=7.7, 1H), 7.45-7.36 (m, 1H), 7.13-6.99 (m, 3H), 6.86 (d, J=8.2 Hz, 1H), 6.68 (d, J=8.5 Hz, 1H), 2.73-2.36 (m, 12H), 2.34 (s, 6H), 1.88-1.72 (m, 2H). ¹³C NMR (75 MHz, CDCl₃) δ 160.5 (q), 150.1 (q), 135.5 (q), 134.4 (q), 133.9 (t), 133.9 (t), 131.7 (t), 128.4 (t), 122.2 (t), 120.7 (t), 116.3 (q), 115.4 (t), 102.7 (q), 57.7 (d), 54.6 (d), 52.5 (d), 45.7 (s), 27.9 (d), 27.1 (d), 21.0 (s).

### Example 141: 2-(2-(2-hydroxy-3-morpholinopropyl)-5-methylphenoxy)benzonitrile

A mixture of 2-(5-methyl-2-(oxiran-2-ylmethyl)phenoxy)benzonitrile (0.20 g, 0.72 mmol) (obtained by preparing 2-(2-allyl-5-methylphenoxy)benzonitrile in an analogous sequence as described in Example 136a-c, followed by epoxidation as described in Example 63) and morpholine (0.19 g, 2.15 mmol) in ethanol (20 mL) was heated to reflux for overnight. Then the solvent was removed, water was added, DCM was used to extract the solution, the solvent was removed and the residue was purified by column chromatography on silica gel (PE:MTBE=3:7) to get the title product (0.20 g, 0.57 mmol, yield 71%) as a viscous liquid. GC/MS (EI): m/z (%): 352 (1) [M⁺], 281 (1), 223 (1), 207 (4), 194 (1), 152 (1), 130 (4), 100 (100), 77 (2), 56 (5). ¹H NMR (300 MHz, CDCl₃) δ 7.65 (s, 1H), 7.44 (s, 1H), 7.27 (s, 1H), 7.18-7.05 (m, 2H), 6.92-6.62 (m, 2H), 3.95 (d, J=23.6 Hz, 1H), 3.67 (s, 4H), 3.50 (s, 1H), 2.85 (d, J=13.6 Hz, 1H), 2.69 (d, J=4.3 Hz, 1H), 2.59 (s, 2H), 2.47 (d, J=13.8 Hz, 3H), 2.35 (d, J=11.7 Hz, 4H), 1.26 (s, 2H), 0.87 (s, 1H). ¹³C NMR (75 MHz, CDCl₃) δ 159.8 (s), 152.9 (s), 140.2 (s), 134.2 (d), 133.9 (d), 132.1 (d), 129.1 (s), 127.5 (d), 126.4 (d), 122.4 (d), 121.0 (d), 118.0 (d), 115.9 (d), 102.9 (s), 67.1 (t), 66.2 (d), 64.2 (t), 53.5 (t), 35.2 (s), 32.1 (t), 21.1 (q).

### Example 142: 2-(3,5-dimethyl-2-((1-(4-methylpiperazin-1-yl)propan-2-yl)oxy)benzyl)benzonitrile

### Example 142a: ethyl 2-(2-(2-cyanobenzyl)-4,6-dimethylphenoxy)propanoate

A solution of 2-(2-hydroxy-3,5-dimethylbenzyl)benzonitrile (0.50 g, 2.1 mmol) in DMF (10 mL) was treated with potassium carbonate (0.87 g, 6.3 mmol), followed by addition of ethyl 2-bromopropanoate (0.57 g, 3.2 mmol). The suspension was stirred at. rt. for overnight. Water (50 mL) was added, MTBE (30 mL×3) was used to extract the solution, the solvent was removed and the residue was purified by column chromatography on silica gel (PE:MTBE=8:1) to get the title product (0.65 g, yield 90%) as a viscous liquid. GC/MS (EI): m/z (%): 337 (30) [M⁺], 291 (5), 264 (25), 236 (100), 220(10), 208 (75), 182 (55), 121 (10), 91 (5), 77 (4).

### Example 142b: 2-(2-((1-hydroxypropan-2-yl)oxy)-3,5-dimethylbenzyl)benzonitrile

Sodium borohydride (0.20 g, 5.3 mmol) was added portionwise to the solution of ethyl 2-(2-(2-cyanobenzyl)-4,6-dimethylphenoxy)propanoate (0.60 g, 1.9 mmol in Methanol (10 mL) at 0° C. and the resulting mixture was stirred at room temperature for overnight. The solvent was removed, water (50 mL) was added, EA (40 mL×3) was used to extract the solution, the mixture was concentrated and the residue was purified by column chromatography on silica gel (PE:MTBE=1:1) to get the title product (0.52 g, yield 94%) as a viscous liquid. GC/MS (EI): m/z (%): 295 (10) [M⁺], 264 (8), 237 (100), 222 (15), 208 (55), 194 (30), 182 (55), 121 (25). ¹H NMR (300 MHz, CDCl₃) δ 7.63 (d, J=7.7 Hz, 1H), 7.50-7.41 (m, 1H), 7.33-7.24 (m, 1H), 7.18 (d, J=7.8 Hz, 1H), 6.89 (s, 1H), 6.68 (s, 1H), 4.35-4.10 (m, 3H), 3.77 (s, 2H), 2.28 (s, 3H), 2.21 (s, 3H), 1.19 (d, J=6.4 Hz, 3H). ¹³C NMR (75 MHz, CDCl₃) δ 151.3 (q), 145.4 (q), 133.4 (q), 132.9 (t), 132.8 (t), 132.1 (q), 131.3 (t), 131.2 (q), 130.1 (t), 129.1 (t), 126.7 (t), 118.6 (q), 112.6 (q), 78.5 (t), 66.9 (d), 34.8 (d), 20.8 (s), 17.5 (s), 16.2 (s).

### Example 142c: 2-(3,5-dimethyl-2-((1-oxopropan-2-yl)oxy)benzyl)benzonitrile

To a solution of oxalyl dichloride (0.50 g, 3.94 mmol) in dichloromethane (Volume: 15 mL) was add (methylsulfinyl)methane (0.513 g, 6.6 mmol) dropwise at −78° C. Then a solution of 2-(2-((1-hydroxypropan-2-yl)oxy)-3,5-dimethylbenzyl)benzonitrile (0.17 g, 0.55 mmol) in dichloromethane (5 mL) was added dropwise via syringe after 5 minutes of the reaction. The solution was stirred at −78° C. for 30 minutes. Triethylamine (1.33 g, 13 mmol) was added to the reaction mixture. The resulting mixture was allowed to stir at room temperature over 30 minutes. Water (25 mL) was added and dichloromethane (3×25 mL) was used to extract the solution. The combined organic phase was washed with 0.1 M aq. HCl solution (100 mL), H₂O (100 mL), then sat. aq. NaHCO₃ solution (100 mL) and brine (100 mL). The organic phase was dried over MgSO₄ and filtered through a small pad of silica gel. The solvent was removed to obtain the title product (0.18 g, yield 95%) as a colorless liquid. GC/MS (EI): m/z (%): 293 (65) [M⁺], 264 (98), 236 (45), 221 (20), 208 (100), 190 (35), 165 (24), 116 (20).

### Example 142d: 2-(3,5-dimethyl-2-((1-(4-methylpiperazin-1-yl)propan-2-yl)oxy)benzyl)benzonitrile

A flask was charged with 2-(3,5-dimethyl-2-((1-oxopropan-2-yl)oxy)benzyl)benzonitrile (0.17 g, 0.49 mmol) and 1-methylpiperazine (0.060 g, 0.64 mmol) in dichloromethane (10 mL). Sodium triacetoxyborohydride (0.13 g, 0.59 mmol) was added and the resulting mixture was stirred at ambient temperature for overnight. The reaction mixture was cooled to 5° C., a NaHCO₃ solution (30 mL) was added dropwise, the mixture was extracted with DCM (30 mL×3), the organic phase was concentrated and the residue was purified by column chromatography on silica gel (DCM:Methanol:TEA=95:5:1) to get the title product (0.12 g, yield 61%) as a viscous liquid. GC/MS (EI): m/z (%): 377 (1) [M⁺], 261 (1), 208 (3), 190 (3), 165 (2), 141 (26), 113 (100), 98 (6), 84 (1), 70 (28). ¹H NMR (300 MHz, CDCl₃) δ 7.62 (d, J=7.7 Hz, 1H), 7.45-7.41 (m, 1H), 7.29-7.22 (m, 1H), 7.16 (d, J=7.9 Hz, 1H), 6.86 (s, 1H), 6.69 (s, 1H), 4.25 (s, 2H), 2.78-2.66 (m, 1H), 2.62-2.34 (m, 8H), 2.27 (s, 3H), 2.24 (s, 3H), 2.20 (s, 3H), 1.19 (d, J=6.2 Hz, 3H). ¹³C NMR (75 MHz, CDCl₃) δ 152.2 (q), 145.7 (q), 132.9 (t), 132.6 (t), 132.1 (q), 131.0 (q), 131.0 (t), 130.1 (t), 129.1 (t), 126.5 (t), 118.6 (q), 112.5 (q), 76.2 (t), 64.4 (d), 55.2 (d), 54.1 (d), 46.1 (s), 34.8 (d), 20.8 (s), 18.9 (s), 17.5 (s).

### Example 143: 2-(3,5-dimethyl-2-((1-(4-methylpiperazin-1-yl)propan-2-yl)oxy)benzyl)benzonitrile hydrochloride

To 2-(3,5-dimethyl-2-((1-(4-methylpiperazin-1-yl)propan-2-yl)oxy)benzyl)benzonitrile (0.050 g, 0.13 mmol) in Methanol (10 mL) was added hydrogen chloride (0.065 g, 0.63 mmol) and the resulting mixture was standing for half an hour. Then the solvent was removed by rotary evaporation and the residue was dried over Kugelrohr (90° C., 0.1 mbar) to get the title product (0.050 mg, yield 91%) as a solid. GC/MS (EI): m/z (%): 377 (1) [M⁺], 237 (2), 208 (5), 141 (35), 113 (100), 98 (8), 84 (1), 70 (30).

### Example 144: (1-(2-(2-benzyl-4-methylphenoxy)ethyl)piperidin-4-yl)methanol

The flask was charged with 2-(2-benzyl-4-methylphenoxy)acetaldehyde (0.4 g, 1.17 mmol) and piperidin-4-yl-methanol (0.17 g, 1.52 mmol) in dichloromethane (10 mL). Sodium triacetoxyborohydride (0.37 g, 1.75 mmol) was added and the resulting mixture was stirred at ambient temperature for overnight. The reaction mixture was cooled to 5° C., NaHCO₃ solution was added dropwise, the mixture was extracted with DCM, the organic phase was concentrated and the residue was purified by column chromatography on silica gel (DCM:Methanol=90:10) to get the title product (0.24 g, 0.64 mmol, yield 54.6%) as a viscous liquid. GC/MS (EI): m/z (%): 339 (15) [M⁺], 224 (5), 195 (5), 165 (5), 142 (4), 128(100), 115 (4), 91 (10), 67 (4). ¹H NMR (300 MHz, CDCl₃) δ 7.25-7.10 (m, 5H), 6.95 (d, J=8.2 Hz, 1H), 6.89 (s, 1H), 6.73 (d, J=8.2 Hz, 1H), 4.05 (t, J=5.9 Hz, 2H), 3.92 (s, 2H), 3.45 (d, J=6.4 Hz, 2H), 2.96 (d, J=11.6 Hz, 2H), 2.75 (t, J=5.8 Hz, 2H), 2.22 (s, 3H), 2.12-2.00 (m, 2H), 1.97 (s, 1H), 1.68 (d, J=13.1 Hz, 2H), 1.54-1.36 (m, 1H), 1.35-1.20 (m, 2H). ¹³C NMR (75 MHz, CDCl₃) δ 154.5 (q), 141.4 (q), 131.4 (t), 129.9 (q), 129.6 (q), 128.9 (t), 128.3 (t), 127.8 (t), 125.8 (t), 111.6 (t), 67.9 (d), 66.6 (d), 57.7 (d), 54.0 (d), 38.4 (t), 36.2 (d), 28.9 (d), 20.6 (s).

### Example 145: 1-(2-(2-benzyl-4-methylphenoxy)ethyl)piperidin-4-ol

The flask was charged with 2-(2-benzyl-4-methylphenoxy)acetaldehyde (0.50 g, 1.5 mmol) and piperidin-4-ol (0.19 g, 1.9 mmol) in Dichloromethane (10 mL). Sodium triacetoxyborohydride (0.46 g, 2.2 mmol) was added and the resulting mixture was stirred at ambient temperature for overnight. The reaction mixture was cooled to 5° C., NaHCO₃ solution was added dropwise, the mixture was extracted with DCM, the organic phase was concentrated and the residue was purified by column chromatography on silica gel (DCM:Methanol=90:10) to get the title product (0.43 g, yield 82%) as a viscous liquid. GC/MS (EI): m/z (%): 325 (10) [M⁺], 224 (10), 195 (10), 165 (11), 128 (8), 114(100), 91 (13), 70 (2). ¹H NMR (300 MHz, CDCl₃) δ 7.26-7.19 (m, 2H), 7.14 (t, J=8.6 Hz, 3H), 6.98 (d, J=8.2 Hz, 1H), 6.92 (s, 1H), 6.73 (d, J=8.2 Hz, 1H), 5.41 (s, 1H), 4.16 (t, J=5.0 Hz, 2H), 3.92 (s, 2H), 3.10-2.84 (m, 4H), 2.66-2.46 (m, 2H), 2.24 (s, 3H), 2.03-1.92 (m, 3H), 1.72-1.55 (m, 2H). ¹³C NMR (75 MHz, CDCl₃) δ 154.0 (q), 141.3 (q), 131.9 (t), 130.4 (q), 128.9 (q), 128.7 (t), 128.4 (t), 128.1 (t), 125.9 (t), 111.4 (t), 64.9 (d), 56.5 (d), 50.1 (d), 36.3 (d), 32.5 (d), 20.6 (s).

### Example 146: 1-(2-(2-benzyl-4-methylphenoxy)ethyl)piperazine

The flask was charged with 2-(2-benzyl-4-methylphenoxy)acetaldehyde (0.33 g, 0.96 mmol) and piperazine (0.17 g, 1.92 mmol) in Dichloromethane (Volume: 10 mL). Sodium triacetoxyborohydride (0.27 g, 1.25 mmol) was added and the resulting mixture was stirred at ambient temperature for overnight. The reaction mixture was cooled to 5° C., NaHCO₃ solution was added dropwise, the mixture was extracted with DCM, the organic phase was concentrated and the residue was purified by column chromatography on silica gel (DCM:Methanol=80:20) to get the title product (0.10 g, 0.31 mmol, yield 31.8%) as a viscous colorless liquid. GC/MS (EI): m/z (%): 310 (25) [M⁺], 268 (10), 195 (10), 165 (20), 152 (10), 113(30), 99 (100), 86 (20), 70 (30), 56 (50).

### Example 147: 1-(2-(2-benzyl-4,6-dimethylphenoxy)ethyl)-4-methylpiperazine

At room temperature, to the mixture of sodium hydride (1.34 g, 31.3 mmol, 56% purity) in DMF (20 mL) was added subsequently 2-benzyl-4,6-dimethylphenol (1.00 g, 4.5 mmol), 1-(2-chloroethyl)-4-methylpiperazine dihydrochloride (1.58 g, 6.7 mmol) and potassium iodide (0.15 g, 0.89 mmol) and then the mixture was stirred at 70° C. for overnight. The mixture was then heated to 100° C. for 1 hour. Water (60 mL) was added to quench the reaction, EA (50 mL×3) was used to extract the solution three times, the solvent was removed and the residue was purified by column chromatography on silica gel (DCM:Methanol=10:1) carefully to get the title product (0.32 g, yield 21%) as a viscous liquid. GC/MS (EI): m/z (%): 338 (4) [M⁺], 295 (1), 209 (5), 178 (4), 165 (7), 127 (52), 113 (100), 100 (19), 84 (7), 70 (41). ¹H NMR (300 MHz, CDCl₃) δ 7.25-7.10 (m, 5H), 6.83 (s, 1H), 6.71 (s, 1H), 3.97 (s, 2H), 3.77 (t, J=5.9 Hz, 2H), 2.70 (t, J=5.9 Hz, 2H), 2.56 (s, 8H), 2.28 (s, 3H), 2.24 (s, 3H), 2.18 (s, 3H). ¹³C NMR (75 MHz, CDCl₃) δ 153.6 (q), 141.4 (q), 133.4 (q), 133.3 (q), 130.7 (q), 130.2 (t), 129.3 (t), 128.9 (t), 128.4 (t), 125.9 (t), 70.2 (d), 58.1 (d), 55.0 (d), 53.5 (d), 45.9 (s), 35.8 (d), 20.8 (s), 16.5 (s).

### Example 148: 1-methyl-4-(2-(4-methyl-2-(3-methylbenzyl)phenoxy)ethyl)piperazine

At room temperature, to the mixture of sodium hydride (1.20 g, 28.0 mmol) in DMF (20 mL) was added subsequently 4-methyl-2-(3-methylbenzyl)phenol (1.00 g, 4.0 mmol), 1-(2-chloroethyl)-4-methylpiperazine dihydrochloride (1.41 g, 6.0 mmol) and potassium iodide (0.13 g, 0.80 mmol). The resulting mixture was stirred at 40° C. for two days. It was the heated to 100° C. for 1 hour. Water was added to quench the reaction, EA was used to extract the solution for three times, the solvent was removed and the residue was purified by column chromatography on silica gel (PE:MTBE=1:1 to MTBE to DCM:Methanol=10:1) to separate the title product (0.28 g, yield 20%) as light yellow viscous liquid. GC/MS (EI): m/z (%): 338 (3) [M⁺], 295 (1), 209 (2), 178 (3), 165 (6), 127 (43), 113 (100), 100 (6), 84 (4), 70 (37). ¹H NMR (300 MHz, CDCl₃) δ 7.20-7.10 (m, 1H), 7.06-6.87 (m, 5H), 6.74 (d, J=8.2 Hz, 1H), 4.06 (t, J=5.7 Hz, 2H), 3.90 (s, 2H), 2.77 (t, J=5.7 Hz, 2H), 2.75-2.41 (m, 8H), 2.32 (s, 3H), 2.30 (s, 3H), 2.24 (s, 3H). ¹³C NMR (75 MHz, CDCl₃) δ 154.5 (q), 141.2 (q), 137.7 (q), 131.4 (t), 129.9 (q), 129.7 (t), III 129.6 (t), 128.2 (t), 127.7 (t), 126.5 (t), 125.9 (t), 111.6 (t), 66.7 (d), 57.3 (d), 55.0 (d), 53.3 (d), 45.8 (s), 36.1 (d), 21.5 (s), 20.6 (s).

### Example 149: 1-(2-(2-isobutyl-4-methylphenoxy)ethyl)-4-methylpiperazine

At 0° C., to the suspension of sodium hydride (0.22 g, 9.1 mmol) in DMF (10 mL) was added 2-isobutyl-4-methylphenol (0.30 g, 1.8 mmol) and 1-(2-chloroethyl)-4-methylpiperazine dihydrochloride (0.45 g, 1.8 mmol) and the reaction mixture was stirred at room temperature for overnight. Then it was poured into ice water, the mixture was extracted with EA, the solvent was removed and the residue was purified by column chromatography on silica gel (DCM:methanol=3:1) to get the title product (0.13 g, yield 23%) as viscous brown liquid. GC/MS (EI): m/z (%): 290 (1) [M⁺], 275 (1), 207 (2), 176 (1), 147 (1), 127 (38), 113 (100), 98 (5), 84 (4), 70 (31). ¹H NMR (300 MHz, CDCl₃) δ 6.94-6.84 (m, 2H), 6.69 (d, J=8.1 Hz, 1H), 4.05 (t, J=5.7 Hz, 2H), 2.80 (t, J=5.7 Hz, 2H), 2.68-2.55 (m, 8H), 2.52 (s, 4H), 2.42 (d, J=7.1 Hz, 2H), 2.32 (s, 3H), 2.24 (s, 3H), 1.95-1.80 (m, 1H), 0.87 (d, J=6.6 Hz, 6H). ¹³C NMR (75 MHz, CDCl₃) δ 154.8 (q), 131.8 (t), 130.2 (q), 129.4 (q), 127.1 (t), 111.3 (t), 66.6 (d), 57.4 (d), 55.1 (d), 53.5 (d), 45.9 (s), 39.7 (d), 29.9 (s), 28.9 (t), 22.7 (s), 20.6 (s).

### Example 150: 1-(2-(2-isobutyl-4-methylphenoxyethyl)-4-methylpiperazine dihydrochloride

At 0° C., to the suspension of sodium hydride (0.22 g, 9.1 mmol) in DMF (10 mL) was added 2-isobutyl-4-methylphenol (0.30 g, 1.8 mmol) and 1-(2-chloroethyl)-4-methylpiperazine dihydrochloride (0.45 g, 1.8 mmol) and the reaction mixture was stirred at room temperature for overnight. Then it was poured into ice water, the mixture was extracted with EA, the solvent was removed and the residue was purified by column chromatography on silica gel (DCM:methanol=3:1) to get the title product (0.13 g, yield 23%) as viscous brown liquid. GC/MS (EI): m/z (%): 331 (5) [M⁺], 244 (8), 207 (10), 171 (2), 145 (3), 114(15), 100 (100), 91 (3), 70 (4). GC/MS (EI): m/z (%): 290 (1) [M⁺], 275 (1), 147 (1), 127 (40), 113 (100), 98(5), 84 (5), 70 (35).

### Example 151: 1-(2-(2-(2-iodobenzyl)-4,6-dimethylphenoxy)ethyl)-4-methylpiperazine

The mixture of 1-(2-(2-(2-bromobenzyl)-4,6-dimethylphenoxy)ethyl)-4-methylpiperazine (0.15 g, 0.36 mmol), sodium 2,2,2-trifluoroacetate (0.29 g, 2.2 mmol) and copper(I) iodide (0.41 g, 2.16 mmol) in DMF (10 mL) and Toluene (3 mL) was heated to reflux for 6 hours. The mixture was then kept at 150° C. for two days. Water (50 mL) was added, DCM (50 mL×3) was used to extract the solution, the solvent was removed and the residue was purified by column chromatography on silica gel (DCM:Methanol=10:1) to get the title product (0.089 g, yield 27%) as a viscous liquid. GC/MS (EI): m/z (%): 464 (5) [M⁺], 209 (10), 195 (8), 179 (11), 165 (10), 152(5), 127 (74), 113 (100), 100 (20). ¹H NMR (300 MHz, CDCl₃) δ 7.88-7.50 (m, 1H), 7.23-7.18 (m, 1H), 7.11-6.85 (m, 3H), 6.64 (d, J=5.7 Hz, 1H), 4.04 (d, J=12.8 Hz, 2H), 3.79 (t, J=5.3 Hz, 2H), 2.99-2.66 (m, 10H), 2.54 (s, 3H), 2.26 (s, 3H), 2.20 (s, 3H). ¹³C NMR (75 MHz, CDCl₃) δ 153.5 (q), 143.7 (q), 140.5 (q), 139.3 (t), 133.7 (q), 132.6 (t), 131.9 (q), 131.6 (q), 130.8 (q), 130.7 (t), 130.7 (t), 130.6 (t), 129.9 (t), 129.3 (t), 128.5 (t), 128.1 (t), 127.9 (t), 127.6 (t), 124.8 (q), 101.5 (q), 70.0 (d), 57.7 (d), 54.4 (d), 51.6 (d), 44.8 (s), 41.4 (d), 36.2 (d), 20.9 (s), 16.5 (s).

### Example 152: 2-(2-hydroxy-5-methylbenzylidene)cyclohexan-1-one

A solution of 2-hydroxy-5-methylbenzaldehyde (5.00 g, 36.7 mmol) in 4% aqueous sodium hydroxide (5.88 g, 147 mmol)) was added dropwise to a mixture of 4% aqueous sodium hydroxide (1.47 g, 36.7 mmol) and cyclohexanone (18.02 g, 184 mmol) under stirring. The reaction mixture was stirred at room temperature for 40 h and then neutralized with HCl 6 N (40 mL). The HCl solution was added dropwise to avoid heating. The product was extracted into CHCl₃ (100 mL), washed with water (4×100 mL) and dried over Na₂SO₄. The chloroform was evaporated and the resulting dark brown liquid was extracted with hot petroleum ether. the crude product was purified by biotage to get the title product (3.60 g, yield 45%). GC/MS (EI): m/z (%): 210 (1) [M⁺], 197 (100), 183 (40), 168 (10), 152 (10), 91(10), 76 (8).

### Example 153: 2-(2-hydroxy-5-methylbenzyl)cyclohexan-1-one

To a suspension of Pd—C (palladium on charcoal) (0.050 g, 0.46 mmol) in MeOH (50 mL) was added (E)-2-(2-hydroxy-5-methylbenzylidene)cyclohexan-1-one (1.00 g, 4.6 mmol) under stirring and the mixture set under hydrogen atmosphere (5 bar). The reaction mixture was stirred at room temperature for 40 h. The product was extracted into CHCl₃ (100 mL), washed with water (4×100 mL) and dried over Na₂SO₄. The chloroform was evaporated and the resulting brown liquid was purified with silicagel column to obtain the title product (0.84 g, yield 82%). GC/MS (EI): m/z (%): 218 (40) [M⁺], 199 (20), 171 (40), 121 (100), 108 (40), 91(20), 77 (20).

### Example 154: (E)-2-(5-methyl-2-(2-morpholinoethoxy)benzylidene)cyclohexan-1-one

Following the general procedure from Example 80: The mixture of (E)-2-(2-hydroxy-5-methylbenzylidene)cyclohexan-1-one (0.30 g, 1.4 mmol), 4-(2-chloroethyl)morpholine dihydrochloride (0.62 g, 2.8 mmol), potassium carbonate (0.58 g, 4.2 mmol) and potassium iodide (0.046 g, 0.28 mmol) in DMF (20 mL) was reacted to give the title product (0.21 g, yield 46%) as a viscous liquid. GC/MS (EI): m/z (%): 329 (10) [M⁺], 242 (2), 199 (5), 171 (3), 145 (5), 114(5), 100 (100), 91(4), 70 (5). ¹H NMR (300 MHz, CDCl₃) δ 7.61 (s, 1H), 7.13-7.00 (m, 2H), 6.79 (d, J=8.2 Hz, 1H), 4.09 (t, J=5.9 Hz, 2H), 3.75-3.69 (m, 4H), 2.83-2.70 (m, 4H), 2.59-2.48 (m, 6H), 2.29 (s, 3H), 1.94-1.84 (m, 2H), 1.79-1.68 (m, 2H). ¹³C NMR (75 MHz, CDCl₃) δ 201.9 (q), 155.6 (q), 136.8 (q), 131.4 (t), 130.9 (t), 130.5 (t), 129.5 (q), 124.9 (q), 112.1 (t), 67.1 (d), 57.7 (d), 54.3 (d), 40.6 (d), 29.3 (d), 24.2 (d), 23.8 (d), 20.7 (s).

### Example 155: (E)-2-(5-methyl-2-(2-(4-methylpiperazin-1-yl)ethoxy)benzylidene)-cyclohexan-1-one

A mixture of (E)-2-(2-hydroxy-5-methylbenzylidene)cyclohexan-1-one (0.30 g, 1.4 mmol), 1-(2-chloroethyl)-4-methylpiperazine dihydrochloride (0.65 g, 2.8 mmol), potassium carbonate (0.58 g, 4.2 mmol) and potassium iodide (0.046 g, 0.28 mmol) in DMF (20 mL) was heated to 100° C. for 4 hours. It was then diluted with water (50 mL), then extracted with CH₂Cl₂ (50 mL×2). The combined organic layers were dried with Na₂SO₄. The mixture was filtered and the filtrate was concentrated to give the crude product. The crude product was purified by column chromatography on silica gel eluted with (Hexane:MTBE=20:1-+10:1) to give the title product (0.13 g, 0.38 mmol, yield 27.1%) as colorless liquid. GC/MS (EI): m/z (%): 365 (8) [M⁺], 350 (5), 319 (3), 290 (11), 262 (100), 235 (11), 207 (24), 75 (9). ¹H NMR (300 MHz, CDCl₃) δ 6.91 (d, J=9.0 Hz, 2H), 6.68 (d, J=8.0 Hz, 1H), 4.11-3.95 (m, 2H), 3.15 (dd, J=13.5, 4.6 Hz, 1H), 2.76 (t, J=5.7 Hz, 2H), 2.70-2.44 (m, 8H), 2.39-2.28 (m, 5H), 2.22 (s, 3H), 2.09-1.86 (m, 2H), 1.82-1.45 (m, 3H), 1.40-1.21 (m, 1H). ¹³C NMR (75 MHz, CDCl₃) δ 213.2 (q), 154.6 (q), 132.0 (t), 129.6 (q), 128.7 (q), 127.5 (t), 111.3 (t), 66.3 (d), 57.3 (d), 55.0 (d), 53.4 (d), 50.8 (t), 45.9 (s), 42.2 (d), 33.6 (d), 30.3 (d), 28.2 (d), 25.1 (d), 20.5 (s).

### Example 156: 2-(5-methyl-2-(2-morpholinoethoxy)benzyl)cvclohexan-1-one

Following the general procedure from Example 80: The mixture of 2-(2-hydroxy-5-methylbenzyl)cyclohexan-1-one (0.30 g, 1.4 mmol), 4-(2-chloroethyl)morpholine dihydrochloride (0.61 g, 2.8 mmol), potassium carbonate (0.95 g, 6.9 mmol) and potassium iodide (0.046 g, 0.28 mmol) in DMF (20 mL) were reacted to give the title product (0.24 g, yield 53%) as a viscous liquid. GC/MS (EI): m/z (%): 331 (5) [M⁺], 244 (8), 207 (10), 171 (2), 145 (3), 114(15), 100 (100), 91 (3), 70 (4). ¹H NMR (300 MHz, CDCl₃) δ 6.92 (d, J=8.0 Hz, 1H), 6.90 (s, 1H), 6.69 (d, J=8.0 Hz, 1H), 4.12-3.95 (m, 2H), 3.74-3.64 (m, 4H), 3.20-3.15 (m, 1H), 2.75 (t, J=5.6 Hz, 2H), 2.67-2.48 (m, 5H), 2.45-2.17 (m, 6H), 2.10-1.91 (m, 2H), 1.84-1.46 (m, 3H), 1.41-1.30 (m, 1H). ¹³C NMR (75 MHz, CDCl₃) δ 212.9 (q), 154.6 (q), 132.0 (t), 129.6 (q), 128.6 (q), 127.5 (t), 111.2 (t), 67.0 (d), 66.1 (d), 57.9 (d), 54.1 (d), 50.8 (t), 42.2 (d), 33.6 (d), 30.3 (d), 28.1 (d), 25.1 (d), 20.5 (s).

### Example 157: (5-methyl-2-(2-(4-methylpiperazin-1-yl)ethoxy)phenyl)(phenyl)methanol

To the suspension of LiAlH₄ (0.17 g, 4.4 mmol) in THF (20 mL) was added (5-methyl-2-(2-(4-methylpiperazin-1-yl)ethoxy)phenyl)(phenyl)methanone (0.15 g, 0.44 mmol) and the resulting mixture was stirred at room temperature for 1 hour. Water was added to quench the reaction, DCM was used to extract the solution, the solvent was removed and the residue was purified by column chromatography on silica gel (DCM:Methanol=10:1) to get the title product (0.10 g, yield 63%) as a viscous liquid. GC/MS (EI): m/z (%): 340 (30) [M⁺], 296 (5), 252 (4), 195 (8), 127 (4), 127(55), 113 (100), 100 (15), 84 (10), 70 (55). ¹H NMR (300 MHz, CDCl₃) δ 7.42-7.35 (m, 2H), 7.32-7.28 (m, 2H), 7.25-7.17 (m, 1H), 7.02 (d, J=8.1 Hz, 1H), 6.93 (s, 1H), 6.78 (d, J=8.2 Hz, 1H), 5.87 (s, 1H), 4.16-3.98 (m, 2H), 2.73-2.41 (m, 10H), 2.32 (s, 3H), 2.25 (s, 3H). ¹³C NMR (75 MHz, CDCl₃) δ 154.5 (q), 144.1 (q), 133.8 (q), 130.6 (q), 129.7 (t), 129.0 (t), 128.0 (t), 126.7 (t), 126.3 (t), 113.5 (t), 73.4 (t), 65.4 (d), 57.3 (d), 54.6 (d), 52.9 (d), 45.8 (s), 20.7 (s).

### Example 158: 1-methyl-4-(2-(4-methyl-2-(1-phenylvinyl)phenoxy)ethyl)piperazine

### Example 158a: p-tolyl benzoate

To a solution of p-cresol (200 g, 1.85 mol) in DCM (Volume: 150 mL) at 0° C. was added triethylamine (387 mL, 2774 mmol) and DMAP (0.23 g, 1.85 mmol) followed by benzoyl chloride (312 g, 2.22 mol). The reaction mixture was warmed to room temperature and stirred for 3 h. At this point, the reaction mixture was diluted with DCM 500 mL and quenched with aqueous NH₄Cl 500 mL. The aqueous layer was extracted with DCM (2×100) and the combined organic layers were subsequently washed with H₂O and brine, and concentrated in vacuo. The resulting residue was recrystallized in 300 mL PE to get the title product (347 g, yield 87%) as a white powder. GC/MS (EI): m/z (%): 212 (15) [M⁺], 105 (100), 77 (35), 51 (5).

### Example 158b: (2-hydroxy-5-methylphenyl)(phenyl)methanone

The mixture of p-tolyl benzoate (300 g, 1.41 mol) and aluminum chloride (283 g, 2.12 mol) was slowly (for ca. 0.5 hr) heated to 120-125° C. in chloroBenzene (Volume: 200 mL). This mixture was stirred at this temperature for 20 hr, and then HCl (172 mL, 5.65 mol) 2M HCl was added at 0° C. The product was extracted with 3×200 mL of DCM, the combined extract was dried over Na₂SO₄ and then evaporated to dryness. This procedure gave the title product (240 g, 1.07 mol, yield 76%) which was further used without an additional purification. GC/MS (EI): m/z (%): 212 (75) [M⁺], 211 (100), 135 (40), 105 (25), 77 (35).

### Example 158c: 2-(1-hydroxy-1-phenylethyl)-4-methylphenol

At 0° C., to methylmagnesium bromide (3M solution in ethyl ether 14.2 mmol, 4.7 mL) in THF (Volume: 10 mL) was added (2-hydroxy-5-methylphenyl)(phenyl)methanone (1.00 g, 4.7 mmol) and the resulting mixture was allowed to stir at room temperature for 1 hour. A saturated NH₄Cl solution (50 mL) was used to quench the reaction, MTBE (30 mL×3) was used to extract the solution, the solvent was removed and the residue was used directly as the title product (1.10 g, yield 87%). GC/MS (EI): m/z (%): 211 (100) [M⁺], 210 (100), 195 (95), 165 (55), 152 (20), 77 (20).

### Example 158d: 4-methyl-2-(1-phenylvinyl)phenol

To a stirred solution of 2-(1-hydroxy-1-phenylethyl)-4-methylphenol (1.10 g, 4.8 mmol) and MgSO₄ in DCM (30 mL) was added acetic acid (0.060 g, 0.96 mmol). Then the resulting mixture was stirred at room temperature for overnight. Upon completion, the reaction mixture was filtered and washed with DCM. The filtrate was concentrated and the residue was purified by silica gel chromatography (PE:MTBE=10:1) to afford the title product (1.00 g, yield 96%) as a viscous liquid. GC/MS (EI): m/z (%): 210 (100) [M⁺], 209 (100), 195 (95), 178 (15), 165 (50), 152(20), 115 (13), 103 (10), 77 (20).

### Example 158e: 1-methyl-4-(2-(4-methyl-2-(1-phenylvinyl)phenoxy)ethyl)piperazine

Following the general procedure from Example 80: A mixture of 4-methyl-2-(1-phenylvinyl)phenol (0.25 g, 1.2 mmol) 1-(2-chloroethyl)-4-methylpiperazine dihydrochloride (0.56 g, 2.4 mmol), potassium carbonate (1.64 g, 12 mmol) and potassium iodide (0.040 g, 0.24 mmol) in DMF (10 mL) was reacted to give the title product (0.20 g, yield 47%) as a viscous liquid. GC/MS (EI): m/z (%): 336 (98) [M⁺], 209 (10), 195 (5), 178 (12), 165 (20), 127(35), 113 (100), 98 (20), 70 (85). ¹H NMR (300 MHz, CDCl₃) δ 7.31-7.20 (m, 5H), 7.13-7.06 (m, 2H), 6.79-6.72 (m, 1H), 5.62 (s, 1H), 5.29 (s, 1H), 3.89 (t, J=5.7 Hz, 2H), 2.45-2.32 (m, 13H), 2.25 (s, 3H). ¹³C NMR (75 MHz, CDCl₃) δ 154.3 (q), 147.8 (q), 141.8 (q), 132.0 (t), 131.4 (q), 130.2 (q), 129.4 (t), 128.1 (t), 127.3 (t), 126.5 (t), 115.3 (d), 112.6 (t), 67.3 (d), 57.0 (d), 55.1 (d), 53.4 (d), 46.1 (s), 20.6 (s).

### Example 159: 1-methyl-4-(2-(4-methyl-2-(1-phenylethyl)phenoxy)ethyl)piperazine

### Example 159a: 4-methyl-2-(1-phenylethyl)phenol

The mixture of 4-methyl-2-(1-phenylvinyl)phenol (0.40 g, 1.9 mmol) and 10% Pd/C (0.15 g) in MeOH (30 mL) was stirred under H₂ for overnight. The mixture was filtered through a small pad of silica gel and the solvent was removed to get the title product (0.40 g, yield 94%) as a viscous liquid, which was used for next step directly. GC/MS (EI): m/z (%): 212 (98) [M⁺], 197 (100), 182 (50), 165 (20), 134 (30), 91(40), 77 (40), 65 (15).

### Example 159b: 1-methyl-4-(2-(4-methyl-2-(1-phenylethyl)phenoxy)ethyl)piperazine

Following the general procedure from Example 80: The mixture of 4-methyl-2-(1-phenylethyl)phenol (0.25 g, 1.2 mmol), 1-(2-chloroethyl)-4-methylpiperazine dihydrochloride (0.56 g, 2.4 mmol), potassium carbonate (1.63 g, 11 mmol) and potassium iodide (0.040 g, 0.24 mmol) in DMF (10 mL) were reacted to give the title product (0.20 g, yield 48%) as a viscous liquid. GC/MS (EI): m/z (%): 338 (25) [M⁺], 209 (5), 195 (10), 165 (12), 127 (90), 113(100), 100 (20), 84 (10), 70 (80). ¹H NMR (300 MHz, CDCl₃) δ 7.25-7.17 (m, 4H), 7.16-7.09 (m, 1H), 7.00 (s, 1H), 6.95 (d, J=8.2 Hz, 1H), 6.70 (d, J=8.2 Hz, 1H), 4.50 (q, J=7.3 Hz, 1H), 4.10-3.88 (m, 2H), 2.76-2.65 (m, 2H), 2.59-2.55 (m, 4H), 2.54-2.45 (m, 4H), 2.30 (s, 3H), 2.26 (s, 3H), 1.56 (d, J=7.3 Hz, 3H). ¹³C NMR (75 MHz, CDCl₃) δ 154.1 (q), 146.8 (q), 134.5 (q), 129.8 (q), 128.6 (t), 128.1 (t), 127.7 (t), 127.4 (t), 125.7 (t), 111.7 (t), 66.7 (d), 57.3 (d), 55.1 (d), 53.4 (d), 46.0 (s), 37.8 (t), 21.2 (s), 20.9 (s).

### Example 160: 4-(2-(2-(1-(2-fluorophenyl)ethyl)-4,6-dimethylphenoxy)ethyl)morpholine

A mixture of 2-(1-(2-fluorophenyl)ethyl)-4,6-dimethylphenol (0.37 g, 1.51 mmol) (prepared in an analogous sequence as described in Example 158 followed by Example 159a). 4-(2-chloroethyl)morpholine hydrochloride (0.85 g, 4.54 mmol), potassium carbonate (1.26 g, 9.09 mmol) and potassium iodide (0.03 g, 0.15 mmol) in DMF (20 mL) was heated to reflux for 3 hours. Water was added to the cooled solution, DCM was used to extract the solution, the solvent was removed and the residue was purified by column chromatography on silica gel (PE:MTBE=1:1) to get the title product (0.46 g, 1.25 mmol, yield 82%) as a light yellow liquid. GC/MS (EI): m/z (%): 357 (10) [M⁺], 270 (30), 255 (15), 241 (5), 227 (5), 114(30), 100 (100), 91 (3), 70 (10), 56 (15).

### Example 161: 4-(2-(2-(1-(2-fluorophenyl)vinyl)-4,6-dimethylphenoxy)ethyl)morpholine

A mixture of 2-(1-(2-fluorophenyl)vinyl)-4,6-dimethylphenol (0.5 g, 2.06 mmol) (prepared in an analogous sequence as described in Example 158), 4-(2-chloroethyl)morpholine hydrochloride (1.15 g, 6.19 mmol), potassium carbonate (1.71 g, 12.38 mmol) and potassium iodide (0.03 g, 0.21 mmol) in DMF (20 mL) was heated to reflux for 3 hours.

GCMS showed that sm was consumed completely and a mixture of =7:3 was observed in GCMS. Water was added to the cooled solution, DCM was used to extract the mixture, the solvent was removed and the residue was purified by column chromatography on silica gel (PE:MTBE=5:1) to get the title product (0.43 g, 1.25 mmol, yield 57%) as a light yellow liquid. GC/MS (EI): m/z (%): 355 (20) [M⁺], 270 (30), 255 (35), 241 (10), 183 (10), 114(20), 100 (100), 85 (3), 70 (10), 56 (15).

### Example 162: (5-methyl-2-(2-(4-methylpiperazin-1-yl)ethoxy)phenyl)(phenyl)methanone

A mixture of (2-hydroxy-5-methylphenyl)(phenyl)methanone (0.50 g, 2.4 mmol), 1-(2-chloroethyl)-4-methylpiperazine dihydrochloride (1.11 g, 4.7 mmol), potassium carbonate (3.26 g, 23 mmol) and potassium iodide (0.080 g, 0.47 mmol) in DMF (Volume: 10 mL) was heated to reflux for 3 hours. Water was added, DCM was used to extract the solution for three times, the solvent was removed and the residue was purified by column chromatography on silica gel (DCM:Methanol=10:1) to get the title product (0.58 g, 1.54 mmol, yield 65.5%) as a viscous liquid. GC/MS (EI): m/z (%): 338 (3) [M⁺], 282 (3), 268 (5), 211 (10), 165 (5), 127(55), 113 (100), 98 (10), 70 (85). ¹H NMR (300 MHz, CDCl₃) δ 7.79-7.71 (m, 2H), 7.55-7.50 (m, 1H), 7.44-7.35 (m, 2H), 7.24-7.15 (m, 2H), 6.81 (d, J=6.3 Hz, 1H), 3.97 (t, J=5.6 Hz, 2H), 2.39 (t, J=5.6 Hz, 2H), 2.35-2.24 (m, 11H), 2.21 (s, 3H). ¹³C NMR (75 MHz, CDCl₃) δ 197.1 (q), 154.6 (q), 138.4 (q), 132.8 (t), 132.5 (t), 130.4 (q), 130.1 (t), 129.7 (t), 129.1 (q), 128.2 (t), 112.6 (t), 67.5 (d), 56.8 (d), 55.0 (d), 53.4 (d), 46.0 (s), 20.5 (s).

### Example 163: 1-(2-((2-benzyl-4-methylcyclohexyl)oxy)ethyl)-4-methylpiperazine

Sodium hydride (489 mg, 12.24 mmol) was added into 2-benzyl-4-methylcyclohexanol (0.50 mg, 2.5 mmol) in DMF (15 mL) in an ice bath for 2 h, 1-(2-chloroethyl)-4-methylpiperazine dihydrochloride (0.87 g, 3.7 mmol) was added dropwise to the above solution. After completion of dropwise addition, it was allowed to react at room temperature for 12 hours. After completion of the reaction, it was washed twice with water. The organic phase was dried over anhydrous sodium sulfate, and evaporated to dryness to obtain the title product (0.20 g, yield 25%). GC/MS (EI): m/z (%): 330 (3) [M⁺], 127 (35), 113 (100), 98 (50), 84 (4), 70(20), 56(5).

### Example 164: 1-(2-((2-benzyl-4-methylcyclohexyl)oxy)ethyl)-4-methylpiperazine dihydrochloride

Prepared according to the procedure described in Example 96 from 1-(2-((2-benzyl-4-methylcyclohexyl)oxy)ethyl)-4-methylpiperazine to obtain the title product (0.15 g, yield 18%) as a brown solid. GC/MS (EI): m/z (%): 330 (2) [M⁺], 127 (40), 113 (100), 98 (50), 84 (4), 70(20), 56(5).

### Example 165: 2-(3,5-dimethyl-2-(2-morpholinoethoxy)benzyl)aniline

A mixture of 4-(2-(2,4-dimethyl-6-(2-nitrobenzyl)phenoxy)ethyl)morpholine (0.45 g, 0.85 mmol) and 10% Pd/C (150 mg) in Methanol (15 mL) was stirred under H₂ for overnight. The mixture was filtered, the solvent was removed and the residue was purified by column chromatography on silica gel (DCM:Methanol=95:5) to get the title product (0.21 g, 0.06 mmol, yield 36.3%) as a viscous liquid. GC/MS (EI): m/z (%): 340 (3) [M⁺], 253 (13), 227 (29), 210 (14), 194 (4), 165 (2), 114 (26), 100 (100), 87 (1), 70 (5).

### Example 166: Assay on TRPM8 Modulators

A HEK293 cell line stably expressing hTRPM8 was generated according to Klein et al., (Chem. Senses 36: 649-658, 2011) and receptor activation was monitored by calcium imaging in a Flexstation. For Ca-imaging assays of TRPM8 channel activation, cells were seeded on day 0 at a density of 12000 cells per well in Dulbecco's modified Eagle medium (DMEM) containing 9% foetal bovine serum in black, clear bottom 96-well plates that had been coated with 0.001% polyethyleneimine (molecular weight=60 000, Acros Organics). On day 2, agonists were evaluated via calcium imaging using Fluo-4. Briefly, growth medium was discarded, and the cells were incubated in the dark for 1 h at 37° C. in 50 μL loading buffer consisting of 2.7 μM Fluo-4 AM (Invitrogen) and 2.5 μM probenecid (Sigma-Aldrich) in DMEM (without serum). After incubation, the plates were washed five times with 100 μL of assay buffer (in mM: 130 NaCl, 5 KCl, 10 HEPES, 2 CaCl₂, and 10 glucose, pH7.4.) and further incubated in the dark at room temperature for 30 min. The cells were then washed five times with 100 μL assay buffer and then calcium influx to serial dilutions of inventive compounds were measured in a Flexstation 3 (Molecular Devices). Receptor activation was initiated following addition of 20 μl of a 10-fold concentrated ligand stock solution, which is also prepared in assay buffer. Fluorescence was continuously monitored for 15 seconds prior to ligand addition and for 105 seconds after ligand addition, for a total of 120 seconds. Maximal receptor activation in relation to solvent control and relative to 31.6 μM menthol is determined. Data from serial dilutions were processed with a KNIME workflow to fit a sigmoidal dose-response curve and to extrapolate EC₅₀ values.

TRPM8 agonist exhibiting an EC₅ value below 35 μM are presented in Table 1 below.

TABLE 1

| Comp. | | |
|---|---|---|
| No. | Chemical Name | EC50 |
| 40 | (1-(2-(2-benzyl-4-methylphenoxy)ethyl)piperidin-4-yl)methanol | + |
| 41 | (5-methyl-2-(2-(4-methylpiperazin-1-yl)ethoxy)phenyl)(phenyl)- | + |
| | methanol | |
| 42 | (5-methyl-2-(2-(4-methylpiperazin-1-yl)ethoxy)phenyl)(phenyl)- | + |
| | methanone | |
| 43 | (E)-1-(2-((6-benzylidene-2,4-dimethylcyclohex-1-en-1- | + |
| | yl)oxy)ethyl)-4-methylpiperazine | |
| 44 | (E)-1-(3-(2-benzyl-4-methylphenyl)allyl)-4-methylpiperazine | + |
| 45 | (E)-1-(3-(2-benzyl-4-methylphenyl)allyl)-4-methylpiperazine and 1- | ++ |
| | (3-(2-benzyl-4-methylphenyl)propyl)-4-methylpiperazine (4:1) | |
| 46 | (E)-2-(5-methyl-2-(2-(4-methylpiperazin-1- | ++ |
| | yl)ethoxy)benzylidene)cyclohexan-1-one | |
| 47 | (E)-2-(5-methyl-2-(2-morpholinoethoxy)benzylidene)cyclohexan-1- | +++ |
| | one | |
| 48 | 2-(3,5-dimethyl-2-(2-morpholinopropoxy)benzyl)benzonitrile | +++ |
| 49 | 2-(3,5-dimethyl-2-((1-morpholinopropan-2- | +++ |
| | yl)oxy)benzyl)benzonitrile | |
| 50 | 1-((8-benzylchroman-2-yl)methyl)-4-methylpiperazine | + |
| 51 | 1-(2-((2-benzyl-4-methylcyclohexyl)oxy)ethyl)-4-methylpiperazine | + |
| 52 | 1-(2-((2-benzyl-4-methylcyclohexyl)oxy)ethyl)-4-methylpiperazine | + |
| | dihydrochloride | |
| 53 | 1-(2-(2-((5-chlorothiophen-2-yl)methyl)-4-methylphenoxy)ethyl)-4- | + |
| | methylpiperazine | |
| 54 | 1-(2-(2-(2-bromobenzyl)-4,6-dimethylphenoxy)ethyl)-4- | ++ |
| | methylpiperazine | |
| 55 | 1-(2-(2-(2-chlorobenzyl)-4-methylphenoxy)ethyl)-4- | +++ |
| | methylpiperazine | |
| 56 | 1-(2-(2-(2-fluorobenzyl)-4,6-dimethylphenoxy)ethyl)-4- | +++ |
| | methylpiperazine | |
| 57 | 1-(2-(2-(2-iodobenzyl)-4,6-dimethylphenoxy)ethyl)-4- | ++ |
| | methylpiperazine | |
| 58 | 1-(2-(2-(3-bromobenzyl)-4,6-dimethylphenoxy)ethyl)-4- | + |
| | methylpiperazine | |
| 59 | 1-(2-(2-(3-chlorobenzyl)-4-methylphenoxy)ethyl)-4- | + |
| | methylpiperazine | |
| 60 | 1-(2-(2-(4-chlorobenzyl)-4-methylphenoxy)ethyl)-4- | + |
| | methylpiperazine hydrochloride | |
| 61 | 1-(2-(2-(furan-2-ylmethyl)-4-methylphenoxy)ethyl)-4- | + |
| | methylpiperazine | |
| 62 | 1-(2-(2,4-dimethyl-6-(2-(trifluoromethyl)benzyl)phenoxy)ethyl)-4- | ++ |
| | methylpiperazine | |
| 63 | 1-(2-(2,4-dimethyl-6-(pyridin-2-ylmethyl)phenoxy)ethyl)-4- | + |
| | methylpiperazine | |
| 64 | 1-(2-(2,4-dimethyl-6-(pyridin-3-ylmethyl)phenoxy)ethyl)-4- | + |
| | methylpiperazine | |
| 65 | 1-(2-(2,4-dimethyl-6-(pyridin-4-ylmethyl)phenoxy)ethyl)-4- | + |
| | methylpiperazine | |
| 66 | 1-(2-(2-benzyl-3,6-dimethylphenoxy)ethyl)-4-methylpiperazine | + |
| 67 | 1-(2-(2-benzyl-3-methoxyphenoxy)ethyl)-4-methylpiperazine | + |
| 68 | 1-(2-(2-benzyl-3-methylphenoxy)ethyl)-4-methylpiperazine | + |
| 69 | 1-(2-(2-benzyl-4-(tert-butyl)-6-methoxyphenoxy)ethyl)-4- | + |
| | methylpiperazine | |
| 70 | 1-(2-(2-benzyl-4,6-dimethylphenoxy)ethyl)-4-methylpiperazine | ++ |
| 71 | 1-(2-(2-benzyl-4,6-dimethylphenoxy)ethyl)-4-methylpiperazine | ++ |
| | dihydrochloride | |
| 72 | 1-(2-(2-benzyl-4-chlorophenoxy)ethyl)-4-ethylpiperazine | + |
| | hydrochloride | |
| 73 | 1-(2-(2-benzyl-4-chlorophenoxy)ethyl)-4-methylpiperazine | ++ |
| | hydrochloride | |
| 74 | 1-(2-(2-benzyl-4-ethylphenoxy)ethyl)-4-methylpiperazine | ++ |
| | hydrochloride | |
| 75 | 1-(2-(2-benzyl-4-isopropylphenoxy)ethyl)-4-methylpiperazine | + |
| 76 | 1-(2-(2-benzyl-4-methylphenoxy)ethyl)-4-ethylpiperazine | + |
| | hydrochloride | |
| 77 | 1-(2-(2-benzyl-4-methylphenoxy)ethyl)-4-methylpiperazine | ++ |
| 78 | 1-(2-(2-benzyl-4-methylphenoxy)ethyl)-4-methylpiperazine 2,3- | ++ |
| | dihydroxysuccinate | |
| 79 | 1-(2-(2-benzyl-4-methylphenoxy)ethyl)-4-methylpiperazine 2- | ++ |
| | hydroxypropane-1,2,3-tricarboxylate | |
| 80 | 1-(2-(2-benzyl-4-methylphenoxy)ethyl)-4-methylpiperazine | ++ |
| | bis(2,2,2-trifluoroacetate) | |
| 81 | 1-(2-(2-benzyl-4-methylphenoxy)ethyl)-4-methylpiperazine bis(2- | ++ |
| | hydroxysuccinate) | |
| 82 | 1-(2-(2-benzyl-4-methylphenoxy)ethyl)-4-methylpiperazine | ++ |
| | bis(phosphate) | |
| 83 | 1-(2-(2-benzyl-4-methylphenoxy)ethyl)-4-methylpiperazine | ++ |
| | dimaleate | |
| 84 | 1-(2-(2-benzyl-4-methylphenoxy)ethyl)-4-methylpiperazine | ++ |
| | dimethanesulfonate | |
| 85 | 1-(2-(2-benzyl-4-methylphenoxy)ethyl)-4-methylpiperazine | ++ |
| | hydrochloride | |
| 86 | 1-(2-(2-benzyl-4-methylphenoxy)ethyl)-4-methylpiperazine oxalate | ++ |
| 87 | 1-(2-(2-benzyl-4-methylphenoxy)ethyl)-4-methylpiperazine sulfate | ++ |
| 88 | 1-(2-(2-benzyl-4-methylphenoxy)ethyl)piperazine | + |
| 89 | 1-(2-(2-benzyl-4-methylphenoxy)ethyl)piperidin-4-ol | + |
| 90 | 1-(2-(2-benzyl-4-methylphenoxy)propyl)-4-methylpiperazine | ++ |
| 91 | 1-(2-(2-benzyl-4-methylphenoxy)propyl)-4-methylpiperazine | ++ |
| | hydrochloride | |
| 92 | 1-(2-(2-benzyl-6-methylphenoxy)ethyl)-4-methylpiperazine | + |
| 93 | 1-(2-(2-benzylphenoxy)ethyl)-4-methylpiperazine hydrochloride | + |
| 94 | 1-(2-(2-isobutyl-4-methylphenoxy)ethyl)-4-methylpiperazine | + |
| 95 | 1-(2-(2-isobutyl-4-methylphenoxy)ethyl)-4-methylpiperazine | + |
| | dihydrochloride | |
| 96 | 1-(2-benzyl-4-methylphenethyl)-4-methylpiperazine | + |
| 97 | 1-(2-benzyl-4-methylphenyl)-3-(4-methylpiperazin-1-yl)propan-2-ol | ++ |
| 98 | 1-(2-benzyl-4-methylphenyl)-3-(4-methylpiperazin-1-yl)propan-2- | + |
| | one | |
| 99 | 1-(3-(2-benzyl-4-methylphenoxy)propyl)-4-methylpiperazine | + |
| | hydrochloride | |
| 100 | 1-(3-(2-benzyl-4-methylphenyl)propyl)-4-methylpiperazine | ++ |
| 101 | 1-methyl-4-(2-(4-methyl-2-(1-phenylethyl)phenoxy)ethyl)piperazine | ++ |
| 102 | 1-methyl-4-(2-(4-methyl-2-(1-phenylvinyl)phenoxy)ethyl)piperazine | ++ |
| 103 | 1-methyl-4-(2-(4-methyl-2-(2- | ++ |
| | methylbenzyl)phenoxy)ethyl)piperazine | |
| 104 | 1-methyl-4-(2-(4-methyl-2-(3- | + |
| | methylbenzyl)phenoxy)ethyl)piperazine | |
| 105 | 1-methyl-4-(2-(4-methyl-2-(thiophen-2- | ++ |
| | ylmethyl)phenoxy)ethyl)piperazine hydrochloride | |
| 106 | 2-(2-((1-hydroxypropan-2-yl)oxy)-3,5-dimethylbenzyl)benzonitrile | ++ |
| 107 | 2-(2-(2-hydroxy-3-morpholinopropyl)-5-methylphenoxy)benzonitrile | +++ |
| 108 | 2-(2-(3,5-dimethyl-2-(2- | +++ |
| | morpholinoethoxy)benzyl)phenyl)acetonitrile | |
| 109 | 2-(2,4-dimethyl-5-(2-(4-methylpiperazin-1- | + |
| | yl)ethoxy)benzyl)benzonitrile | |
| 110 | 2-(2,6-difluorobenzyl)-4-methyl-N-(2-morpholinoethyl)aniline | ++ |
| 111 | 2-(2,6-difluorobenzyl)-N,4,6-trimethyl-N-(2-morpholinoethyl)aniline | ++ |
| 112 | 2-(2,6-difluorobenzyl)-N,4-dimethyl-N-(2-morpholinoethyl)aniline | ++++ |
| 113 | 2-(2-fluorobenzyl)-N,4,6-trimethyl-N-(2-morpholinoethyl)aniline | ++ |
| 114 | 2-(2-fluorobenzyl)-N,4-dimethyl-N-(2-morpholinoethyl)aniline | +++ |
| 115 | 2-(3,5-dimethyl-2-((1-(4-methylpiperazin-1-yl)propan-2- | +++ |
| | yl)oxy)benzyl)benzonitrile | |
| 116 | 2-(3,5-dimethyl-2-((1-(4-methylpiperazin-1-yl)propan-2- | +++ |
| | yl)oxy)benzyl)benzonitrile hydrochloride | |
| 117 | 2-(3,5-dimethyl-2-(2-(4-methylpiperazin-1- | +++ |
| | yl)ethoxy)benzyl)benzonitrile | |
| 118 | 2-(3,5-dimethyl-2-(2-(4-methylpiperazin-1- | ++++ |
| | yl)ethoxy)benzyl)benzonitrile hydrochloride | |
| 119 | 2-(3,5-dimethyl-2-(2-(tetrahydro-2H-pyran-4- | +++ |
| | yl)ethoxy)benzyl)benzonitrile | |
| 120 | 2-(3,5-dimethyl-2-(2-morpholinoethoxy)benzyl)-3-fluorobenzonitrile | +++ |
| 121 | 2-(3,5-dimethyl-2-(2-morpholinoethoxy)benzyl)aniline | + |
| 122 | 2-(3,5-dimethyl-2-(2-morpholinoethoxy)benzyl)benzonitrile | ++++ |
| 123 | 2-(3,5-dimethyl-2-(2-morpholinoethoxy)benzyl)benzonitrile | ++++ |
| | hydrochloride | |
| 124 | 2-(3,5-dimethyl-2-(3-morpholinopropoxy)benzyl)benzonitrile | +++ |
| 125 | 2-(3,5-dimethyl-2-(3-morpholinopropyl)phenoxy)benzonitrile | ++++ |
| 126 | 2-(3,5-dimethyl-2-(methyl(2- | ++++ |
| | morpholinoethyl)amino)benzyl)benzonitrile | |
| 127 | 2-(4-methyl-2-(3-(4-methylpiperazin-1- | + |
| | yl)propyl)phenoxy)benzonitrile | |
| 128 | 2-(5-methyl-2-(2-(1-methylpiperidin-4-yl)ethoxy)benzyl)benzonitrile | + |
| 129 | 2-(5-methyl-2-(2-(4-methylpiperazin-1- | +++ |
| | yl)ethoxy)benzyl)benzonitrile | |
| 130 | 2-(5-methyl-2-(2-(4-methylpiperazin-1- | +++ |
| | yl)ethoxy)benzyl)benzonitrile dihydrochloride | |
| 131 | 2-(5-methyl-2-(2-morpholinoethoxy)benzyl)cyclohexan-1-one | + |
| 132 | 2-(5-methyl-2-(2-morpholinopropoxy)benzyl)benzonitrile | ++ |
| 133 | 2-(5-methyl-2-(3-(4-methylpiperazin-1- | +++ |
| | yl)propyl)phenoxy)benzonitrile | |
| 134 | 2-(5-methyl-2-(3-(4-methylpiperazin-1- | +++ |
| | yl)propyl)phenoxy)benzonitrile hydrochloride | |
| 135 | 2-benzyl-4-methyl-N-(2-(4-methylpiperazin-1-yl)ethyl)aniline | + |
| 136 | 2-benzyl-4-methylphenyl 2-(4-methylpiperazin-1-yl)acetate | + |
| 137 | 2-benzyl-N,4-dimethyl-N-(2-(4-methylpiperazin-1-yl)ethyl)aniline | +++ |
| 138 | 2-benzyl-N,4-dimethyl-N-(2-morpholinoethyl)aniline | +++ |
| 139 | 3-(3,5-dimethyl-2-(2-(4-methylpiperazin-1- | + |
| | yl)ethoxy)benzyl)benzonitrile dihydrochloride | |
| 140 | 3-(3,5-dimethyl-2-(2-morpholinoethoxy)benzyl)aniline | + |
| 141 | 3-bromo-2-(3,5-dimethyl-2-(2- | ++ |
| | morpholinoethoxy)benzyl)benzonitrile | |
| 142 | 3-chloro-2-(3,5-dimethyl-2-(2- | ++ |
| | morpholinoethoxy)benzyl)benzonitrile | |
| 143 | 4-(1-(2-(2,6-difluorobenzyl)-4,6-dimethylphenoxy)propan-2- | +++ |
| | yl)morpholine | |
| 144 | 4-(2-(2-(1-(2-fluorophenyl)ethyl)-4,6- | ++ |
| | dimethylphenoxy)ethyl)morpholine | |
| 145 | 4-(2-(2-(1-(2-fluorophenyl)vinyl)-4,6- | ++ |
| | dimethylphenoxy)ethyl)morpholine | |
| 146 | 4-(2-(2-(2,4-difluorobenzyl)-4,6-dimethylphenoxy)ethyl)morpholine | ++ |
| 147 | 4-(2-(2-(2,5-difluorobenzyl)-4,6-dimethylphenoxy)ethyl)morpholine | +++ |
| 148 | 4-(2-(2-(2,6-dichlorobenzyl)-4,6-dimethylphenoxy)ethyl)morpholine | ++ |
| 149 | 4-(2-(2-(2,6-difluorobenzyl)-4,6-dimethylphenoxy)ethyl)morpholine | +++ |
| 150 | 4-(2-(2-(2,6-difluorobenzyl)-4,6- | ++ |
| | dimethylphenoxy)propyl)morpholine | |
| 151 | 4-(2-(2-(2,6-difluorobenzyl)-4-methylphenoxy)ethyl)morpholine | +++ |
| 152 | 4-(2-(2-(2-chlorobenzyl)-4,6-dimethylphenoxy)ethyl)morpholine | +++ |
| 153 | 4-(2-(2-(2-fluorobenzyl)-4,6-dimethylphenoxy)ethyl)morpholine | +++ |
| 154 | 4-(2-(2-(3,5-difluorobenzyl)-4,6-dimethylphenoxy)ethyl)morpholine | ++ |
| 155 | 4-(2-(2,4-dimethyl-5- | + |
| | ((perfluorophenyl)methyl)phenoxy)ethyl)morpholine | |
| 156 | 4-(2-(2,4-dimethyl-6-(2,4,5- | + |
| | trifluorobenzyl)phenoxy)ethyl)morpholine | |
| 157 | 4-(2-(2,4-dimethyl-6-(2-nitrobenzyl)phenoxy)ethyl)morpholine | ++ |
| 158 | 4-(2-(2-benzyl-4-methylphenoxy)ethyl)-1,2,6-trimethylpiperazine | ++ |
| 159 | 4-(2-(2-benzyl-4-methylphenoxy)ethyl)morpholine hydrochloride | + |
| 160 | 4-(2-(2-benzyl-4-methylphenoxy)ethyl)thiomorpholine 1-oxide | + |
| 161 | 4-(2-(2-ethyl-6-(2-fluorobenzyl)-4-methylphenoxy)ethyl)morpholine | +++ |
| 162 | 4-(3-(2-(2-fluorophenoxy)-4,6-dimethylphenyl)propyl)morpholine | ++++ |
| 163 | 4-methyl-2-(5-methyl-2-(2-(4-methylpiperazin-1- | + |
| | yl)ethoxy)benzyl)phenol | |
| 164 | ethyl 4-(3,5-dimethyl-2-(2-(4-methylpiperazin-1- | + |
| | yl)ethoxy)benzyl)benzoate dihydrochloride | |
| 165 | methyl 3-benzyl-4-(2-(4-methylpiperazin-1-yl)ethoxy)benzoate | + |

++++ EC₅₀ Value in the range of 0.05 μM and lower
+++ EC₅₀ value in the range of 0.05-0.3 μM
++ EC₅₀ value in the range of 0.3-1.00 μM
+ EC₅₀ value in the range of 1.00-35 μM

### Example 167: Sensory Studies in Aqueous Solution

The compounds as listed below in Table 2 were dissolved at a concentration of 1 wt-% in propylene glycol. These solutions were then dispensed in appropriate quantities into deionized water containing 0.5 wt-% Poloxamer 407 (which is a hydrophilic non-ionic surfactant, e.g., commercially available from SigmaAldrich) and 0.25 wt-% Cremaphor® RH40 (obtained from BASF) as solubilizer, to obtain the desired final concentration of 40 ppm (parts per million) of the respective compound.

A trained group of panelists evaluated the aqueous solutions containing test compound by swilling 20 mL of the solution in the mouth for 60 seconds, followed by spitting, without rinsing the mouth afterwards for the duration of the evaluation. Panelists evaluated and recorded the cooling performance as well as other sensorial and organoleptic attributes at different timepoints over a period of two hours. Cooling performance was rated on a scale from 0 to 10 with 0 being no effect and 10 being freezing. Scores were averaged for all panelists and the cooling intensity qualified as “N” for “none” (score of 0), “L” for “low” (score above 0, up to 1), “M” for “medium” (score above 1, up to 4), “S” for “strong” (score above 4, up to 8) and “E” for “extreme” (score above 8), which are provided in Table 2. None of the tested compounds showed any statistically significant bitterness or negative organoleptic features.

TABLE 2

| Comp. | | | | |
|---|---|---|---|---|
| No. | Chemical Name | A | B | C |
| 85 | 1-(2-(2-benzyl-4-methylphenoxy)ethyl)-4- | S | 20 | M |
| | methylpiperazine hydrochloride | | | |
| 159 | 4-(2-(2-benzyl-4- | M | 5 | L |
| | methylphenoxy)ethyl)morpholine hydrochloride | | | |
| 100 | 1-(3-(2-benzyl-4-methylphenyl)propyl)-4- | M | 10 | M |
| | methylpiperazine | | | |
| 130 | 2-(5-methyl-2-(2-(4-methylpiperazin-1- | S | 30 | S |
| | yl)ethoxy)benzyl)benzonitrile dihydrochloride | | | |
| 71 | 1-(2-(2-benzyl-4,6-dimethylphenoxy)ethyl)-4- | S | 20 | M |
| | methylpiperazine dihydrochloride | | | |
| 77 | 1-(2-(2-benzyl-4-methylphenoxy)ethyl)-4- | M | 10 | M |
| | methylpiperazine | | | |
| 118 | 2-(3,5-dimethyl-2-(2-(4-methylpiperazin-1- | S | 20 | S |
| | yl)ethoxy)benzyl)benzonitrile hydrochloride | | | |
| 122 | 2-(3,5-dimethyl-2-(2- | S | 10 | M |
| | morpholinoethoxy)benzyl)benzonitrile | | | |
| 137 | 2-benzyl-N,4-dimethyl-N-(2-(4- | S | 10 | M |
| | methylpiperazin-1-yl)ethyl)aniline | | | |
| 90 | 1-(2-(2-benzyl-4-methylphenoxy)propyl)-4- | M | 10 | M |
| | methylpiperazine | | | |
| 149 | 4-(2-(2-(2,6-difluorobenzyl)-4,6- | S | 20 | M |
| | dimethylphenoxy)ethyl)morpholine | | | |
| 153 | 4-(2-(2-(2-fluorobenzyl)-4,6- | M | 20 | M |
| | dimethylphenoxy)ethyl)morpholine | | | |
| 158 | 4-(2-(2-benzyl-4-methylphenoxy)ethyl)-1,2,6- | M | 30 | M |
| | trimethylpiperazine | | | |
| 119 | 2-(3,5-dimethyl-2-(2-(tetrahydro-2H-pyran-4- | M | 10 | L |
| | yl)ethoxy)benzyl)benzonitrile | | | |
| 125 | 2-(3,5-dimethyl-2-(3- | M | 10 | M |
| | morpholinopropyl)phenoxy)benzonitrile | | | |

Column A: Max. cooling
Column B: Time of max. cooling in minutes
Column C: Cooling after 1 hour

### Example 168: Sensory Studies in Model Dentifrice

The compounds listed in Table 3 were dissolved at a concentration of 1 wt-% in propylene glycol. These solutions were then dispersed in appropriate quantities to obtain the desired final concentration of 100 ppm (parts per million) of the test compound into a model, unflavored dentifrice, the formula of which is given below (ingredients obtained from the suppliers respectively indicated in parenthesis).

| | wt % |
|---|---|
| Trisodium Phosphate dodecahydrate | 0.2 |
| Sorbitol | 50.0 |
| Precipitated Silica - Sorbosil ™ AC77 (Surfachem) | 8.0 |
| Precipitated Silica - Sorbosil ™ TC15 (Surfachem) | 9.0 |
| Sodium Carboxy Methyl Cellulose 9M31XF (Ashland) | 0.8 |
| Titanium Dioxide | 1.0 |
| Sodium Lauryl Sulphate | 1.5 |
| PEG 1500 (Kilo) | 5.0 |
| De Water | 24.5 |

A trained group of panelists evaluated the dentifrice containing a compound of formula (I), by brushing their teeth with 1 g of the dentifrice using a toothbrush for 60 seconds, followed by spitting, without rinsing the mouth afterwards for the duration of the evaluation. Panelists evaluated and recorded the cooling performance as well as other sensorial and organoleptic attributes at different timepoints over a period of two hours. Cooling performance was rated on a scale from 0 to 10 with 0 being no effect and 10 being freezing. Scores were averaged for all panelists and the cooling intensity qualified as “N” for “none” (score of 0), “L” for “low” (score above 0, up to 1), “M” for “medium” (score above 1, up to 4), “S” for “strong” (score above 4, up to 8) and “E” for “extreme” (score above 8), which are summarized below in Table 3. None of the tested compounds showed any statistically significant bitterness or negative organoleptic features.

TABLE 3

| Comp. | | | | |
|---|---|---|---|---|
| No. | Chemical Name | A | B | C |
| 85 | 1-(2-(2-benzyl-4-methylphenoxy)ethyl)-4- | L | 20 | L |
| | methylpiperazine hydrochloride | | | |
| 130 | 2-(5-methyl-2-(2-(4-methylpiperazin-1- | M | 30 | M |
| | yl)ethoxy)benzyl)benzonitrile dihydrochloride | | | |
| 118 | 2-(3,5-dimethyl-2-(2-(4-methylpiperazin-1- | M | 30 | M |
| | yl)ethoxy)benzyl)benzonitrile hydrochloride | | | |
| 71 | 1-(2-(2-benzyl-4,6-dimethylphenoxy)ethyl)-4- | M | 10 | L |
| | methylpiperazine dihydrochloride | | | |
| 122 | 2-(3,5-dimethyl-2-(2- | S | 10 | M |
| | morpholinoethoxy)benzyl)benzonitrile | | | |
| 137 | 2-benzyl-N,4-dimethyl-N-(2-(4-methylpiperazin- | M | 10 | L |
| | 1-yl)ethyl)aniline | | | |
| 149 | 4-(2-(2-(2,6-difluorobenzyl)-4,6- | M | 30 | M |
| | dimethylphenoxy)ethyl)morpholine | | | |
| 153 | 4-(2-(2-(2-fluorobenzyl)-4,6- | M | 5 | L |
| | dimethylphenoxy)ethyl)morpholine | | | |
| 125 | 2-(3,5-dimethyl-2-(3- | M | 30 | M |
| | morpholinopropyl)phenoxy)benzonitrile | | | |

Column A: Max. cooling
Column B: Time of max. cooling in minutes
Column C: Cooling after 1 hour

1. A method of modulating transient receptor potential channel melastatin member 8 (TRPM8) comprising bringing the receptor into contact with a compound of formula (I), or a salt or solvate thereof 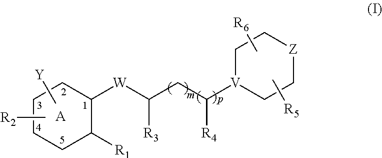
wherein
ring A represents a phenyl ring, cyclohexyl, or cyclohexenyl ring;
R₁ is selected from hydrogen, C₁-C₆ alkyl, and C₁-C₆ alkyloxy;
R₂ is connected to position 3, 4, or 5 of ring A and is selected from hydrogen, halogen, C₁-C₆alkyl, C₁-C₆ alkyloxy, and C(O)O—C₁-C₃-alkyl;
R₃ is selected from hydrogen, C₁-C₆ alkyl, C₂-C₆ alkenyl, OH, and ═O, and R₄ is selected from hydrogen, C₁-C₆ alkyl, C₂-C₆ alkenyl, and OH, with the proviso that at least one of R₃ and R₄ is hydrogen; or
R₃ and R₁ form together with the carbon atoms to which they are attached a six membered ring system, and R₄ is hydrogen;
R₅ is selected from hydrogen, F and C₁-C₆ alkyl;
R₆ is selected from hydrogen, F and C₁-C₆ alkyl;
m is 0 or 1;
p is 0 or 1;
W is selected from —CH₂—, —O—, >C═O, —CH═, and >NR₁₃ wherein R₁₃ is selected from hydrogen and C₁-C₃ alkyl;
V is selected from >CH—, and >N—;
Z is selected from —O—, >S═O, >CHR₁₂, and >NR₁₂ wherein R₁₂ is hydrogen, C₁-C₃ alkyl, OH, or CH₂OH;
Y is connected to position 2 or 3 of ring A and is selected from C₄-C₆ alkyl, 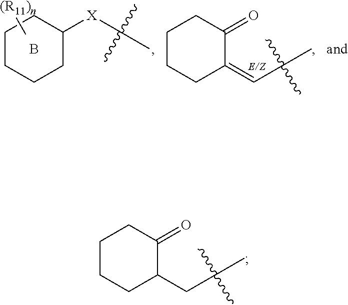
wherein
ring B represents a phenyl, thiophen, furan, or pyridine ring;
R₁₁ is selected from CN, halogen, CH₂CN, NO₂, NH₂, CF₃, C(O)OC₂H₅, C₁-C₃ alkyl, C₁-C₃ alkyloxy, C₂-C₃ alkenyl, and OH, and n is an integer from 0-5, with the proviso that if n>1, R₁₁ can be the same or different; and
X is selected from —CH₂—, —O—, >CH—CH₃, >CH═CH₂, and >CHOH.

2. A method according to claim 1 wherein the compound of formula (I) is selected from the group consisting of (1-(2-(2-benzyl-4-methylphenoxy)ethyl)piperidin-4-yl)methanol; (5-methyl-2-(2-(4-methylpiperazin-1-yl)ethoxy)phenyl)(phenyl)methanol; (E)-1-(2-((6-benzylidene-2,4-dimethylcyclohex-1-en-1-yl)oxy)ethyl)-4-methylpiperazine; (E)-1-(3-(2-benzyl-4-methylphenyl)allyl)-4-methylpiperazine; 1-(3-(2-benzyl-4-methylphenyl)propyl)-4-methylpiperazine; (E)-2-(5-methyl-2-(2-(4-methylpiperazin-1-yl)ethoxy)benzylidene)cyclohexan-1-one; (E)-2-(5-methyl-2-(2-morpholinoethoxy)benzylidene)cyclohexan-1-one; 2-(3,5-dimethyl-2-(2-morpholinopropoxy)benzyl)benzonitrile; 2-(3,5-dimethyl-2-((1-morpholinopropan-2-yl)oxy)benzyl)benzonitrile; 1-((8-benzylchroman-2-yl)methyl)-4-methylpiperazine; 1-(2-((2-benzyl-4-methylcyclohexyl)oxy)ethyl)-4-methylpiperazine; 1-(2-((2-benzyl-4-methylcyclohexyl)oxy)ethyl)-4-methylpiperazine dihydrochloride; 1-(2-(2-((5-chlorothiophen-2-yl)methyl)-4-methylphenoxy)ethyl)-4-methylpiperazine; 1-(2-(2-(2-bromobenzyl)-4,6-dimethylphenoxy)ethyl)-4-methylpiperazine; 1-(2-(2-(2-chlorobenzyl)-4-methylphenoxy)ethyl)-4-methylpiperazine; 1-(2-(2-(2-fluorobenzyl)-4,6-dimethylphenoxy)ethyl)-4-methylpiperazine; 1-(2-(2-(2-iodobenzyl)-4,6-dimethylphenoxy)ethyl)-4-methylpiperazine; 1-(2-(2-(3-bromobenzyl)-4,6-dimethylphenoxy)ethyl)-4-methylpiperazine: 1-(2-(2-(3-chlorobenzyl)-4-methylphenoxy)ethyl)-4-methylpiperazine; 1-(2-(2-(4-chlorobenzyl)-4-methylphenoxy)ethyl)-4-methylpiperazine hydrochloride; 1-(2-(2-(furan-2-ylmethyl)-4-methylphenoxy)ethyl)-4-methylpiperazine; 1-(2-(2,4-dimethyl-6-(2-(trifluoromethyl)benzyl)phenoxy)ethyl)-4-methylpiperazine; 1-(2-(2,4-dimethyl-6-(pyridin-2-ylmethyl)phenoxy)ethyl)-4-methylpiperazine; 1-(2-(2,4-dimethyl-6-(pyridin-3-ylmethyl)phenoxy)ethyl)-4-methylpiperazine; 1-(2-(2,4-dimethyl-6-(pyridin-4-ylmethyl)phenoxy)ethyl)-4-methylpiperazine; 1-(2-(2-benzyl-3,6-dimethylphenoxy)ethyl)-4-methylpiperazine; 1-(2-(2-benzyl-3-methoxyphenoxy)ethyl)-4-methylpiperazine; 1-(2-(2-benzyl-3-methylphenoxy)ethyl)-4-methylpiperazine; 1-(2-(2-benzyl-4-(tert-butyl)-6-methoxyphenoxy)ethyl)-4-methylpiperazine; 1-(2-(2-benzyl-4,6-dimethylphenoxy)ethyl)-4-methylpiperazine; 1-(2-(2-benzyl-4,6-dimethylphenoxy)ethyl)-4-methylpiperazine dihydrochloride; 1-(2-(2-benzyl-4-chlorophenoxy)ethyl)-4-ethylpiperazine hydrochloride; 1-(2-(2-benzyl-4-chlorophenoxy)ethyl)-4-methylpiperazine hydrochloride; 1-(2-(2-benzyl-4-ethylphenoxy)ethyl)-4-methylpiperazine hydrochloride; 1-(2-(2-benzyl-4-isopropylphenoxy)ethyl)-4-methylpiperazine; 1-(2-(2-benzyl-4-methylphenoxy)ethyl)-4-ethylpiperazine hydrochloride; 1-(2-(2-benzyl-4-methylphenoxy)ethyl)-4-methylpiperazine; 1-(2-(2-benzyl-4-methylphenoxy)ethyl)-4-methylpiperazine 2,3-dihydroxysuccinate; 1-(2-(2-benzyl-4-methylphenoxy)ethyl)-4-methylpiperazine 2-hydroxypropane-1,2,3-tricarboxylate; 1-(2-(2-benzyl-4-methylphenoxy)ethyl)-4-methylpiperazine bis(2,2,2-trifluoroacetate); 1-(2-(2-benzyl-4-methylphenoxy)ethyl)-4-methylpiperazine bis(2-hydroxysuccinate); 1-(2-(2-benzyl-4-methylphenoxy)ethyl)-4-methylpiperazine bis(phosphate); 1-(2-(2-benzyl-4-methylphenoxy)ethyl)-4-methylpiperazine dimaleate; 1-(2-(2-benzyl-4-methylphenoxy)ethyl)-4-methylpiperazine dimethanesulfonate; 1-(2-(2-benzyl-4-methylphenoxy)ethyl)-4-methylpiperazine hydrochloride; 1-(2-(2-benzyl-4-methylphenoxy)ethyl)-4-methylpiperazine oxalate; 1-(2-(2-benzyl-4-methylphenoxy)ethyl)-4-methylpiperazine sulfate; 1-(2-(2-benzyl-4-methylphenoxy)ethyl)piperazine; 1-(2-(2-benzyl-4-methylphenoxy)ethyl)piperidin-4-ol; 1-(2-(2-benzyl-4-methylphenoxy)propyl)-4-methylpiperazine; 1-(2-(2-benzyl-4-methylphenoxy)propyl)-4-methylpiperazine hydrochloride; 1-(2-(2-benzyl-6-methylphenoxy)ethyl)-4-methylpiperazine; 1-(2-(2-benzylphenoxy)ethyl)-4-methylpiperazine hydrochloride; 1-(2-(2-isobutyl-4-methylphenoxy)ethyl)-4-methylpiperazine; 1-(2-(2-isobutyl-4-methylphenoxy)ethyl)-4-methylpiperazine dihydrochloride; 1-(2-benzyl-4-methylphenethyl)-4-methylpiperazine; 1-(2-benzyl-4-methylphenyl)-3-(4-methylpiperazin-1-yl)propan-2-ol; 1-(2-benzyl-4-methylphenyl)-3-(4-methylpiperazin-1-yl)propan-2-one; 1-(3-(2-benzyl-4-methylphenoxy)propyl)-4-methylpiperazine hydrochloride; 1-(3-(2-benzyl-4-methylphenyl)propyl)-4-methylpiperazine; 1-methyl-4-(2-(4-methyl-2-(1-phenylethyl)phenoxy)ethyl)piperazine; 1-methyl-4-(2-(4-methyl-2-(1-phenylvinyl)phenoxy)ethyl)piperazine; 1-methyl-4-(2-(4-methyl-2-(2-methylbenzyl)phenoxy)ethyl)piperazine; 1-methyl-4-(2-(4-methyl-2-(3-methylbenzyl)phenoxy)ethyl)piperazine; 1-methyl-4-(2-(4-methyl-2-(thiophen-2-ylmethyl)phenoxy)ethyl)piperazine hydrochloride; 2-(2-(2-hydroxy-3-morpholinopropyl)-5-methylphenoxy)benzonitrile; 2-(2-(3,5-dimethyl-2-(2-morpholinoethoxy)benzyl)phenyl)acetonitrile; 2-(2,4-dimethyl-5-(2-(4-methylpiperazin-1-yl)ethoxy)benzyl)benzonitrile; 2-(2,6-difluorobenzyl)-4-methyl-N-(2-morpholinoethyl)aniline; 2-(2,6-difluorobenzyl)-N,4,6-trimethyl-N-(2-morpholinoethyl)aniline; 2-(2,6-difluorobenzyl)-N,4-dimethyl-N-(2-morpholinoethyl)aniline; 2-(2-fluorobenzyl)-N,4,6-trimethyl-N-(2-morpholinoethyl)aniline; 2-(2-fluorobenzyl)-N,4-dimethyl-N-(2-morpholinoethyl)aniline; 2-(3,5-dimethyl-2-((1-(4-methylpiperazin-1-yl)propan-2-yl)oxy)benzyl)benzonitrile; 2-(3,5-dimethyl-2-((1-(4-methylpiperazin-1-yl)propan-2-yl)oxy)benzyl)benzonitrile hydrochloride; 2-(3,5-dimethyl-2-(2-(4-methylpiperazin-1-yl)ethoxy)benzyl)benzonitrile; 2-(3,5-dimethyl-2-(2-(4-methylpiperazin-1-yl)ethoxy)benzyl)benzonitrile hydrochloride; 2-(3,5-dimethyl-2-(2-(tetrahydro-2H-pyran-4-yl)ethoxy)benzyl)benzonitrile; 2-(3,5-dimethyl-2-(2-morpholinoethoxy)benzyl)-3-fluorobenzonitrile; 2-(3,5-dimethyl-2-(2-morpholinoethoxy)benzyl)aniline; 2-(3,5-dimethyl-2-(2-morpholinoethoxy)benzyl)benzonitrile; 2-(3,5-dimethyl-2-(2-morpholinoethoxy)benzyl)benzonitrile hydrochloride; 2-(3,5-dimethyl-2-(3-morpholinopropoxy)benzyl)benzonitrile; 2-(3,5-dimethyl-2-(3-morpholinopropyl)phenoxy)benzonitrile; 2-(3,5-dimethyl-2-(methyl(2-morpholinoethyl)amino)benzyl)benzonitrile; 2-(4-methyl-2-(3-(4-methylpiperazin-1-yl)propyl)phenoxy)benzonitrile; 2-(5-methyl-2-(2-(1-methylpiperidin-4-yl)ethoxy)benzyl)benzonitrile; 2-(5-methyl-2-(2-(4-methylpiperazin-1-yl)ethoxy)benzyl)benzonitrile; 2-(5-methyl-2-(2-(4-methylpiperazin-1-yl)ethoxy)benzyl)benzonitrile dihydrochloride; 2-(5-methyl-2-(2-morpholinoethoxy)benzyl)cyclohexan-1-one; 2-(5-methyl-2-(2-morpholinopropoxy)benzyl)benzonitrile; 2-(5-methyl-2-(3-(4-methylpiperazin-1-yl)propyl)phenoxy)benzonitrile; 2-(5-methyl-2-(3-(4-methylpiperazin-1-yl)propyl)phenoxy)benzonitrile hydrochloride; 2-benzyl-4-methyl-N-(2-(4-methylpiperazin-1-yl)ethyl)aniline; 2-benzyl-4-methylphenyl 2-(4-methylpiperazin-1-yl)acetate; 2-benzyl-N,4-dimethyl-N-(2-(4-methylpiperazin-1-yl)ethyl)aniline; 2-benzyl-N,4-dimethyl-N-(2-morpholinoethyl)aniline; 3-(3,5-dimethyl-2-(2-(4-methylpiperazin-1-yl)ethoxy)benzyl)benzonitrile dihydrochloride;
3-(3,5-dimethyl-2-(2-morpholinoethoxy)benzyl)aniline; 3-bromo-2-(3,5-dimethyl-2-(2-morpholinoethoxy)benzyl)benzonitrile; 3-chloro-2-(3,5-dimethyl-2-(2-morpholinoethoxy)benzyl)benzonitrile; 4-(1-(2-(2,6-difluorobenzyl)-4,6-dimethylphenoxy)propan-2-yl)morpholine; 4-(2-(2-(1-(2-fluorophenyl)ethyl)-4,6-dimethylphenoxy)ethyl)morpholine; 4-(2-(2-(1-(2-fluorophenyl)vinyl)-4,6-dimethylphenoxy)ethyl)morpholine; 4-(2-(2-(2,4-difluorobenzyl)-4,6-dimethylphenoxy)ethyl)morpholine; 4-(2-(2-(2,5-difluorobenzyl)-4,6-dimethylphenoxy)ethyl)morpholine; 4-(2-(2-(2,6-dichlorobenzyl)-4,6-dimethylphenoxy)ethyl)morpholine; 4-(2-(2-(2,6-difluorobenzyl)-4,6-dimethylphenoxy)ethyl)morpholine; 4-(2-(2-(2,6-difluorobenzyl)-4,6-dimethylphenoxy)propyl)morpholine; 4-(2-(2-(2,6-difluorobenzyl)-4-methylphenoxy)ethyl)morpholine; 4-(2-(2-(2-chlorobenzyl)-4,6-dimethylphenoxy)ethyl)morpholine; 4-(2-(2-(2-fluorobenzyl)-4,6-dimethylphenoxy)ethyl)morpholine; 4-(2-(2-(3,5-difluorobenzyl)-4,6-dimethylphenoxy)ethyl)morpholine; 4-(2-(2,4-dimethyl-5-((perfluorophenyl)methyl)phenoxy)ethyl)morpholine; 4-(2-(2,4-dimethyl-6-(2,4,5-trifluorobenzyl)phenoxy)ethyl)morpholine; 4-(2-(2,4-dimethyl-6-(2-nitrobenzyl)phenoxy)ethyl)morpholine; 4-(2-(2-benzyl-4-methylphenoxy)ethyl)-1,2,6-trimethylpiperazine; 4-(2-(2-benzyl-4-methylphenoxy)ethyl)morpholine hydrochloride; 4-(2-(2-benzyl-4-methylphenoxy)ethyl)thiomorpholine 1-oxide;
4-(2-(2-ethyl-6-(2-fluorobenzyl)-4-methylphenoxy)ethyl)morpholine; 4-(3-(2-(2-fluorophenoxy)-4,6-dimethylphenyl)propyl)morpholine; 4-methyl-2-(5-methyl-2-(2-(4-methylpiperazin-1-yl)ethoxy)benzyl)phenol; ethyl 4-(3,5-dimethyl-2-(2-(4-methylpiperazin-1-yl)ethoxy)benzyl)benzoate dihydrochloride;
methyl 3-benzyl-4-(2-(4-methylpiperazin-1-yl)ethoxy)benzoate; and mixtures thereof.

3. A non-medical method of inducing a cooling sensation in a human or animal comprising contacting the human or animal with a compound of formula (I) or a salt or solvate thereof as defined in claim 1.

4. A method of achieving a cooling effect on the skin or mucosa comprising contacting the skin or mucosa with a product comprising one or more compounds of formula (I) as defined in claim 1.

5. A consumer product comprising one or more compounds of formula (I) as defined in claim 1.

6. A method of using the compound of formula (I) as defined in claim 1 for providing a cooling sensation.

7. A composition comprising a cooling sensation wherein the composition comprises a compound of formula (I) as defined in claim 1.

8. A compound of formula (I), a salt, or solvate thereof 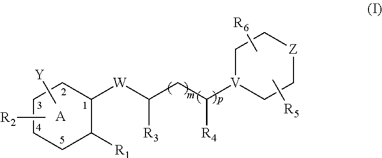
wherein
ring A represents a phenyl ring, cyclohexyl, or cyclohexenyl ring;
R₁ is selected from hydrogen, C₁-C₆ alkyl, and C₁-C₆ alkyloxy;
R₂ is connected to position 3, 4 or 5 of ring A and is selected from C₁-C₆ alkyl, C₁-C₆ alkyloxy, and C(O)O—C₁-C₃-alkyl;
R₃ is selected from hydrogen, C₁-C₆ alkyl, C₂-C₆ alkenyl, OH, and ═O, and R₄ is selected from hydrogen, C₁-C₆ alkyl, C₂-C₆ alkenyl, and OH, with the proviso that at least one of R₃ and R₄ is hydrogen; or
R₃ and R₁ form together with the carbon atoms to which they are attached a six membered ring system, and R₄ is hydrogen;
R₅ is selected from hydrogen, F, and C₁-C₆ alkyl;
R₆ is selected from hydrogen, F, and C₁-C₆ alkyl;
m is 0;
p is 1;
W is selected from —CH₂—, —O—, >C═O, —CH═, and >NR₁₃ wherein R₁₃ is selected from hydrogen and C₁-C₃ alkyl;
V is selected from >CH—, and >N—;
Z is selected from —O—, >S═O, >CHR₁₂, and >NR₁₂, wherein R₁₂ is hydrogen, C₁-C₃ alkyl, OH, or CH₂OH;
Y is connected to position 2 or 3 of ring A and is selected from C₄-C₆ alkyl 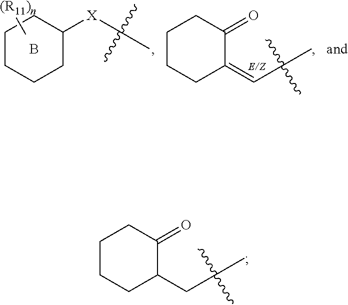
wherein
ring B represents a phenyl, thiophen, furan, or pyridine ring;
R₁₁ is selected from CN, halogen, CH₂CN, NO₂, NH₂, CF₃, C(O)OC₂H₅, C₁-C₃ alkyl, C₁-C₃ alkyloxy, C₂-C₃ alkenyl, and OH, and n is an integer from 0-5, with the proviso that if n>1, R₁₁ can be the same or different; and
X is selected from —CH₂—, —O—, >CH—CH₃, >CH═CH₂, and >CHOH,
with the proviso that the compound of formula (I) is not 1-(3-(2-benzyl-4-methylphenyl)propyl)-4-methylpiperazine or 1-(2-(2-benzyl-4-methylphenoxy)ethyl)-4-methylpiperazine.

9. The compound of formula (I), a salt or solvate thereof according to claim 8, selected from (1-(2-(2-benzyl-4-methylphenoxy)ethyl)piperidin-4-yl)methanol,
(5-methyl-2-(2-(4-methylpiperazin-1-yl)ethoxy)phenyl)(phenyl)methanol,
(E)-1-(2-((6-benzylidene-2,4-dimethylcyclohex-1-en-1-yl)oxy)ethyl)-4-methylpiperazine,
(E)-1-(3-(2-benzyl-4-methylphenyl)allyl)-4-methylpiperazine,
(E)-2-(5-methyl-2-(2-(4-methylpiperazin-1-yl)ethoxy)benzylidene)cyclohexan-1-one,
(E)-2-(5-methyl-2-(2-morpholinoethoxy)benzylidene)cyclohexan-1-one,
2-(3,5-dimethyl-2-(2-morpholinopropoxy)benzyl)benzonitrile,
2-(3,5-dimethyl-2-((1-morpholinopropan-2-yl)oxy)benzyl)benzonitrile,
1-((8-benzylchroman-2-yl)methyl)-4-methylpiperazine,
1-(2-((2-benzyl-4-methylcyclohexyl)oxy)ethyl)-4-methylpiperazine,
1-(2-((2-benzyl-4-methylcyclohexyl)oxy)ethyl)-4-methylpiperazine dihydrochloride,
1-(2-(2-((5-chlorothiophen-2-yl)methyl)-4-methylphenoxy)ethyl)-4-methylpiperazine,
1-(2-(2-(2-bromobenzyl)-4,6-dimethylphenoxy)ethyl)-4-methylpiperazine,
1-(2-(2-(2-chlorobenzyl)-4-methylphenoxy)ethyl)-4-methylpiperazine,
1-(2-(2-(2-fluorobenzyl)-4,6-dimethylphenoxy)ethyl)-4-methylpiperazine,
1-(2-(2-(2-iodobenzyl)-4,6-dimethylphenoxy)ethyl)-4-methylpiperazine,
1-(2-(2-(3-bromobenzyl)-4,6-dimethylphenoxy)ethyl)-4-methylpiperazine,
1-(2-(2-(3-chlorobenzyl)-4-methylphenoxy)ethyl)-4-methylpiperazine,
1-(2-(2-(4-chlorobenzyl)-4-methylphenoxy)ethyl)-4-methylpiperazine hydrochloride,
1-(2-(2-(furan-2-ylmethyl)-4-methylphenoxy)ethyl)-4-methylpiperazine,
1-(2-(2,4-dimethyl-6-(2-(trifluoromethyl)benzyl)phenoxy)ethyl)-4-methylpiperazine,
1-(2-(2,4-dimethyl-6-(pyridin-2-ylmethyl)phenoxy)ethyl)-4-methylpiperazine,
1-(2-(2,4-dimethyl-6-(pyridin-3-ylmethyl)phenoxy)ethyl)-4-methylpiperazine,
1-(2-(2,4-dimethyl-6-(pyridin-4-ylmethyl)phenoxy)ethyl)-4-methylpiperazine,
1-(2-(2-benzyl-3,6-dimethylphenoxy)ethyl)-4-methylpiperazine,
1-(2-(2-benzyl-3-methoxyphenoxy)ethyl)-4-methylpiperazine,
1-(2-(2-benzyl-3-methylphenoxy)ethyl)-4-methylpiperazine,
1-(2-(2-benzyl-4-(tert-butyl)-6-methoxyphenoxy)ethyl)-4-methylpiperazine,
1-(2-(2-benzyl-4,6-dimethylphenoxy)ethyl)-4-methylpiperazine,
1-(2-(2-benzyl-4,6-dimethylphenoxy)ethyl)-4-methylpiperazine dihydrochloride,
1-(2-(2-benzyl-4-chlorophenoxy)ethyl)-4-ethylpiperazine hydrochloride,
1-(2-(2-benzyl-4-chlorophenoxy)ethyl)-4-methylpiperazine hydrochloride,
1-(2-(2-benzyl-4-ethylphenoxy)ethyl)-4-methylpiperazine hydrochloride,
1-(2-(2-benzyl-4-methylphenoxy)ethyl)-4-ethylpiperazine hydrochloride,
1-(2-(2-benzyl-4-methylphenoxy)ethyl)-4-methylpiperazine 2,3-dihydroxysuccinate,
1-(2-(2-benzyl-4-methylphenoxy)ethyl)-4-methylpiperazine 2-hydroxypropane-1,2,3-tricarboxylate,
1-(2-(2-benzyl-4-methylphenoxy)ethyl)-4-methylpiperazine bis(2,2,2-trifluoroacetate),
1-(2-(2-benzyl-4-methylphenoxy)ethyl)-4-methylpiperazine bis(2-hydroxysuccinate),
1-(2-(2-benzyl-4-methylphenoxy)ethyl)-4-methylpiperazine bis(phosphate),
1-(2-(2-benzyl-4-methylphenoxy)ethyl)-4-methylpiperazine dimaleate,
1-(2-(2-benzyl-4-methylphenoxy)ethyl)-4-methylpiperazine dimethanesulfonate,
1-(2-(2-benzyl-4-methylphenoxy)ethyl)-4-methylpiperazine hydrochloride,
1-(2-(2-benzyl-4-methylphenoxy)ethyl)-4-methylpiperazine oxalate,
1-(2-(2-benzyl-4-methylphenoxy)ethyl)-4-methylpiperazine sulfate,
1-(2-(2-benzyl-4-methylphenoxy)ethyl)piperazine,
1-(2-(2-benzyl-4-methylphenoxy)ethyl)piperidin-4-ol,
1-(2-(2-benzyl-4-methylphenoxy)propyl)-4-methylpiperazine,
1-(2-(2-benzyl-4-methylphenoxy)propyl)-4-methylpiperazine hydrochloride,
1-(2-(2-benzyl-6-methylphenoxy)ethyl)-4-methylpiperazine,
1-(2-(2-benzylphenoxy)ethyl)-4-methylpiperazine hydrochloride,
1-(2-(2-isobutyl-4-methylphenoxy)ethyl)-4-methylpiperazine,
1-(2-(2-isobutyl-4-methylphenoxy)ethyl)-4-methylpiperazine dihydrochloride,
1-(2-benzyl-4-methylphenethyl)-4-methylpiperazine,
1-(2-benzyl-4-methylphenyl)-3-(4-methylpiperazin-1-yl)propan-2-ol,
1-(2-benzyl-4-methylphenyl)-3-(4-methylpiperazin-1-yl)propan-2-one,
1-(3-(2-benzyl-4-methylphenoxy)propyl)-4-methylpiperazine hydrochloride,
1-(3-(2-benzyl-4-methylphenyl)propyl)-4-methylpiperazine,
1-methyl-4-(2-(4-methyl-2-(1-phenylethyl)phenoxy)ethyl)piperazine,
1-methyl-4-(2-(4-methyl-2-(1-phenylvinyl)phenoxy)ethyl)piperazine,
1-methyl-4-(2-(4-methyl-2-(2-methylbenzyl)phenoxy)ethyl)piperazine,
1-methyl-4-(2-(4-methyl-2-(3-methylbenzyl)phenoxy)ethyl)piperazine,
1-methyl-4-(2-(4-methyl-2-(thiophen-2-ylmethyl)phenoxy)ethyl)piperazine hydrochloride,
2-(2-(2-hydroxy-3-morpholinopropyl)-5-methylphenoxy)benzonitrile,
2-(2-(3,5-dimethyl-2-(2-morpholinoethoxy)benzyl)phenyl)acetonitrile,
2-(2,4-dimethyl-5-(2-(4-methylpiperazin-1-yl)ethoxy)benzyl)benzonitrile,
2-(2,6-difluorobenzyl)-4-methyl-N-(2-morpholinoethyl)aniline,
2-(2,6-difluorobenzyl)-N,4,6-trimethyl-N-(2-morpholinoethyl)aniline,
2-(2,6-difluorobenzyl)-N,4-dimethyl-N-(2-morpholinoethyl)aniline,
2-(2-fluorobenzyl)-N,4,6-trimethyl-N-(2-morpholinoethyl)aniline,
2-(2-fluorobenzyl)-N,4-dimethyl-N-(2-morpholinoethyl)aniline,
2-(3,5-dimethyl-2-((1-(4-methylpiperazin-1-yl)propan-2-yl)oxy)benzyl)benzonitrile,
2-(3,5-dimethyl-2-((1-(4-methylpiperazin-1-yl)propan-2-yl)oxy)benzyl)benzonitrile hydrochloride,
2-(3,5-dimethyl-2-(2-(4-methylpiperazin-1-yl)ethoxy)benzyl)benzonitrile,
2-(3,5-dimethyl-2-(2-(4-methylpiperazin-1-yl)ethoxy)benzyl)benzonitrile hydrochloride,
2-(3,5-dimethyl-2-(2-(tetrahydro-2H-pyran-4-yl)ethoxy)benzyl)benzonitrile,
2-(3,5-dimethyl-2-(2-morpholinoethoxy)benzyl)-3-fluorobenzonitrile,
2-(3,5-dimethyl-2-(2-morpholinoethoxy)benzyl)aniline,
2-(3,5-dimethyl-2-(2-morpholinoethoxy)benzyl)benzonitrile,
2-(3,5-dimethyl-2-(2-morpholinoethoxy)benzyl)benzonitrile hydrochloride,
2-(3,5-dimethyl-2-(3-morpholinopropoxy)benzyl)benzonitrile,
2-(3,5-dimethyl-2-(3-morpholinopropyl)phenoxy)benzonitrile,
2-(3,5-dimethyl-2-(methyl(2-morpholinoethyl)amino)benzyl)benzonitrile,
2-(4-methyl-2-(3-(4-methylpiperazin-1-yl)propyl)phenoxy)benzonitrile,
2-(5-methyl-2-(2-(1-methylpiperidin-4-yl)ethoxy)benzyl)benzonitrile,
2-(5-methyl-2-(2-(4-methylpiperazin-1-yl)ethoxy)benzyl)benzonitrile,
2-(5-methyl-2-(2-(4-methylpiperazin-1-yl)ethoxy)benzyl)benzonitrile dihydrochloride,
2-(5-methyl-2-(2-morpholinoethoxy)benzyl)cyclohexan-1-one,
2-(5-methyl-2-(2-morpholinopropoxy)benzyl)benzonitrile,
2-(5-methyl-2-(3-(4-methylpiperazin-1-yl)propyl)phenoxy)benzonitrile,
2-(5-methyl-2-(3-(4-methylpiperazin-1-yl)propyl)phenoxy)benzonitrile hydrochloride,
2-benzyl-4-methyl-N-(2-(4-methylpiperazin-1-yl)ethyl)aniline,
2-benzyl-4-methylphenyl 2-(4-methylpiperazin-1-yl)acetate,
2-benzyl-N,4-dimethyl-N-(2-(4-methylpiperazin-1-yl)ethyl)aniline,
2-benzyl-N,4-dimethyl-N-(2-morpholinoethyl)aniline,
3-(3,5-dimethyl-2-(2-(4-methylpiperazin-1-yl)ethoxy)benzyl)benzonitrile dihydrochloride,
3-(3,5-dimethyl-2-(2-morpholinoethoxy)benzyl)aniline,
3-bromo-2-(3,5-dimethyl-2-(2-morpholinoethoxy)benzyl)benzonitrile,
3-chloro-2-(3,5-dimethyl-2-(2-morpholinoethoxy)benzyl)benzonitrile,
4-(1-(2-(2,6-difluorobenzyl)-4,6-dimethylphenoxy)propan-2-yl)morpholine,
4-(2-(2-(1-(2-fluorophenyl)ethyl)-4,6-dimethylphenoxy)ethyl)morpholine,
4-(2-(2-(1-(2-fluorophenyl)vinyl)-4,6-dimethylphenoxy)ethyl)morpholine,
4-(2-(2-(2,4-difluorobenzyl)-4,6-dimethylphenoxy)ethyl)morpholine,
4-(2-(2-(2,5-difluorobenzyl)-4,6-dimethylphenoxy)ethyl)morpholine,
4-(2-(2-(2,6-dichlorobenzyl)-4,6-dimethylphenoxy)ethyl)morpholine,
4-(2-(2-(2,6-difluorobenzyl)-4,6-dimethylphenoxy)ethyl)morpholine,
4-(2-(2-(2,6-difluorobenzyl)-4,6-dimethylphenoxy)propyl)morpholine,
4-(2-(2-(2,6-difluorobenzyl)-4-methylphenoxy)ethyl)morpholine,
4-(2-(2-(2-chlorobenzyl)-4,6-dimethylphenoxy)ethyl)morpholine,
4-(2-(2-(2-fluorobenzyl)-4,6-dimethylphenoxy)ethyl)morpholine,
4-(2-(2-(3,5-difluorobenzyl)-4,6-dimethylphenoxy)ethyl)morpholine,
4-(2-(2,4-dimethyl-5-((perfluorophenyl)methyl)phenoxy)ethyl)morpholine,
4-(2-(2,4-dimethyl-6-(2,4,5-trifluorobenzyl)phenoxy)ethyl)morpholine,
4-(2-(2,4-dimethyl-6-(2-nitrobenzyl)phenoxy)ethyl)morpholine,
4-(2-(2-benzyl-4-methylphenoxy)ethyl)-1,2,6-trimethylpiperazine,
4-(2-(2-benzyl-4-methylphenoxy)ethyl)morpholine hydrochloride,
4-(2-(2-benzyl-4-methylphenoxy)ethyl)thiomorpholine 1-oxide,
4-(2-(2-ethyl-6-(2-fluorobenzyl)-4-methylphenoxy)ethyl)morpholine,
4-(3-(2-(2-fluorophenoxy)-4,6-dimethylphenyl)propyl)morpholine,
4-methyl-2-(5-methyl-2-(2-(4-methylpiperazin-1-yl)ethoxy)benzyl)phenol,
ethyl 4-(3,5-dimethyl-2-(2-(4-methylpiperazin-1-yl)ethoxy)benzyl)benzoate dihydrochloride,
methyl 3-benzyl-4-(2-(4-methylpiperazin-1-yl)ethoxy)benzoate; and mixtures thereof.

10. A pharmaceutical composition comprising one or more compounds of formula (I) as defined in claim 8.

11. The compound of formula (I) as defined in claim 8 for use as medicament.

12. The method according to claim 1, wherein Y is 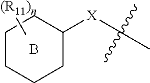
wherein
ring B represents a phenyl, thiophen, furan, or pyridine ring;
R₁₁ is selected from CN, halogen, CH₂CN, NO₂, NH₂, CF₃, C(O)OC₂H₅, C₁-C₃ alkyl, C₁-C₃ alkyloxy, C₂-C₃ alkenyl, and OH, and n is an integer from 0-5, with the proviso that if n>1, R₁₁ can be the same or different; and
X is selected from —CH₂—, —O—, >CH—CH₃, >CH═CH₂, and >CHOH.

13. The method according to claim 12, wherein ring B represents a phenyl ring.

14. The method according to claim 12, wherein the compound of formula (I) is selected from the group consisting of
(1-(2-(2-benzyl-4-methylphenoxy)ethyl)piperidin-4-yl)methanol;
(E)-1-(2-((6-benzylidene-2,4-dimethylcyclohex-1-en-1-yl)oxy)ethyl)-4-methylpiperazine;
(E)-1-(3-(2-benzyl-4-methylphenyl)allyl)-4-methylpiperazine;
1-(3-(2-benzyl-4-methylphenyl)propyl)-4-methylpiperazine;
2-(3,5-dimethyl-2-(2-morpholinopropoxy)benzyl)benzonitrile;
2-(3,5-dimethyl-2-((1-morpholinopropan-2-yl)oxy)benzyl)benzonitrile;
1-((8-benzylchroman-2-yl)methyl)-4-methylpiperazine;
1-(2-((2-benzyl-4-methylcyclohexyl)oxy)ethyl)-4-methylpiperazine;
1-(2-((2-benzyl-4-methylcyclohexyl)oxy)ethyl)-4-methylpiperazine dihydrochloride;
1-(2-(2-(2-bromobenzyl)-4,6-dimethylphenoxy)ethyl)-4-methylpiperazine;
1-(2-(2-(2-chlorobenzyl)-4-methylphenoxy)ethyl)-4-methylpiperazine;
1-(2-(2-(2-fluorobenzyl)-4,6-dimethylphenoxy)ethyl)-4-methylpiperazine;
1-(2-(2-(2-iodobenzyl)-4,6-dimethylphenoxy)ethyl)-4-methylpiperazine;
1-(2-(2-(3-bromobenzyl)-4,6-dimethylphenoxy)ethyl)-4-methylpiperazine:
1-(2-(2-(3-chlorobenzyl)-4-methylphenoxy)ethyl)-4-methylpiperazine;
1-(2-(2-(4-chlorobenzyl)-4-methylphenoxy)ethyl)-4-methylpiperazine hydrochloride;
1-(2-(2,4-dimethyl-6-(2-(trifluoromethyl)benzyl)phenoxy)ethyl)-4-methylpiperazine;
1-(2-(2,4-dimethyl-6-(pyridin-2-ylmethyl)phenoxy)ethyl)-4-methylpiperazine;
1-(2-(2,4-dimethyl-6-(pyridin-3-ylmethyl)phenoxy)ethyl)-4-methylpiperazine;
1-(2-(2,4-dimethyl-6-(pyridin-4-ylmethyl)phenoxy)ethyl)-4-methylpiperazine;
1-(2-(2-benzyl-3,6-dimethylphenoxy)ethyl)-4-methylpiperazine;
1-(2-(2-benzyl-3-methoxyphenoxy)ethyl)-4-methylpiperazine;
1-(2-(2-benzyl-3-methylphenoxy)ethyl)-4-methylpiperazine;
1-(2-(2-benzyl-4-(tert-butyl)-6-methoxyphenoxy)ethyl)-4-methylpiperazine;
1-(2-(2-benzyl-4,6-dimethylphenoxy)ethyl)-4-methylpiperazine;
1-(2-(2-benzyl-4,6-dimethylphenoxy)ethyl)-4-methylpiperazine dihydrochloride; 1-(2-(2-benzyl-4-chlorophenoxy)ethyl)-4-ethylpiperazine hydrochloride;
1-(2-(2-benzyl-4-chlorophenoxy)ethyl)-4-methylpiperazine hydrochloride;
1-(2-(2-benzyl-4-ethylphenoxy)ethyl)-4-methylpiperazine hydrochloride;
1-(2-(2-benzyl-4-isopropylphenoxy)ethyl)-4-methylpiperazine;
1-(2-(2-benzyl-4-methylphenoxy)ethyl)-4-ethylpiperazine hydrochloride;
1-(2-(2-benzyl-4-methylphenoxy)ethyl)-4-methylpiperazine;
1-(2-(2-benzyl-4-methylphenoxy)ethyl)-4-methylpiperazine 2,3-dihydroxysuccinate;
1-(2-(2-benzyl-4-methylphenoxy)ethyl)-4-methylpiperazine 2-hydroxypropane-1,2,3-tricarboxylate;
1-(2-(2-benzyl-4-methylphenoxy)ethyl)-4-methylpiperazine bis(2,2,2-trifluoroacetate);
1-(2-(2-benzyl-4-methylphenoxy)ethyl)-4-methylpiperazine bis(2-hydroxysuccinate);
1-(2-(2-benzyl-4-methylphenoxy)ethyl)-4-methylpiperazine bis(phosphate);
1-(2-(2-benzyl-4-methylphenoxy)ethyl)-4-methylpiperazine dimaleate;
1-(2-(2-benzyl-4-methylphenoxy)ethyl)-4-methylpiperazine dimethanesulfonate; 1-(2-(2-benzyl-4-methylphenoxy)ethyl)-4-methylpiperazine hydrochloride;
1-(2-(2-benzyl-4-methylphenoxy)ethyl)-4-methylpiperazine oxalate;
1-(2-(2-benzyl-4-methylphenoxy)ethyl)-4-methylpiperazine sulfate;
1-(2-(2-benzyl-4-methylphenoxy)ethyl)piperazine;
1-(2-(2-benzyl-4-methylphenoxy)ethyl)piperidin-4-ol;
1-(2-(2-benzyl-4-methylphenoxy)propyl)-4-methylpiperazine;
1-(2-(2-benzyl-4-methylphenoxy)propyl)-4-methylpiperazine hydrochloride;
1-(2-(2-benzyl-6-methylphenoxy)ethyl)-4-methylpiperazine;
1-(2-(2-benzylphenoxy)ethyl)-4-methylpiperazine hydrochloride;
1-(2-benzyl-4-methylphenethyl)-4-methylpiperazine;
1-(2-benzyl-4-methylphenyl)-3-(4-methylpiperazin-1-yl)propan-2-ol;
1-(2-benzyl-4-methylphenyl)-3-(4-methylpiperazin-1-yl)propan-2-one;
1-(3-(2-benzyl-4-methylphenoxy)propyl)-4-methylpiperazine hydrochloride;
1-(3-(2-benzyl-4-methylphenyl)propyl)-4-methylpiperazine;
1-methyl-4-(2-(4-methyl-2-(1-phenylethyl)phenoxy)ethyl)piperazine;
1-methyl-4-(2-(4-methyl-2-(1-phenylvinyl)phenoxy)ethyl)piperazine;
1-methyl-4-(2-(4-methyl-2-(2-methylbenzyl)phenoxy)ethyl)piperazine;
1-methyl-4-(2-(4-methyl-2-(3-methylbenzyl)phenoxy)ethyl)piperazine;
2-(2-(2-hydroxy-3-morpholinopropyl)-5-methylphenoxy)benzonitrile;
2-(2-(3,5-dimethyl-2-(2-morpholinoethoxy)benzyl)phenyl)acetonitrile;
2-(2,4-dimethyl-5-(2-(4-methylpiperazin-1-yl)ethoxy)benzyl)benzonitrile;
2-(2,6-difluorobenzyl)-4-methyl-N-(2-morpholinoethyl)aniline;
2-(2,6-difluorobenzyl)-N,4,6-trimethyl-N-(2-morpholinoethyl)aniline;
2-(2,6-difluorobenzyl)-N,4-dimethyl-N-(2-morpholinoethyl)aniline;
2-(2-fluorobenzyl)-N,4,6-trimethyl-N-(2-morpholinoethyl)aniline;
2-(2-fluorobenzyl)-N,4-dimethyl-N-(2-morpholinoethyl)aniline;
2-(3,5-dimethyl-2-((1-(4-methylpiperazin-1-yl)propan-2-yl)oxy)benzyl)benzonitrile;
2-(3,5-dimethyl-2-((1-(4-methylpiperazin-1-yl)propan-2-yl)oxy)benzyl)benzonitrile hydrochloride;
2-(3,5-dimethyl-2-(2-(4-methylpiperazin-1-yl)ethoxy)benzyl)benzonitrile;
2-(3,5-dimethyl-2-(2-(4-methylpiperazin-1-yl)ethoxy)benzyl)benzonitrile hydrochloride;
2-(3,5-dimethyl-2-(2-(tetrahydro-2H-pyran-4-yl)ethoxy)benzyl)benzonitrile;
2-(3,5-dimethyl-2-(2-morpholinoethoxy)benzyl)-3-fluorobenzonitrile;
2-(3,5-dimethyl-2-(2-morpholinoethoxy)benzyl)aniline;
2-(3,5-dimethyl-2-(2-morpholinoethoxy)benzyl)benzonitrile;
2-(3,5-dimethyl-2-(2-morpholinoethoxy)benzyl)benzonitrile hydrochloride;
2-(3,5-dimethyl-2-(3-morpholinopropoxy)benzyl)benzonitrile;
2-(3,5-dimethyl-2-(3-morpholinopropyl)phenoxy)benzonitrile;
2-(3,5-dimethyl-2-(methyl(2-morpholinoethyl)amino)benzyl)benzonitrile;
2-(4-methyl-2-(3-(4-methylpiperazin-1-yl)propyl)phenoxy)benzonitrile;
2-(5-methyl-2-(2-(1-methylpiperidin-4-yl)ethoxy)benzyl)benzonitrile;
2-(5-methyl-2-(2-(4-methylpiperazin-1-yl)ethoxy)benzyl)benzonitrile;
2-(5-methyl-2-(2-(4-methylpiperazin-1-yl)ethoxy)benzyl)benzonitrile dihydrochloride;
2-(5-methyl-2-(2-morpholinopropoxy)benzyl)benzonitrile;
2-(5-methyl-2-(3-(4-methylpiperazin-1-yl)propyl)phenoxy)benzonitrile;
2-(5-methyl-2-(3-(4-methylpiperazin-1-yl)propyl)phenoxy)benzonitrile hydrochloride; 2-benzyl-4-methyl-N-(2-(4-methylpiperazin-1-yl)ethyl)aniline;
2-benzyl-4-methylphenyl 2-(4-methylpiperazin-1-yl)acetate;
2-benzyl-N,4-dimethyl-N-(2-(4-methylpiperazin-1-yl)ethyl)aniline;
2-benzyl-N,4-dimethyl-N-(2-morpholinoethyl)aniline;
3-(3,5-dimethyl-2-(2-(4-methylpiperazin-1-yl)ethoxy)benzyl)benzonitrile dihydrochloride;
3-(3,5-dimethyl-2-(2-morpholinoethoxy)benzyl)aniline;
3-bromo-2-(3,5-dimethyl-2-(2-morpholinoethoxy)benzyl)benzonitrile;
3-chloro-2-(3,5-dimethyl-2-(2-morpholinoethoxy)benzyl)benzonitrile;
4-(1-(2-(2,6-difluorobenzyl)-4,6-dimethylphenoxy)propan-2-yl)morpholine;
4-(2-(2-(1-(2-fluorophenyl)ethyl)-4,6-dimethylphenoxy)ethyl)morpholine;
4-(2-(2-(1-(2-fluorophenyl)vinyl)-4,6-dimethylphenoxy)ethyl)morpholine;
4-(2-(2-(2,4-difluorobenzyl)-4,6-dimethylphenoxy)ethyl)morpholine;
4-(2-(2-(2,5-difluorobenzyl)-4,6-dimethylphenoxy)ethyl)morpholine;
4-(2-(2-(2,6-dichlorobenzyl)-4,6-dimethylphenoxy)ethyl)morpholine;
4-(2-(2-(2,6-difluorobenzyl)-4,6-dimethylphenoxy)ethyl)morpholine;
4-(2-(2-(2,6-difluorobenzyl)-4,6-dimethylphenoxy)propyl)morpholine;
4-(2-(2-(2,6-difluorobenzyl)-4-methylphenoxy)ethyl)morpholine;
4-(2-(2-(2-chlorobenzyl)-4,6-dimethylphenoxy)ethyl)morpholine;
4-(2-(2-(2-fluorobenzyl)-4,6-dimethylphenoxy)ethyl)morpholine;
4-(2-(2-(3,5-difluorobenzyl)-4,6-dimethylphenoxy)ethyl)morpholine;
4-(2-(2,4-dimethyl-5-((perfluorophenyl)methyl)phenoxy)ethyl)morpholine;
4-(2-(2,4-dimethyl-6-(2,4,5-trifluorobenzyl)phenoxy)ethyl)morpholine;
4-(2-(2,4-dimethyl-6-(2-nitrobenzyl)phenoxy)ethyl)morpholine;
4-(2-(2-benzyl-4-methylphenoxy)ethyl)-1,2,6-trimethylpiperazine;
4-(2-(2-benzyl-4-methylphenoxy)ethyl)morpholine hydrochloride;
4-(2-(2-benzyl-4-methylphenoxy)ethyl)thiomorpholine 1-oxide;
4-(2-(2-ethyl-6-(2-fluorobenzyl)-4-methylphenoxy)ethyl)morpholine;
4-(3-(2-(2-fluorophenoxy)-4,6-dimethylphenyl)propyl)morpholine;
4-methyl-2-(5-methyl-2-(2-(4-methylpiperazin-1-yl)ethoxy)benzyl)phenol;
ethyl 4-(3,5-dimethyl-2-(2-(4-methylpiperazin-1-yl)ethoxy)benzyl)benzoate dihydrochloride;
methyl 3-benzyl-4-(2-(4-methylpiperazin-1-yl)ethoxy)benzoate, and mixtures thereof.

15. The compound of formula (I) according to claim 8, wherein the compound is a compound of formula (Ib) 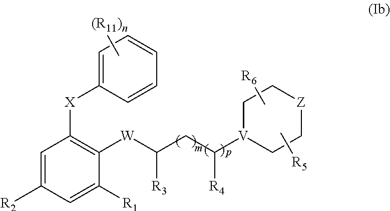
wherein
R₁ is selected from hydrogen, C₁-C₆ alkyl and C₁-C₆ alkyloxy;
R₂ is selected from hydrogen, halogen, C₁-C₆ alkyl, C₁-C₆ alkyloxy, and C(O)O—C₁-C₃-alkyl;
R₃ is selected from hydrogen, C₁-C₆ alkyl, C₂-C₆ alkenyl, OH, and ═O, and R₄ is selected from hydrogen, C₁-C₆ alkyl, C₂-C₆ alkenyl, and OH, with the proviso that at least one of R₃ and R₄ is hydrogen; or
R₃ and R₁ form together with the carbon atoms to which they are attached a six membered ring system, and R₄ is hydrogen;
R₅ is selected from hydrogen, F and C₁-C₆ alkyl;
R₆ is selected from hydrogen, F and C₁-C₆ alkyl;
m is 0 or 1;
p is 0 or 1;
W is selected from —CH₂—, —O—, >C═O, —CH═, and >NR₁₃ wherein R₁₃ is selected from hydrogen and C₁-C₃ alkyl;
V is selected from >CH—, and >N—;
Z is selected from —O—, >S═O, >CHR₁₂, >NR₁₂ wherein R₁₂ is hydrogen, C₁-C₃ alkyl, OH, or CH₂OH;
R₁₁ is selected from CN, halogen, CH₂CN, NO₂, NH₂, CF₃, C(O)OC₂H₅, C₁-C₃ alkyl, C₁-C₃ alkyloxy, C₂-C₃ alkenyl, and OH, and n is an integer from 0-5 with the proviso that if n>1, R₁₁ can be the same or different; and
X is selected from —CH₂—, —O—, >CH—CH₃, >CH═CH₂, and >CHOH.

16. The compound of formula (Ib), a salt, or solvate thereof according to claim 15 selected from selected from (1-(2-(2-benzyl-4-methylphenoxy)ethyl)piperidin-4-yl)methanol,
(5-methyl-2-(2-(4-methylpiperazin-1-yl)ethoxy)phenyl)(phenyl)methanol,
(E)-1-(2-((6-benzylidene-2,4-dimethylcyclohex-1-en-1-yl)oxy)ethyl)-4-methylpiperazine,
(E)-1-(3-(2-benzyl-4-methylphenyl)allyl)-4-methylpiperazine,
2-(3,5-dimethyl-2-(2-morpholinopropoxy)benzyl)benzonitrile,
2-(3,5-dimethyl-2-((1-morpholinopropan-2-yl)oxy)benzyl)benzonitrile,
1-((8-benzylchroman-2-yl)methyl)-4-methylpiperazine,
1-(2-((2-benzyl-4-methylcyclohexyl)oxy)ethyl)-4-methylpiperazine,
1-(2-((2-benzyl-4-methylcyclohexyl)oxy)ethyl)-4-methylpiperazine dihydrochloride,
1-(2-(2-(2-bromobenzyl)-4,6-dimethylphenoxy)ethyl)-4-methylpiperazine,
1-(2-(2-(2-chlorobenzyl)-4-methylphenoxy)ethyl)-4-methylpiperazine,
1-(2-(2-(2-fluorobenzyl)-4,6-dimethylphenoxy)ethyl)-4-methylpiperazine,
1-(2-(2-(2-iodobenzyl)-4,6-dimethylphenoxy)ethyl)-4-methylpiperazine,
1-(2-(2-(3-bromobenzyl)-4,6-dimethylphenoxy)ethyl)-4-methylpiperazine,
1-(2-(2-(3-chlorobenzyl)-4-methylphenoxy)ethyl)-4-methylpiperazine,
1-(2-(2-(4-chlorobenzyl)-4-methylphenoxy)ethyl)-4-methylpiperazine hydrochloride,
1-(2-(2,4-dimethyl-6-(2-(trifluoromethyl)benzyl)phenoxy)ethyl)-4-methylpiperazine,
1-(2-(2,4-dimethyl-6-(pyridin-2-ylmethyl)phenoxy)ethyl)-4-methylpiperazine,
1-(2-(2,4-dimethyl-6-(pyridin-3-ylmethyl)phenoxy)ethyl)-4-methylpiperazine,
1-(2-(2,4-dimethyl-6-(pyridin-4-ylmethyl)phenoxy)ethyl)-4-methylpiperazine,
1-(2-(2-benzyl-3,6-dimethylphenoxy)ethyl)-4-methylpiperazine,
1-(2-(2-benzyl-3-methoxyphenoxy)ethyl)-4-methylpiperazine,
1-(2-(2-benzyl-3-methylphenoxy)ethyl)-4-methylpiperazine,
1-(2-(2-benzyl-4-(tert-butyl)-6-methoxyphenoxy)ethyl)-4-methylpiperazine,
1-(2-(2-benzyl-4,6-dimethylphenoxy)ethyl)-4-methylpiperazine,
1-(2-(2-benzyl-4,6-dimethylphenoxy)ethyl)-4-methylpiperazine dihydrochloride,
1-(2-(2-benzyl-4-chlorophenoxy)ethyl)-4-ethylpiperazine hydrochloride,
1-(2-(2-benzyl-4-chlorophenoxy)ethyl)-4-methylpiperazine hydrochloride,
1-(2-(2-benzyl-4-ethylphenoxy)ethyl)-4-methylpiperazine hydrochloride,
1-(2-(2-benzyl-4-isopropylphenoxy)ethyl)-4-methylpiperazine,
1-(2-(2-benzyl-4-methylphenoxy)ethyl)-4-ethylpiperazine hydrochloride,
1-(2-(2-benzyl-4-methylphenoxy)ethyl)-4-methylpiperazine 2,3-dihydroxysuccinate,
1-(2-(2-benzyl-4-methylphenoxy)ethyl)-4-methylpiperazine 2-hydroxypropane-1,2,3-tricarboxylate,
1-(2-(2-benzyl-4-methylphenoxy)ethyl)-4-methylpiperazine bis(2,2,2-trifluoroacetate),
1-(2-(2-benzyl-4-methylphenoxy)ethyl)-4-methylpiperazine bis(2-hydroxysuccinate),
1-(2-(2-benzyl-4-methylphenoxy)ethyl)-4-methylpiperazine bis(phosphate),
1-(2-(2-benzyl-4-methylphenoxy)ethyl)-4-methylpiperazine dimaleate,
1-(2-(2-benzyl-4-methylphenoxy)ethyl)-4-methylpiperazine dimethanesulfonate,
1-(2-(2-benzyl-4-methylphenoxy)ethyl)-4-methylpiperazine hydrochloride,
1-(2-(2-benzyl-4-methylphenoxy)ethyl)-4-methylpiperazine oxalate,
1-(2-(2-benzyl-4-methylphenoxy)ethyl)-4-methylpiperazine sulfate,
1-(2-(2-benzyl-4-methylphenoxy)ethyl)piperazine,
1-(2-(2-benzyl-4-methylphenoxy)ethyl)piperidin-4-ol,
1-(2-(2-benzyl-4-methylphenoxy)propyl)-4-methylpiperazine,
1-(2-(2-benzyl-4-methylphenoxy)propyl)-4-methylpiperazine hydrochloride,
1-(2-(2-benzyl-6-methylphenoxy)ethyl)-4-methylpiperazine,
1-(2-(2-benzylphenoxy)ethyl)-4-methylpiperazine hydrochloride,
1-(2-benzyl-4-methylphenethyl)-4-methylpiperazine,
1-(2-benzyl-4-methylphenyl)-3-(4-methylpiperazin-1-yl)propan-2-ol,
1-(2-benzyl-4-methylphenyl)-3-(4-methylpiperazin-1-yl)propan-2-one,
1-(3-(2-benzyl-4-methylphenoxy)propyl)-4-methylpiperazine hydrochloride,
1-(3-(2-benzyl-4-methylphenyl)propyl)-4-methylpiperazine,
1-methyl-4-(2-(4-methyl-2-(1-phenylethyl)phenoxy)ethyl)piperazine,
1-methyl-4-(2-(4-methyl-2-(1-phenylvinyl)phenoxy)ethyl)piperazine,
1-methyl-4-(2-(4-methyl-2-(2-methylbenzyl)phenoxy)ethyl)piperazine,
1-methyl-4-(2-(4-methyl-2-(3-methylbenzyl)phenoxy)ethyl)piperazine,
2-(2-(2-hydroxy-3-morpholinopropyl)-5-methylphenoxy)benzonitrile,
2-(2-(3,5-dimethyl-2-(2-morpholinoethoxy)benzyl)phenyl)acetonitrile,
2-(2,4-dimethyl-5-(2-(4-methylpiperazin-1-yl)ethoxy)benzyl)benzonitrile,
2-(2,6-difluorobenzyl)-4-methyl-N-(2-morpholinoethyl)aniline,
2-(2,6-difluorobenzyl)-N,4,6-trimethyl-N-(2-morpholinoethyl)aniline,
2-(2,6-difluorobenzyl)-N,4-dimethyl-N-(2-morpholinoethyl)aniline,
2-(2-fluorobenzyl)-N,4,6-trimethyl-N-(2-morpholinoethyl)aniline,
2-(2-fluorobenzyl)-N,4-dimethyl-N-(2-morpholinoethyl)aniline,
2-(3,5-dimethyl-2-((1-(4-methylpiperazin-1-yl)propan-2-yl)oxy)benzyl)benzonitrile,
2-(3,5-dimethyl-2-((1-(4-methylpiperazin-1-yl)propan-2-yl)oxy)benzyl)benzonitrile hydrochloride,
2-(3,5-dimethyl-2-(2-(4-methylpiperazin-1-yl)ethoxy)benzyl)benzonitrile,
2-(3,5-dimethyl-2-(2-(4-methylpiperazin-1-yl)ethoxy)benzyl)benzonitrile hydrochloride,
2-(3,5-dimethyl-2-(2-(tetrahydro-2H-pyran-4-yl)ethoxy)benzyl)benzonitrile,
2-(3,5-dimethyl-2-(2-morpholinoethoxy)benzyl)-3-fluorobenzonitrile,
2-(3,5-dimethyl-2-(2-morpholinoethoxy)benzyl)aniline,
2-(3,5-dimethyl-2-(2-morpholinoethoxy)benzyl)benzonitrile,
2-(3,5-dimethyl-2-(2-morpholinoethoxy)benzyl)benzonitrile hydrochloride,
2-(3,5-dimethyl-2-(3-morpholinopropoxy)benzyl)benzonitrile,
2-(3,5-dimethyl-2-(3-morpholinopropyl)phenoxy)benzonitrile,
2-(3,5-dimethyl-2-(methyl(2-morpholinoethyl)amino)benzyl)benzonitrile,
2-(4-methyl-2-(3-(4-methylpiperazin-1-yl)propyl)phenoxy)benzonitrile,
2-(5-methyl-2-(2-(1-methylpiperidin-4-yl)ethoxy)benzyl)benzonitrile,
2-(5-methyl-2-(2-(4-methylpiperazin-1-yl)ethoxy)benzyl)benzonitrile,
2-(5-methyl-2-(2-(4-methylpiperazin-1-yl)ethoxy)benzyl)benzonitrile dihydrochloride,
2-(5-methyl-2-(2-morpholinopropoxy)benzyl)benzonitrile,
2-(5-methyl-2-(3-(4-methylpiperazin-1-yl)propyl)phenoxy)benzonitrile,
2-(5-methyl-2-(3-(4-methylpiperazin-1-yl)propyl)phenoxy)benzonitrile hydrochloride,
2-benzyl-4-methyl-N-(2-(4-methylpiperazin-1-yl)ethyl)aniline,
2-benzyl-4-methylphenyl 2-(4-methylpiperazin-1-yl)acetate,
2-benzyl-N,4-dimethyl-N-(2-(4-methylpiperazin-1-yl)ethyl)aniline,
2-benzyl-N,4-dimethyl-N-(2-morpholinoethyl)aniline,
3-(3,5-dimethyl-2-(2-(4-methylpiperazin-1-yl)ethoxy)benzyl)benzonitrile dihydrochloride,
3-(3,5-dimethyl-2-(2-morpholinoethoxy)benzyl)aniline,
3-bromo-2-(3,5-dimethyl-2-(2-morpholinoethoxy)benzyl)benzonitrile,
3-chloro-2-(3,5-dimethyl-2-(2-morpholinoethoxy)benzyl)benzonitrile,
4-(1-(2-(2,6-difluorobenzyl)-4,6-dimethylphenoxy)propan-2-yl)morpholine,
4-(2-(2-(1-(2-fluorophenyl)ethyl)-4,6-dimethylphenoxy)ethyl)morpholine,
4-(2-(2-(1-(2-fluorophenyl)vinyl)-4,6-dimethylphenoxy)ethyl)morpholine,
4-(2-(2-(2,4-difluorobenzyl)-4,6-dimethylphenoxy)ethyl)morpholine,
4-(2-(2-(2,5-difluorobenzyl)-4,6-dimethylphenoxy)ethyl)morpholine,
4-(2-(2-(2,6-dichlorobenzyl)-4,6-dimethylphenoxy)ethyl)morpholine,
4-(2-(2-(2,6-difluorobenzyl)-4,6-dimethylphenoxy)ethyl)morpholine,
4-(2-(2-(2,6-difluorobenzyl)-4,6-dimethylphenoxy)propyl)morpholine,
4-(2-(2-(2,6-difluorobenzyl)-4-methylphenoxy)ethyl)morpholine,
4-(2-(2-(2-chlorobenzyl)-4,6-dimethylphenoxy)ethyl)morpholine,
4-(2-(2-(2-fluorobenzyl)-4,6-dimethylphenoxy)ethyl)morpholine,
4-(2-(2-(3,5-difluorobenzyl)-4,6-dimethylphenoxy)ethyl)morpholine,
4-(2-(2,4-dimethyl-5-((perfluorophenyl)methyl)phenoxy)ethyl)morpholine,
4-(2-(2,4-dimethyl-6-(2,4,5-trifluorobenzyl)phenoxy)ethyl)morpholine,
4-(2-(2,4-dimethyl-6-(2-nitrobenzyl)phenoxy)ethyl)morpholine,
4-(2-(2-benzyl-4-methylphenoxy)ethyl)-1,2,6-trimethylpiperazine,
4-(2-(2-benzyl-4-methylphenoxy)ethyl)morpholine hydrochloride,
4-(2-(2-benzyl-4-methylphenoxy)ethyl)thiomorpholine 1-oxide,
4-(2-(2-ethyl-6-(2-fluorobenzyl)-4-methylphenoxy)ethyl)morpholine,
4-(3-(2-(2-fluorophenoxy)-4,6-dimethylphenyl)propyl)morpholine,
4-methyl-2-(5-methyl-2-(2-(4-methylpiperazin-1-yl)ethoxy)benzyl)phenol,
ethyl 4-(3,5-dimethyl-2-(2-(4-methylpiperazin-1-yl)ethoxy)benzyl)benzoate dihydrochloride,
methyl 3-benzyl-4-(2-(4-methylpiperazin-1-yl)ethoxy)benzoate, and mixtures thereof.

17. A pharmaceutical composition comprising one or more compounds of formula (Ib) as defined in claim 15.

18. The compound of formula (Ib) as defined in claim 15 for use as a medicament.

19. A method of using the compound of formula (Ib) as defined in claim 15 for providing a cooling sensation.

20. A composition comprising a cooling sensation wherein the composition comprises a compound of formula (Ib) as defined in claim 15.
